(19) United States
(12) Patent Application Publication
(10) Pub. No.: US 2025/0250630 A1
(43) Pub. Date: Aug. 7, 2025

(54) CALIBRATION PANELS AND METHODS FOR DESIGNING THE SAME

(71) Applicant: Life Technologies Corporation, Carlsbad, CA (US)

(72) Inventors: Vadim Mozhayskiy, San Diego, CA (US); Yutao Fu, San Marcos, CA (US); Earl Hubbell, Palo Alto, CA (US)

(21) Appl. No.: 19/015,117

(22) Filed: Jan. 9, 2025

Related U.S. Application Data
(63) Continuation of application No. 17/811,192, filed on Jul. 7, 2022, now abandoned.
Application No. 17/811,192 is a continuation of application No. 14/975,001, filed on Dec. 18, 2015, now abandoned.
(60) Provisional application No. 62/093,754, filed on Dec. 18, 2014.

(51) Int. Cl. C12Q 1/6874; C12N 15/10; C12Q 1/6825

(52) U.S. Cl. CPC C12Q 1/6874; C12N 15/1058; C12Q 1/6825

(57) ABSTRACT

A method for preparing a homopolymer recalibration panel includes: extracting, from a set of amplicons used in sequencing-by-synthesis, a set of candidate amplicons satisfying a first set of criteria, wherein the first set of criteria includes amplicons known to belong to high-confidence regions of a reference genome with no variants; and selecting, from the set of candidate amplicons, a reduced set of amplicons satisfying a second set of criteria, wherein the second set of criteria includes amplicons that together comprise at least a minimal threshold number of homopolymers of each homopolymer length between a predetermined minimal homopolymer length and a predetermined maximal homopolymer length for one or more of homopolymer types A, T, C, and G.

### RELATED APPLICATIONS

This application is a continuation of U.S. application Ser. No. 17/811,192 filed Jul. 7, 2022, which is a continuation of U.S. application Ser. No. 14/975,001 filed Dec. 18, 2015, which claims priority to U.S. application No. 62/093,754 filed Dec. 18, 2014, which disclosures are herein incorporated by reference in their entirety.

### FIELD

This application generally relates to calibration panels and methods for designing the same. More specifically, the application relates to panels of amplicons for homopolymer calibration or recalibration for use with nucleic acid sequencing data and methods for preparing the same.

### BACKGROUND

Nucleic acid sequencing data may be obtained in various ways, including using next-generation sequencing systems, for example, the Ion PGM™ and Ion Proton™ systems implementing Ion Torrent™ sequencing technology (see, e.g., U.S. Pat. No. 7,948,015 and U.S. Pat. Appl. Publ. Nos. 2010/0137143, 2009/0026082, and 2010/0282617, each of which is incorporated by reference herein in its entirety). In some cases, such nucleic acid sequencing data may be processed and/or analyzed to obtain base calls using one or more calibration or recalibration processes. Such calibration or recalibration processes may be based on measurement values obtained for randomly selected subsets of nucleic acid templates undergoing sequencing. In some cases, a random selection of nucleic acid templates may result in subsets of nucleic acid templates that lack sufficient representation of long homopolymers. Thus, a desire exists for new and improved methods for designing or selecting sets of amplicons that improve calibration or recalibration processes.

### BRIEF DESCRIPTION OF THE DRAWINGS

The accompanying drawings, which are incorporated into and form a part of the specification, illustrate one or more exemplary embodiments and serve to explain the principles of various exemplary embodiments. The drawings are exemplary and explanatory only and are not to be construed as limiting or restrictive in any way.

FIG. 1 illustrates an exemplary system for nucleic acid sequencing and/or analysis.

FIG. 2 illustrates exemplary components of an apparatus for nucleic acid sequencing.

FIG. 3 illustrates an exemplary flow cell for nucleic acid sequencing.

FIG. 4 illustrates an exemplary computer system for performing nucleic acid sequencing.

FIG. 5 illustrates an exemplary process for label-free, pH-based sequencing.

FIG. 6 illustrates an exemplary method for performing nucleic acid sequencing using a homopolymer calibration panel.

FIG. 7 illustrates an exemplary method for preparing a homopolymer calibration panel.

FIG. 8 shows homopolymer representation statistics for an exemplary recalibration panel.

FIGS. 9A and 9B illustrate exemplary distributions of amplicon length for a two-exome sequencing run.

FIGS. 10A and 10B show exemplary distributions of GC content for a two-exome sequencing run.

FIGS. 11A and 11B show exemplary distributions of coverage, expressed as percentage of median coverage, for a two-exome sequencing run.

FIGS. 12A and 12B illustrate exemplary distributions of percentage forward end-to-end for a two-exome sequencing run.

FIGS. 13A and 13B illustrate exemplary distributions of percentage reverse end-to-end for a two-exome sequencing run.

FIGS. 14A and 14B illustrate exemplary distributions of strand bias.

FIG. 15 illustrates an exemplary plot showing the correlation between forward and reverse end-to-end coverage.

FIG. 16 illustrates an exemplary plot showing the estimated per-homopolymer coverage relative to the median panel coverage.

FIGS. 17A-17Z, 17AA-17AZ, and 17BA-17BY include an exemplary table, which lists a chromosome, insert start, insert end, and insert for each of the amplicons of the exemplary recalibration panel of FIG. 8, according to one embodiment.

### SUMMARY

According to an exemplary embodiment, there is provided a method for nucleic acid sequencing, comprising: (a) disposing a plurality of template polynucleotide strands in a plurality of defined spaces of a sensor array, the template polynucleotide strands comprising a set of homopolymer recalibration template polynucleotide strands; (b) exposing a plurality of the template polynucleotide strands, including the set of homopolymer recalibration template polynucleotide strands in the defined spaces, to a series of flows of nucleotide species flowed according to a predetermined ordering; and (c) determining sequence information for the plurality of the template polynucleotide strands, including the set of homopolymer recalibration template polynucleotide strands in the defined spaces, based on the flows of nucleotide species, to generate a plurality of sequencing reads corresponding to the template polynucleotide strands. The homopolymer recalibration template polynucleotide strands may comprise amplicon sequences that together comprise at least a minimal threshold number of homopolymers of each homopolymer length between a predetermined minimal homopolymer length and a predetermined maximal homopolymer length for one or more of homopolymer types A, T, C, and G. In one embodiment of the method, the predetermined minimal homopolymer length and the predetermined maximal homopolymer length is for each of homopolymer types A, T, C, and G.

In such a method, the minimal threshold number may be 10, for example. Alternately or in addition, the minimal threshold number may be 25, for example. Alternately or in addition, the minimal threshold number may be 50, for example. The homopolymer recalibration template polynucleotide strands may comprise amplicon sequences that are comprised in high-confidence regions of a reference genome (e.g., NIST NA12878) with no variants, for example. The homopolymer recalibration template polynucleotide strands may comprise amplicon sequences that include, at most, one homopolymer of length 6, 7, 8, 9, or 10 per amplicon sequence, for example. The homopolymer recalibration template polynucleotide strands may comprise amplicon sequences having a minimal distance of 7 bases between any homopolymers of length 4, 5, 6, 7, 8, 9, or 10, for example. The homopolymer recalibration template polynucleotide strands may comprise amplicon sequences that do not overlap. The predetermined minimal homopolymer length may be 5, for example. The predetermined maximal homopolymer length may be 10, for example.

According to an exemplary embodiment, there is provided a system, including: a plurality of template polynucleotide strands disposed in a plurality of defined spaces of a sensor array, the template polynucleotide strands comprising a set of homopolymer recalibration template polynucleotide strands, wherein the homopolymer recalibration template polynucleotide strands comprise amplicon sequences that together comprise at least a minimal threshold number of homopolymers of each homopolymer length between a predetermined minimal homopolymer length and a predetermined maximal homopolymer length for one or more of homopolymer types A, T, C, and G; a machine-readable memory; and a processor configured to execute machine-readable instructions, which, when executed by the processor, cause the system to perform a method for nucleic acid sequencing, comprising: (a) exposing the plurality of the template polynucleotide strands, including the set of homopolymer recalibration template polynucleotide strands in the defined spaces, to a series of flows of nucleotide species flowed according to a predetermined ordering; and (b) determining sequence information for the plurality of the template polynucleotide strands, including the set of homopolymer recalibration template polynucleotide strands in the defined spaces, based on the flows of nucleotide species, e.g., to generate a plurality of sequencing reads corresponding to the template polynucleotide strands.

In such a system, the homopolymer recalibration template polynucleotide strands may include amplicon sequences that are comprised in high-confidence regions of a reference genome (e.g., NIST NA12878) with no variants. In one embodiment of the system, the predetermined minimal homopolymer length and the predetermined maximal homopolymer length is for each of homopolymer types A, T, C, and G.

According to an exemplary embodiment, there is provided a method for preparing a homopolymer recalibration panel, comprising: extracting, from a set of amplicons used in sequencing-by-synthesis, a set of candidate amplicons satisfying a first set of criteria, wherein the first set of criteria includes amplicons known to belong to high-confidence regions of a reference genome with no variants; and selecting, from the set of candidate amplicons, a reduced set of amplicons satisfying a second set of criteria, wherein the second set of criteria includes amplicons that together comprise at least a minimal threshold number of homopolymers of each homopolymer length between a predetermined minimal homopolymer length and a predetermined maximal homopolymer length for one or more of homopolymer types A, T, C, and G.

In such a method, the minimal threshold number may be 10, for example. Alternately or in addition, the minimal threshold number may be 25, for example. Alternately or in addition, the minimal threshold number may be 50, for example. The reference genome with no variants may be NIST NA12878, for example. In one embodiment, the predetermined minimal homopolymer length and the predetermined maximal homopolymer length is for each of homopolymer types A, T, C, and G.

The reduced set of amplicons may comprise at most one homopolymer of length 6, 7, 8, 9, or 10 per amplicon, for example. The reduced set of amplicons may comprise amplicons having a minimal distance of 7 bases between any homopolymers of length 4, 5, 6, 7, 8, 9, or 10, for example. The reduced set of amplicons may comprise amplicons that do not overlap. The predetermined minimal homopolymer length may be 5, for example. The predetermined maximal homopolymer length may be 10, for example.

The method may further comprise determining underrepresented homopolymers of the set of candidate amplicons; and augmenting the set of candidate amplicons with a predetermined number of the underrepresented homopolymers. The method may further comprise disposing the reduced set of amplicons in a plurality of defined spaces of a sensor array. The method may also further comprise exposing the reduced set of amplicons to a series of flows of nucleotide species flowed according to a predetermined ordering; and determining sequence information for the reduced set of amplicons based on the flows of nucleotide species, to generate a plurality of sequencing reads corresponding to the reduced set of amplicons.

According to an exemplary embodiment, there is provided a homopolymer recalibration panel, comprising: a set of candidate amplicons extracted from a set of amplicons used in sequencing-by-synthesis, wherein the amplicons in the set of candidate amplicons: (a) are known to belong to high-confidence regions of a reference genome with no variants; and (b) together comprise at least a minimal threshold number of homopolymers of each homopolymer length between a predetermined minimal homopolymer length and a predetermined maximal homopolymer length for one or more of homopolymer types A, T, C, and G.

In various embodiments, a panel comprising amplicons with predetermined base sequences as described herein may be synthesized using any suitable nucleic acid synthesis methods known in the art.

In some embodiments, recalibration may include a single-pass calibration process in which a recalibration engine changes or modifies a set of default/initial parameters (e.g., homopolymers of various lengths being treated/weighed the same or according to some factory pre-determined set of initial homopolymer-specific parameters or weights). In other embodiments, recalibration may include a multi-pass or iterative process in which previously calibrated or recalibrated parameters may be further changed or modified by the calibration process.

The reference genome with no variants may be NIST NA12878, for example. The minimal threshold number may be 10, for example. Alternately or in addition, the minimal threshold number may be 25, for example. Alternately or in addition, the minimal threshold number may be 50, for example. In one embodiment of the method, the predetermined minimal homopolymer length and the predetermined maximal homopolymer length is for each of homopolymer types A, T, C, and G.

The set of candidate amplicons may include amplicon sequences that include, at most, one homopolymer of length 6, 7, 8, 9, or 10 per amplicon sequence, for example. The set of candidate amplicons may comprise amplicon sequences having a minimal distance of 7 bases between any homopolymers of length 4, 5, 6, 7, 8, 9, or 10, for example. The set of candidate amplicons may comprise amplicon sequences that do not overlap. The predetermined minimal homopolymer length may be 5, for example. The predetermined maximal homopolymer length may be 10, for example.

### EXEMPLARY EMBODIMENTS

The following description and the various embodiments described herein are exemplary and explanatory only and are not to be construed as limiting or restrictive in any way. Other embodiments, features, objects, and advantages of the present teachings will be apparent from the description and accompanying drawings, and from the claims.

According to various exemplary embodiments, panels of amplicons for homopolymer calibration or recalibration for use with nucleic acid sequencing data and methods for designing the same, are disclosed herein. Such panels of amplicons may improve downstream processing (including variant calling), since such panels may improve calibration and recalibration of nucleic acid sequencing data and/or reduce certain systematic errors and improve overall sequencing accuracy (especially in the case of long homopolymers).

### Design of Calibration Panel

In various embodiments, a homopolymer calibration panel may be designed to have a substantially uniform representation of homopolymers of various lengths. Homopolymers of relatively short lengths (e.g., 2, 3, and 4 bases) may be well represented in a sufficiently large set of sufficiently long sequences selected using any suitable arbitrary or random approach. However, homopolymers of relatively long lengths (e.g., 5, 6, 7, 8, 9, and 10, or more) are naturally rarer than homopolymers of relatively short lengths. Thus, homopolymers of relatively long lengths may be insufficiently represented (or at least under-represented compared with shorter homopolymers) among sequences selected using any suitable arbitrary or random approach.

In an embodiment, a set of sequences containing a desired uniform representation across homopolymers may be defined by computationally and combinatorially populating a set of sequences with desired types and quantities of homopolymers. For example, a set of sequences may be populated to include exactly or at least n(MinL, MaxL, NumT) homopolymers, each homopolymer having a length between lengths MinL and MaxL (e.g., each length between MinL=1 and MaxL=10, or each length between MinL=5 and MaxL=10, etc.). The set of sequences may further be populated to include at least one type of nucleotide among NumT types of nucleotides (e.g., each type among NumT=4 types A, C, G, and T), where n is an integer (e.g., 10, 25, 50, 100, etc.) that may be a function of parameters MinL, MaxL, and NumT.

In some cases, one or more sets of sequences containing such homopolymers populated computationally and combinatorially may not have been empirically tested and may not be ideally suited for a given underlying sequencing technology. One method to address this may include designing a set of sequences containing a substantially uniform representation across homopolymers using steps including: (1) identifying an initial set of candidate amplicons or oligonucleotides known to function properly on a particular sequencing platform or technology (e.g., a set of amplicons from the Ion AmpliSeq™ Exome Panel or any suitable panel used with some given underlying sequencing technology); (2) selecting, from the initial set of candidate amplicons or oligonucleotides, a subset of amplicons or oligonucleotides meeting one or more selection criteria (e.g., one or more minimal numbers of occurrences of homopolymers of certain lengths and types, for example, those having at least 50 (or some suitable integer, such as 10, 25, 50, 75, etc., for example) homopolymers of each length 5, 6, 7, 8, 9, and 10, and/or of each type A, C, G, and T); and (3) augment the subset of amplicons or oligonucleotides with additional amplicons or oligonucleotides comprising a desired number of under-represented homopolymers (e.g., by adding a substantial number of homopolymers of length 9 and/or 10 or other comparatively rare and under-represented length(s)). Augmenting the subset of amplicons or oligonucleotides with additional amplicons or oligonucleotides may help achieve a desired level of representation uniformity across homopolymers.

In an embodiment, a set of sequences containing a substantially uniform representation across homopolymers may be designed using steps including: (1) identifying an initial set of candidate amplicons or oligonucleotides known to have been used with a particular sequencing platform or technology; (2) selecting from the initial set of candidate amplicons or oligonucleotides a subset of amplicons or oligonucleotides that together include all n-mers up to a predetermined maximal homopolymer length (e.g., up to n=5, 6, 7, 8, 9, or 10) for bases A, C, G, and T with a predetermined minimum of n-mers of each length and/or type of nucleotide (e.g., at least 10, 25, 50, 75, or more, of each length and/or type); and (3) performing an empirically-based pruning or refinement selection to reduce the impact on throughput. Such pruning or refinement selection may include minimizing the number of amplicons or oligonucleotides by selecting amplicons that have several n-mers but maintaining the quality of the selected amplicons (e.g., in an example further discussed below, a final panel may have 384 amplicons, a 23% reduction from a starting point of 500 candidate amplicons, where 500 may be the product of 50 n-mers times 4 bases times 5 (for n=6, 7, 8, 9, and 10, with shorter n-mers being automatically included), divided by 2 (for strands that produce complementary homopolymers)).

In various embodiments, a homopolymer calibration panel may be designed using steps including: (1) identifying an initial set of candidate amplicons or oligonucleotides that are known to have been used with a particular sequencing platform or technology (e.g., a substantial number, such as 300, 400, 500, or more, amplicons or oligonucleotides from the Ion AmpliSeq™ Exome Panel or any suitable panel used with some given underlying sequencing technology) and that are inside high-confidence regions of a reference genome (e.g., NIST NA12878) with no variants so that the real homopolymer length(s) may be known; (2) selecting, from the initial set of candidate amplicons or oligonucleotides, a subset of amplicons or oligonucleotides that together include a predetermined minimum of n-mers of each homopolymer length from a predetermined minimal length to a predetermined maximal length for all bases A, C, G, and T; (3) filtering out amplicons or oligonucleotides that violate one or more of the following constraints: (a) having more than one same-base n-mer with n=6 or more, (b) having more than a minimal separation of 7 bases between n-mers with n=4 or more bases to obviate or reduce additional errors and de-phasing that may be introduced with neighboring homopolymers (except that several G or C n-mers may be on the same strand if they are separated by at least 7 bases and at least 3 bases from A/T n-mers, given that long C and G homopolymers may be rare and GC-rich regions may be particularly difficult to sequence), (c) having an overlap with another amplicon or oligonucleotide in the set, and (d) having a homopolymer of length longer than 10; and (4) if desired to achieve a determined or selected level of representation uniformity across homopolymers, augment the subset of amplicons or oligonucleotides with additional amplicons or oligonucleotides comprising a desired number of under-represented homopolymers (e.g., by adding a substantial number of G and C homopolymers of length 9 and/or 10 to represent problematic homopolymers in the set).

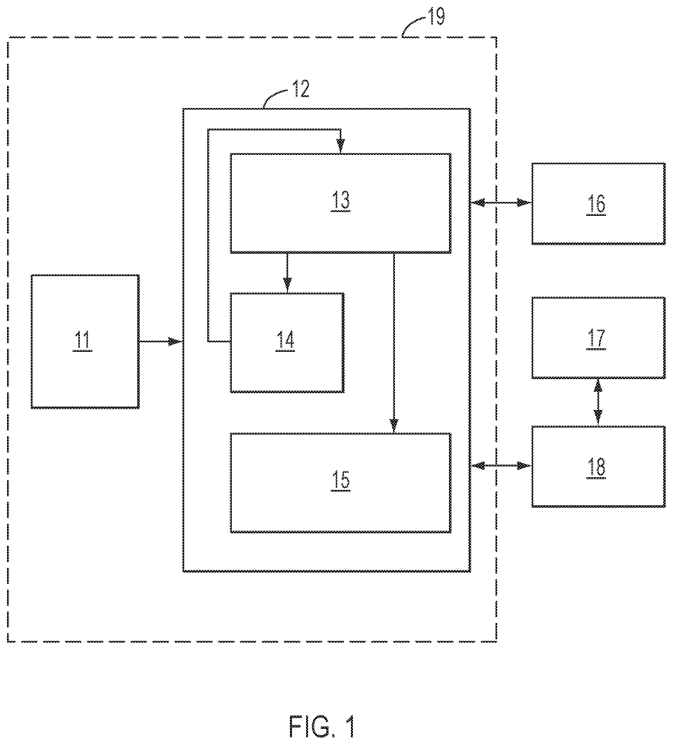

FIG. 1 illustrates an exemplary system for nucleic acid sequencing and/or analysis. The system includes an apparatus or sub-system for nucleic acid sequencing and/or analysis 11, a computing server/node/device 12 including a base calling engine 13, a recalibration engine 14, a post-processing engine 15, and a display 16, which may be internal and/or external. The apparatus or sub-system for nucleic acid sequencing and/or analysis 11 may be any type of instrument that can generate nucleic acid sequence data from nucleic acid samples, for example, a nucleic acid sequencing instrument, a real-time/digital/quantitative PCR instrument, a microarray scanner, etc. The nucleic acid samples may include amplicons for calibration or recalibration as further described herein. The computing server/node/device 12 may include a workstation, mainframe computer, distributed computing node (part of a “cloud computing” or distributed networking system), personal computer, mobile device, etc. The base calling engine 13 may include any suitable base caller and may be configured to include various signal/data processing modules that may be configured to receive signal/data from the apparatus or sub-system for nucleic acid sequencing and/or analysis 11 and perform various processing steps, for example, conversion from flow space to base space, determination of base calls for some or the entirety of a sequencing data set, and/or determination of base call quality values. In an embodiment, the base calling engine 13 may implement one or more features described in Davey et al., U.S. Pat. Appl. Publ. No. 2012/0109598, published on May 3, 2012, and/or Sikora et al., U.S. Pat. Appl. Publ. No. 2013/0060482, published on Mar. 7, 2013, each of which is incorporated by reference herein in its entirety. The base calling engine 13 may also include a mapping or alignment module for mapping or aligning reads to a reference sequence or genome, which may include a whole/partial genome, a whole/partial exome, etc. In an embodiment, the mapping or alignment module may include any suitable aligner, including the Torrent Mapping Alignment Program (TMAP), for example. The recalibration engine 14 may be configured to recalibrate base calls or related intensity values or parameters based on an analysis of base calling and alignment performed by the base calling engine 13. Recalibrated base calls or related intensity values, thresholds, or parameters may be fed back into the base calling engine 13 for improving the accuracy of base calls. In an embodiment, the recalibration engine 14 may implement one or more features described in Jiang et al., U.S. Pat. Appl. Publ. No. 2014/0316716, published on Oct. 23, 2014, which is incorporated by reference herein in its entirety. The exemplary system may also include a client device terminal 17, which may include a data analysis API or module and may be communicatively connected to the computing server/node/device 12 via a network connection 18 that may be a “hardwired” physical network connection (e.g., Internet, LAN, WAN, VPN, etc.) or a wireless network connection (e.g., Wi-Fi, WLAN, etc.). The post-processing engine 15 may be configured to include various signal/data processing modules that may be configured to make variant calls and apply post-processing to variant calls, which may include annotating various variant calls and/or features, converting data from flow space to base space, filtering of variants, and/or formatting the variant data for display or use by client device terminal 17. Variant calls may be made using any suitable variant caller, including the Germ-Line Variant Caller and the Torrent Variant Caller (TVC) Plug-ins for Ion Torrent™ sequencing technology. In an embodiment, the variant caller may implement one or more features described in Hubbell et al., U.S. Pat. Appl. No. 2014/0296080, published on Oct. 2, 2014, which is incorporated by reference herein in its entirety. In an embodiment, the apparatus or sub-system for nucleic acid sequencing and/or analysis 11 and the computing server/node/device 12 may be integrated into a single instrument or system comprising components present in a single enclosure 19. The client device terminal 17 may be configured to communicate information to and/or control the operation of the computing server/node/device 12 and its modules and/or operating parameters.

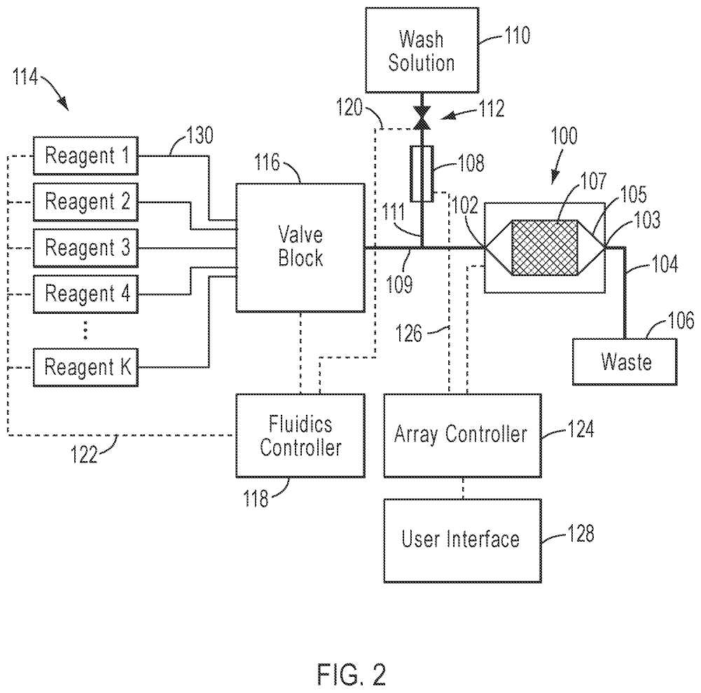

FIG. 2 illustrates exemplary components of an apparatus for nucleic acid sequencing. Such an apparatus could be used as apparatus or sub-system for nucleic acid sequencing and/or analysis 11 of FIG. 1. The components include a flow cell and sensor array 100, a reference electrode 108, a plurality of reagents 114, a valve block 116, a wash solution 110, a valve 112, a fluidics controller 118, lines 120/122/126, passages 104/109/111, a waste container 106, an array controller 124, and a user interface 128. The flow cell and sensor array 100 includes an inlet 102, an outlet 103, a microwell array 107, and a flow chamber 105 defining a flow path of reagents over the microwell array 107. The reference electrode 108 may be of any suitable type or shape, including a concentric cylinder with a fluid passage or a wire inserted into a lumen of passage 111. The reagents 114 may be driven through the fluid pathways, valves, and flow cell by pumps, gas pressure, or other suitable methods, and may be discarded into the waste container 106 after exiting the flow cell and sensor array 100. The reagents 114 may, for example, contain dNTPs to be flowed through passages 130 and through the valve block 116, which may control the flow of the reagents 114 to flow chamber 105 (also referred to herein as a reaction chamber) via passage 109. The system may include a reservoir for containing a wash solution 110 that may be used to wash away dNTPs, for example, that may have previously been flowed. The microwell array 107 may include an array of defined spaces (e.g., microwells), for example, wherein the array may be operationally associated with a sensor array so that each microwell may be associated with a sensor suitable for detecting an analyte or reaction property of interest. The defined spaces may include nucleic acid samples, which may include amplicons for calibration or recalibration as further described herein. The microwell array 107 may be integrated with the sensor array as a single device or chip. The array controller 124 may provide bias voltages, timing, and/or control signals to the sensor, and collect and/or process output signals. The user interface 128 may display information from the flow cell and sensor array 100 as well as instrument settings and controls, and allow a user to enter or set instrument settings and controls. The valve 112 may be shut to prevent any wash solution 110 from flowing into passage 109 as the reagents are flowing. Although the flow of wash solution may be stopped, there may still be uninterrupted fluid and electrical communication between the reference electrode 108, passage 109, and the microwell array 107. The fluidics controller 118 may be programmed to control driving forces for flowing reagents 114 and to control the operation of valve 112 and valve block 116 to deliver reagents to the flow cell and sensor array 100 according to a predetermined reagent flow ordering.

In this application, “defined space” may refer to any space (which may be in one, two, or three dimensions) in which at least some of a molecule, fluid, and/or solid can be confined, retained, and/or localized. A space may be a predetermined area (which may be a flat area) or volume, and may be defined, for example, by a depression or a micro-machined well in or associated with a microwell plate, microtiter plate, microplate, or a chip, or by isolated hydrophobic areas on a generally hydrophobic surface. Defined spaces may be arranged as an array, which may be a substantially planar one-dimensional or two-dimensional arrangement of elements, including sensors or wells. Defined spaces, whether arranged as an array or in some other configuration, may be in electrical communication with at least one sensor to allow detection or measurement of one or more detectable or measurable parameters or characteristics. The sensors may convert changes in the presence, concentration, or amounts of reaction by-products (or changes in ionic character of reactants) into an output signal, which may be registered electronically, for example, as a change in a voltage level or a current level. In one embodiment, the output signal and/or change in voltage or current level, in turn, may be processed to extract information or signal about a chemical reaction or desired association event, for example, a nucleotide incorporation event and/or a related ion concentration (e.g., a pH measurement). The sensors may include at least one ion sensitive field effect transistor (“ISFET”) and/or chemically sensitive field effect transistor (“chemFET”).

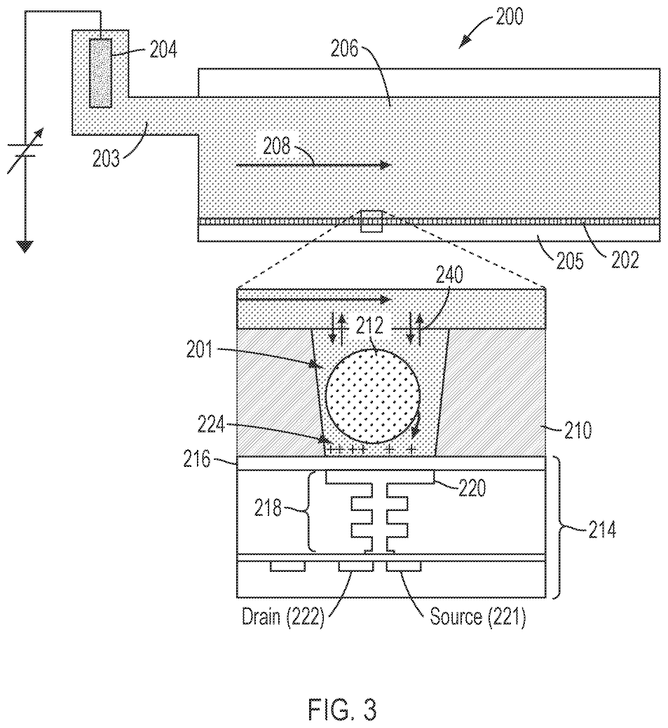

FIG. 3 illustrates an exemplary flow cell for nucleic acid sequencing. The flow cell 200 includes a microwell array 202, a sensor array 205, and a flow chamber 206 in which a reagent flow 208 may move across a surface of the microwell array 202 and/or over open ends of microwells in the microwell array 202. The flow of reagents (e.g., nucleotide species) can be provided in any suitable manner, including delivery by pipettes, or through tubes or passages connected to a flow chamber. A microwell 201 in the microwell array 202 may have any suitable volume, shape, and aspect ratio. A sensor 214 in the sensor array 205 may be an ISFET or a chemFET sensor with a floating gate 218 having a sensor plate 220 separated from the microwell interior by a passivation layer 216, and may be predominantly responsive to (and generate an output signal related to) an amount of charge 224 present on the passivation layer 216 opposite of the sensor plate 220. Changes in the amount of charge 224 may cause changes in a current between a source 221 and a drain 222 of the sensor 214, which may be used directly to provide a current-based output signal, or indirectly with additional circuitry to provide a voltage output signal. Reactants, wash solutions, and other reagents may move into microwells primarily by diffusion 240. One or more analytical reactions to identify or determine characteristics or properties of an analyte of interest may be carried out in one or more microwells of the microwell array 202. Such reactions may directly or indirectly generate by-products that affect the amount of charge 224 adjacent to the sensor plate 220. In an embodiment, a reference electrode 204 may be fluidly connected to the flow chamber 206 via a flow passage 203. In an embodiment, the microwell array 202 and the sensor array 205 may together form an integrated unit forming a bottom wall or floor of the flow cell 200. In an embodiment, one or more copies of an analyte may be attached to a solid phase support 212, which may include microparticles, nanoparticles, beads, or gels, and may be solid and porous, for example. The analyte may include one or more copies of a nucleic acid analyte, which may include nucleic acid samples. Exemplary samples may include amplicons for calibration or recalibration, which may be obtained using any suitable technique, as further described herein.

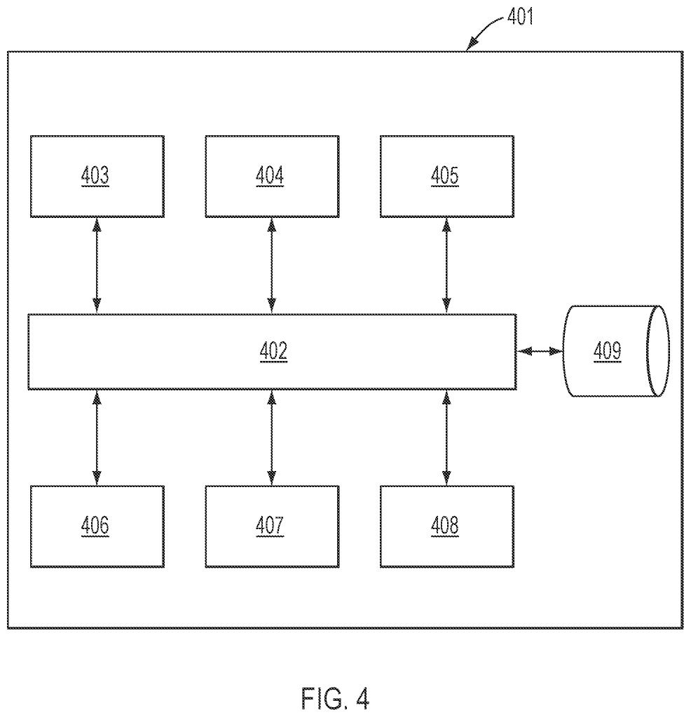

FIG. 4 illustrates an exemplary computer system 401 for performing nucleic acid sequencing. Such a computer system 401 could be used as computing server/node/device 12 of FIG. 1. The computer system 401 may include a bus 402 or other communication mechanism for communicating information, a processor 403 coupled to the bus 402 for processing information, and/or a memory 405 coupled to the bus 402 for dynamically and/or statically storing information. The computer system 401 can also include one or more co-processors 404 coupled to the bus 402 (e.g., GPUs and/or FPGAs) for performing specialized processing tasks; a display 406 coupled to the bus 402 (e.g., a cathode ray tube (CRT) or liquid crystal display (LCD)) for displaying information to a computer user; an input device 407 coupled to the bus 402 (e.g., a keyboard including alphanumeric and other keys) for communicating information and command selections to the processor 403; a cursor control device 408 coupled to the bus 402 (e.g., a mouse, a trackball, or cursor direction keys) for communicating direction information and command selections to the processor 403 and for controlling cursor movement on display 406; and one or more storage devices 409 coupled to the bus 402 (e.g., a magnetic disk or an optical disk) for storing information and instructions. The memory 405 may include a random access memory (RAM) or other dynamic storage device and/or a read only memory (ROM) or other static storage device. Such an exemplary computer system with suitable software may be used to perform the embodiments described herein. More generally, in various embodiments, one or more features of the teachings and/or embodiments described herein may be performed or implemented using appropriately configured and/or programmed hardware and/or software elements.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor devices, chips, microchips, chip sets, and so forth. The local interface may include, for example, one or more buses or other wired or wireless connections, controllers, buffers (caches), drivers, repeaters and receivers, etc., to allow appropriate communications between hardware components. A processor may include a hardware device for executing software, particularly software stored in memory. The processor may include any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (O/S), which may control the execution of other computer programs such as the system, and provide scheduling, input-output control, file and data management, memory management, communication control, etc.

According to various embodiments, one or more features of teachings and/or embodiments described herein may be performed or implemented using an appropriately configured and/or programmed non-transitory machine-readable medium or article that may store an instruction or a set of instructions that, if executed by a machine, may cause the machine to perform a method and/or operations in accordance with the embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, flash memory, hard drive, tape, CDROM, etc.). Moreover, memory can incorporate electronic, magnetic, optical, and/or other types of storage media. Memory can have a distributed, clustered, remote, or cloud architecture where various components may be situated remote from one another, and accessed by the processor. The instructions may include any suitable type of code, including source code, compiled code, interpreted code, executable code, static code, dynamic code, encrypted code, etc., implemented using any suitable high-level, low-level, object-oriented, visual, compiled and/or interpreted programming language.

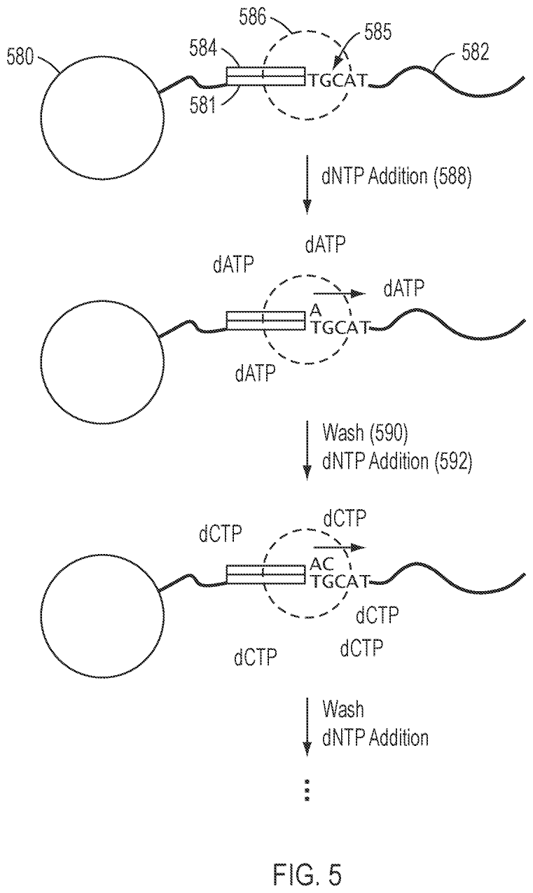

FIG. 5 illustrates an exemplary process 500 for label-free, pH-based sequencing. In one embodiment, a template 582 with sequence 585 and a primer binding site 581 may be attached to a solid phase support 580. The template 582 may include nucleic acid samples comprising amplicons for calibration or recalibration as further described herein. The template 582 may be attached as a clonal population to a solid support (e.g., a microparticle or bead), for example, and may be prepared as disclosed in Leamon et al., U.S. Pat. No. 7,323,305. In an embodiment, the template may be associated with a substrate surface or present in a liquid phase with or without being coupled to a support. A primer 584 and DNA polymerase 586 may be annealed to the template 582 so that the primer's 3′ end may be extended by a polymerase, and a polymerase may be bound to such a primer-template duplex (or in close proximity thereof) so that binding and/or extension may take place when dNTPs are added. In one embodiment, step 588 may include adding dNTP (shown as dATP), and the DNA polymerase 586 may incorporate a nucleotide “A” (since “T” may be the next nucleotide in the template 582, and “T” is complementary to the flowed dATP nucleotide). In one embodiment, step 590 may include performing a wash. In one embodiment, step 592 may include adding the next dNTP (shown as dCTP), and the DNA polymerase 586 may incorporate a nucleotide “C” (since “G” may be the next nucleotide in the template 582). More details about pH-based nucleic acid sequencing may be found in U.S. Pat. No. 7,948,015 and U.S. Pat. Appl. Publ. Nos. 2010/0137143, 2009/0026082, and 2010/0282617, each of which is incorporated by reference herein in its entirety.

In an embodiment, the primer-template-polymerase complex may be subjected to a series of exposures of different nucleotides in a pre-determined sequence or ordering. If one or more nucleotides are incorporated, the signal resulting from the incorporation reaction may be detected. In one embodiment, the nucleotide sequence of the template strand may be determined after repeated cycles of nucleotide addition, primer extension, and/or signal acquisition. The output signals measured throughout this process may depend on the number of nucleotide incorporations. Specifically, in each addition step, the polymerase may extend the primer by incorporating added dNTP only if the next base in the template is complementary to the added dNTP. With each incorporation, a hydrogen ion may be released, and collectively, a population of released hydrogen ions may change a local pH of the respective reaction chamber. The production of hydrogen ions may be monotonically related to the number of contiguous complementary bases (e.g., homopolymers) in the template. Deliveries of nucleotides to a reaction vessel or chamber may be referred to as “flows” of nucleotide triphosphates (or dNTPs). For convenience, a flow of dATP will sometimes be referred to as “a flow of A” or “an A flow,” and a sequence of flows may be represented as a sequence of letters, such as “ATGT” indicating “a flow of dATP, followed by a flow of dTTP, followed by a flow of dGTP, followed by a flow of dTTP.” The predetermined ordering may be based on a cyclical, repeating pattern including consecutive repeats of a short pre-determined reagent flow ordering (e.g., consecutive repeats of pre-determined sequence of four nucleotide reagents, for example, “ACTG ACTG . . . ”). The predetermined ordering may be based in whole or in part on some other pattern of reagent flows (e.g., any of the various reagent flow orderings discussed in Hubbell et al., U.S. Pat. Appl. Publ. No. 2012/0264621, published Oct. 18, 2012, which is incorporated by reference herein in its entirety), and may also be based on some combination thereof.

In various embodiments, output signals due to nucleotide incorporation may be processed, given knowledge of what nucleotide species were flowed and in what order to obtain such signals. The output signals may be processed to make base calls for the flows and/or to compile consecutive base calls associated with a sample nucleic acid template into a read. A base call may refer to a particular nucleotide identification (e.g., dATP (“A”), dCTP (“C”), dGTP (“G”), or dTTP (“T”)). Base calling may include performing one or more signal normalizations, signal phase and signal decay (e.g., enzyme efficiency loss) estimations, signal corrections, and model-based signal predictions. Base calling may also identify or estimate base calls for each flow for each defined space. Any suitable base calling method may be used, including as described in Davey et al., U.S. Pat. Appl. Publ. No. 2012/0109598, published on May 3, 2012, and/or Sikora et al., U.S. Pat. Appl. Publ. No. 2013/0060482, published on Mar. 7, 2013, each of which is incorporated by reference herein in its entirety, taking into account that more accurate base callers may yield better results.

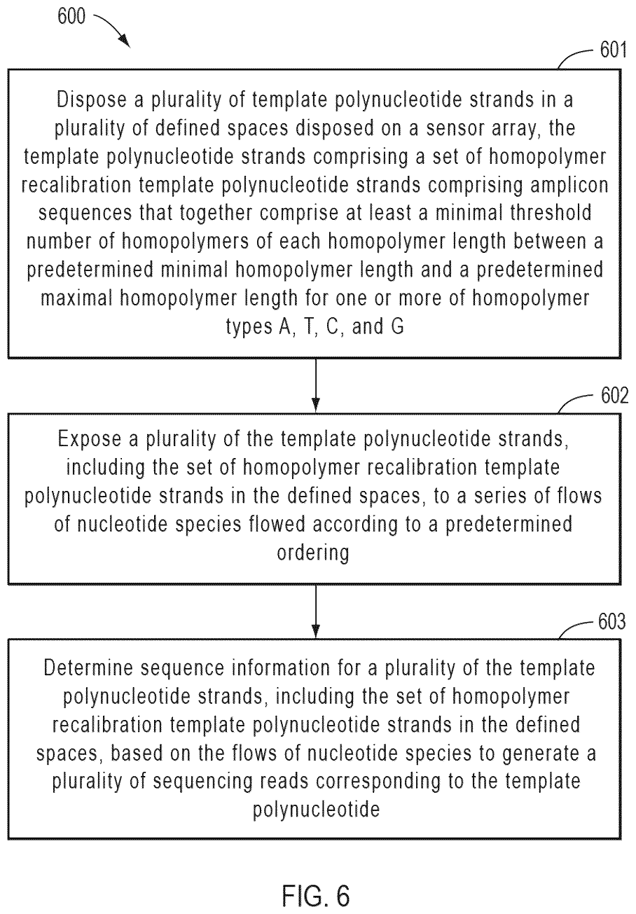

FIG. 6 illustrates an exemplary method 600 for nucleic acid sequencing using a homopolymer calibration panel. In step 601, a plurality of template polynucleotide strands may be disposed in a plurality of defined spaces disposed on a sensor array, the template polynucleotide strands comprising a set of homopolymer recalibration template polynucleotide strands, wherein the homopolymer recalibration template polynucleotide strands may comprise amplicon sequences that together comprise at least a minimal threshold number of homopolymers of each homopolymer length between a predetermined minimal homopolymer length and a predetermined maximal homopolymer length for one or more of homopolymer types A, T, C, and G (e.g., each of homopolymer types A, T, C, and G). In step 602, the plurality of the template polynucleotide strands, including the set of homopolymer recalibration template polynucleotide strands in the defined spaces, may be exposed to a series of flows of nucleotide species flowed according to a predetermined ordering. In step 603, sequence information for a plurality of the template polynucleotide strands, including the set of homopolymer recalibration template polynucleotide strands in the defined spaces, may be determined based on the flows of nucleotide species, e.g., to generate a plurality of sequencing reads corresponding to the template polynucleotide strands.

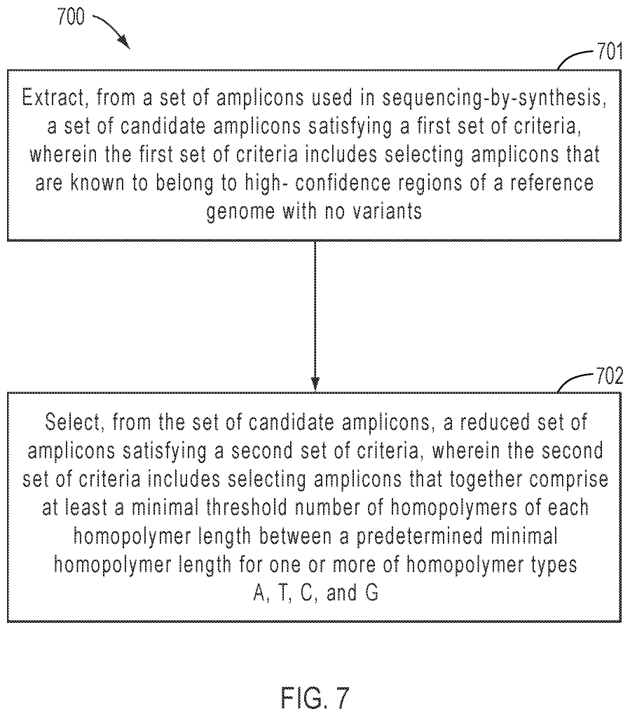

FIG. 7 illustrates an exemplary method 700 for preparing a homopolymer recalibration panel. In one embodiment, the recalibration panel may include amplicons that are 200 base pairs in length. In another embodiment, the recalibration panel may include amplicons longer than 200 base pairs in length (e.g., amplicons that are 600 base pairs in length). In step 701, a set of candidate amplicons satisfying a first set of criteria may be extracted from a set of amplicons used in sequencing-by-synthesis, wherein the first set of criteria may include selecting amplicons known to belong to high-confidence regions of a reference genome with no variants. In step 702, a reduced set of amplicons satisfying a second set of criteria may be selected from the set of candidate amplicons, wherein the second set of criteria may include selecting amplicons that together comprise at least a minimal threshold number of homopolymers of each homopolymer length between a predetermined minimal homopolymer length and a predetermined maximal homopolymer length for one or more of homopolymer types A, T, C, and G (e.g., each of homopolymer types A, T, C, and G).

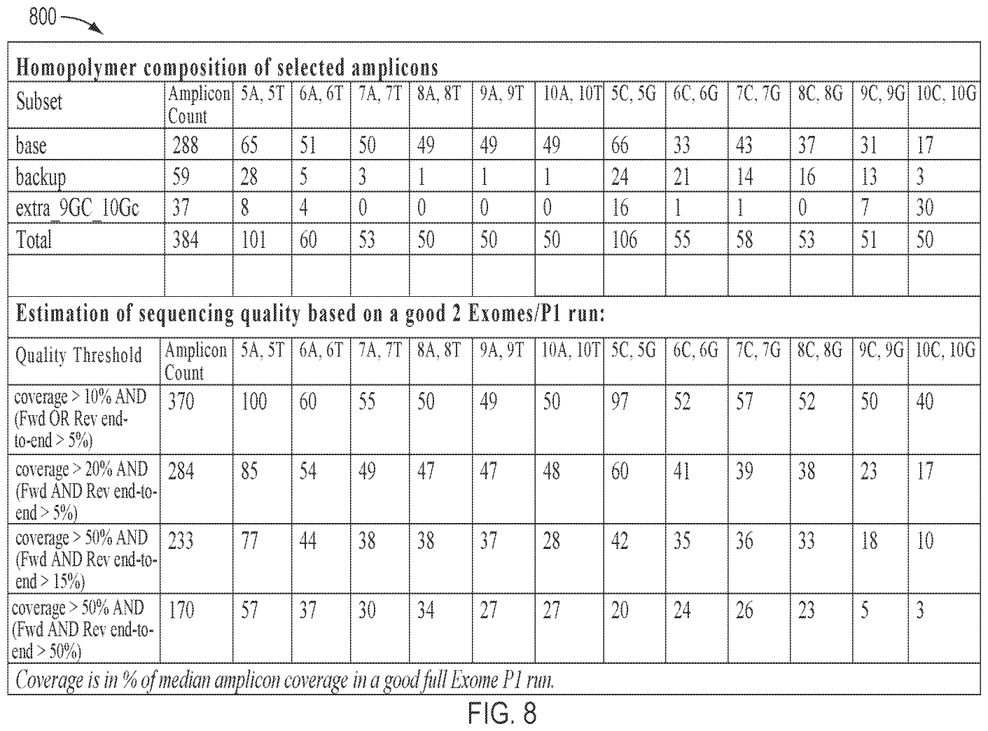

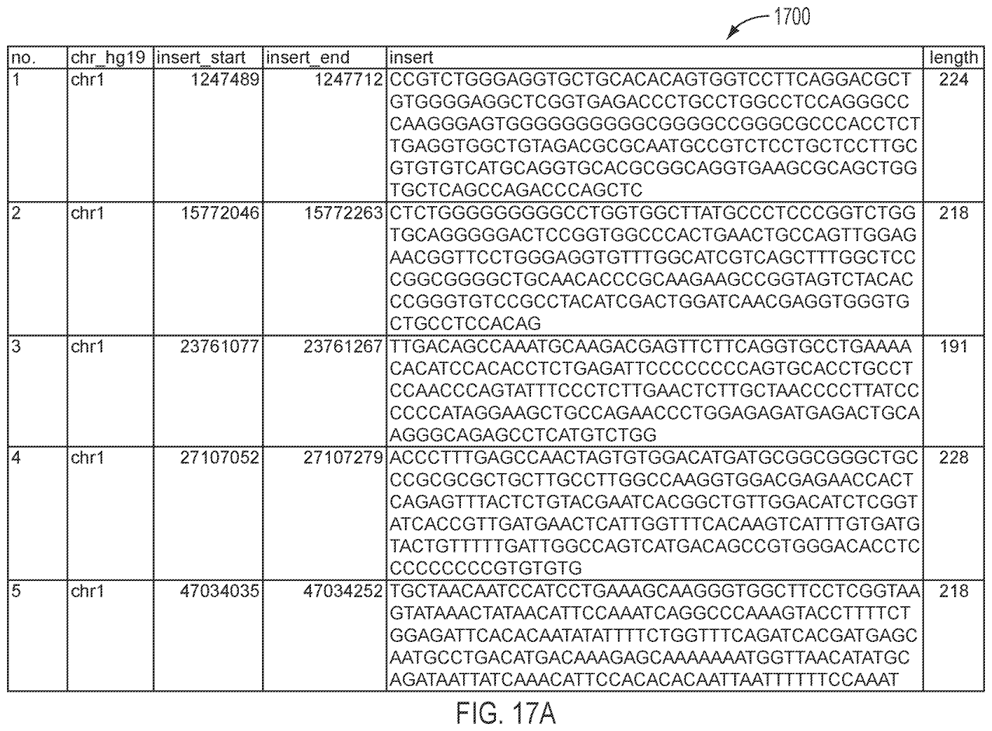

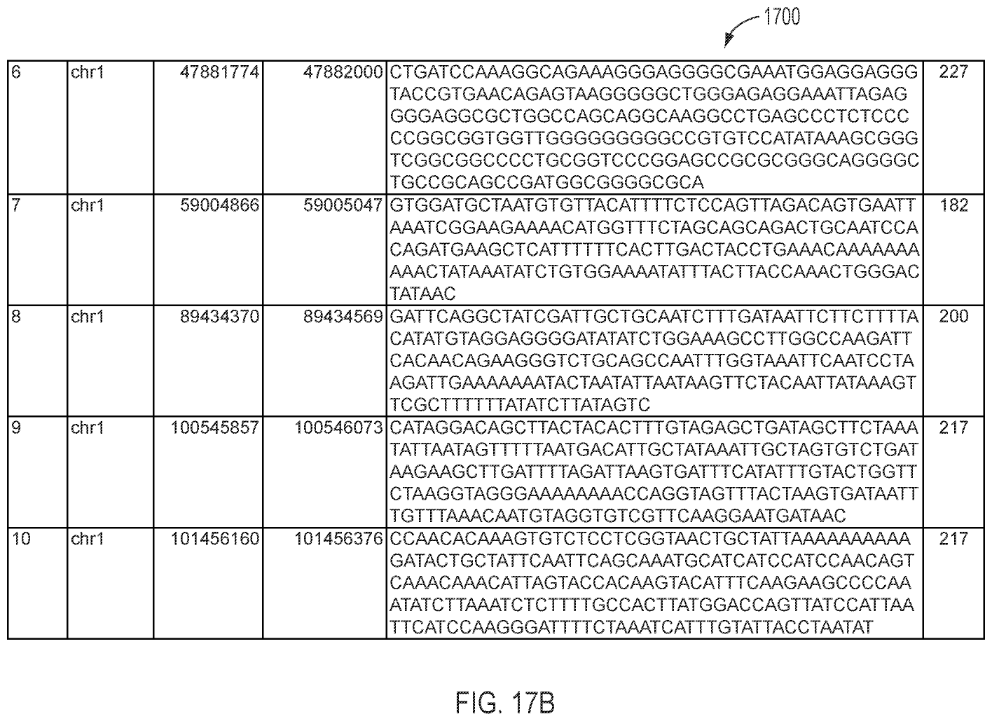

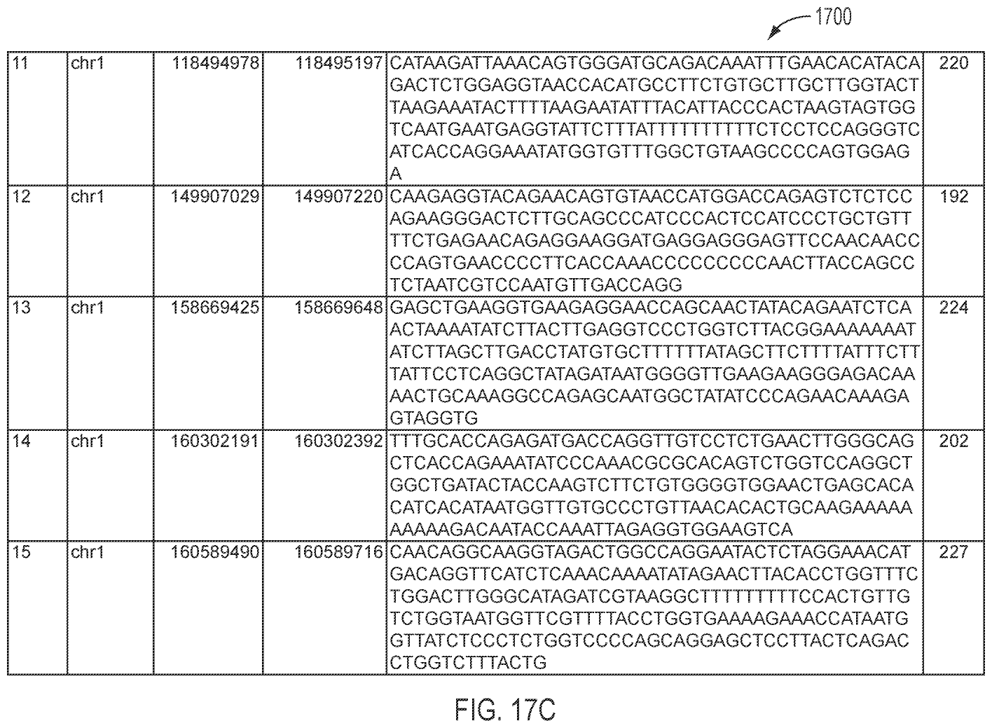

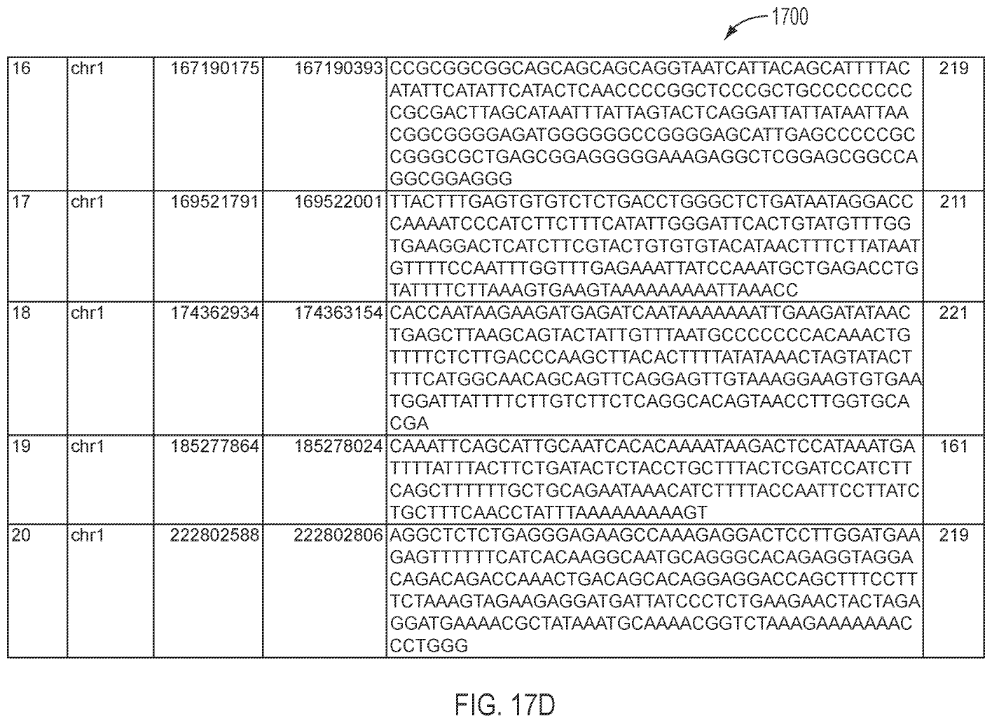

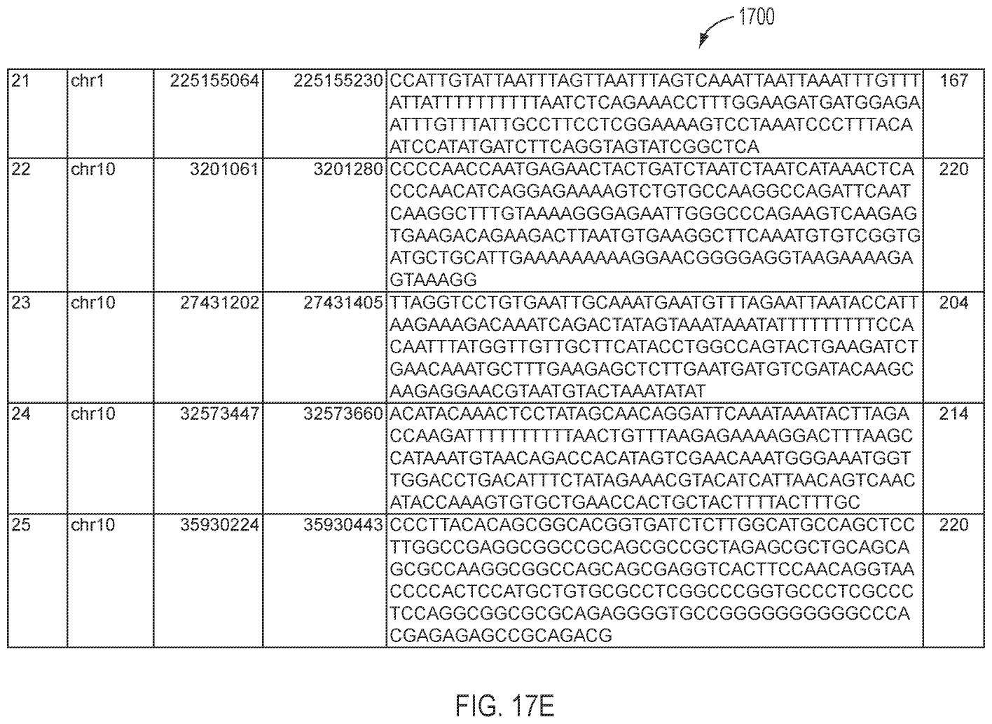

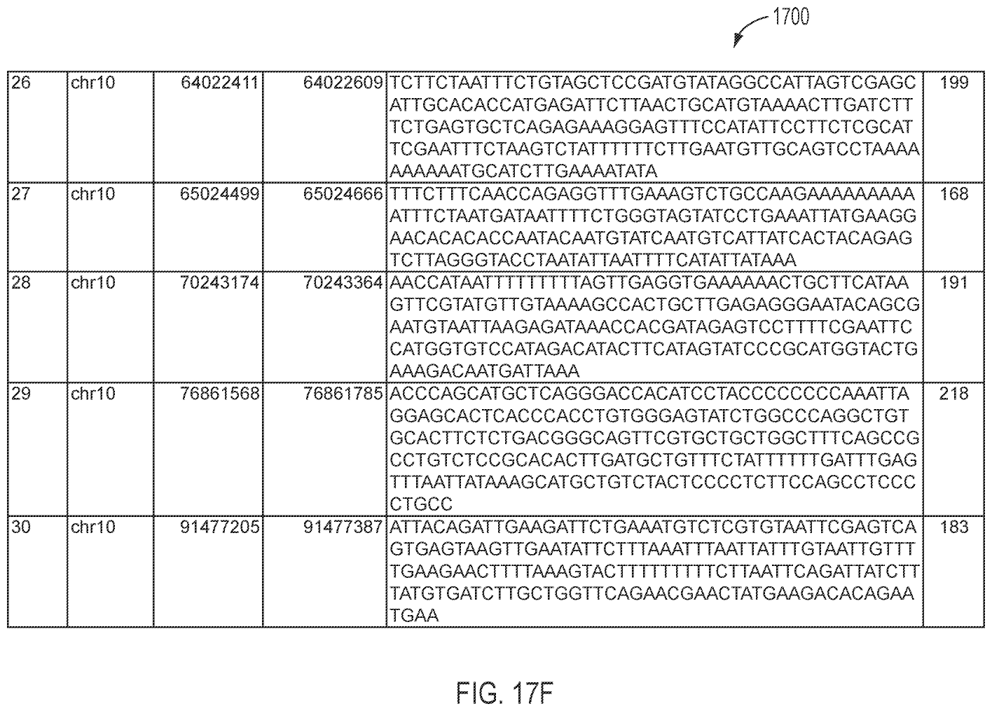

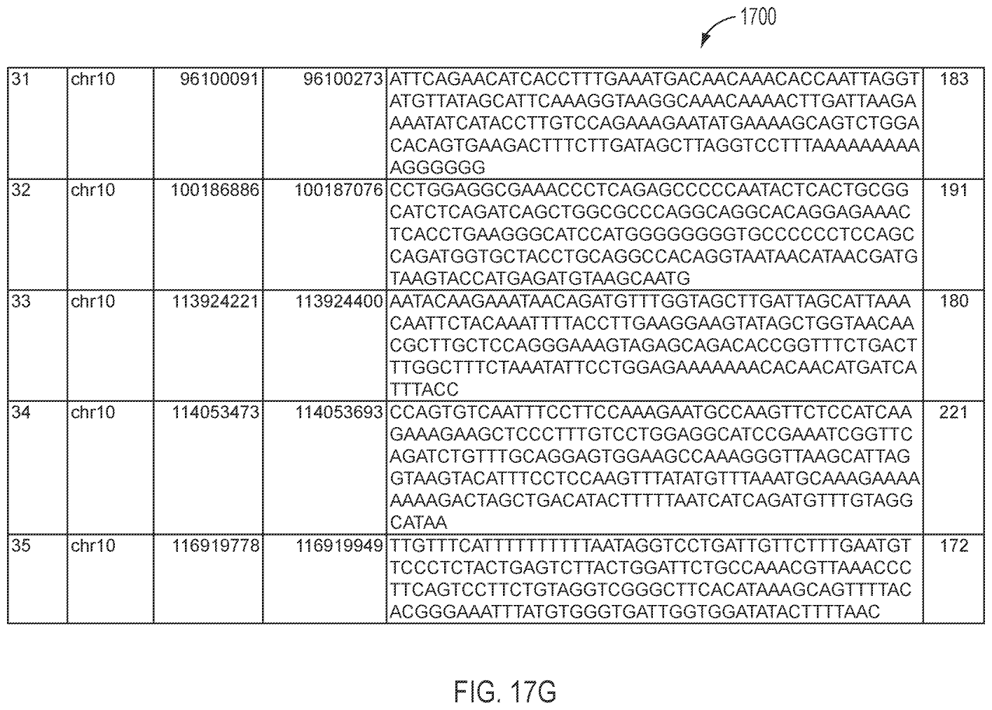

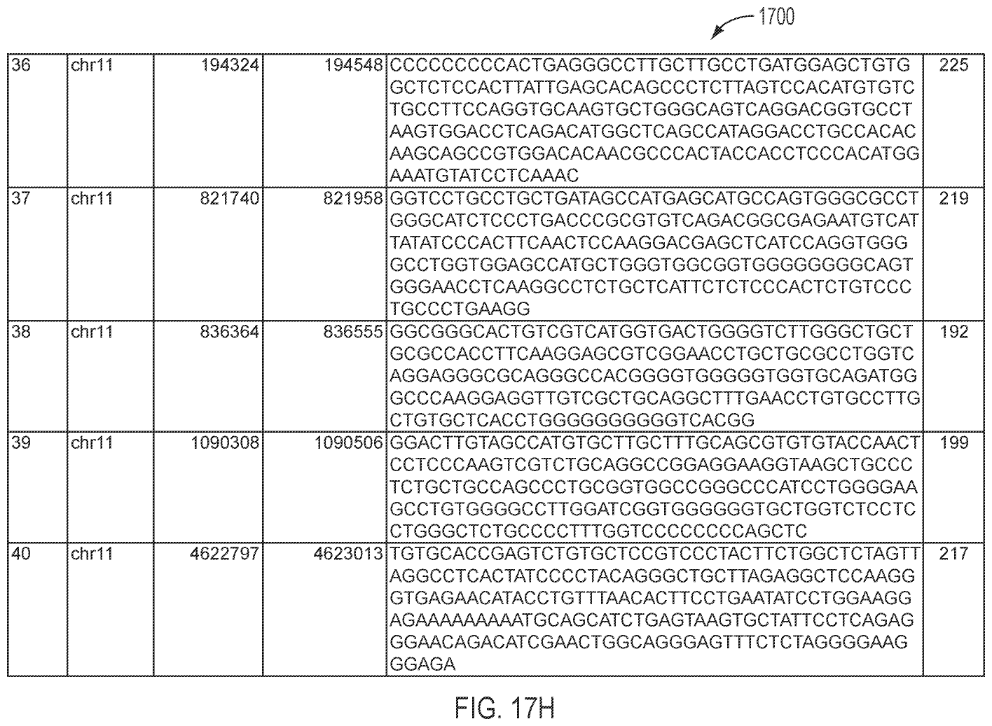

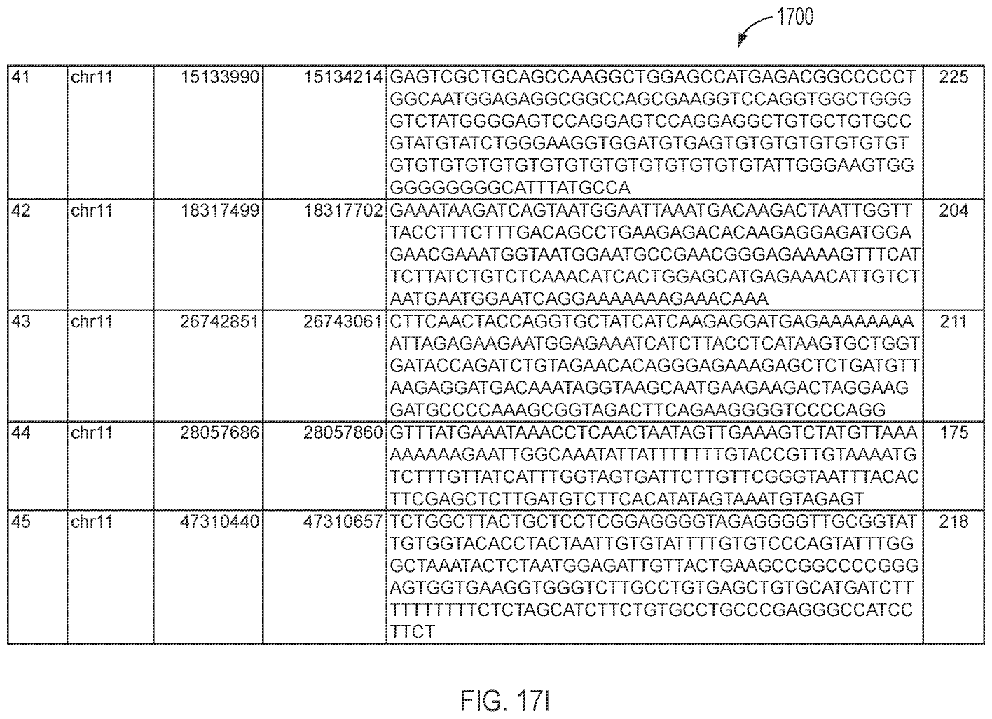

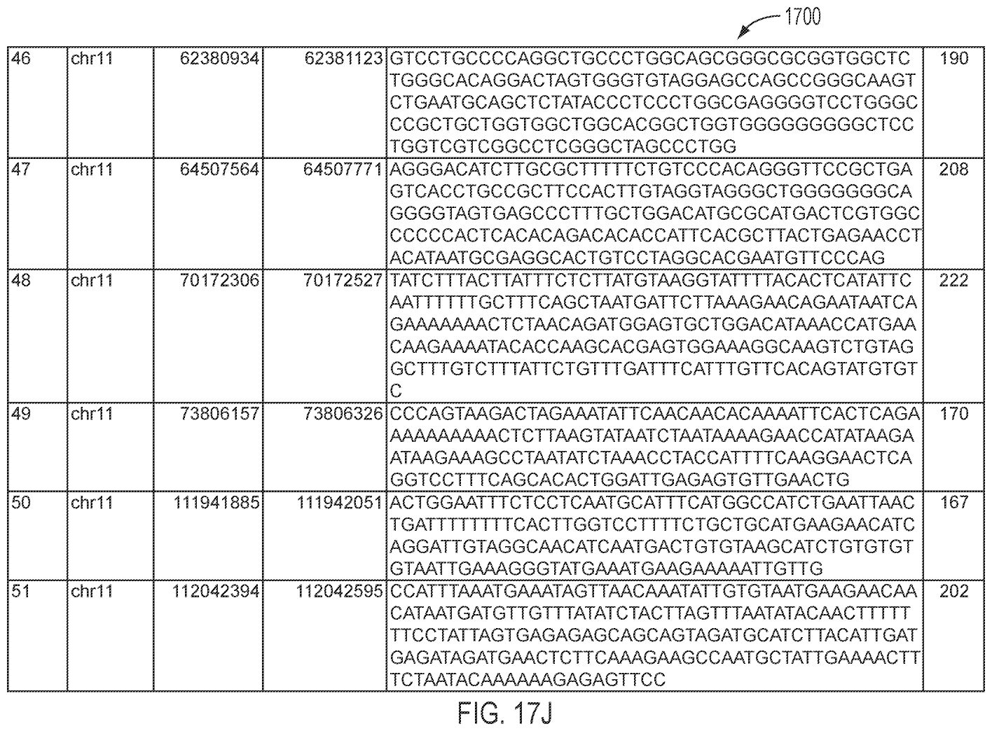

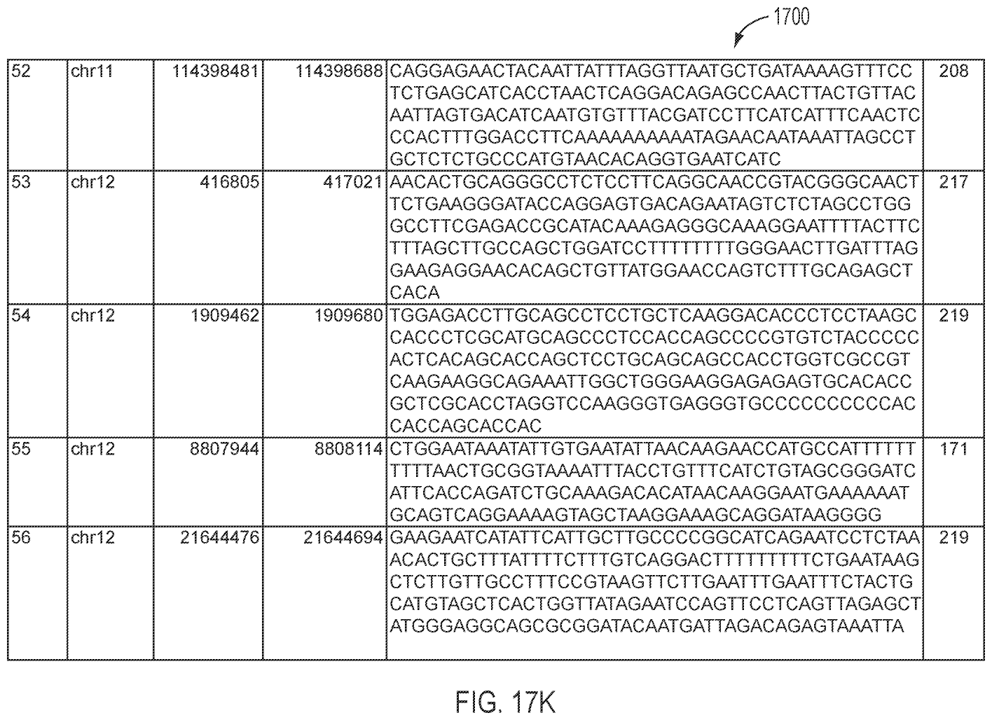

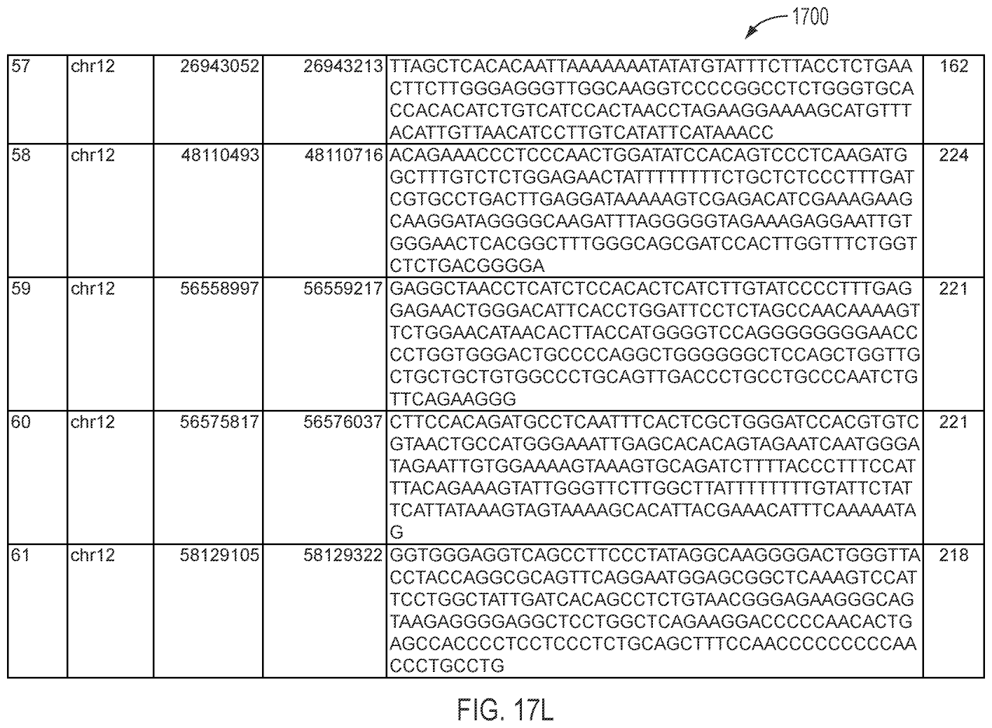

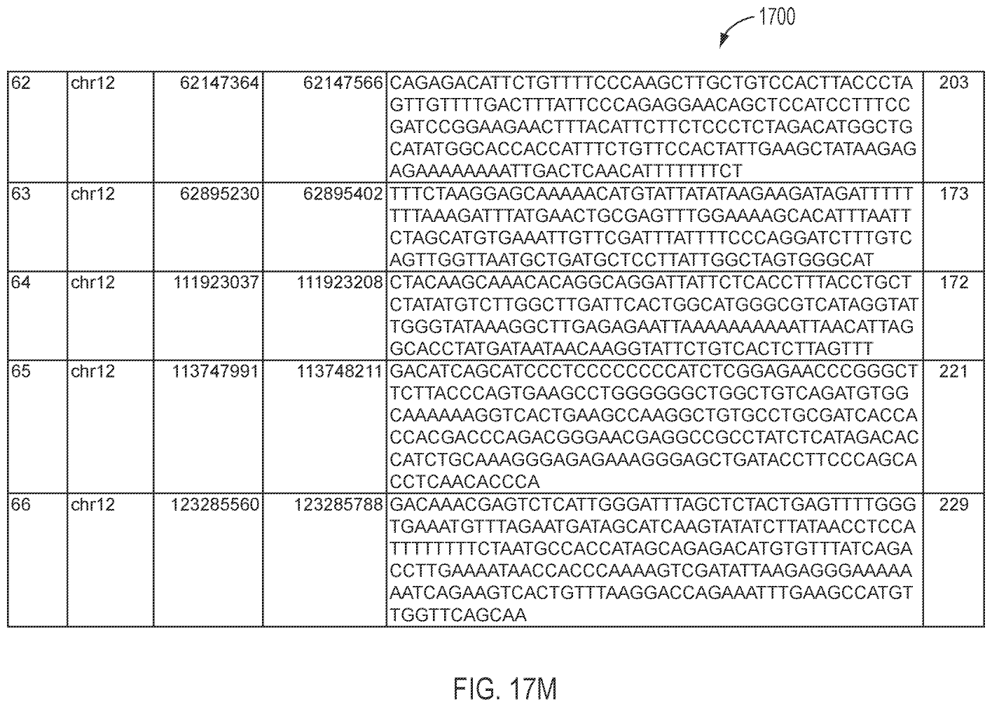

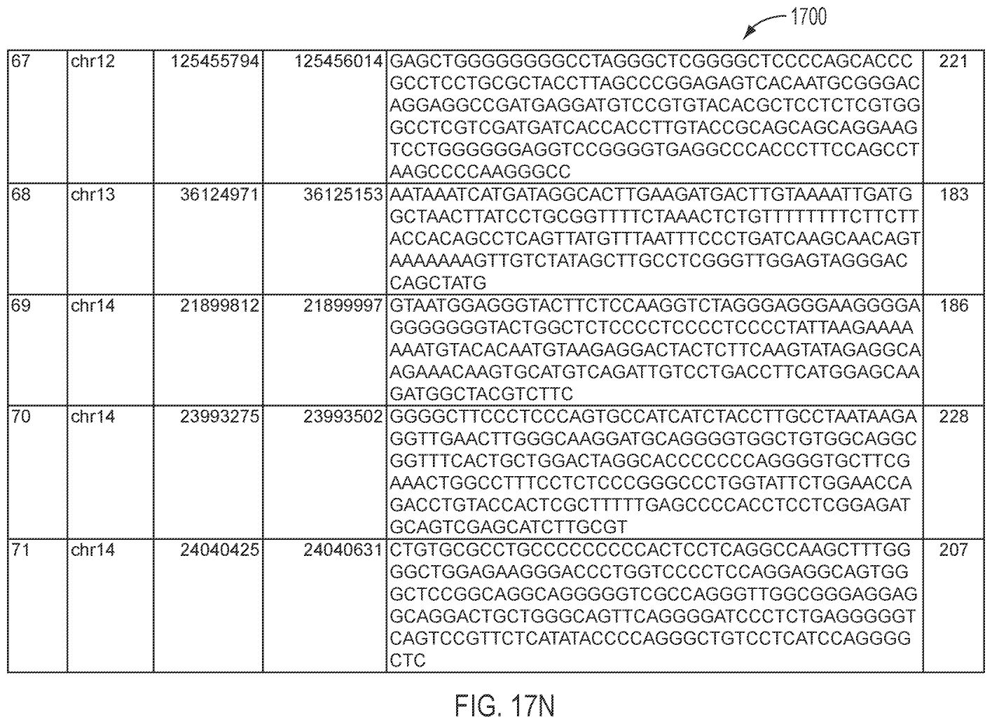

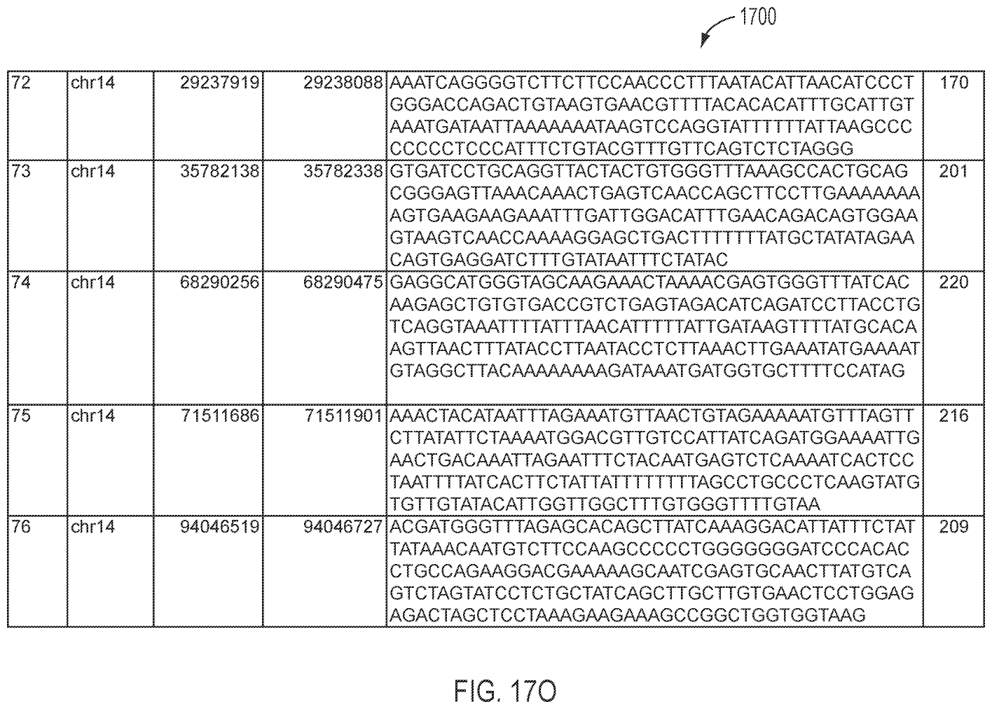

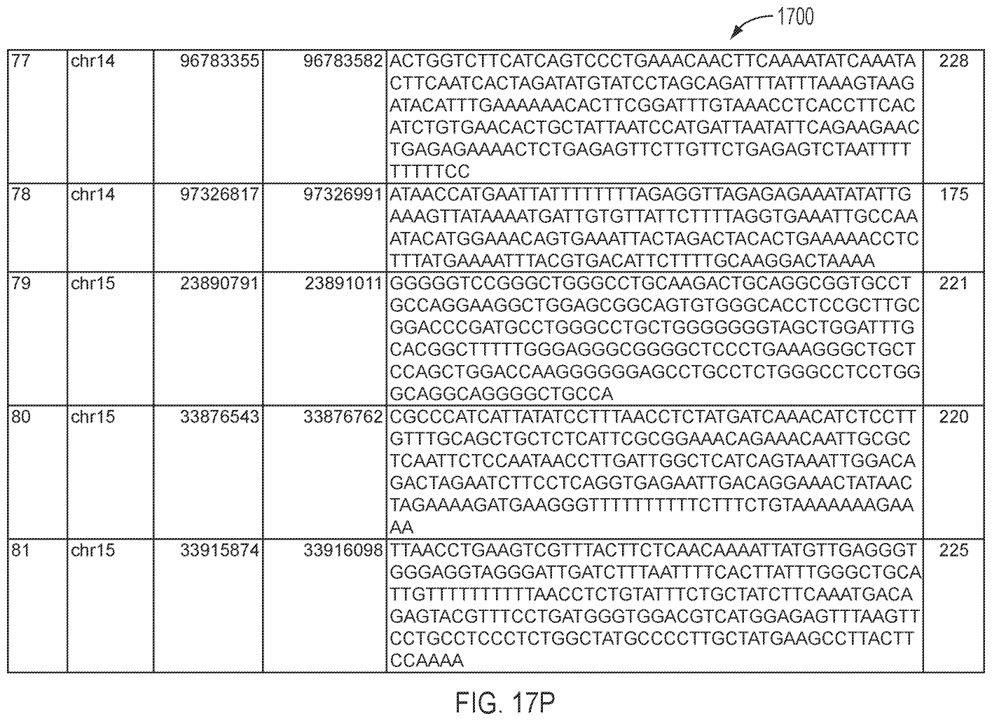

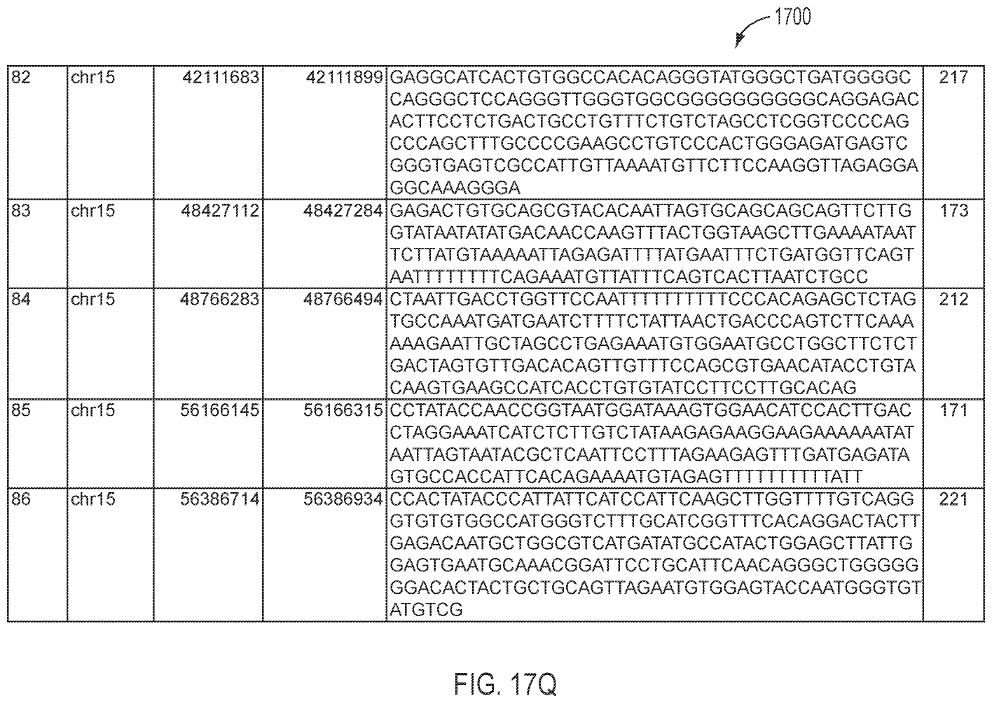

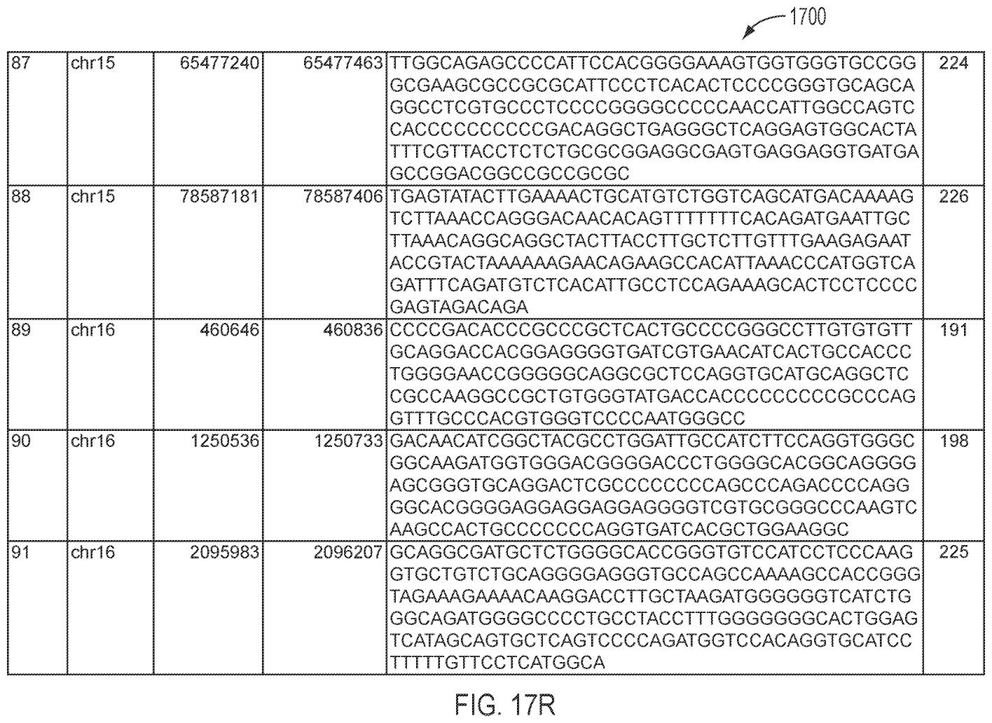

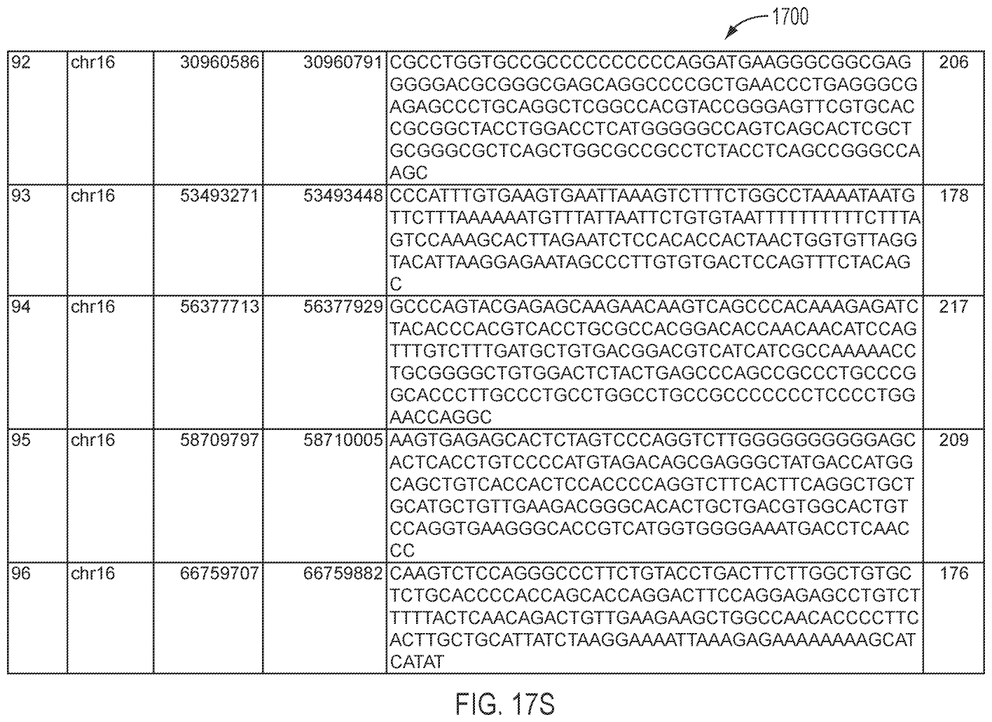

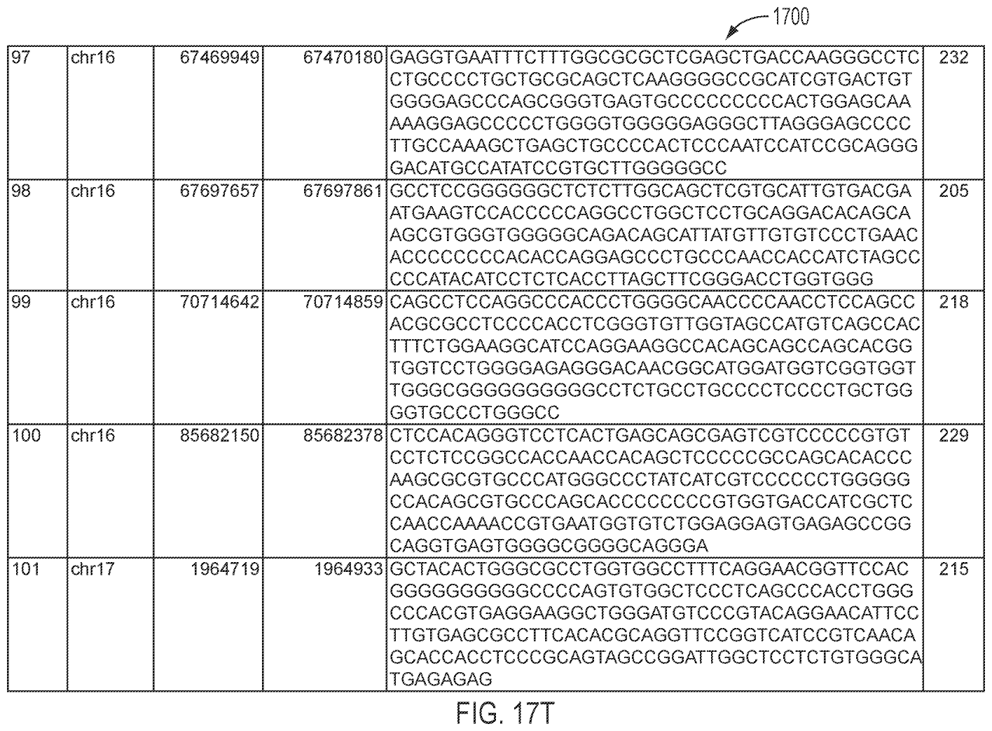

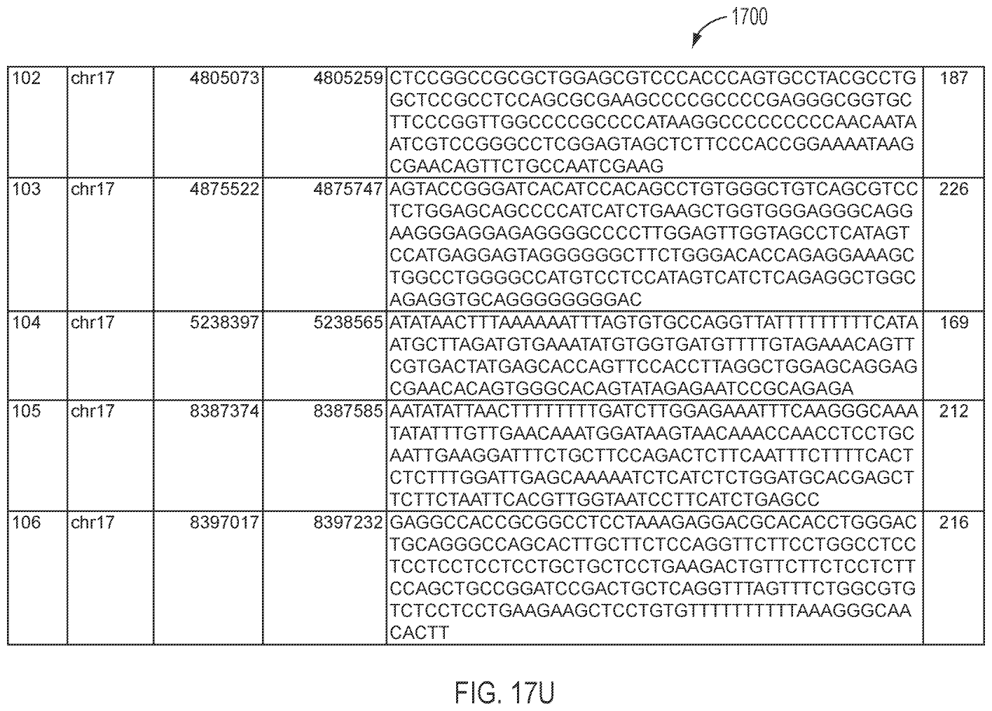

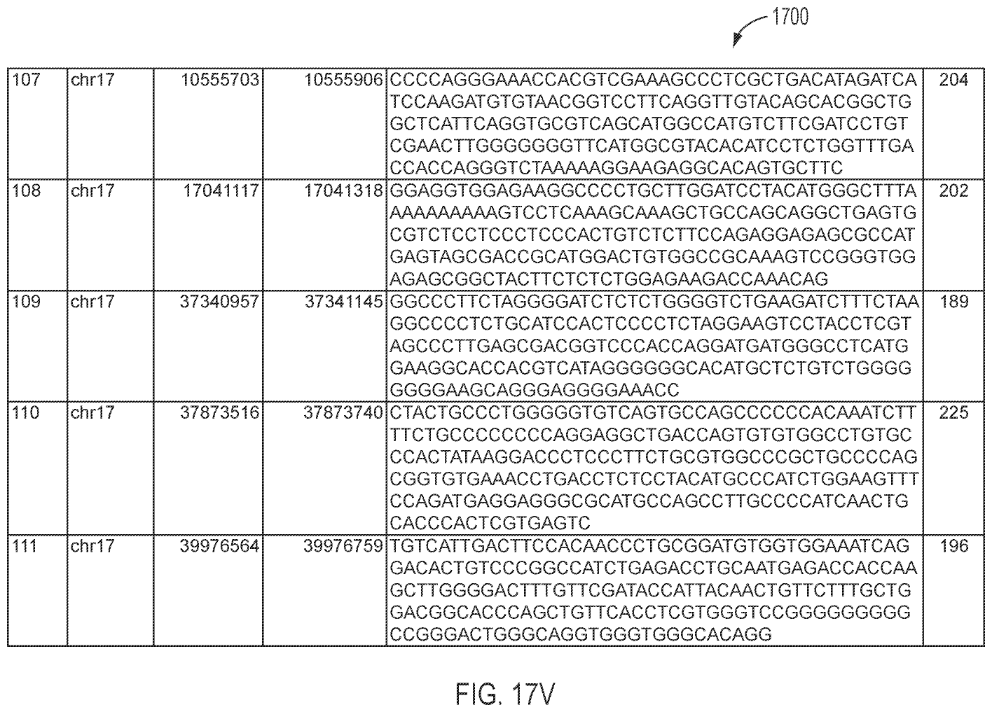

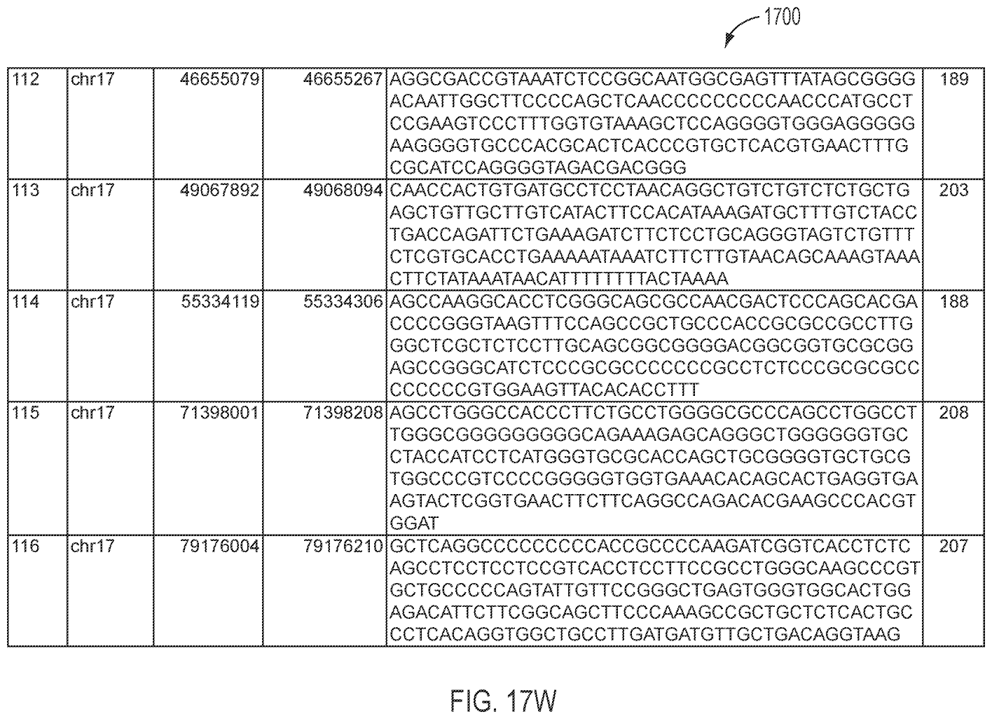

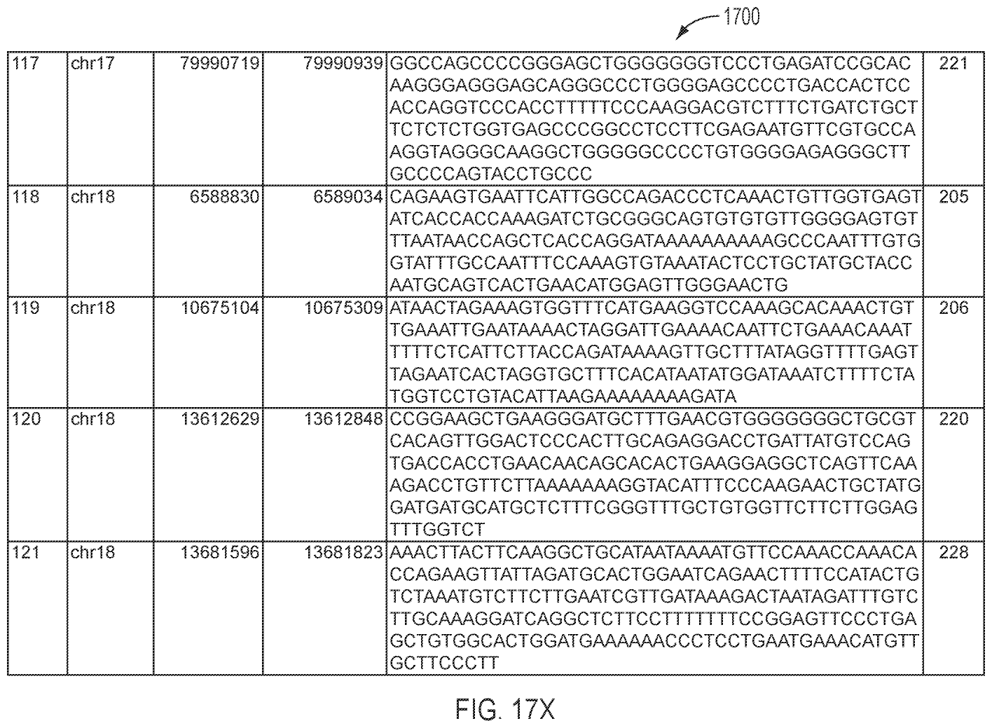

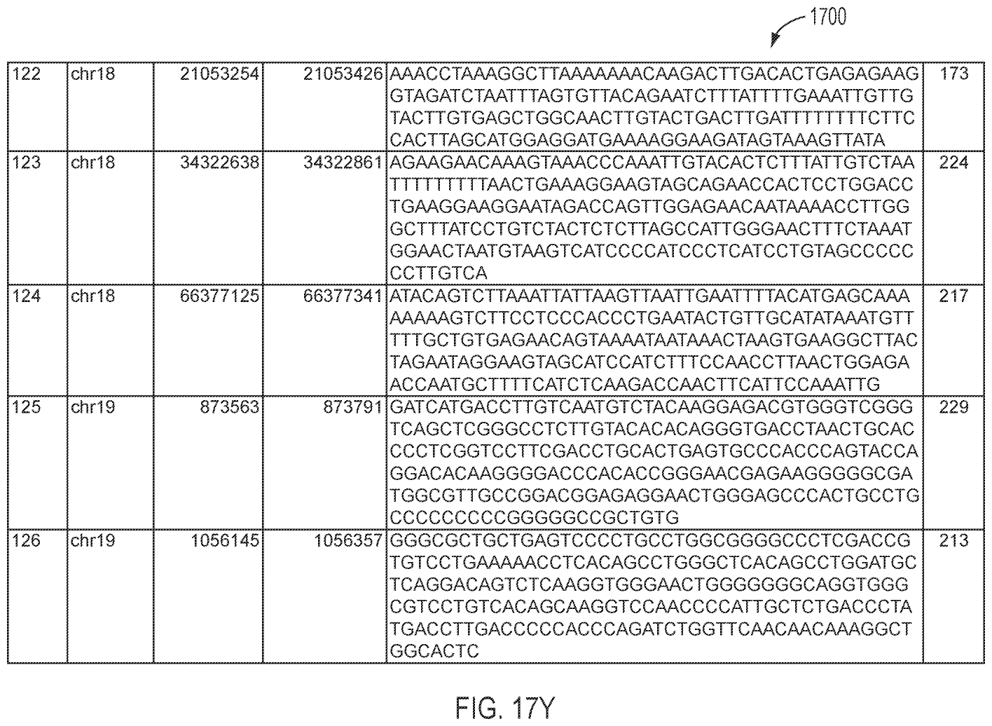

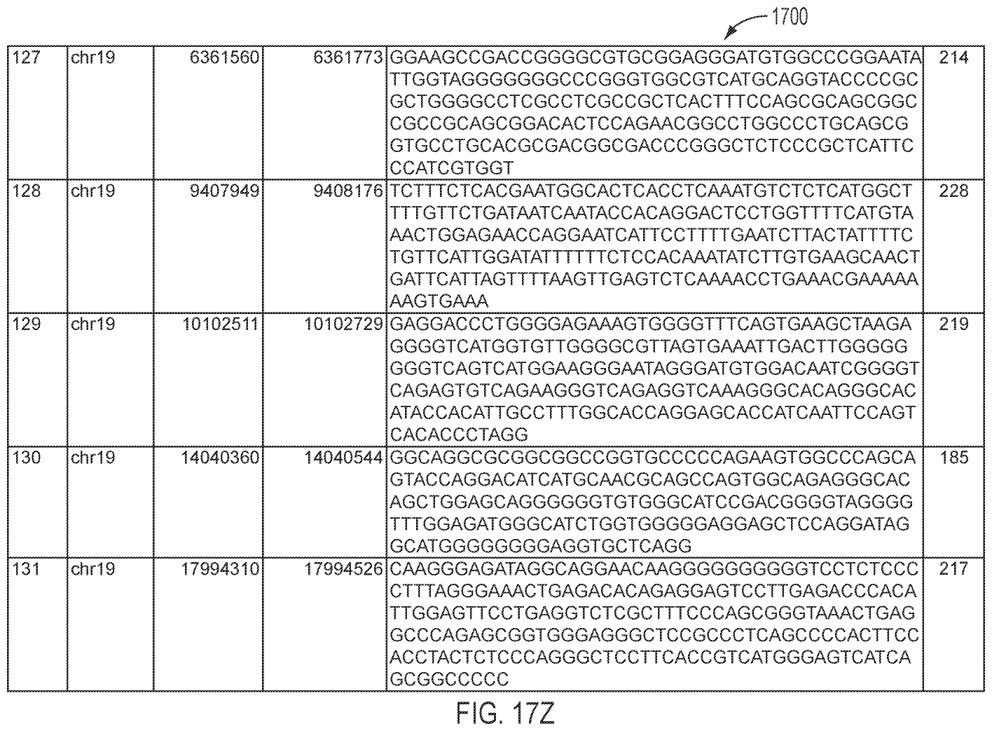

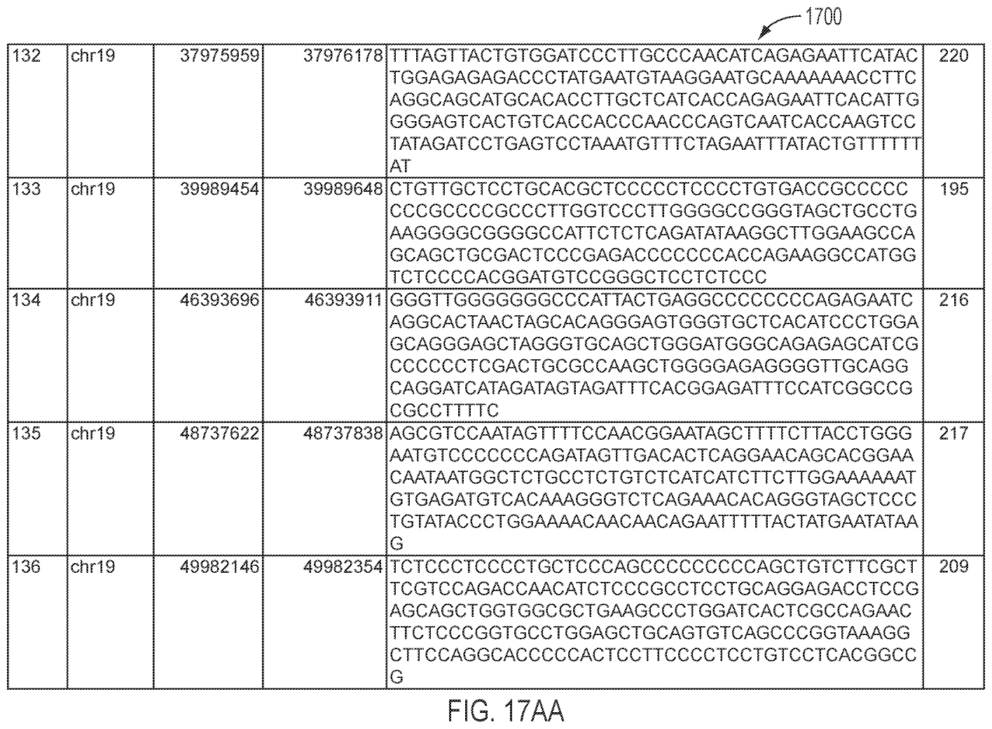

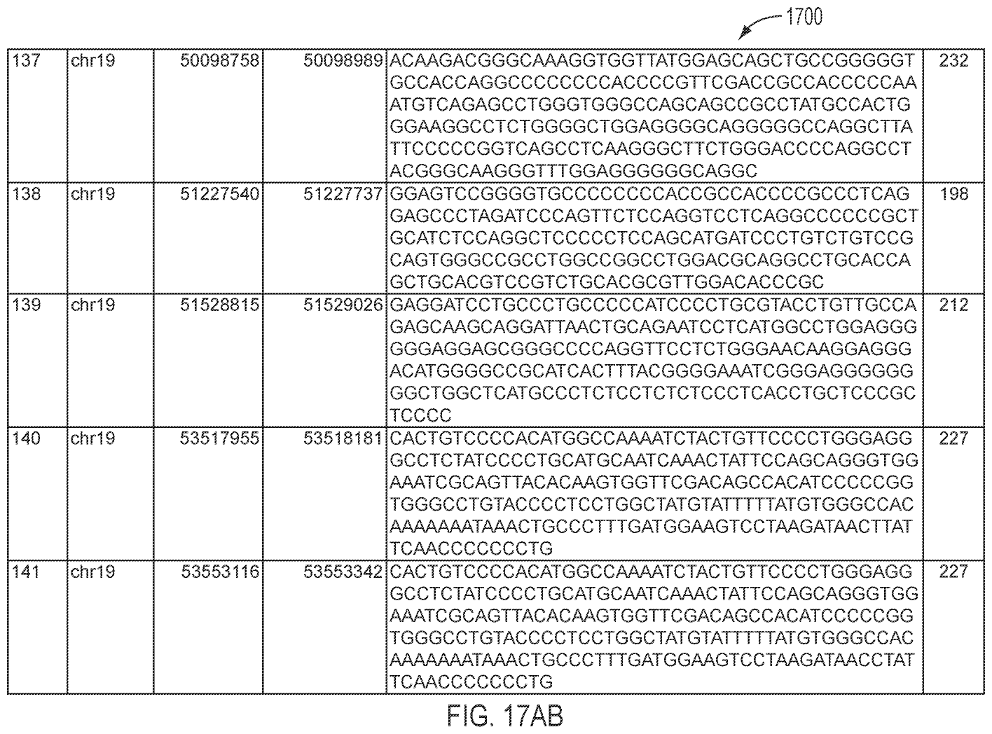

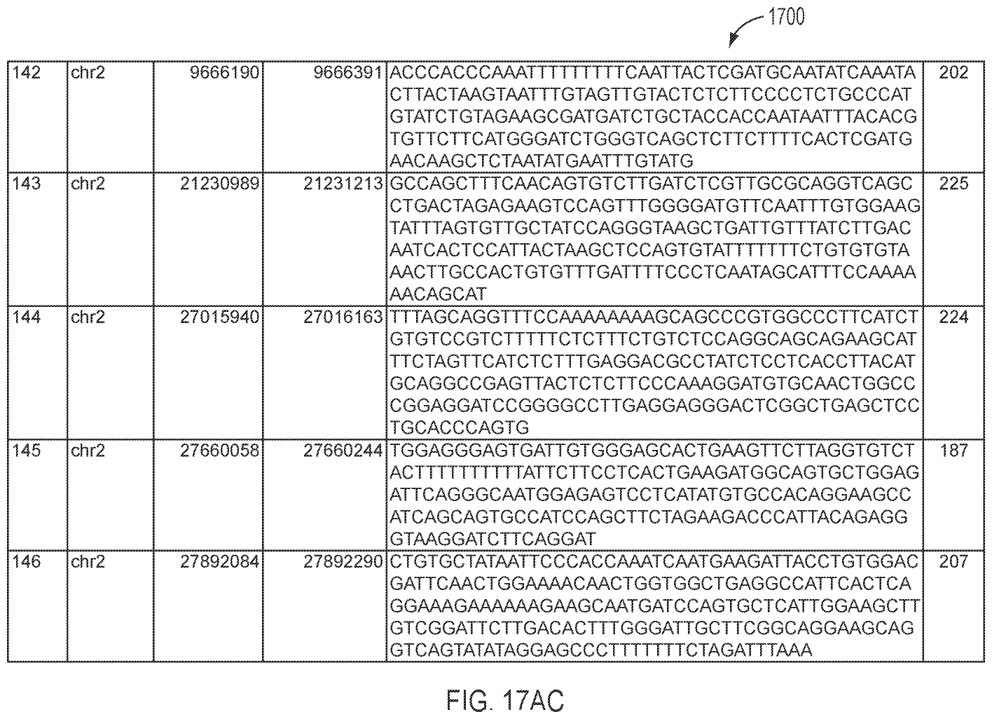

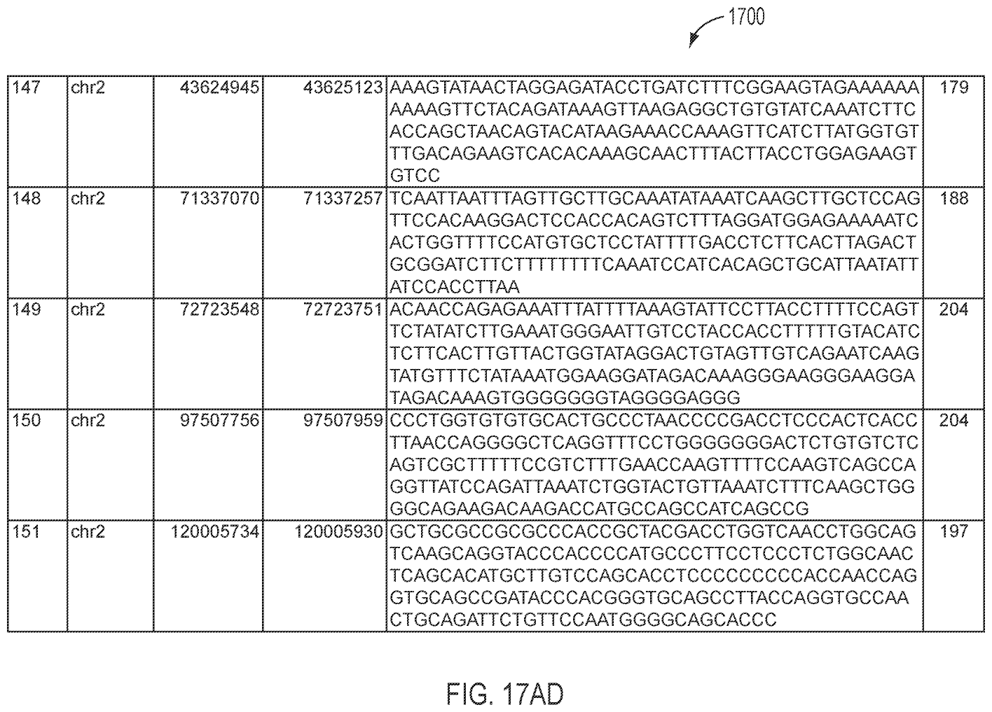

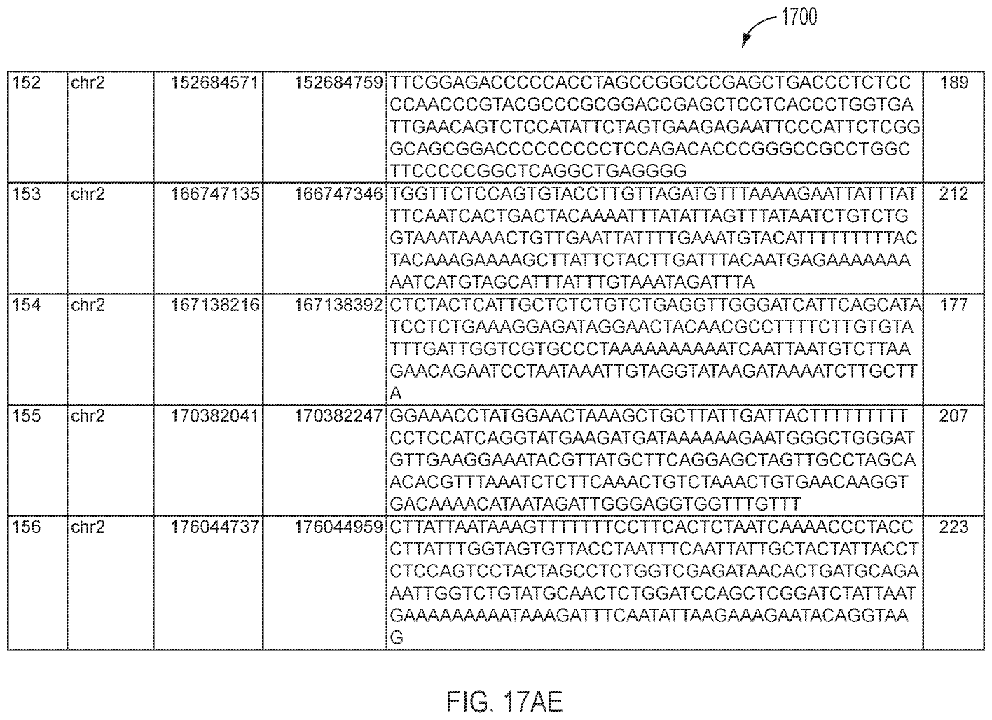

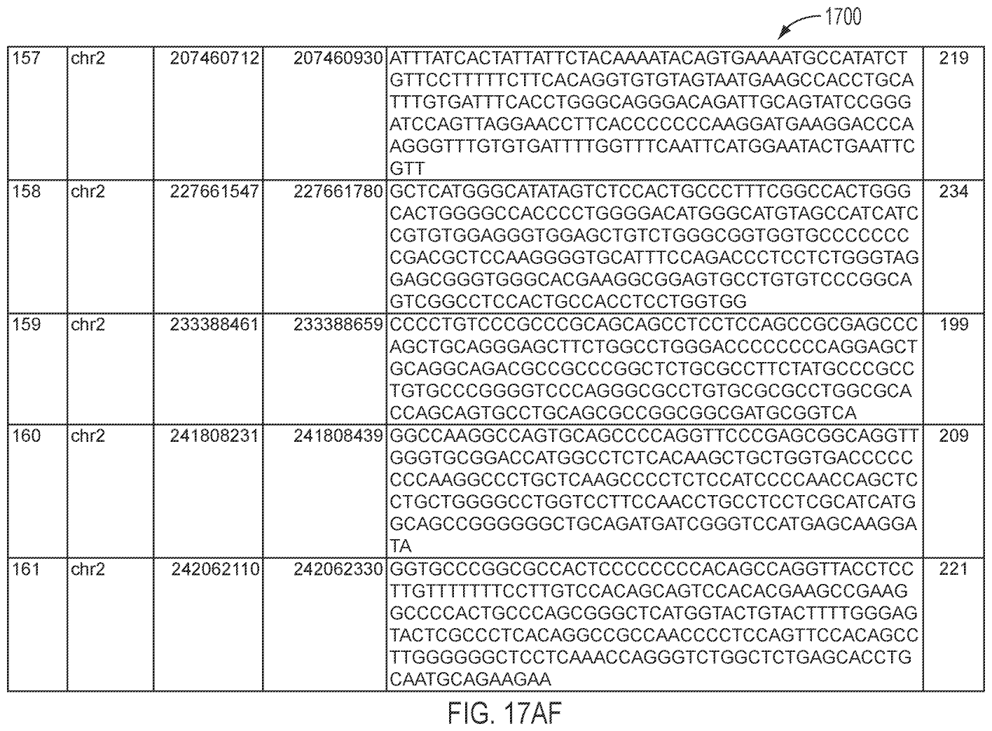

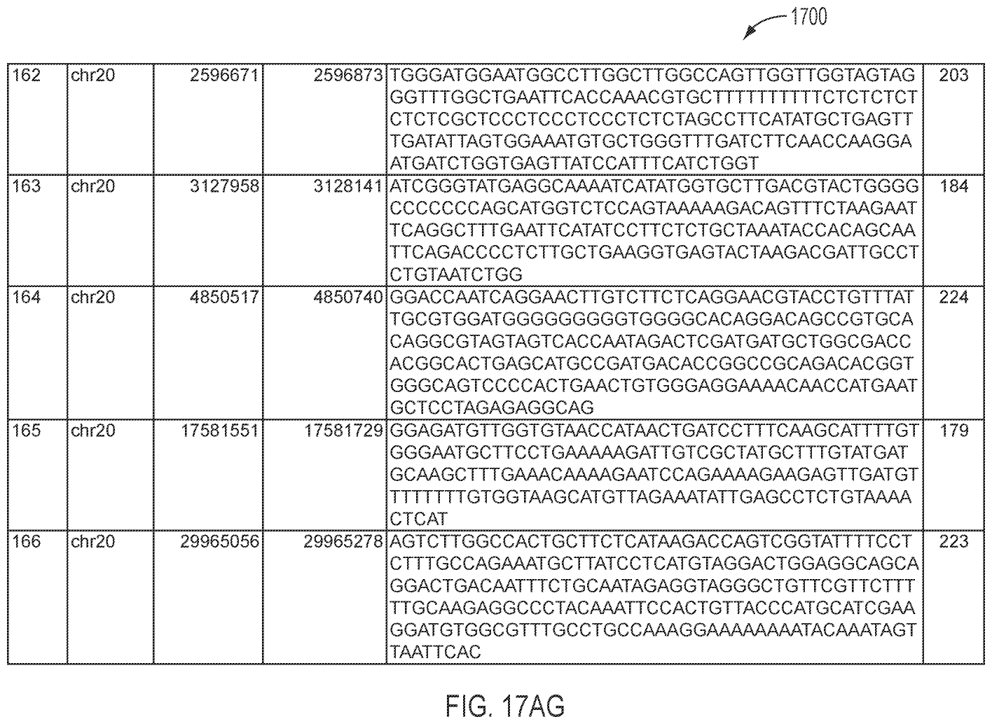

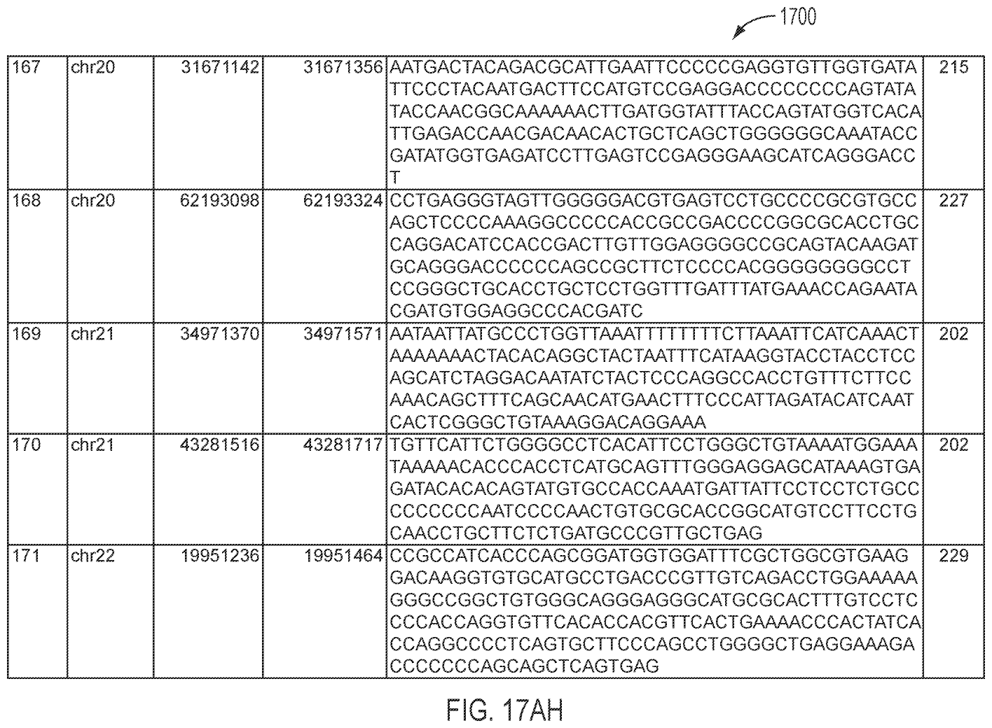

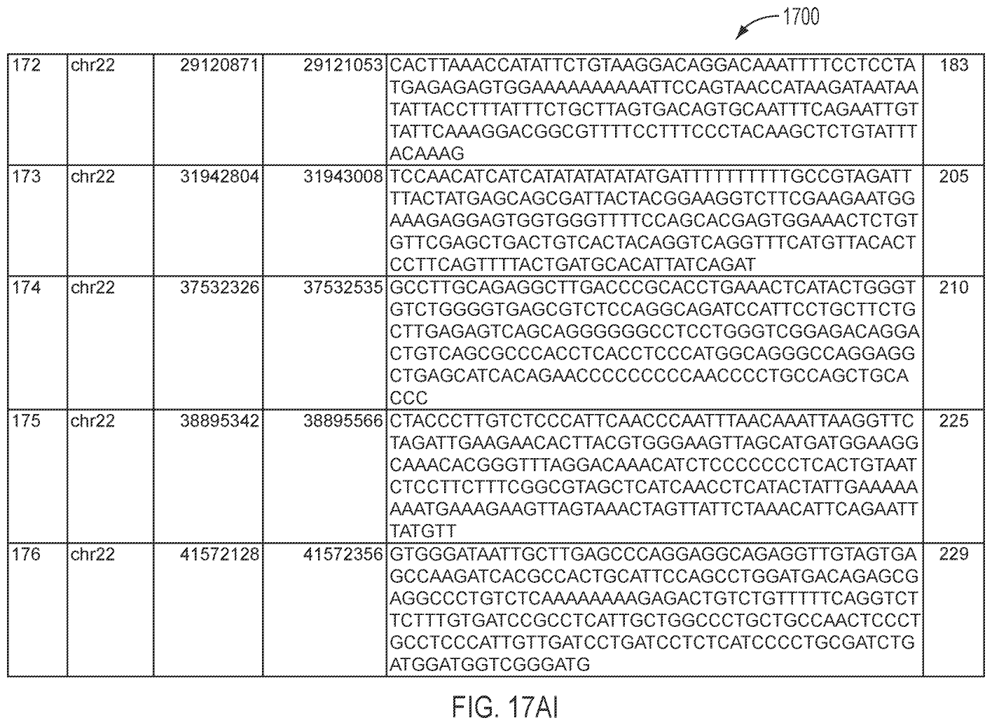

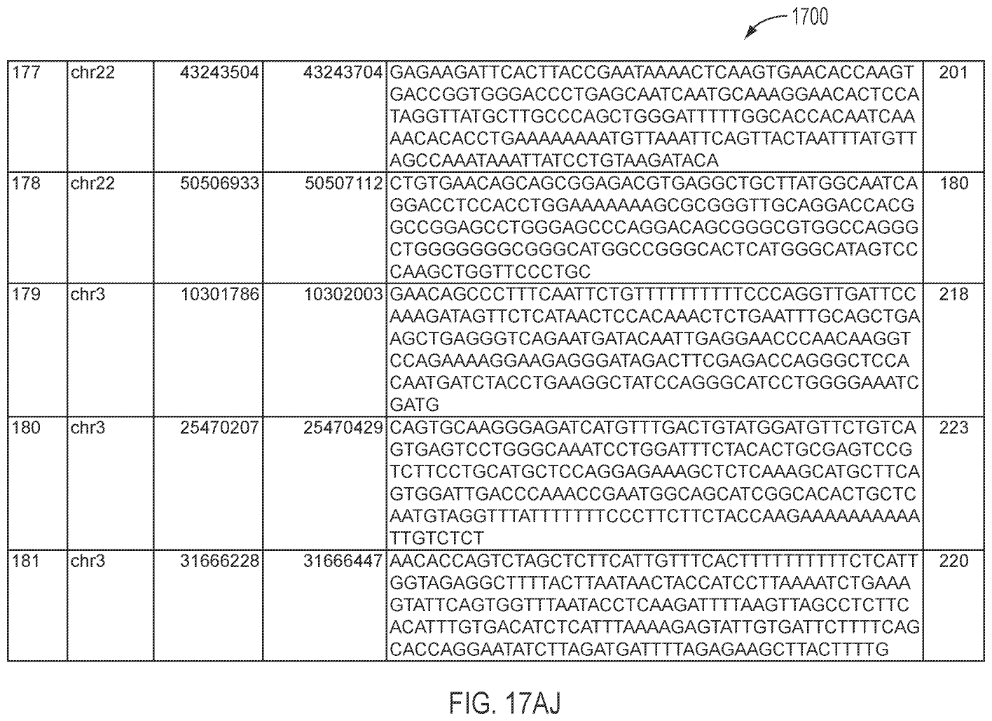

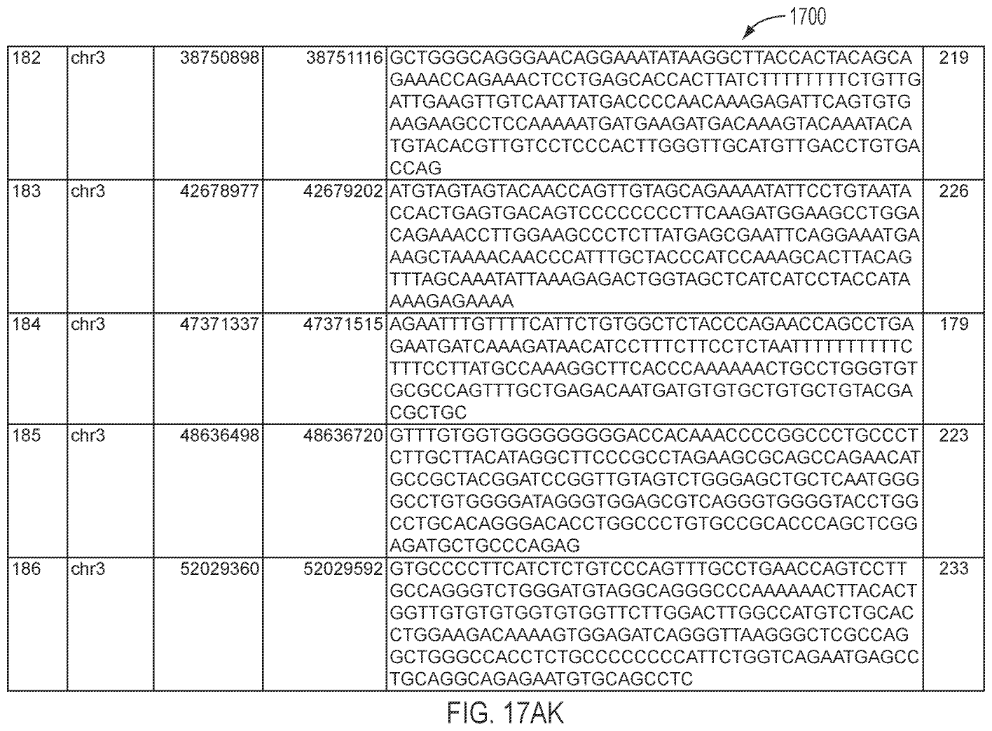

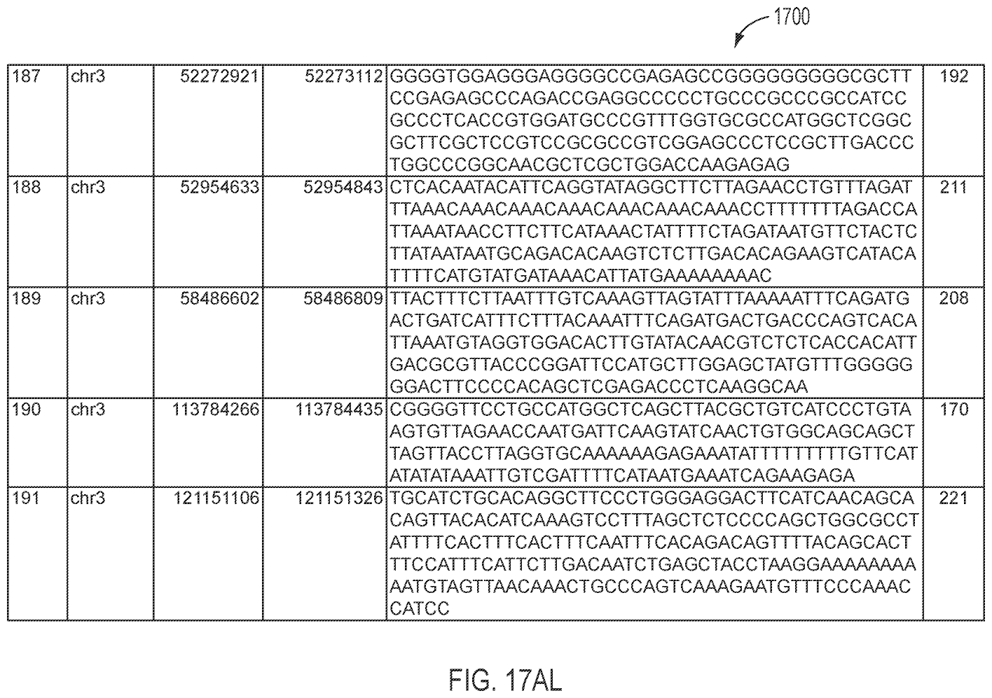

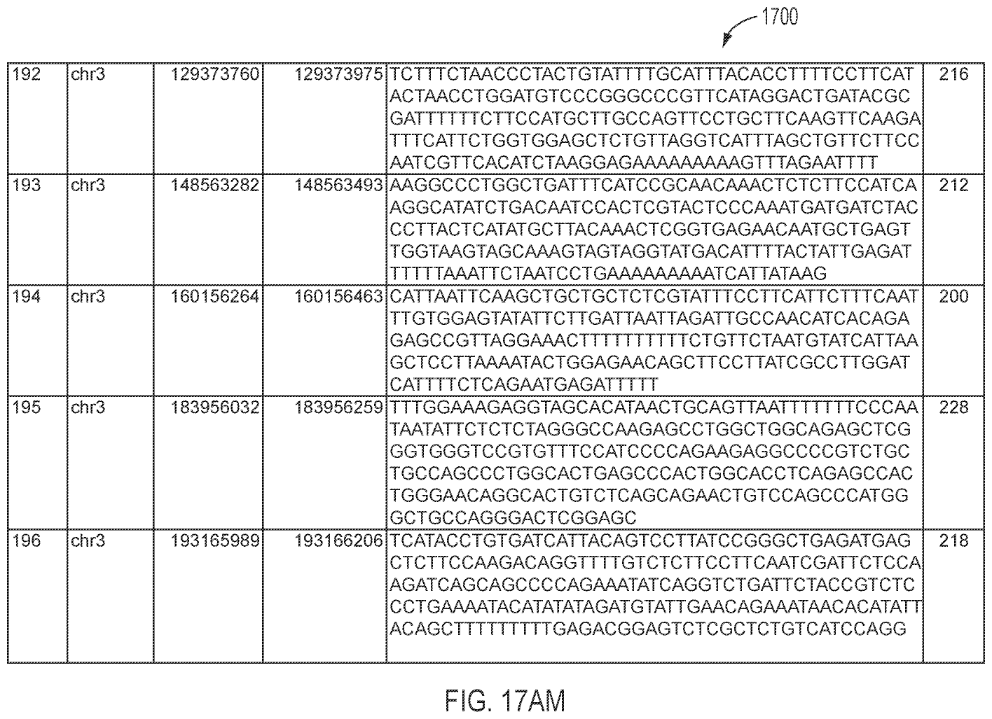

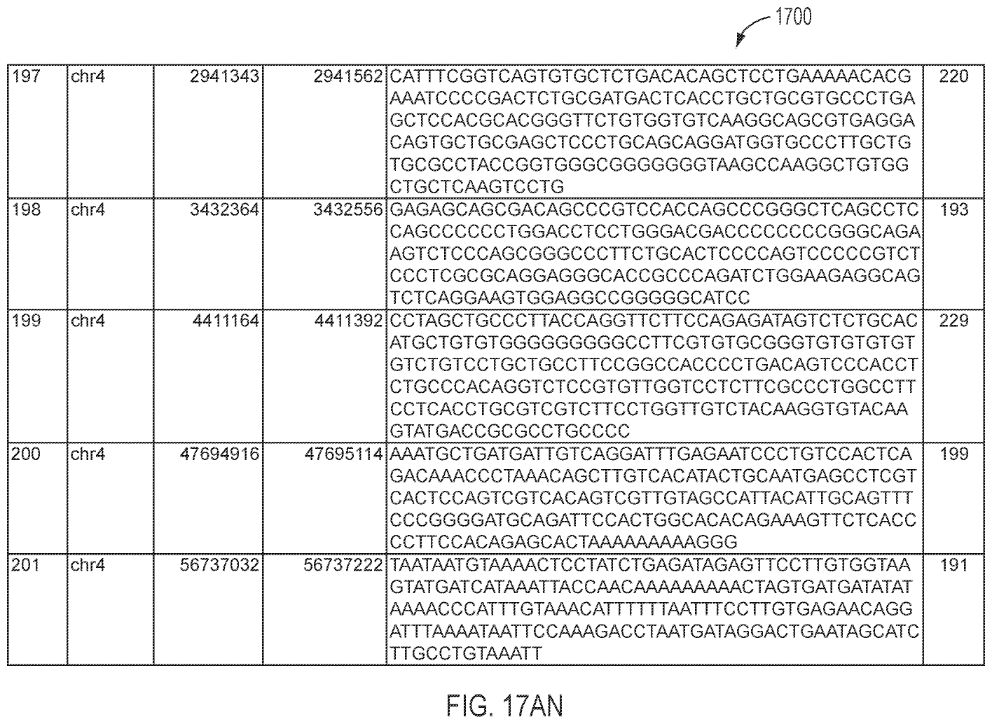

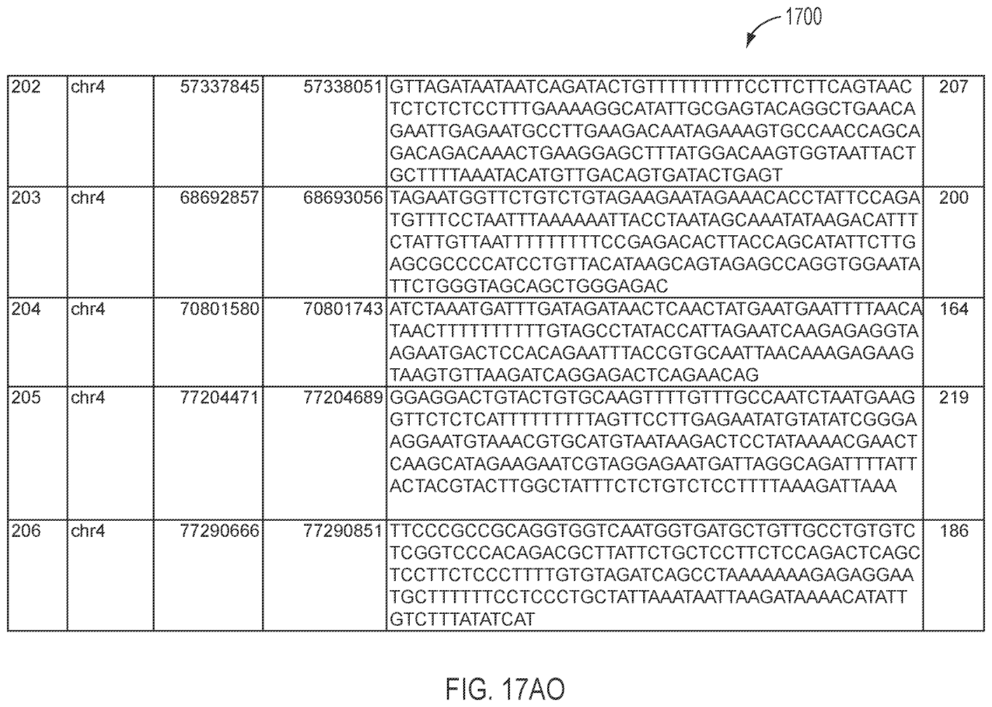

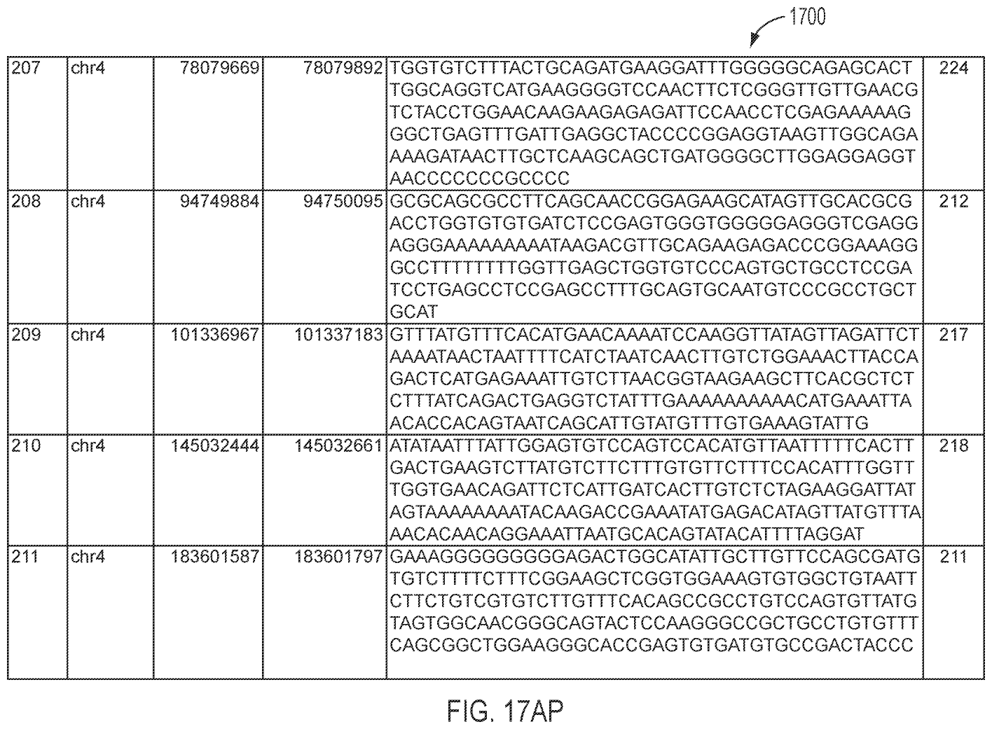

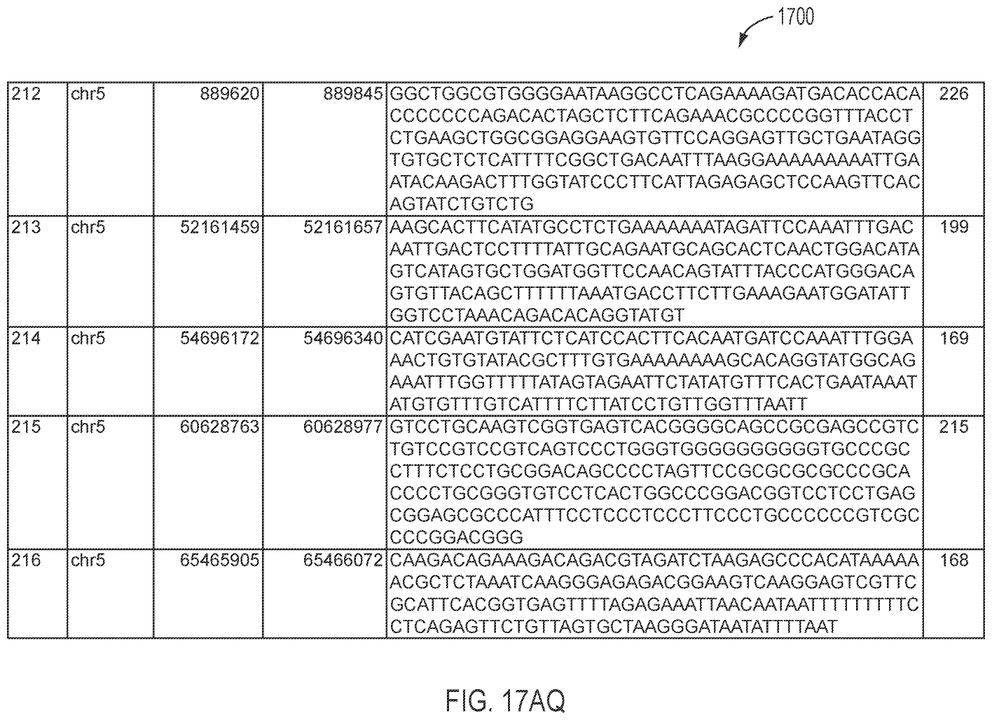

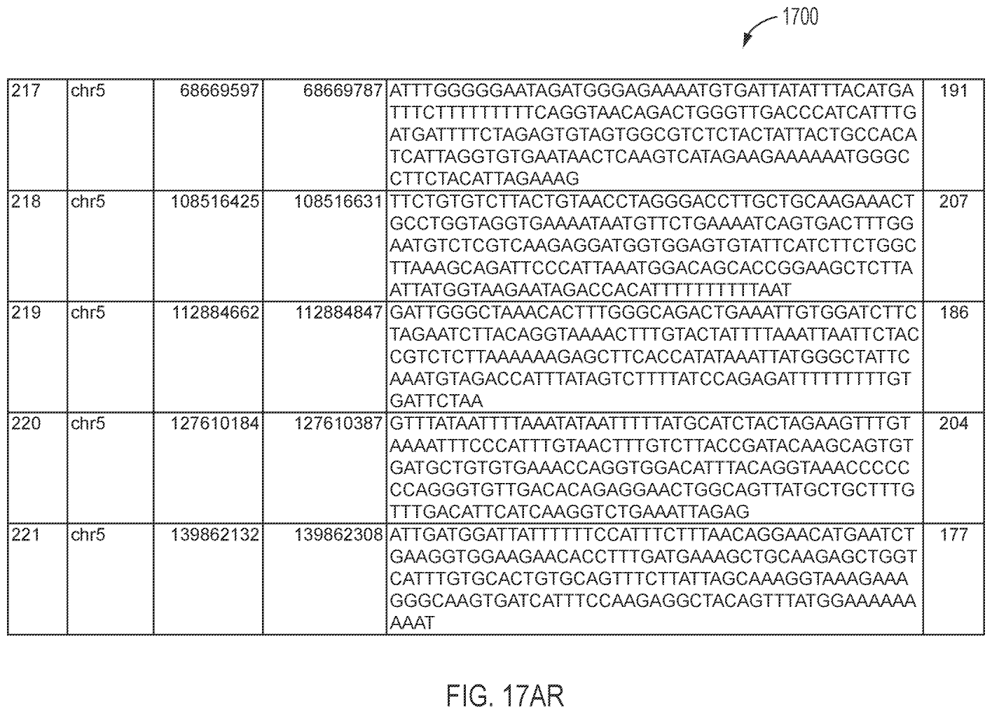

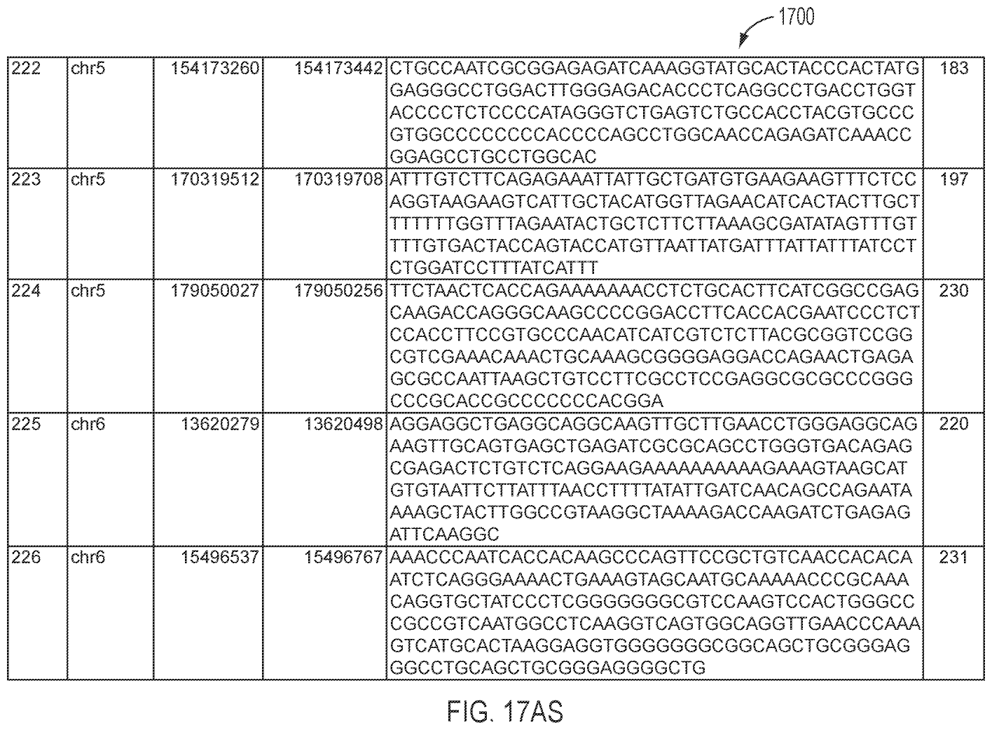

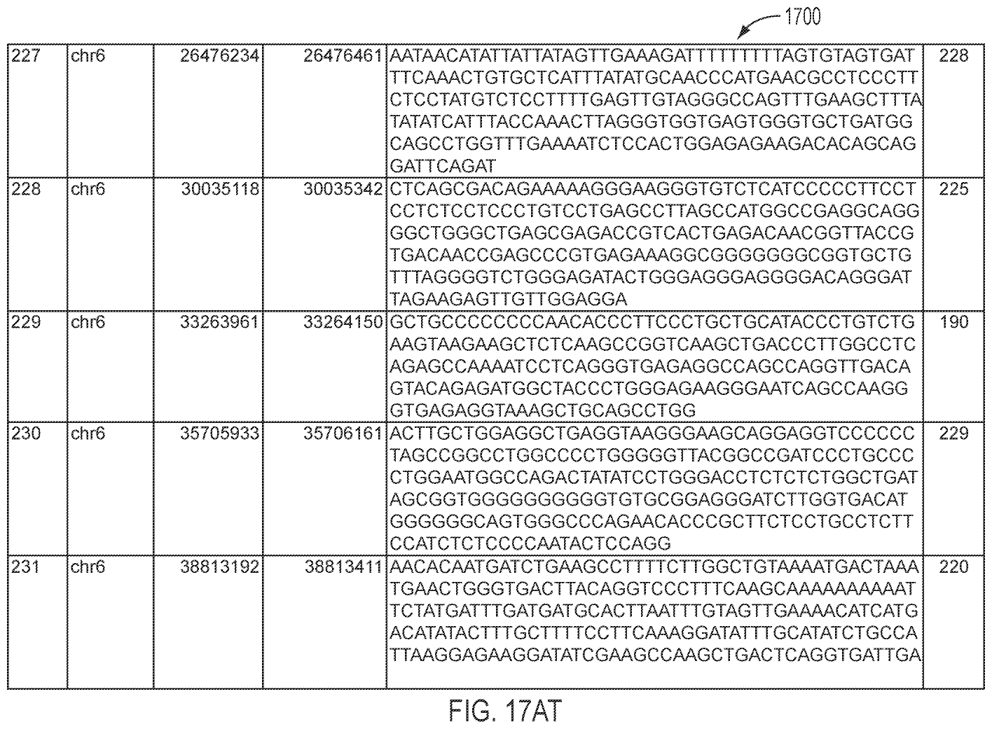

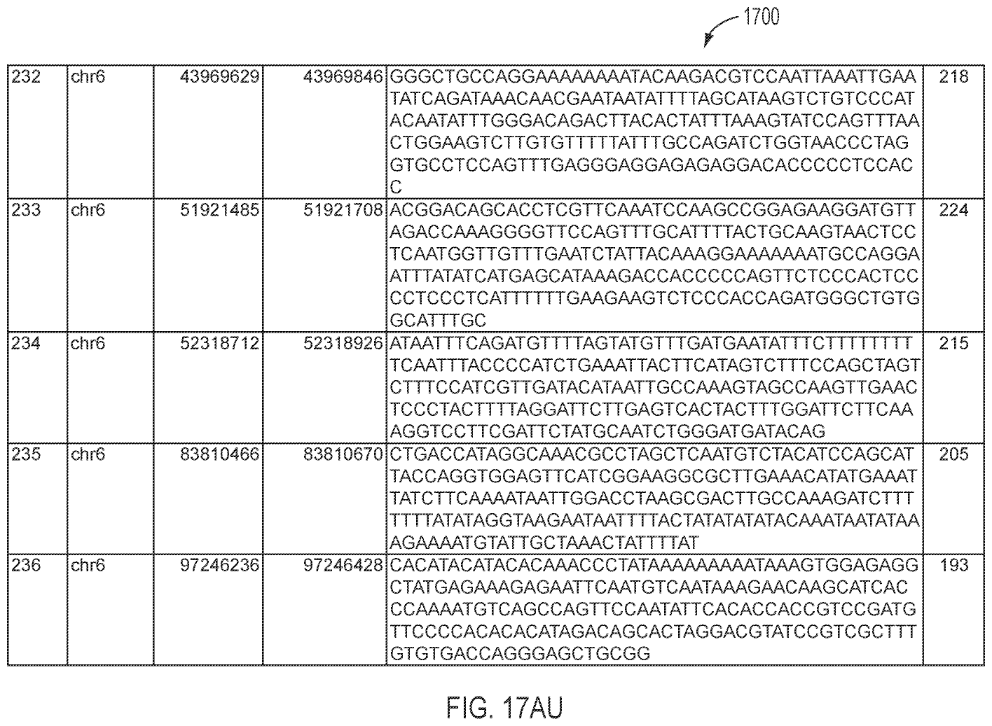

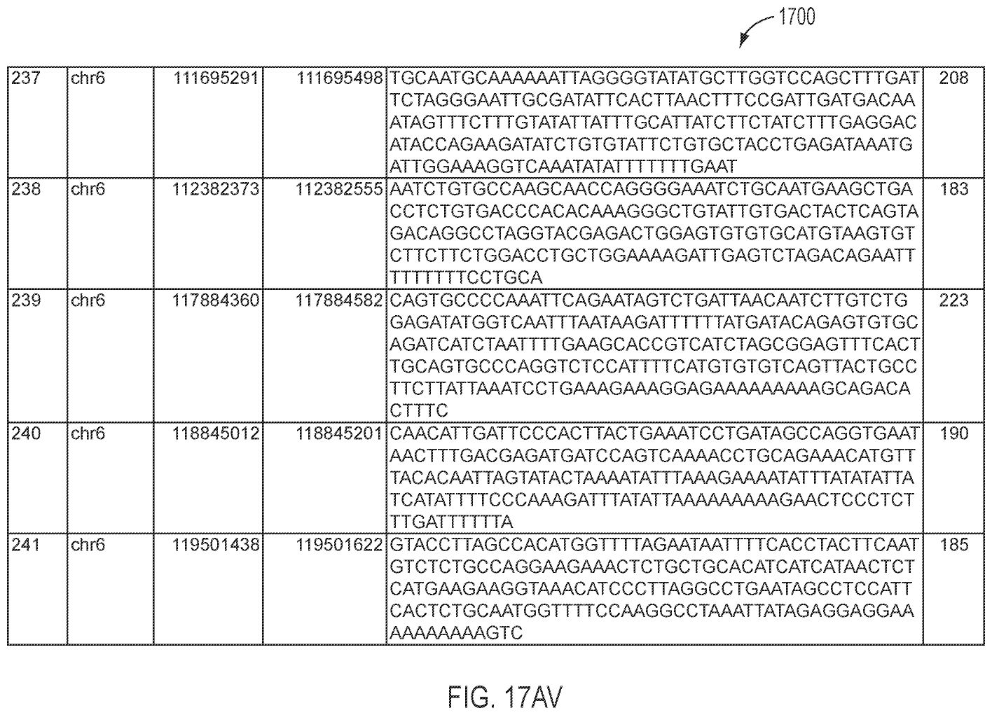

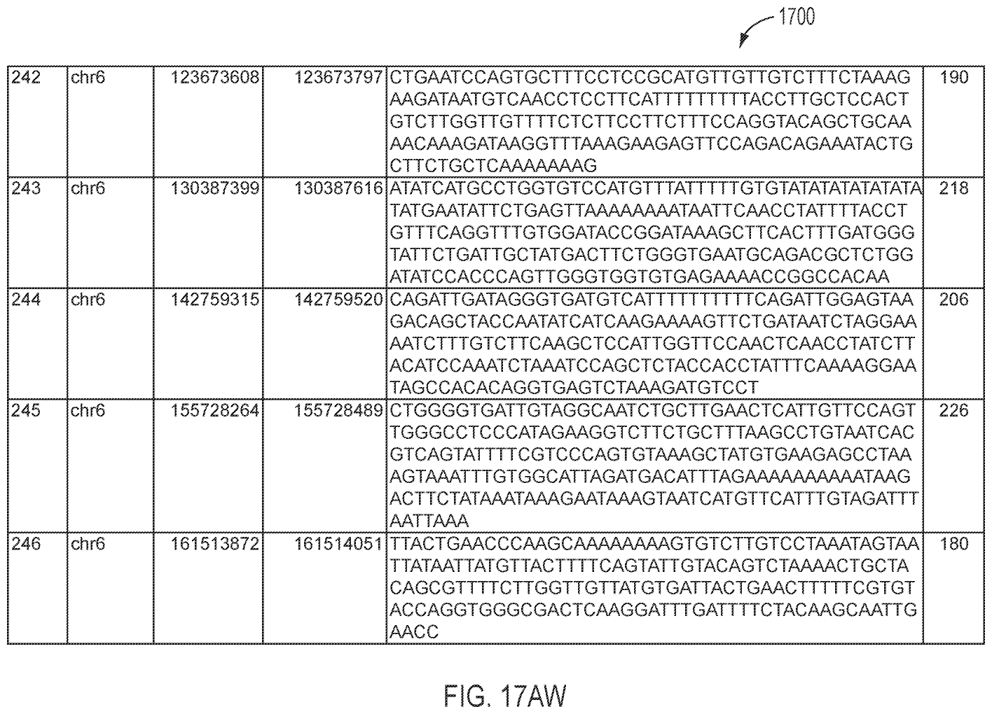

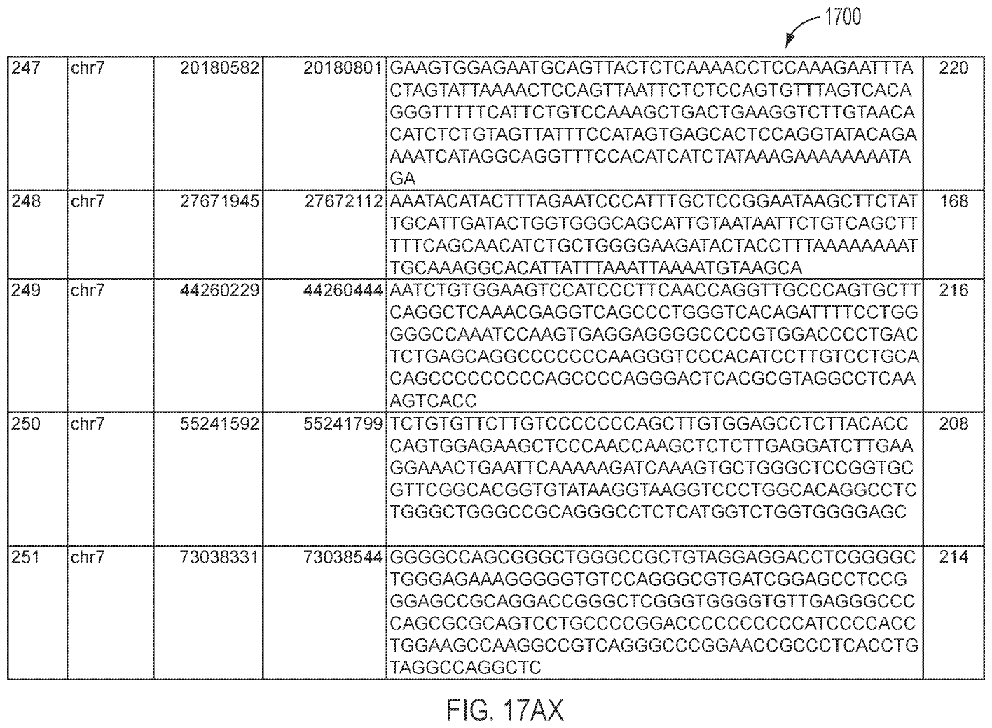

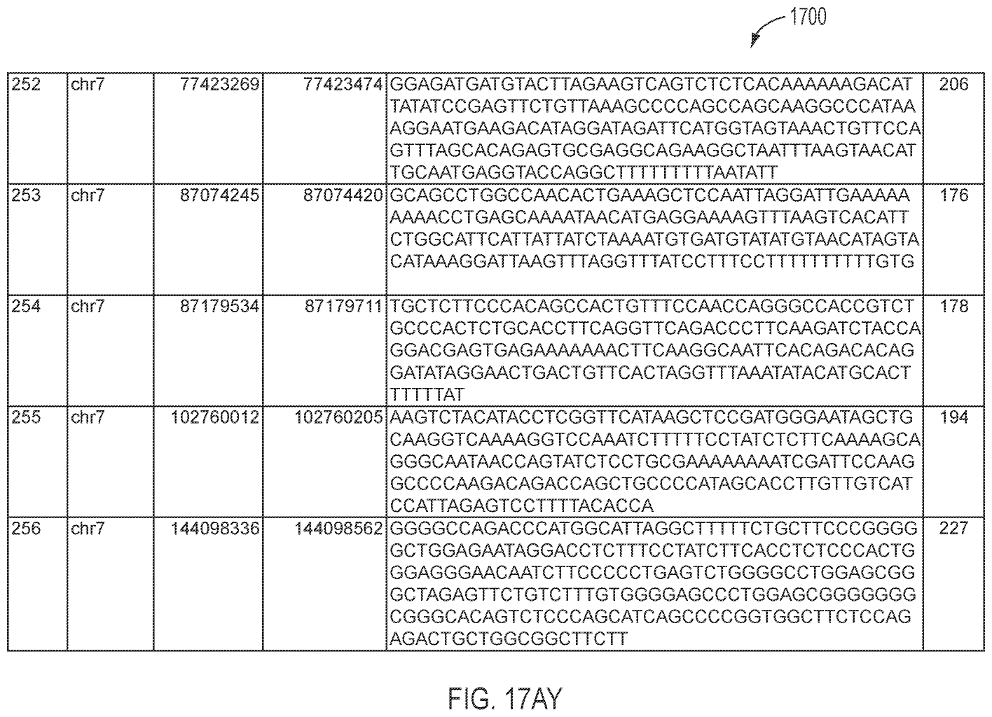

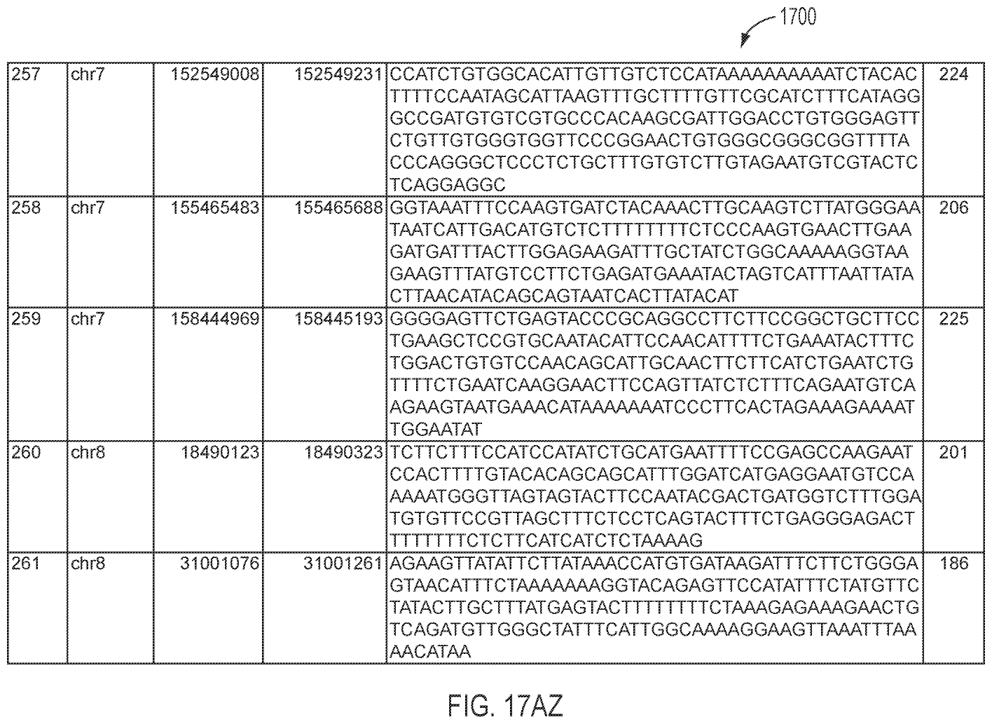

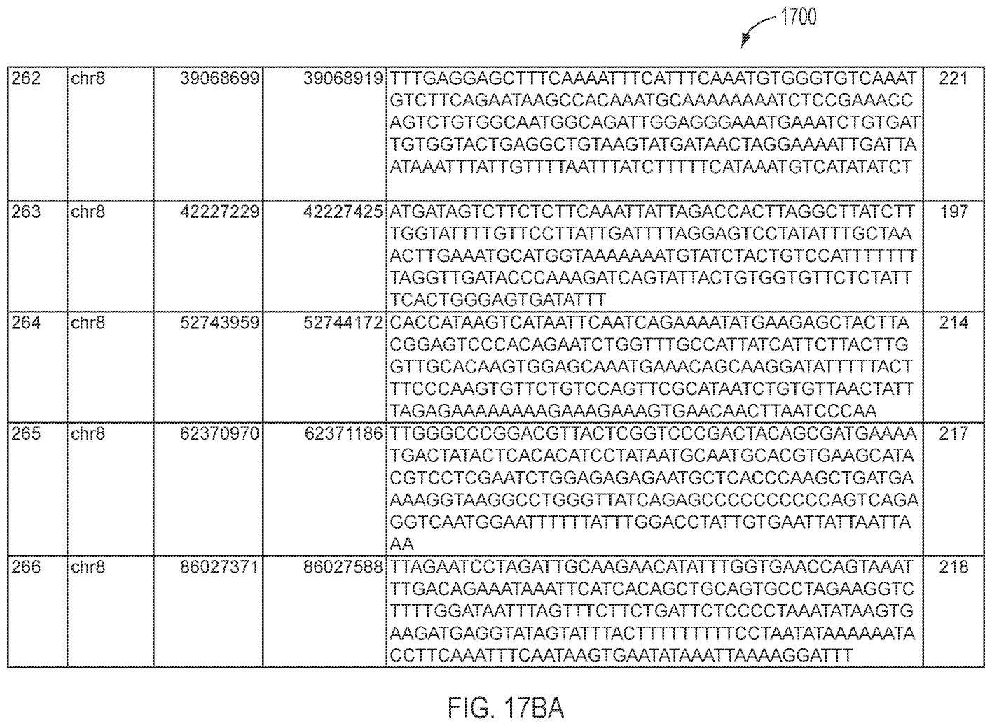

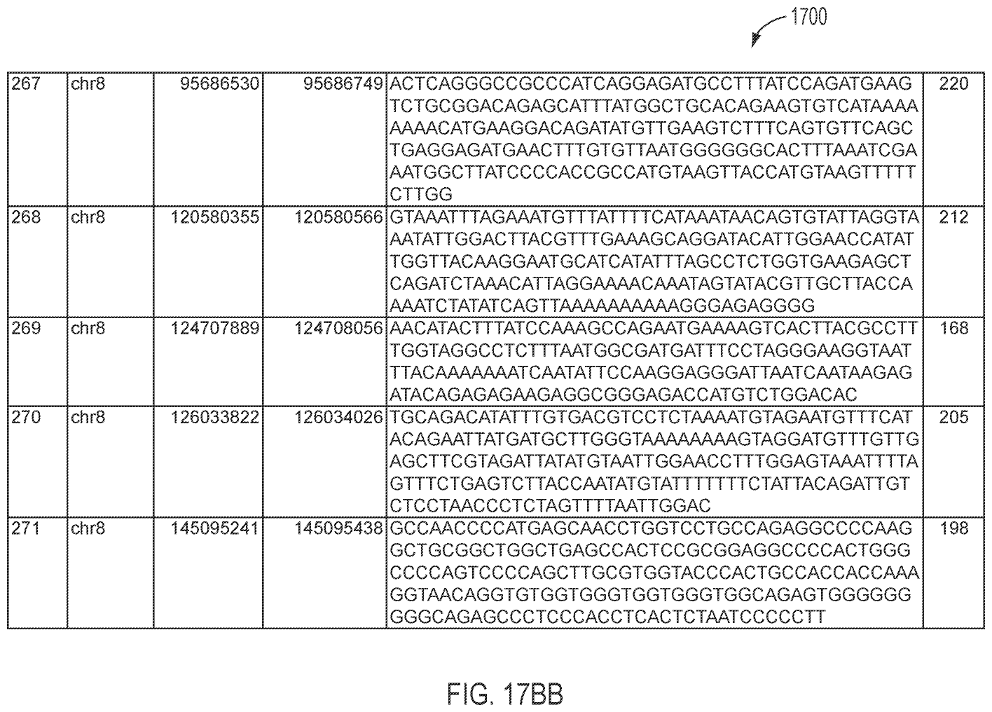

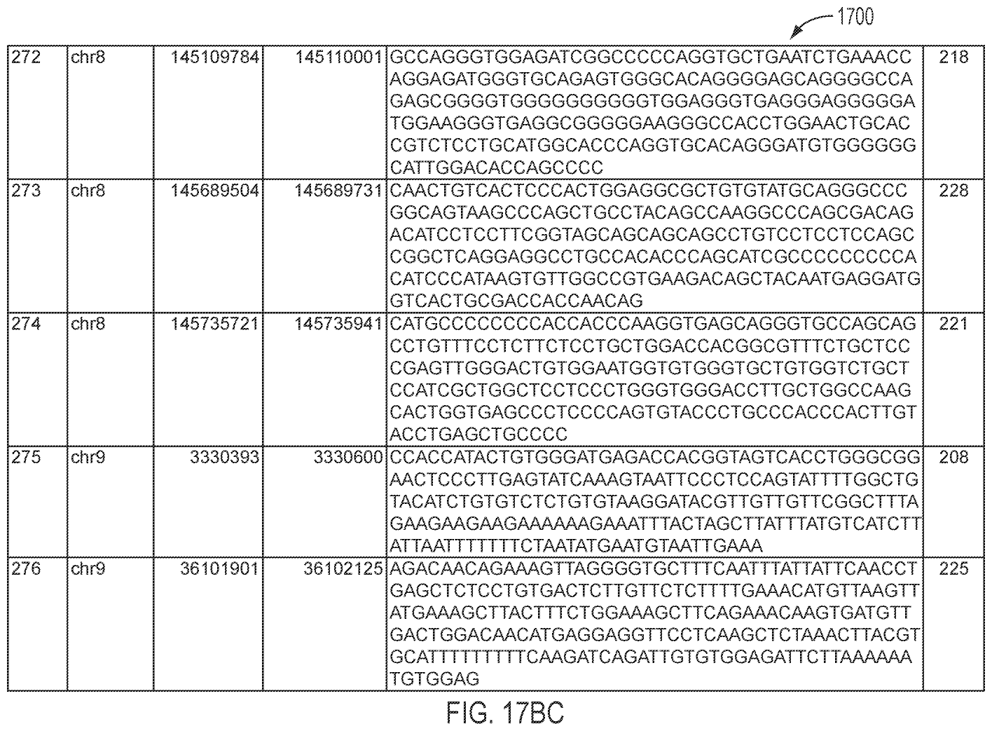

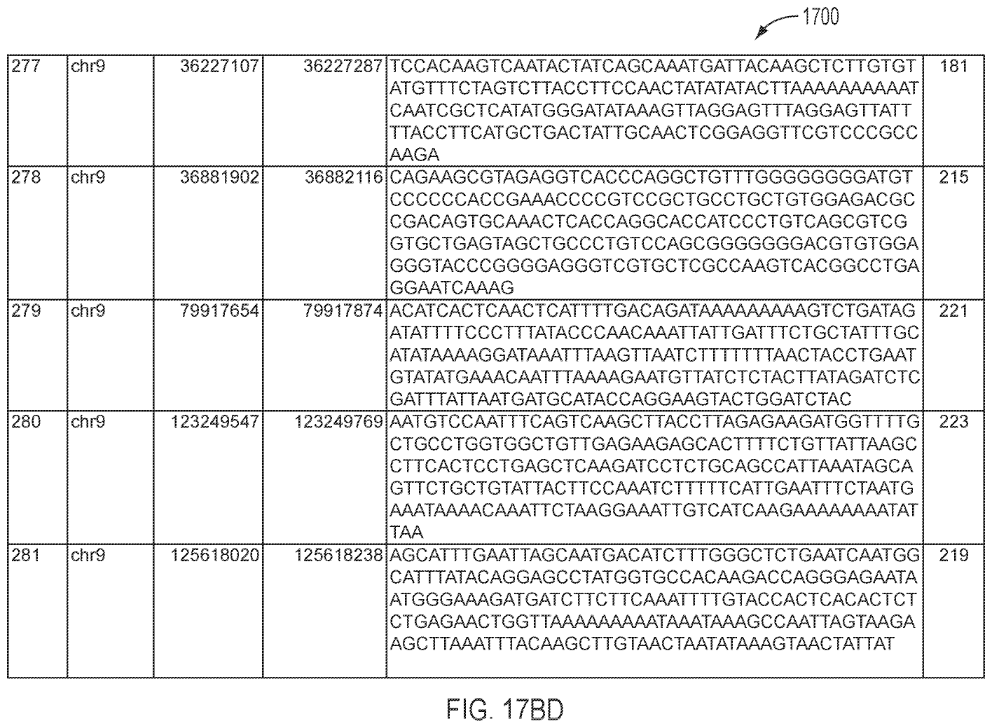

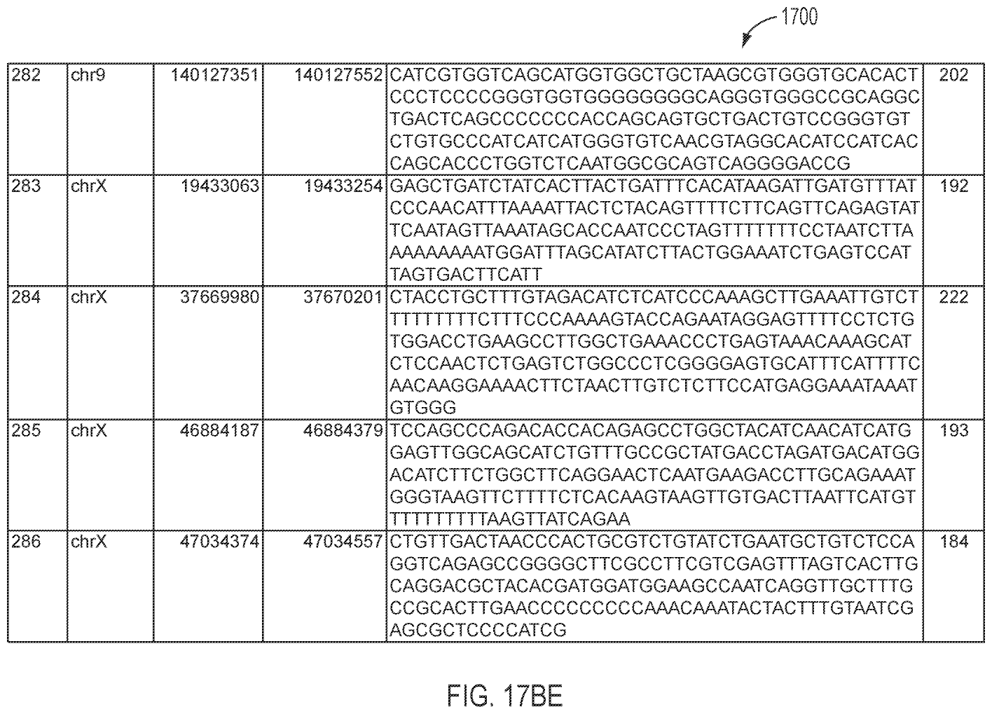

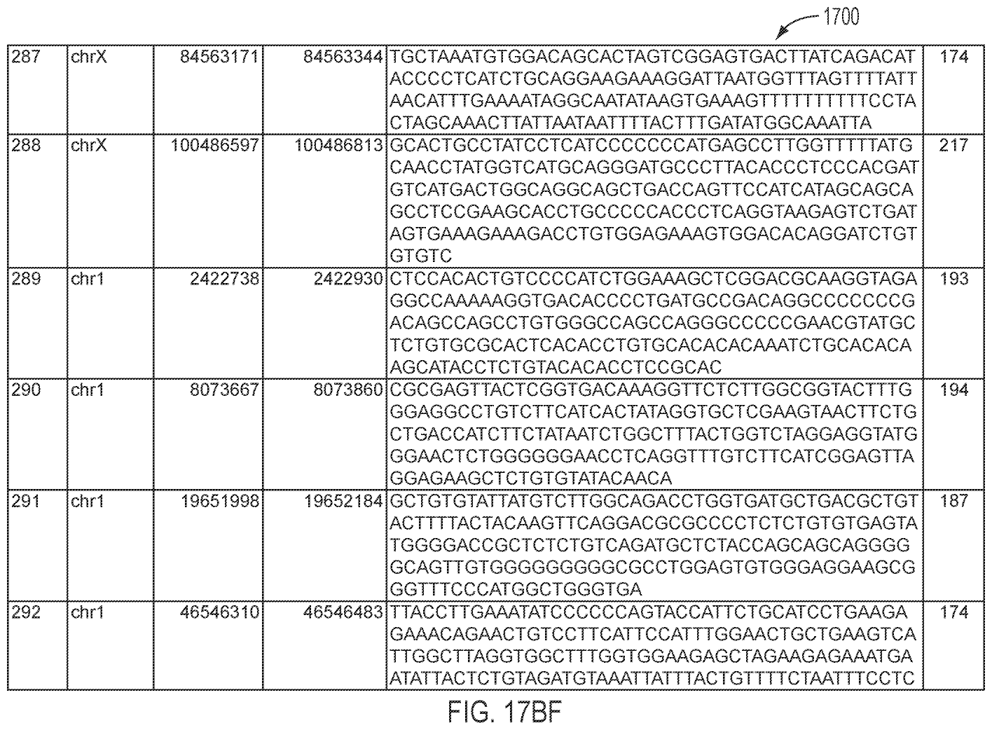

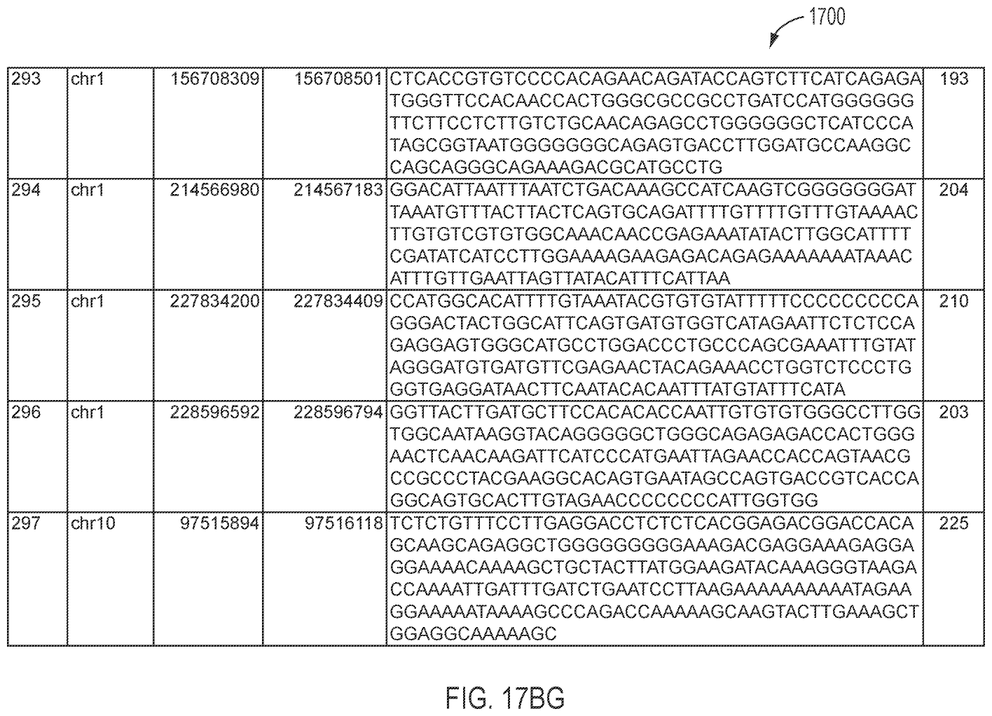

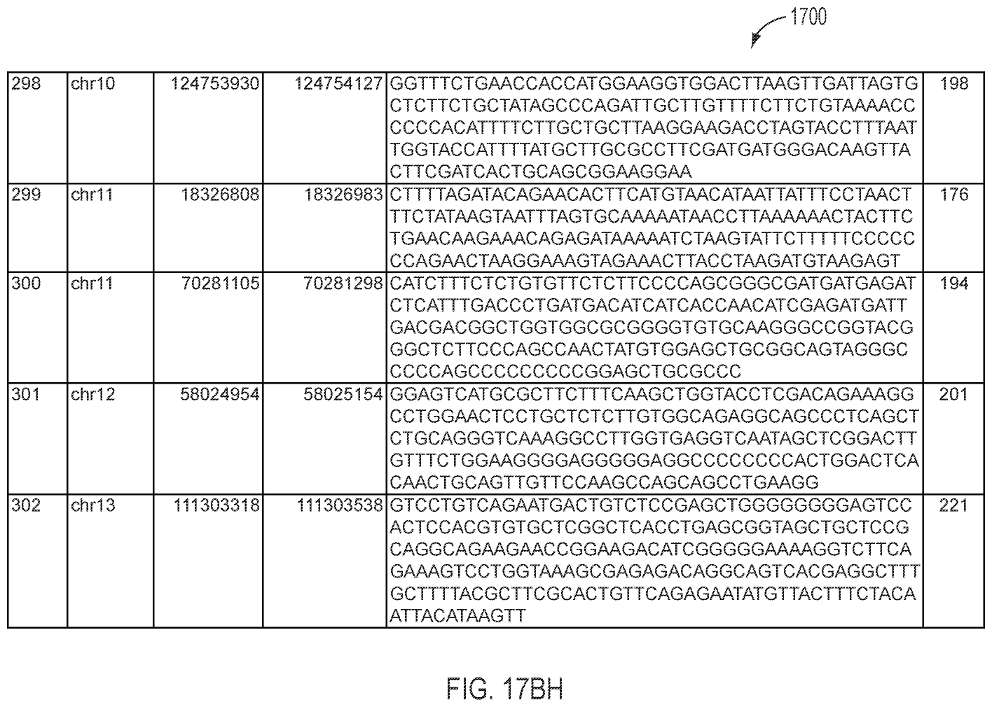

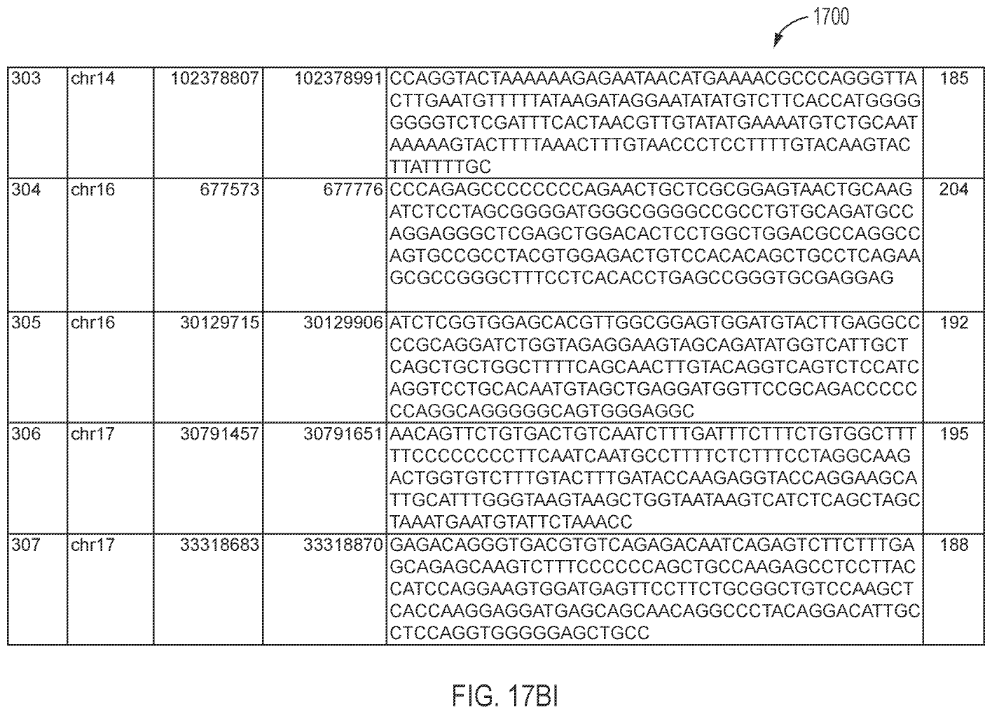

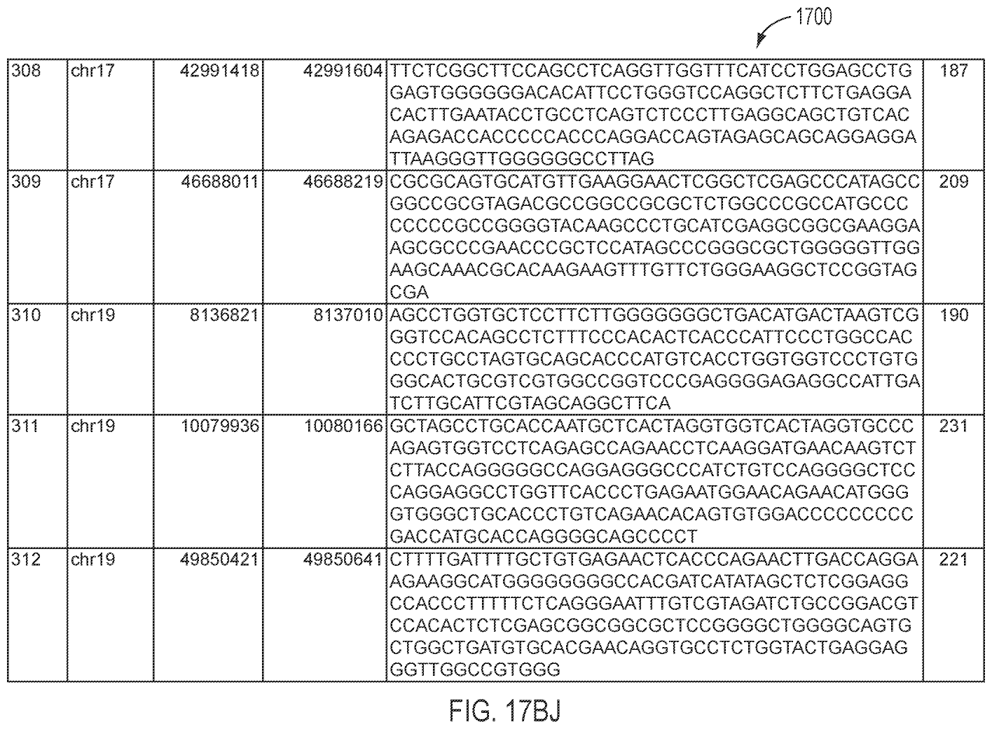

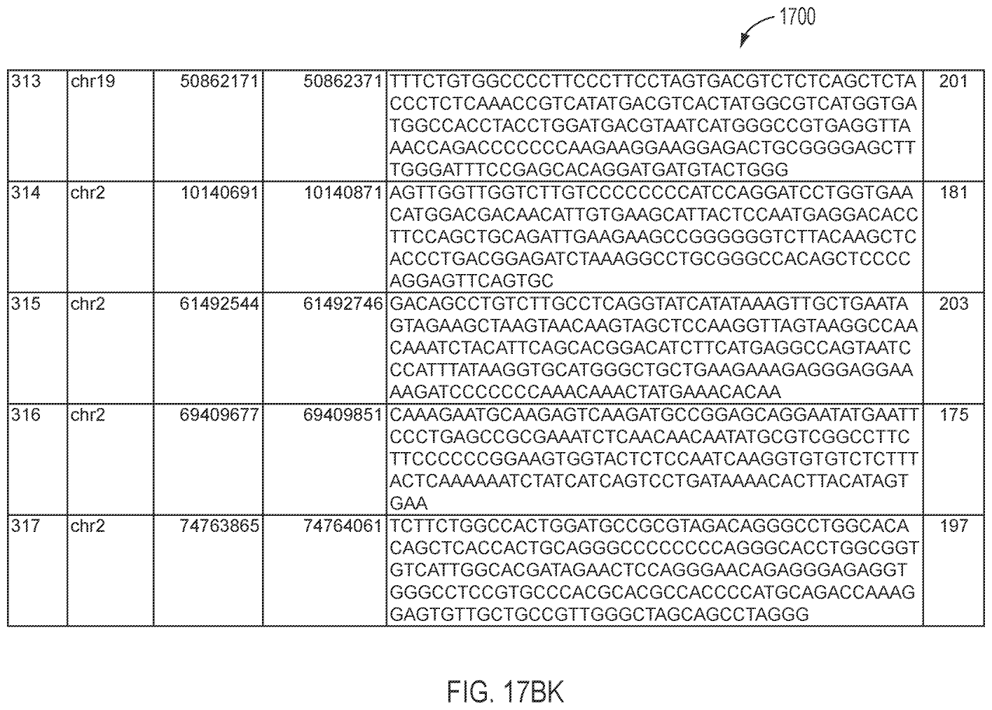

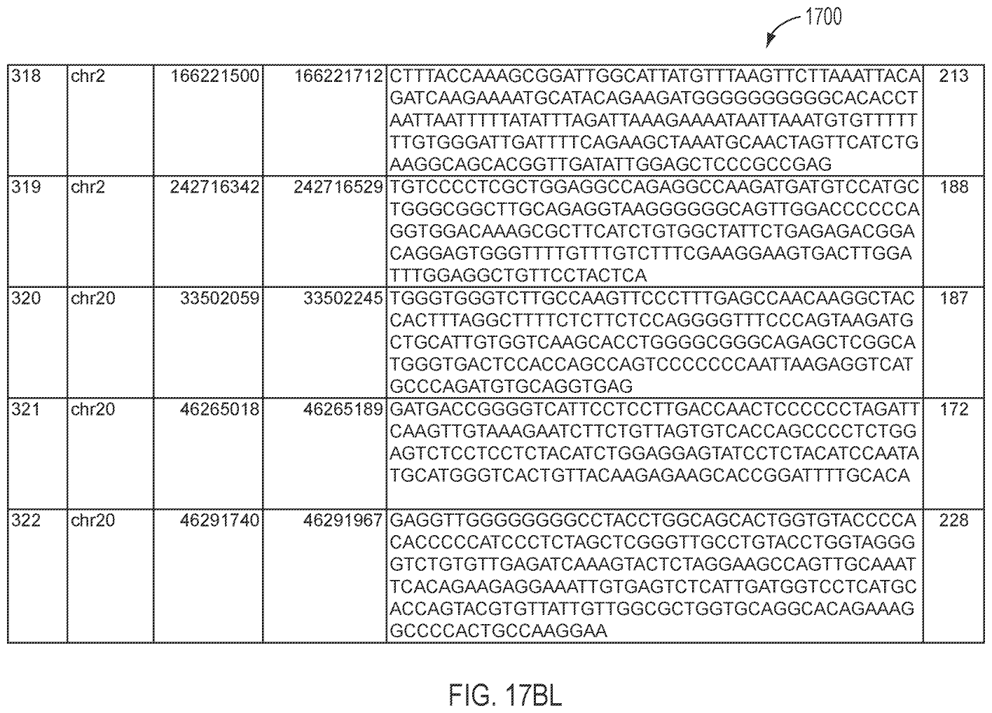

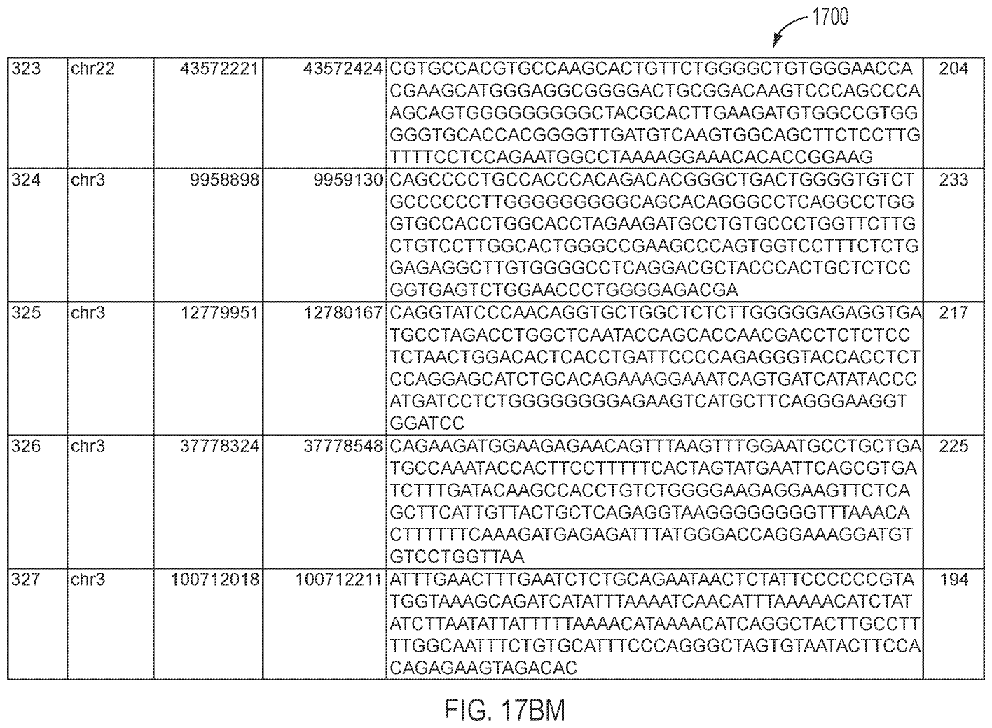

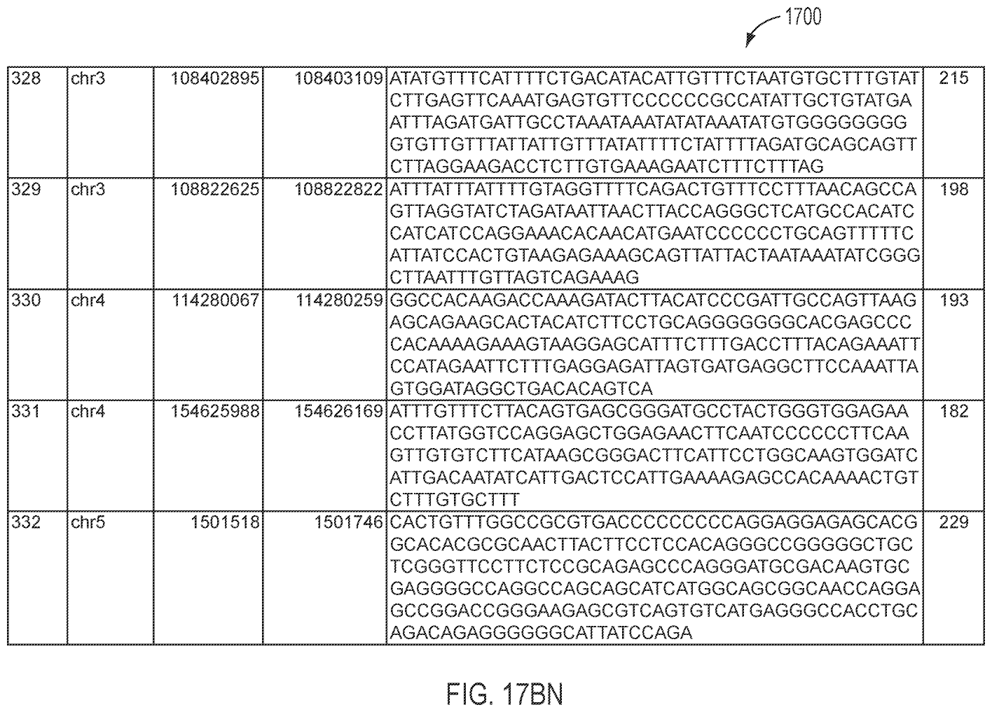

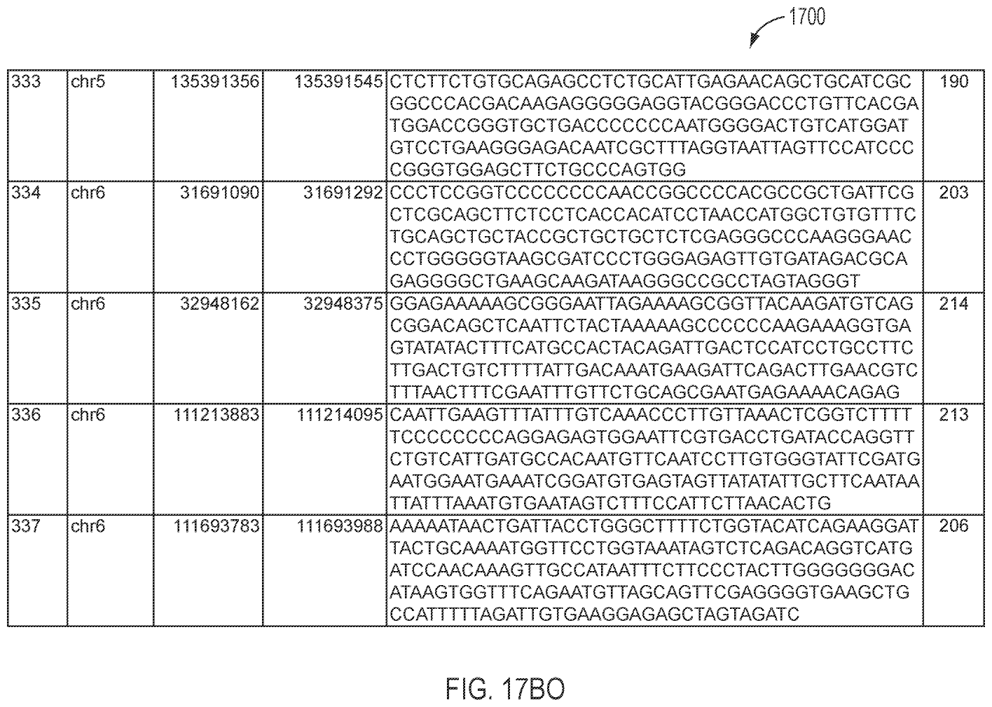

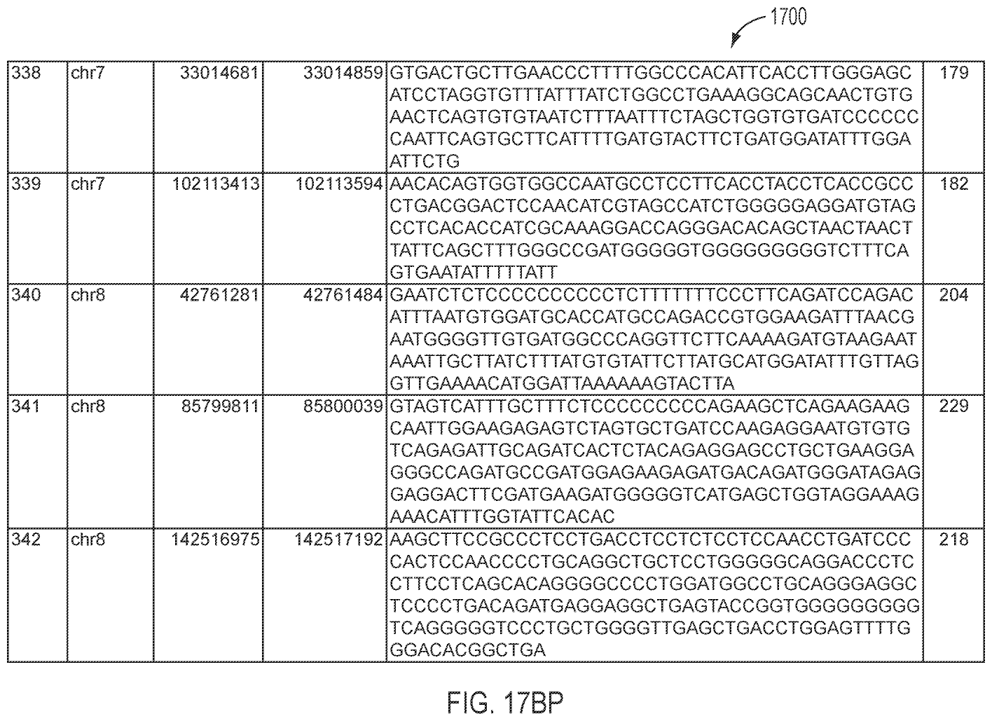

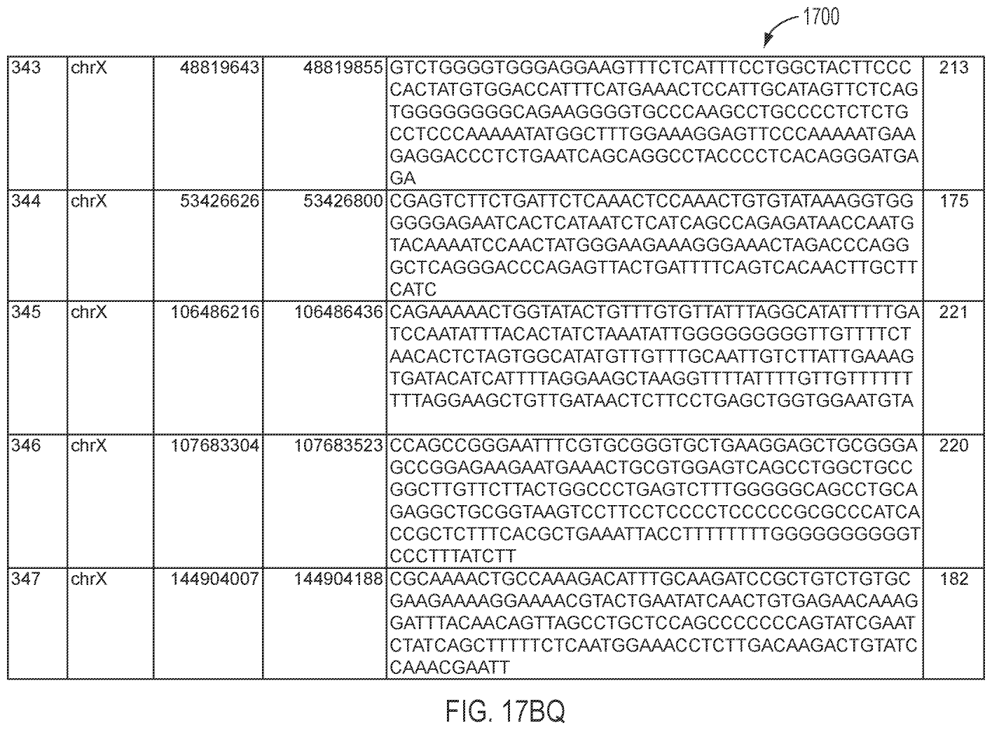

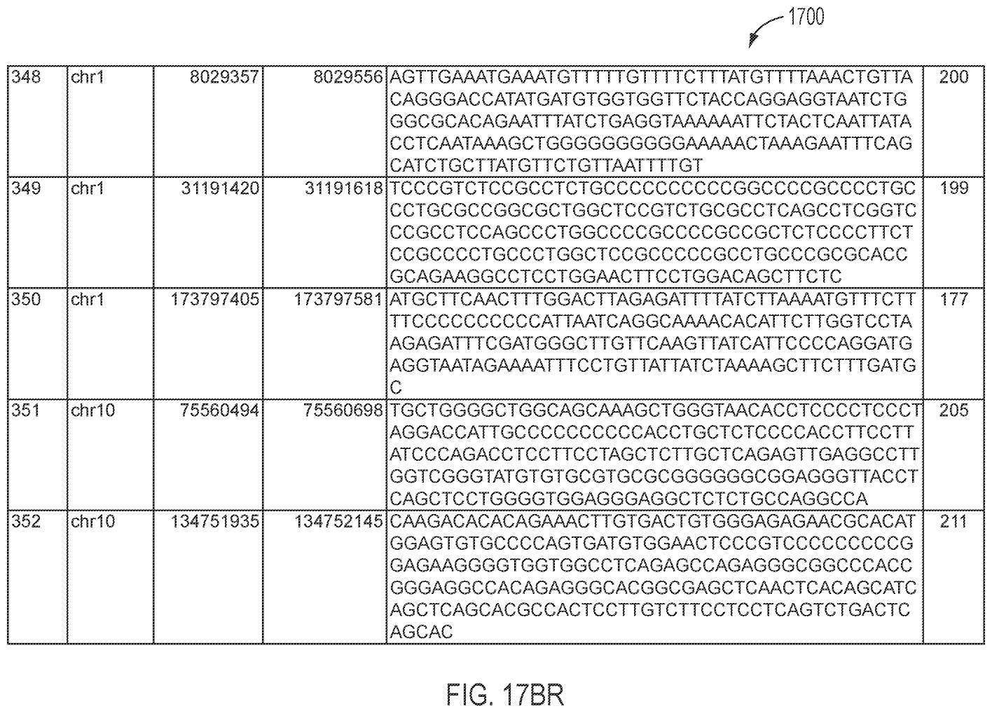

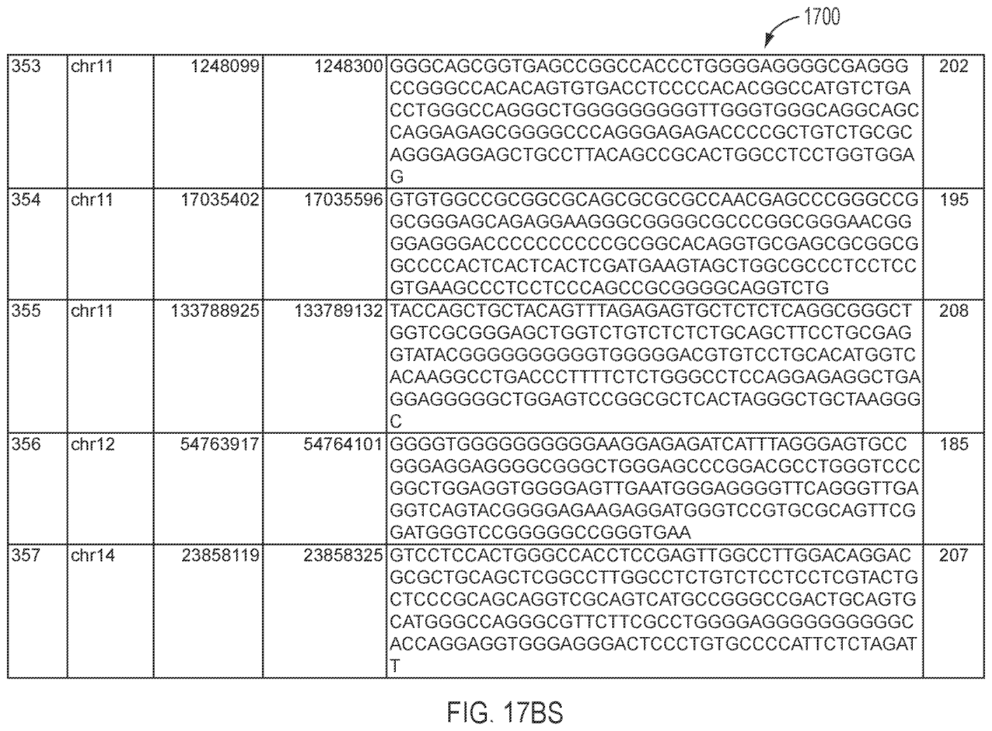

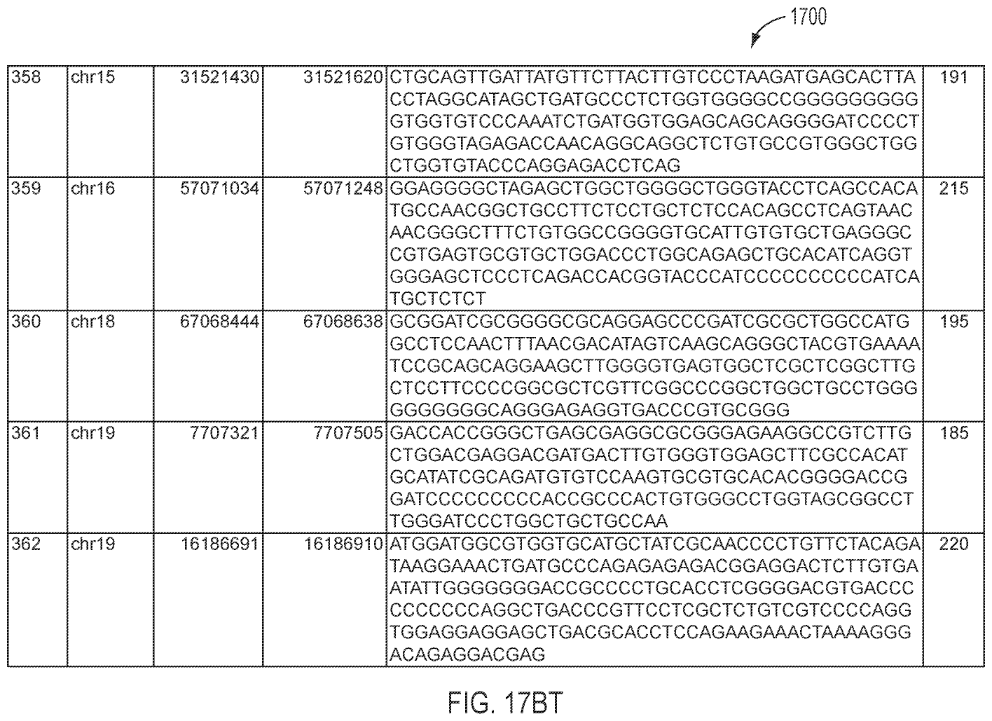

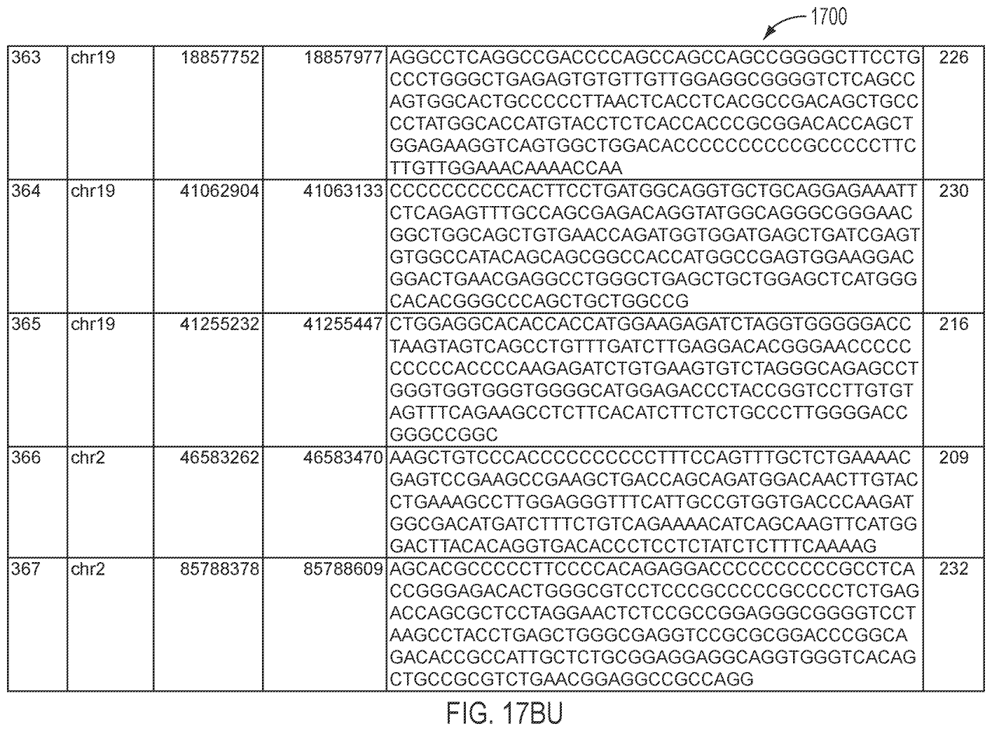

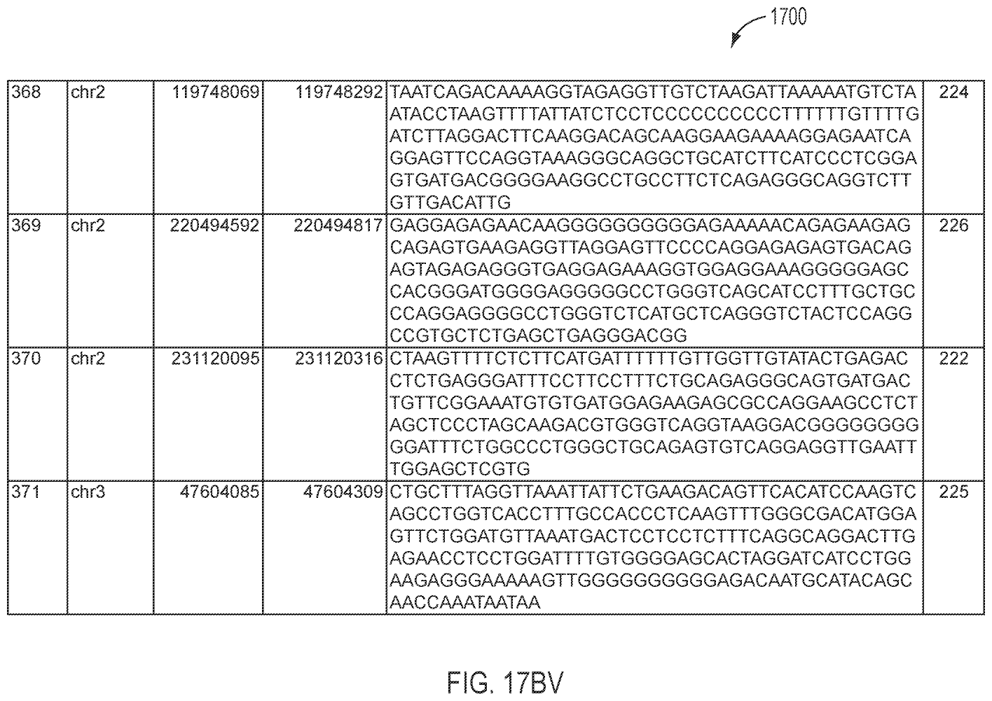

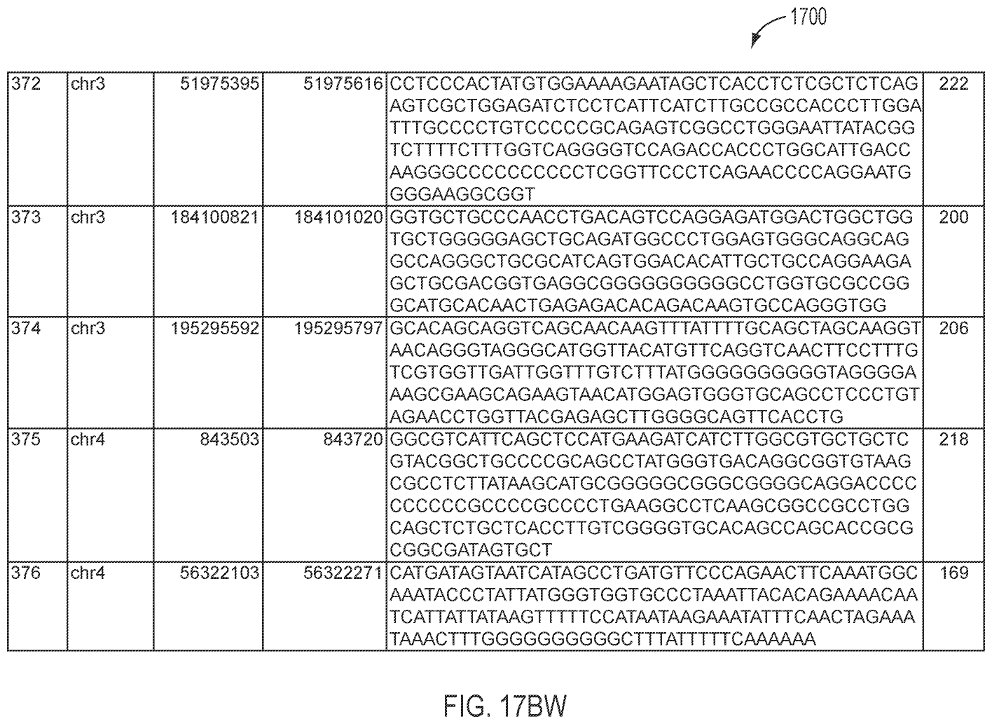

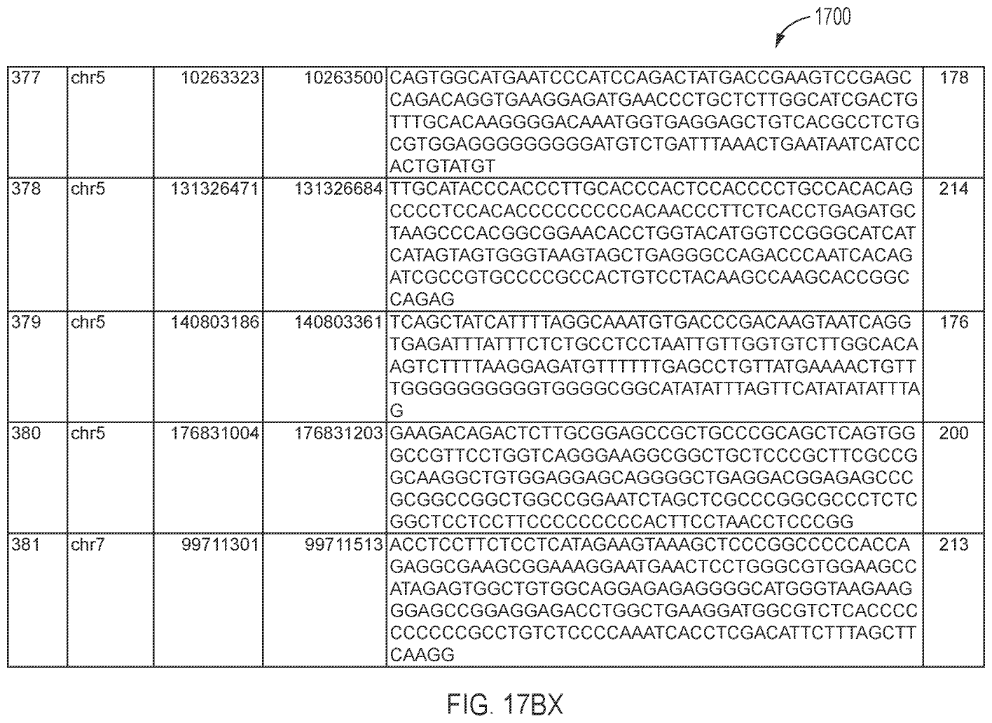

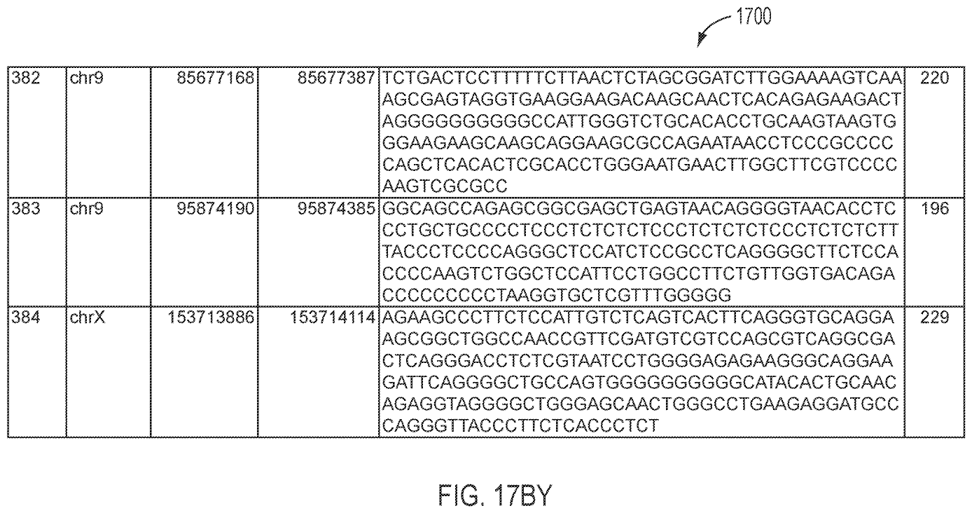

FIG. 8 shows homopolymer representation statistics for an exemplary recalibration panel 800. The upper table includes exemplary homopolymer compositions of amplicons in the exemplary recalibration panel 800. The amplicons shown in the upper table may represent a subset of amplicons on the Ion AmpliSeq™ Exome Panel. The “base” group of amplicons may represent selected amplicons that are inside NIST NA12878 high-confidence regions with no variants and reasonably high sequencing quality that further satisfy one or more of the following exemplary constraints: (1) only one n-mer (where n is 6 or more) may be allowed on a given template; (2) a minimal separation of 7 bases between n-mers, where n is 4 or more; and (3) no overlapping between the amplicons and/or non-mers longer than 10-mers, with a few exceptions (e.g., several G or C n-mers can be on the same strand if they are separated by at least 7 bases and at least 3 bases from A/T n-mers). An exemplary reference genome in addition to NIST NA12878 may include NIST RM 8398. The “backup” group of amplicons may represent additional amplicons with higher sequencing quality (including mostly G/C n-mers). The “extra 9GC_10GC” group of amplicons may represent additional amplicons with 9G/9C/10G/10C n-mers. The lower table includes an estimation of sequencing quality, e.g., based on a good two-exome run on an Ion Proton™ system implementing Ion Torrent™ sequencing technology. FIGS. 17A-17Z, 17AA-17AZ, and 17BA-17BY include table 1700, which lists the chromosome, insert start, insert end, and insert for each of the 384 amplicons discussed in FIG. 8. Among these, inserts 1 through 288 may correspond to the “base” group; inserts 289 through 347 may correspond to the “backup” group, and inserts 348 through 384 may correspond to the “extra_9GC_10GC” group.

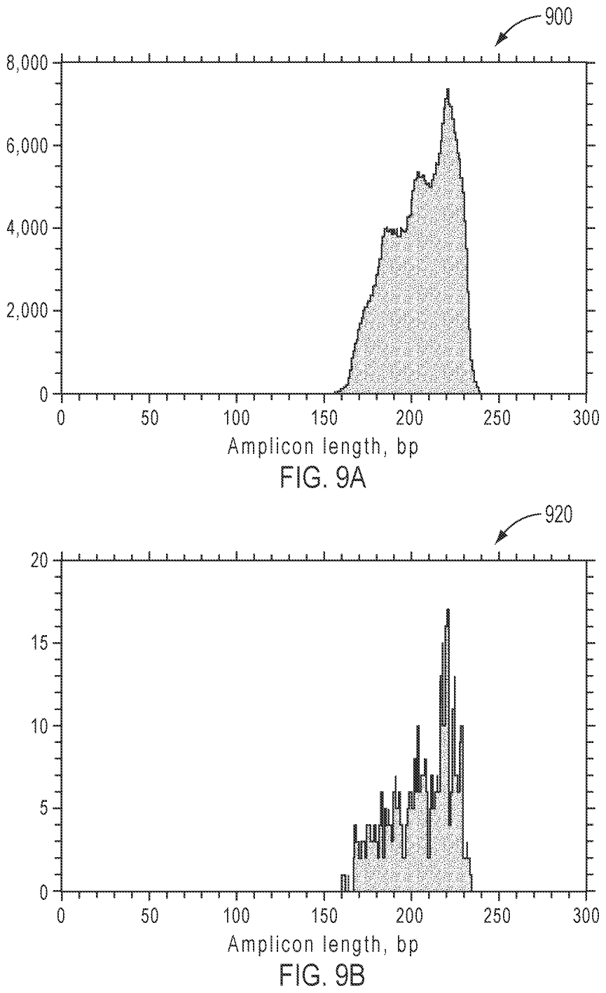

FIGS. 9A and 9B illustrate exemplary distributions of amplicon length for a two-exome sequencing run. FIG. 9A includes an exemplary distribution 900 of amplicon length for a full AmpliSeq™ Exome Panel run (e.g., ½ of an Ion Proton™ system run implementing Ion Torrent™ sequencing technology). FIG. 9B includes an exemplary distribution 920 of amplicon length for the subset of 384 amplicons discussed in FIG. 8 and listed in FIGS. 17A-17Z, 17AA-17AZ, and 17BA-17BY.

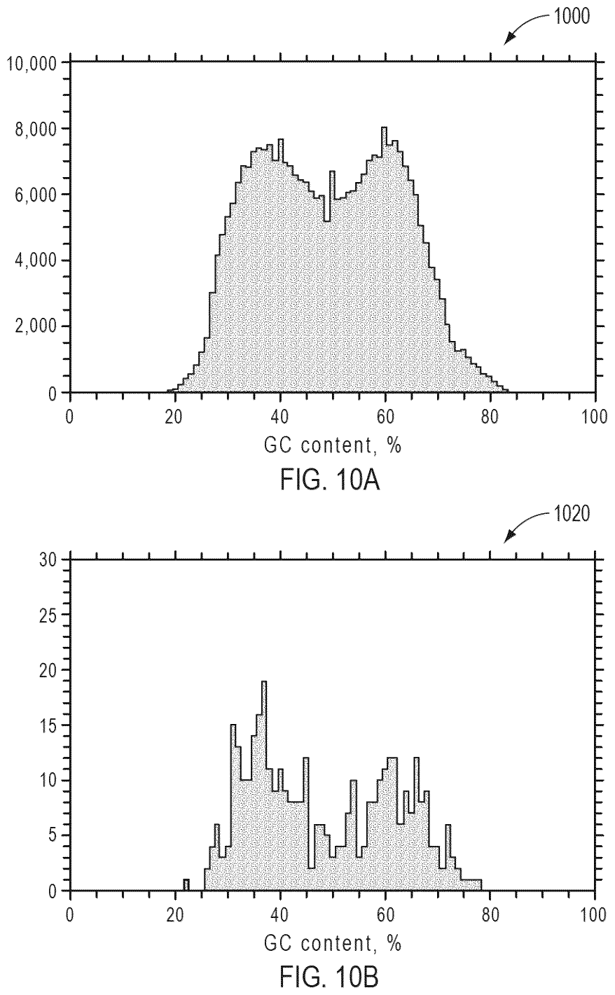

FIGS. 10A and 10B show exemplary distributions of GC content for a two-exome sequencing run. FIG. 10A includes an exemplary distribution 1000 of GC content for the full AmpliSeq™ Exome Panel run (e.g., half of an Ion Proton™ system run implementing Ion Torrent™ sequencing technology). FIG. 10B shows a distribution 1020 of GC content for the subset of 384 amplicons discussed in FIG. 8 and listed in FIGS. 17A-17Z, 17AA-17AZ, and 17BA-17BY.

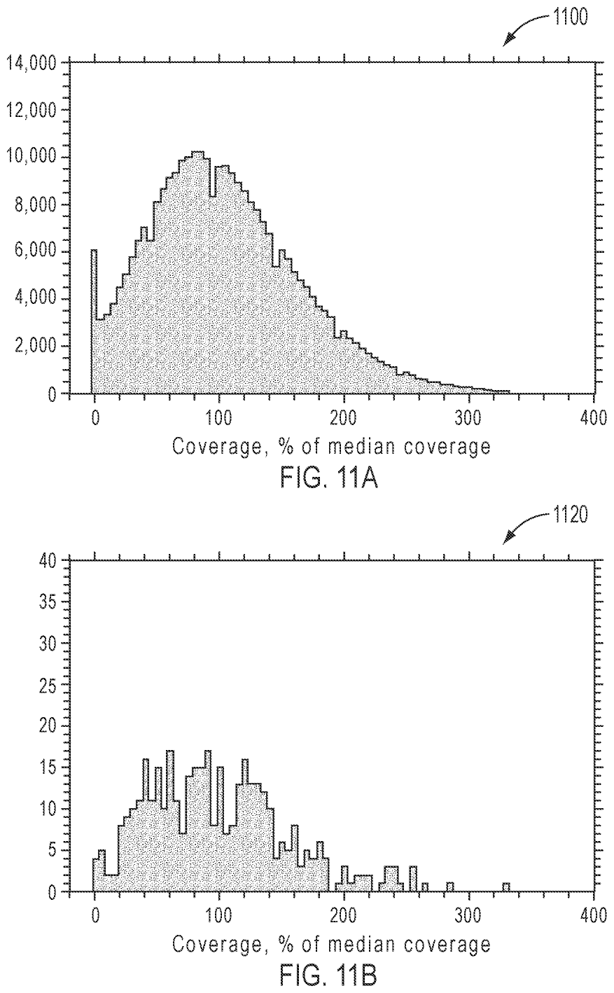

FIGS. 11A and 11B show exemplary distributions of coverage, expressed as percentage of median coverage, for a two-exome sequencing run. FIG. 11A includes an exemplary distribution 1100 of coverage, expressed as percentage of median coverage, for the full AmpliSeq™ Exome Panel run (e.g., ½ of an Ion Proton™ system run implementing Ion Torrent™ sequencing technology). FIG. 11B includes an exemplary distribution 1120 of coverage, expressed as percentage of median coverage, for the subset of 384 amplicons discussed in FIG. 8 and listed in FIGS. 17A-17Z, 17AA-17AZ, and 17BA-17BY. The leftmost peak in FIG. 11B may represent additional 10G/10C n-mers.

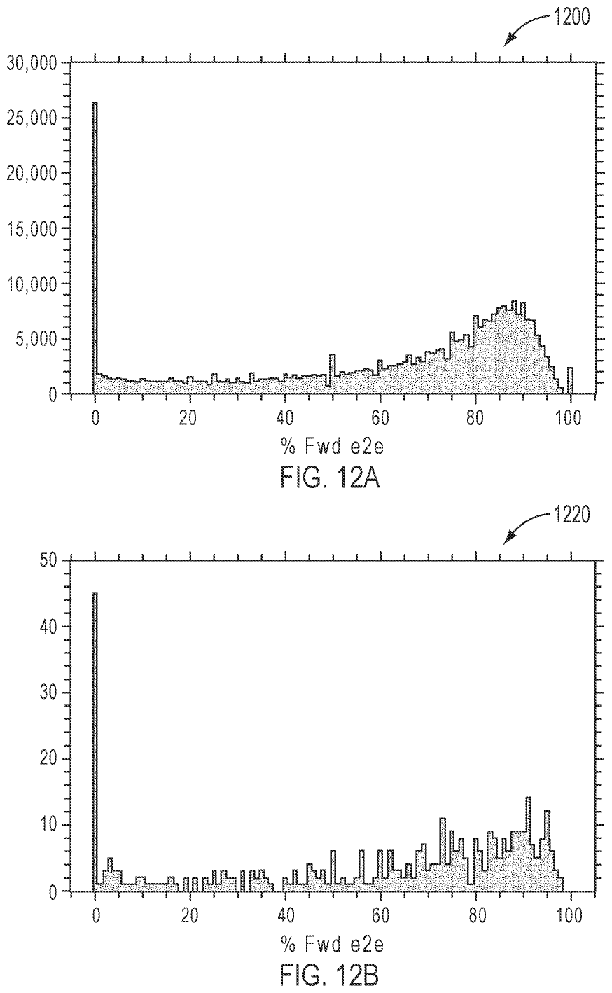

FIGS. 12A and 12B illustrate exemplary distributions of percentage forward end-to-end for a two-exome sequencing run. FIG. 12A includes an exemplary distribution 1200 of percentage forward end-to-end for the full AmpliSeq™ Exome Panel run (e.g., half of an Ion Proton™ system run implementing Ion Torrent™ sequencing technology). FIG. 12B includes an exemplary distribution 1220 of percentage forward end-to-end the subset of 384 amplicons discussed in FIG. 8 and listed in FIGS. 17A-17Z, 17AA-17AZ, and 17BA-17BY. Similarity in the distributions in FIGS. 12A and 12B may indicate that little or no bias is introduced in the recalibration subset by selecting amplicons that over- or under-perform relative to the full set of AmpliSeq™ Exome amplicons.

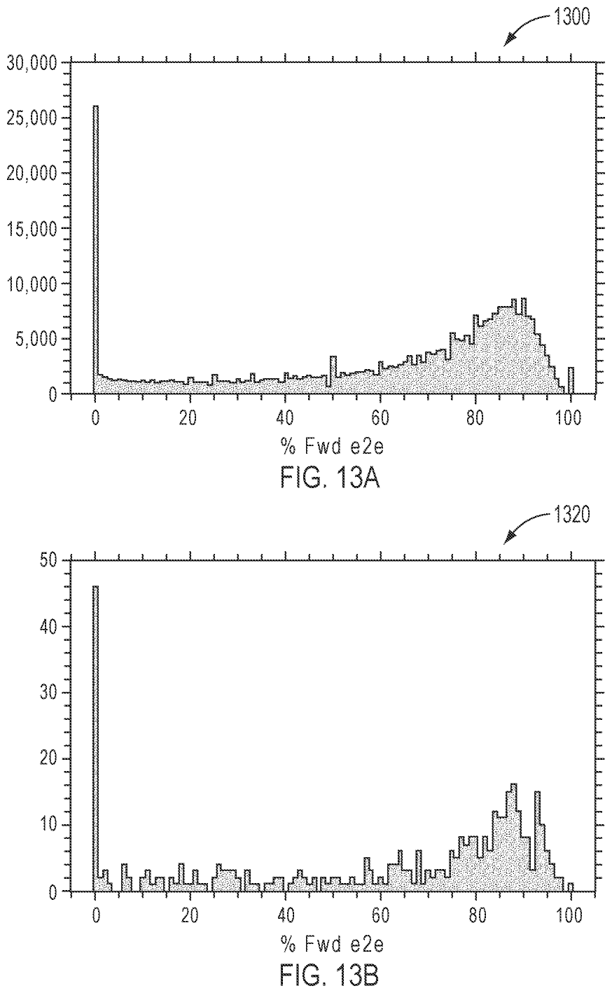

FIGS. 13A and 13B illustrate exemplary distributions of percentage reverse end-to-end for a two-exome sequencing run. FIG. 13A includes an exemplary distribution 1300 of percentage reverse end-to-end for the full AmpliSeq™ Exome Panel run (e.g., ½ of an Ion Proton™ system run implementing Ion Torrent™ sequencing technology). FIG. 13B includes an exemplary distribution 1320 of percentage reverse end-to-end for the subset of 384 amplicons discussed in FIG. 8 and listed in FIGS. 17A-17Z, 17AA-17AZ, and 17BA-17BY. As with FIGS. 12A and 12B, similarities between the distributions in FIGS. 13A and 13B may indicate that little or no bias is introduced in the recalibration subset by selecting amplicons that over- or under-perform relative to full set of AmpliSeq™ Exome amplicons.

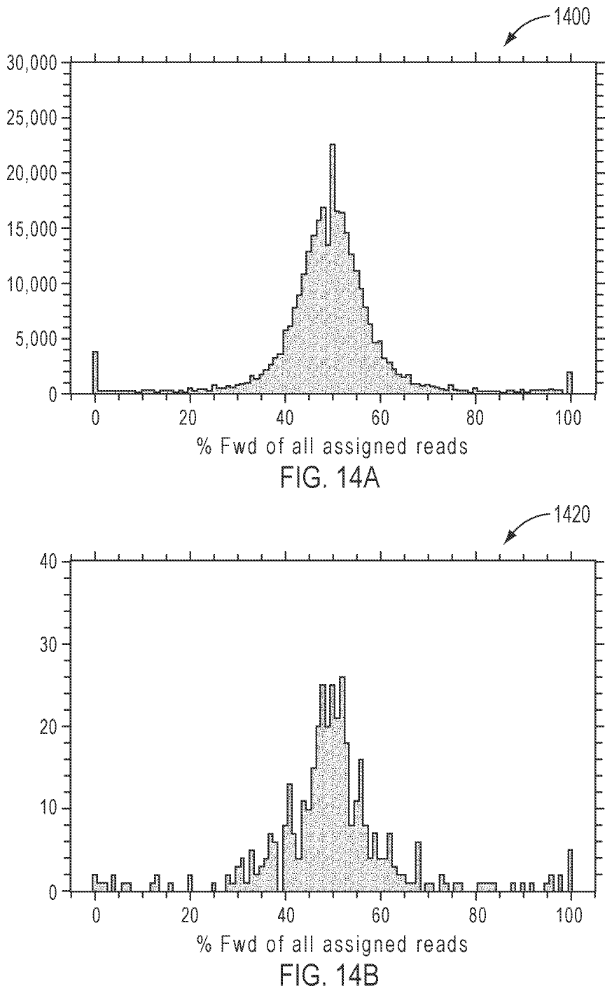

FIGS. 14A and 14B illustrate exemplary distributions of strand bias. FIG. 14A includes an exemplary distribution 1400 of percentage forward of all assigned reads (strand bias) for the full exome run (e.g., ½ of an Ion Proton™ system run implementing Ion Torrent™ sequencing technology). FIG. 14B includes an exemplary distribution 1420 of percentage forward of all assigned reads (strand bias) for a selected subset of recalibration amplicons. Similarity in the distributions in FIGS. 14A and 14B may indicate that little or no bias is introduced in the recalibration subset and that forward and reverse strands may be sequenced equally well (with the exception of a small number of amplicons with long G/C homopolymers).

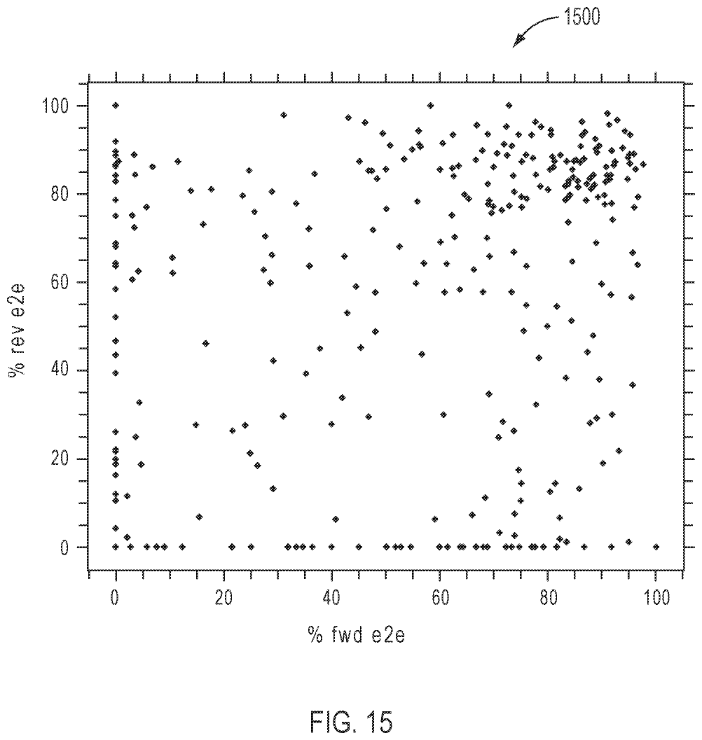

FIG. 15 illustrates an exemplary plot 1500 showing the correlation between forward and reverse end-to-end coverage. The data may pertain to a selected subset of recalibration amplicons. The majority of amplicons may have good end-to-end coverage on both strands. Still, the inclusion of at least some recalibration amplicons of a poorer quality may be desirable to maintain a fair representation (e.g., of typical AmpliSeq™ panels) which may include difficult to sequence amplicons (e.g., amplicons with long G/C homopolymers). The area in the bottom left corner of plot 1500 may include 24 poor end-to-end amplicons having a sum of percentage forward end-to-end and percentage reverse end-to-end that is less than 20%.

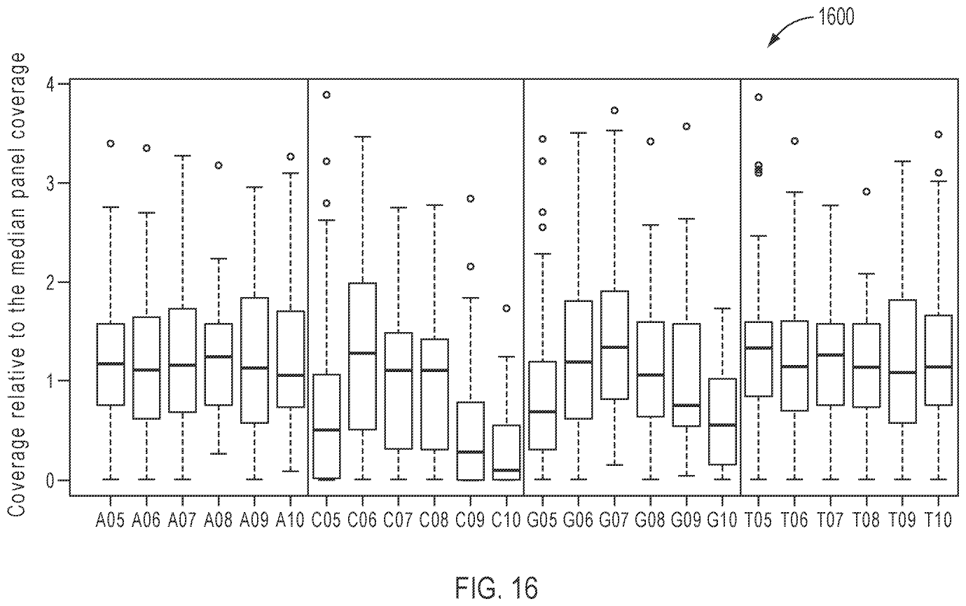

FIG. 16 illustrates an exemplary plot 1600 showing the estimated per-homopolymer coverage relative to the median panel coverage. Here, data may pertain to homopolymers of length 5 through 10 for nucleotide types A, C, G, and T.

In an embodiment, series of measured intensities obtained for panels of amplicons for homopolymer calibration or recalibration as described herein may be used as training subset(s) within the recalibration engine described in Jiang et al., U.S. Pat. Appl. Publ. No. 2014/0316716, published on Oct. 23, 2014, which is incorporated by reference herein in its entirety, instead of (or in addition to) the series of measured intensities obtained for a randomly selected training subset as described in Jiang et al., U.S. Pat. Appl. Publ. No. 2014/0316716, published on Oct. 23, 2014.

Unless otherwise specifically designated herein, terms, techniques, and symbols of biochemistry, cell biology, genetics, molecular biology, nucleic acid chemistry, nucleic acid sequencing, and organic chemistry used herein follow those of standard treatises and texts in the relevant field.

Although the present description described in detail certain embodiments, other embodiments are also possible and within the scope of the present invention. For instance, while described embodiments may include recalibration panels with amplicons that are 200 base pairs in length, the embodiments may also be tailored to recalibration panels with amplicons that are longer in length (e.g., 600 base pairs in length). For example, those skilled in the art may appreciate from the present description that the present teachings may be implemented in a variety of forms, and that the various embodiments may be implemented alone or in combination. Variations and modifications will be apparent to those skilled in the art from consideration of the specification and figures and practice of the teachings described in the specification and figures, and the claims.

1. A method for nucleic acid sequencing, comprising:
(a) exposing a plurality of template polynucleotide strands, including a set of homopolymer recalibration template polynucleotide strands, to a series of flows of nucleotide species flowed one nucleotide species at a time according to a flow ordering; and
(b) determining sequences of bases for the plurality of the template polynucleotide strands, including the set of homopolymer recalibration template polynucleotide strands, using sequencing by synthesis to generate a plurality of sequencing reads.
wherein each homopolymer recalibration template polynucleotide strand comprises an amplicon sequence including at least one homopolymer, the homopolymer having a homopolymer type, A, C, T, or G, wherein a homopolymer length of the homopolymer is between a minimal homopolymer length and a maximal homopolymer length.
wherein the set of homopolymer recalibration template polynucleotide strands comprises a set of amplicon sequences having at least a minimal threshold number of occurrences of each homopolymer length between the minimal homopolymer length and the maximal homopolymer length for one or more of the homopolymer types A, T, C, and G,
wherein the sequencing reads corresponding to the set of homopolymer recalibration template polynucleotide strands include the homopolymers having the homopolymer lengths between the minimal homopolymer length and the maximal homopolymer length for the one or more homopolymer types.

2. The method of claim 1, wherein the minimal threshold number of occurrences of each homopolymer length is 10, 25, or 50 occurrences for the set of amplicon sequences of the set of homopolymer recalibration template polynucleotide strands.

3. The method of claim 1, wherein a number of the amplicon sequences in the set of amplicon sequences is at least 288 amplicon sequences.

4. The method of claim 1, wherein the amplicon sequences of the homopolymer recalibration template polynucleotide strands include at most one homopolymer of length 6, 7, 8, 9, or 10 bases per amplicon sequence.

5. The method of claim 1, wherein the amplicon sequences of the homopolymer recalibration template polynucleotide strands have a minimal distance of 7 bases between separate homopolymers within the amplicon sequence when the homopolymer lengths of the separate homopolymers are 4, 5, 6, 7, 8, 9, or 10 bases.

6. The method of claim 1, wherein the amplicon sequences of the homopolymer recalibration template polynucleotide strands do not overlap.

7. The method of claim 1, wherein the minimal homopolymer length is 5 bases for the amplicon sequences of the homopolymer recalibration template polynucleotide strands.

8. The method of claim 1, wherein the maximal homopolymer length is 10 bases for the amplicon sequences of the set of amplicon sequences of the set of homopolymer recalibration template polynucleotide strands.

9. The method of claim 1, wherein the set of amplicon sequences further comprises a group of amplicon sequences including additional n-mers of bases C and G.

10. A method for preparing a homopolymer recalibration panel, comprising:
extracting, from a set of amplicons used in sequencing-by-synthesis, a set of candidate amplicons satisfying a first set of criteria, wherein the first set of criteria includes amplicons known to belong to regions of a reference genome with no variants; and
selecting, from the set of candidate amplicons, a reduced set of amplicons satisfying a second set of criteria, wherein the second set of criteria includes selecting amplicon sequences that together comprise at least a minimal threshold number of homopolymers of each homopolymer length between a minimal homopolymer length and a maximal homopolymer length for one or more of homopolymer types A, T, C, and G.

11. The method of claim 10, wherein the minimal threshold number of occurrences of each homopolymer length is 10, 25, or 50 occurrences for the reduced set of amplicons.

12. The method of claim 10, wherein the minimal homopolymer length and the maximal homopolymer length is for each of homopolymer types A, T, C, and G.

13. The method of claim 10, further comprising:
determining underrepresented homopolymers of the set of candidate amplicons; and
augmenting the set of candidate amplicons with amplicons including the underrepresented homopolymers.

14. The method of claim 10, wherein the reference genome is NIST NA12878.

15. The method of claim 10, wherein the reduced set of amplicons comprises at most one homopolymer of length 6, 7, 8, 9, or 10 bases per amplicon sequence.

16. The method of claim 10, wherein the reduced set of amplicons comprises amplicon sequences having a minimal distance of 7 bases between separate homopolymers within the amplicon sequence when the homopolymer lengths of the separate homopolymers are 4, 5, 6, 7, 8, 9, or 10 bases.

17. The method of claim 10, wherein the amplicon sequences of the reduced set of amplicons do not overlap.

18. The method of claim 10, wherein the predetermined minimal homopolymer length is 5 bases for the amplicon sequences of reduced set of amplicons.

19. The method of claim 10, wherein the maximal homopolymer length is 10 bases for the amplicon sequences of the reduced set of amplicons.

20. The method of claim 10, wherein the reduced set of amplicons further comprises a group of amplicon sequences including additional n-mers of bases C and G.


SEQUENCE LISTING (384 sequences)

SEQ ID NO: 1 - DNA, 224 bp, synthetic construct
  source 1..224: mol_type = other DNA; note = Description of Artificial Sequence: Synthetic polynucleotide
ccgtctgggaggtgctgcacacagtggtccttcaggacgctgtggggaggctcggtgagaccctgcctggcctccagggcccaagggagtggggggggggcggggccgggcgcccacctcttgaggtggctgtagacgcgcaatgccgtctcctgctccttgcgtgtgtcatgcaggtgcacgcggcaggtgaagcgcagctggtgctcagccagacccagctc

SEQ ID NO: 2 - DNA, 218 bp, synthetic construct
  source 1..218: mol_type = other DNA; note = Description of Artificial Sequence: Synthetic polynucleotide
ctctgggggggggcctggtggcttatgccctcccggtctggtgcagggggactccggtggcccactgaactgccagttggagaacggttcctgggaggtgtttggcatcgtcagctttggctcccggcggggctgcaacacccgcaagaagccggtagtctacacccgggtgtccgcctacatcgactggatcaacgaggtgggtgctgcctccacag

SEQ ID NO: 3 - DNA, 191 bp, synthetic construct
  source 1..191: mol_type = other DNA; note = Description of Artificial Sequence: Synthetic polynucleotide
ttgacagccaaatgcaagacgagttcttcaggtgcctgaaaacacatccacacctctgagattccccccccagtgcacctgcctccaacccagtatttccctcttgaactcttgctaaccccttatccccccataggaagctgccagaaccctggagagatgagactgcaagggcagagcctcatgtctgg

SEQ ID NO: 4 - DNA, 228 bp, synthetic construct
  source 1..228: mol_type = other DNA; note = Description of Artificial Sequence: Synthetic polynucleotide
accctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtg

SEQ ID NO: 5 - DNA, 218 bp, synthetic construct
  source 1..218: mol_type = other DNA; note = Description of Artificial Sequence: Synthetic polynucleotide
tgctaacaatccatcctgaaagcaagggtggcttcctcggtaagtataaactataacattccaaatcaggcccaaagtaccttttctggagattcacacaatatattttctggtttcagatcacgatgagcaatgcctgacatgacaaagagcaaaaaaatggttaacatatgcagataattatcaaacattccacacacaattaattttttccaaat

SEQ ID NO: 6 - DNA, 227 bp, synthetic construct
  source 1..227: mol_type = other DNA; note = Description of Artificial Sequence: Synthetic polynucleotide
ctgatccaaaggcagaaagggaggggcgaaatggaggagggtaccgtgaacagagtaagggggctgggagaggaaattagaggggaggcgctggccagcaggcaaggcctgagccctctcccccggcggtggttgggggggggccgtgtccatataaagcgggtcggcggcccctgcggtcccggagccgcgcgggcaggggctgccgcagccgatggcggggcgca

SEQ ID NO: 7 - DNA, 182 bp, synthetic construct
  source 1..182: mol_type = other DNA; note = Description of Artificial Sequence: Synthetic polynucleotide
gtggatgctaatgtgttacattttctccagttagacagtgaattaaatcggaagaaaacatggtttctagcagcagactgcaatccacagatgaagctcattttttcacttgactacctgaaacaaaaaaaaaactataaatatctgtggaaaatatttacttaccaaactgggactataac

SEQ ID NO: 8 - DNA, 200 bp, synthetic construct
  source 1..200: mol_type = other DNA; note = Description of Artificial Sequence: Synthetic polynucleotide
gattcaggctatcgattgctgcaatctttgataattcttcttttacatatgtaggaggggatatatctggaaagccttggccaagattcacaacagaagggtctgcagccaatttggtaaattcaatcctaagattgaaaaaaatactaatattaataagttctacaattataaagttcgcttttttatatcttatagtc

SEQ ID NO: 9 - DNA, 217 bp, synthetic construct
  source 1..217: mol_type = other DNA; note = Description of Artificial Sequence: Synthetic polynucleotide
cataggacagcttactacactttgtagagctgatagcttctaaatattaatagtttttaatgacattgctataaattgctagtgtctgataagaagcttgattttagattaagtgatttcatatttgtactggttctaaggtagggaaaaaaaaccaggtagtttactaagtgataatttgtttaaacaatgtaggtgtcgttcaaggaatgataac

SEQ ID NO: 10 - DNA, 217 bp, synthetic construct
  source 1..217: mol_type = other DNA; note = Description of Artificial Sequence: Synthetic polynucleotide
ccaacacaaagtgtctcctcggtaactgctattaaaaaaaaaagatactgctattcaattcagcaaatgcatcatccatccaacagtcaaacaaacattagtaccacaagtacatttcaagaagccccaaatatcttaaatctcttttgccacttatggaccagttatccattaattcatccaagggattttctaaatcatttgtattacctaatat

SEQ ID NO: 11 - DNA, 220 bp, synthetic construct
  source 1..220: mol_type = other DNA; note = Description of Artificial Sequence: Synthetic polynucleotide
cataagattaaacagtgggatgcagacaaatttgaacacatacagactctggaggtaaccacatgccttctgtgcttgcttggtacttaagaaatacttttaagaatatttacattacccactaagtagtggtcaatgaatgaggtattctttattttttttttctcctccagggtcatcaccaggaaatatggtgtttggctgtaagccccagtggaga

SEQ ID NO: 12 - DNA, 192 bp, synthetic construct
  source 1..192: mol_type = other DNA; note = Description of Artificial Sequence: Synthetic polynucleotide
caagaggtacagaacagtgtaaccatggaccagagtctctccagaagggactcttgcagcccatcccactccatccctgctgttttctgagaacagaggaaggatgaggagggagttccaacaaccccagtgaaccccttcaccaaacccccccccaacttaccagcctctaatcgtccaatgttgaccagg

SEQ ID NO: 13 - DNA, 224 bp, synthetic construct
  source 1..224: mol_type = other DNA; note = Description of Artificial Sequence: Synthetic polynucleotide
gagctgaaggtgaagaggaaccagcaactatacagaatctcaactaaaatatcttacttgaggtccctggtcttacggaaaaaaatatcttagcttgacctatgtgcttttttatagcttcttttatttctttattcctcaggctatagataatggggttgaagaagggagacaaaactgcaaaggccagagcaatggctatatcccagaacaaagagtaggtg

SEQ ID NO: 14 - DNA, 202 bp, synthetic construct
  source 1..202: mol_type = other DNA; note = Description of Artificial Sequence: Synthetic polynucleotide
tttgcaccagagatgaccaggttgtcctctgaacttgggcagctcaccagaaatatcccaaacgcgcacagtctggtccaggctggctgatactaccaagtcttctgtggggtggaactgagcacacatcacataatggttgtgccctgttaacacactgcaagaaaaaaaaaagacaataccaaattagaggtggaagtca

SEQ ID NO: 15 - DNA, 227 bp, synthetic construct
  source 1..227: mol_type = other DNA; note = Description of Artificial Sequence: Synthetic polynucleotide
caacaggcaaggtagactggccaggaatactctaggaaacatgacaggttcatctcaaacaaaatatagaacttacacctggtttctggacttgggcatagatcgtaaggctttttttttccactgttgtctggtaatggttcgttttacctggtgaaaagaaaccataatggttatctccctctggtccccagcaggagctccttactcagacctggtctttactg

SEQ ID NO: 16 - DNA, 219 bp, synthetic construct
  source 1..219: mol_type = other DNA; note = Description of Artificial Sequence: Synthetic polynucleotide
ccgcggcggcagcagcagcaggtaatcattacagcattttacatattcatattcatactcaaccccggctcccgctgcccccccccgcgacttagcataatttattagtactcaggattattataattaacggcggggagatggggggccggggagcattgagcccccgccgggcgctgagcggagggggaaagaggctcggagcggccaggcggaggg

SEQ ID NO: 17 - DNA, 211 bp, synthetic construct
  source 1..211: mol_type = other DNA; note = Description of Artificial Sequence: Synthetic polynucleotide
ttactttgagtgtgtctctgacctgggctctgataataggacccaaaatcccatcttctttcatattgggattcactgtatgtttggtgaaggactcatcttcgtactgtgtgtacataactttcttataatgttttccaatttggtttgagaaattatccaaatgctgagacctgtattttcttaaagtgaagtaaaaaaaaattaaacc

SEQ ID NO: 18 - DNA, 221 bp, synthetic construct
  source 1..221: mol_type = other DNA; note = Description of Artificial Sequence: Synthetic polynucleotide
caccaataagaagatgagatcaataaaaaaattgaagatataactgagcttaagcagtactattgtttaatgcccccccacaaactgttttctcttgacccaagcttacacttttatataaactagtatacttttcatggcaacagcagttcaggagttgtaaaggaagtgtgaatggattattttcttgtcttctcaggcacagtaaccttggtgcacga

SEQ ID NO: 19 - DNA, 161 bp, synthetic construct
  source 1..161: mol_type = other DNA; note = Description of Artificial Sequence: Synthetic polynucleotide
caaattcagcattgcaatcacacaaaataagactccataaatgattttatttacttctgatactctacctgctttactcgatccatcttcagcttttttgctgcagaataaacatcttttaccaattccttatctgctttcaacctatttaaaaaaaaagt

SEQ ID NO: 20 - DNA, 219 bp, synthetic construct
  source 1..219: mol_type = other DNA; note = Description of Artificial Sequence: Synthetic polynucleotide
aggctctctgagggagaagccaaagaggactccttggatgaagagttttttcatcacaaggcaatgcagggcacagaggtaggacagacagaccaaactgacagcacaggaggaccagctttcctttctaaagtagaagaggatgattatccctctgaagaactactagaggatgaaaacgctataaatgcaaaacggtctaaagaaaaaaaccctggg

SEQ ID NO: 21 - DNA, 167 bp, synthetic construct
  source 1..167: mol_type = other DNA; note = Description of Artificial Sequence: Synthetic polynucleotide
ccattgtattaatttagttaatttagtcaaattaattaaatttgtttattattttttttttaatctcagaaacctttggaagatgatggagaatttgtttattgccttcctcggaaaagtcctaaatccctttacaatccatatgatcttcaggtagtatcggctca

SEQ ID NO: 22 - DNA, 220 bp, synthetic construct
  source 1..220: mol_type = other DNA; note = Description of Artificial Sequence: Synthetic polynucleotide
ccccaaccaatgagaactactgatctaatctaatcataaactcacccaacatcaggagaaaagtctgtgccaaggccagattcaatcaaggctttgtaaaagggagaattgggcccagaagtcaagagtgaagacagaagacttaatgtgaaggcttcaaatgtgtcggtgatgctgcattgaaaaaaaaaggaacggggaggtaagaaaagagtaaagg

SEQ ID NO: 23 - DNA, 204 bp, synthetic construct
  source 1..204: mol_type = other DNA; note = Description of Artificial Sequence: Synthetic polynucleotide
ttaggtcctgtgaattgcaaatgaatgtttagaattaataccattaagaaagacaaatcagactatagtaaataaatatttttttttccacaatttatggttgttgcttcatacctggccagtactgaagatctgaacaaatgctttgaagagctcttgaatgatgtcgatacaagcaagaggaacgtaatgtactaaatatat

SEQ ID NO: 24 - DNA, 214 bp, synthetic construct
  source 1..214: mol_type = other DNA; note = Description of Artificial Sequence: Synthetic polynucleotide
acatacaaactcctatagcaacaggattcaaataaatacttagaccaagattttttttttaactgtttaagagaaaaggactttaagccataaatgtaacagaccacatagtcgaacaaatgggaaatggttggacctgacatttctatagaaacgtacatcattaacagtcaacataccaaagtgtgctgaaccactgctacttttactttgc

SEQ ID NO: 25 - DNA, 220 bp, synthetic construct
  source 1..220: mol_type = other DNA; note = Description of Artificial Sequence: Synthetic polynucleotide
cccttacacagcggcacggtgatctcttggcatgccagctccttggccgaggcggccgcagcgccgctagagcgctgcagcagcgccaaggcggccagcagcgaggtcacttccaacaggtaaccccactccatgctgtgcgcctcggcccggtgccctcgccctccaggcggcgcgcagaggggtgccggggggggggcccacgagagagccgcagacg

SEQ ID NO: 26 - DNA, 199 bp, synthetic construct
  source 1..199: mol_type = other DNA; note = Description of Artificial Sequence: Synthetic polynucleotide
tcttctaatttctgtagctccgatgtataggccattagtcgagcattgcacaccatgagattcttaactgcatgtaaaacttgatctttctgagtgctcagagaaaggagtttccatattccttctcgcattcgaatttctaagtctattttttcttgaatgttgcagtcctaaaaaaaaaatgcatcttgaaaatata

SEQ ID NO: 27 - DNA, 168 bp, synthetic construct
  source 1..168: mol_type = other DNA; note = Description of Artificial Sequence: Synthetic polynucleotide
tttctttcaaccagaggtttgaaagtctgccaagaaaaaaaaaatttctaatgataattttctgggtagtatcctgaaattatgaaggaacacacaccaatacaatgtatcaatgtcattatcactacagagtcttagggtacctaatattaattttcatattataaa

SEQ ID NO: 28 - DNA, 191 bp, synthetic construct
  source 1..191: mol_type = other DNA; note = Description of Artificial Sequence: Synthetic polynucleotide
aaccataatttttttttagttgaggtgaaaaaactgcttcataagttcgtatgttgtaaaagccactgcttgagagggaatacagcgaatgtaattaagagataaaccacgatagagtccttttcgaattccatggtgtccatagacatacttcatagtatcccgcatggtactgaaagacaatgattaaa

SEQ ID NO: 29 - DNA, 218 bp, synthetic construct
  source 1..218: mol_type = other DNA; note = Description of Artificial Sequence: Synthetic polynucleotide
acccagcatgctcagggaccacatcctaccccccccaaattaggagcactcacccacctgtgggagtatctggcccaggctgtgcacttctctgacgggcagttcgtgctgctggctttcagccgcctgtctccgcacacttgatgctgtttctattttttgatttgagtttaattataaagcatgctgtctactcccctcttccagcctcccctgcc

SEQ ID NO: 30 - DNA, 183 bp, synthetic construct
  source 1..183: mol_type = other DNA; note = Description of Artificial Sequence: Synthetic polynucleotide
attacagattgaagattctgaaatgtctcgtgtaattcgagtcagtgagtaagttgaatattctttaaatttaattatttgtaattgttttgaagaacttttaaagtactttttttttcttaattcagattatctttatgtgatcttgctggttcagaacgaactatgaagacacagaatgaa

SEQ ID NO: 31 - DNA, 183 bp, synthetic construct
  source 1..183: mol_type = other DNA; note = Description of Artificial Sequence: Synthetic polynucleotide
attcagaacatcacctttgaaatgacaacaaacaccaattaggtatgttatagcattcaaaggtaaggcaaacaaaacttgattaagaaaatatcataccttgtccagaaagaatatgaaaagcagtctggacacagtgaagactttcttgatagcttaggtcctttaaaaaaaaaagggggg

SEQ ID NO: 32 - DNA, 191 bp, synthetic construct
  source 1..191: mol_type = other DNA; note = Description of Artificial Sequence: Synthetic polynucleotide
cctggaggcgaaaccctcagagcccccaatactcactgcggcatctcagatcagctggcgcccaggcaggcacaggagaaactcacctgaagggcatccatggggggggtgcccccctccagccagatggtgctacctgcaggccacaggtaataacataacgatgtaagtaccatgagatgtaagcaatg

SEQ ID NO: 33 - DNA, 180 bp, synthetic construct
  source 1..180: mol_type = other DNA; note = Description of Artificial Sequence: Synthetic polynucleotide
aatacaagaaataacagatgtttggtagcttgattagcattaaacaattctacaaattttaccttgaaggaagtatagctggtaacaacgcttgctccagggaaagtagagcagacaccggtttctgactttggctttctaaatattcctggagaaaaaaacacaacatgatcatttacc

SEQ ID NO: 34 - DNA, 221 bp, synthetic construct
  source 1..221: mol_type = other DNA; note = Description of Artificial Sequence: Synthetic polynucleotide
ccagtgtcaatttccttccaaagaatgccaagttctccatcaagaaagaagctccctttgtcctggaggcatccgaaatcggttcagatctgtttgcaggagtggaagccaaagggttaagcattaggtaagtacatttcctccaagtttatatgtttaaatgcaaagaaaaaaaagactagctgacatactttttaatcatcagatgtttgtaggcataa

SEQ ID NO: 35 - DNA, 172 bp, synthetic construct
  source 1..172: mol_type = other DNA; note = Description of Artificial Sequence: Synthetic polynucleotide
ttgtttcattttttttttaataggtcctgattgttctttgaatgttccctctactgagtcttactggattctgccaaacgttaaacccttcagtccttctgtaggtcgggcttcacataaagcagttttacacgggaaatttatgtgggtgattggtggatatacttttaac

SEQ ID NO: 36 - DNA, 225 bp, synthetic construct
  source 1..225: mol_type = other DNA; note = Description of Artificial Sequence: Synthetic polynucleotide
cccccccccactgagggccttgcttgcctgatggagctgtggctctccacttattgagcacagccctcttagtccacatgtgtctgccttccaggtgcaagtgctgggcagtcaggacggtgcctaagtggacctcagacatggctcagccataggacctgccacacaagcagccgtggacacaacgcccactaccacctcccacatggaaatgtatcctcaaac

SEQ ID NO: 37 - DNA, 219 bp, synthetic construct
  source 1..219: mol_type = other DNA; note = Description of Artificial Sequence: Synthetic polynucleotide
ggtcctgcctgctgatagccatgagcatgccagtgggcgcctgggcatctccctgacccgcgtgtcagacggcgagaatgtcattatatcccacttcaactccaaggacgagctcatccaggtggggcctggtggagccatgctgggtggcggtggggggggcagtgggaacctcaaggcctctgctcattctctcccactctgtccctgccctgaagg

SEQ ID NO: 38 - DNA, 192 bp, synthetic construct
  source 1..192: mol_type = other DNA; note = Description of Artificial Sequence: Synthetic polynucleotide
ggcgggcactgtcgtcatggtgactggggtcttgggctgctgcgccaccttcaaggagcgtcggaacctgctgcgcctggtcaggagggcgcagggccacggggtgggggtggtgcagatgggcccaaggaggttgtcgctgcaggctttgaacctgtgccttgctgtgctcacctggggggggggtcacgg

SEQ ID NO: 39 - DNA, 199 bp, synthetic construct
  source 1..199: mol_type = other DNA; note = Description of Artificial Sequence: Synthetic polynucleotide
ggacttgtagccatgtgcttgctttgcagcgtgtgtaccaactcctcccaagtcgtctgcaggccggaggaaggtaagctgccctctgctgccagccctgcggtggccgggcccatcctggggaagcctgtggggccttggatcggtggggggtgctggtctcctcctgggctctgcccctttggtccccccccagctc

SEQ ID NO: 40 - DNA, 217 bp, synthetic construct
  source 1..217: mol_type = other DNA; note = Description of Artificial Sequence: Synthetic polynucleotide
tgtgcaccgagtctgtgctccgtccctacttctggctctagttaggcctcactatcccctacagggctgcttagaggctccaagggtgagaacatacctgtttaacacttcctgaatatcctggaaggagaaaaaaaaatgcagcatctgagtaagtgctattcctcagagggaacagacatcgaactggcagggagtttctctaggggaagggaga

SEQ ID NO: 41 - DNA, 225 bp, synthetic construct
  source 1..225: mol_type = other DNA; note = Description of Artificial Sequence: Synthetic polynucleotide
gagtcgctgcagccaaggctggagccatgagacggccccctggcaatggagaggcggccagcgaaggtccaggtggctggggtctatggggagtccaggagtccaggaggctgtgctgtgccgtatgtatctgggaaggtggatgtgagtgtgtgtgtgtgtgtgtgtgtgtgtgtgtgtgtgtgtgtgtgtgtattgggaagtggggggggggcatttatgcca

SEQ ID NO: 42 - DNA, 204 bp, synthetic construct
  source 1..204: mol_type = other DNA; note = Description of Artificial Sequence: Synthetic polynucleotide
gaaataagatcagtaatggaattaaatgacaagactaattggtttacctttctttgacagcctgaagagacacaagaggagatggagaacgaaatggtaatggaatgccgaacgggagaaaagtttcattcttatctgtctcaaacatcactggagcatgagaaacattgtctaatgaatggaatcaggaaaaaaagaaacaaa

SEQ ID NO: 43 - DNA, 211 bp, synthetic construct
  source 1..211: mol_type = other DNA; note = Description of Artificial Sequence: Synthetic polynucleotide
cttcaactaccaggtgctatcatcaagaggatgagaaaaaaaaattagagaagaatggagaaatcatcttacctcataagtgctggtgataccagatctgtagaacacagggagaaagagctctgatgttaagaggatgacaaataggtaagcaatgaagaagactaggaaggatgccccaaagcggtagacttcagaaggggtccccagg

SEQ ID NO: 44 - DNA, 175 bp, synthetic construct
  source 1..175: mol_type = other DNA; note = Description of Artificial Sequence: Synthetic polynucleotide
gtttatgaaataaacctcaactaatagttgaaagtctatgttaaaaaaaaagaattggcaaatattatttttttgtaccgttgtaaaatgtctttgttatcatttggtagtgattcttgttcgggtaatttacacttcgagctcttgatgtcttcacatatagtaaatgtagagt

SEQ ID NO: 45 - DNA, 218 bp, synthetic construct
  source 1..218: mol_type = other DNA; note = Description of Artificial Sequence: Synthetic polynucleotide
tctggcttactgctcctcggaggggtagaggggttgcggtattgtggtacacctactaattgtgtattttgtgtcccagtatttgggctaaatactctaatggagattgttactgaagccggccccgggagtggtgaaggtgggtcttgcctgtgagctgtgcatgatcttttttttttctctagcatcttctgtgcctgcccgagggccatccttct

SEQ ID NO: 46 - DNA, 190 bp, synthetic construct
  source 1..190: mol_type = other DNA; note = Description of Artificial Sequence: Synthetic polynucleotide
gtcctgccccaggctgccctggcagcgggcgcggtggctctgggcacaggactagtgggtgtaggagccagccgggcaagtctgaatgcagctctataccctccctggcgaggggtcctgggcccgctgctggtggctggcacggctggtgggggggggctcctggtcgtcggcctcgggctagccctgg

SEQ ID NO: 47 - DNA, 208 bp, synthetic construct
  source 1..208: mol_type = other DNA; note = Description of Artificial Sequence: Synthetic polynucleotide
agggacatcttgcgctttttctgtcccacagggttccgctgagtcacctgccgcttccacttgtaggtagggctgggggggcaggggtagtgagccctttgctggacatgcgcatgactcgtggccccccactcacacagacacaccattcacgcttactgagaacctacataatgcgaggcactgtcctaggcacgaatgttcccag

SEQ ID NO: 48 - DNA, 222 bp, synthetic construct
  source 1..222: mol_type = other DNA; note = Description of Artificial Sequence: Synthetic polynucleotide
tatctttacttatttctcttatgtaaggtattttacactcatattcaattttttgctttcagctaatgattcttaaagaacagaataatcagaaaaaaactctaacagatggagtgctggacataaaccatgaacaagaaaatacaccaagcacgagtggaaaggcaagtctgtaggctttgtctttattctgtttgatttcatttgttcacagtatgtgtc

SEQ ID NO: 49 - DNA, 170 bp, synthetic construct
  source 1..170: mol_type = other DNA; note = Description of Artificial Sequence: Synthetic polynucleotide
cccagtaagactagaaatattcaacaacacaaaattcactcagaaaaaaaaaactcttaagtataatctaataaaagaaccatataagaataagaaagcctaatatctaaacctaccattttcaaggaactcaggtcctttcagcacactggattgagagtgttgaactg

SEQ ID NO: 50 - DNA, 167 bp, synthetic construct
  source 1..167: mol_type = other DNA; note = Description of Artificial Sequence: Synthetic polynucleotide
actggaatttctcctcaatgcatttcatggccatctgaattaactgattttttttcacttggtccttttctgctgcatgaagaacatcaggattgtaggcaacatcaatgactgtgtaagcatctgtgtgtgtaattgaaagggtatgaaatgaagaaaaattgttg

SEQ ID NO: 51 - DNA, 202 bp, synthetic construct
  source 1..202: mol_type = other DNA; note = Description of Artificial Sequence: Synthetic polynucleotide
ccatttaaatgaaatagttaacaaatattgtgtaatgaagaacaacataatgatgttgtttatatctacttagtttaatatacaactttttttcctattagtgagagagcagcagtagatgcatcttacattgatgagatagatgaactcttcaaagaagccaatgctattgaaaactttctaatacaaaaaagagagttcc

SEQ ID NO: 52 - DNA, 208 bp, synthetic construct
  source 1..208: mol_type = other DNA; note = Description of Artificial Sequence: Synthetic polynucleotide
caggagaactacaattatttaggttaatgctgataaaagtttcctctgagcatcacctaactcaggacagagccaacttactgttacaattagtgacatcaatgtgtttacgatccttcatcatttcaactcccactttggaccttcaaaaaaaaaatagaacaataaattagcctgctctctgcccatgtaacacaggtgaatcatc

SEQ ID NO: 53 - DNA, 217 bp, synthetic construct
  source 1..217: mol_type = other DNA; note = Description of Artificial Sequence: Synthetic polynucleotide
aacactgcagggcctctccttcaggcaaccgtacgggcaacttctgaagggataccaggagtgacagaatagtctctagcctgggccttcgagaccgcatacaaagagggcaaaggaattttacttctttagcttgccagctggatccttttttttgggaacttgatttaggaagaggaacacagctgttatggaaccagtctttgcagagctcaca

SEQ ID NO: 54 - DNA, 219 bp, synthetic construct
  source 1..219: mol_type = other DNA; note = Description of Artificial Sequence: Synthetic polynucleotide
tggagaccttgcagcctcctgctcaaggacaccctcctaagccaccctcgcatgcagccctccaccagccccgtgtctacccccactcacagcaccagctcctgcagcagccacctggtcgccgtcaagaaggcagaaattggctgggaaggagagagtgcacaccgctcgcacctaggtccaagggtgagggtgccccccccccaccaccagcaccac

SEQ ID NO: 55 - DNA, 171 bp, synthetic construct
  source 1..171: mol_type = other DNA; note = Description of Artificial Sequence: Synthetic polynucleotide
ctggaataaatattgtgaatattaacaagaaccatgccattttttttttaactgcggtaaaatttacctgtttcatctgtagcgggatcattcaccagatctgcaaagacacataacaaggaatgaaaaaatgcagtcaggaaaagtagctaaggaaagcaggataagggg

SEQ ID NO: 56 - DNA, 219 bp, synthetic construct
  source 1..219: mol_type = other DNA; note = Description of Artificial Sequence: Synthetic polynucleotide
gaagaatcatattcattgcttgccccggcatcagaatcctctaaacactgctttattttctttgtcaggactttttttttctgaataagctcttgttgcctttccgtaagttcttgaatttgaatttctactgcatgtagctcactggttatagaatccagttcctcagttagagctatgggaggcagcgcggatacaatgattagacagagtaaatta

SEQ ID NO: 57 - DNA, 162 bp, synthetic construct
  source 1..162: mol_type = other DNA; note = Description of Artificial Sequence: Synthetic polynucleotide
ttagctcacacaattaaaaaaatatatgtatttcttacctctgaacttcttgggagggttggcaaggtccccggcctctgggtgcaccacacatctgtcatccactaacctagaaggaaaagcatgtttacattgttaacatccttgtcatattcataaacc

SEQ ID NO: 58 - DNA, 224 bp, synthetic construct
  source 1..224: mol_type = other DNA; note = Description of Artificial Sequence: Synthetic polynucleotide
acagaaaccctcccaactggatatccacagtccctcaagatggctttgtctctggagaactattttttttctgctctccctttgatcgtgcctgacttgaggataaaaagtcgagacatcgaaagaagcaaggataggggcaagatttagggggtagaaagaggaattgtgggaactcacggctttgggcagcgatccacttggtttctggtctctgacgggga

SEQ ID NO: 59 - DNA, 221 bp, synthetic construct
  source 1..221: mol_type = other DNA; note = Description of Artificial Sequence: Synthetic polynucleotide
gaggctaacctcatctccacactcatcttgtatcccctttgaggagaactgggacattcacctggattcctctagccaacaaaagttctggaacataacacttaccatggggtccaggggggggaacccctggtgggactgccccaggctggggggctccagctggttgctgctgctgtggccctgcagttgaccctgcctgcccaatctgttcagaaggg

SEQ ID NO: 60 - DNA, 221 bp, synthetic construct
  source 1..221: mol_type = other DNA; note = Description of Artificial Sequence: Synthetic polynucleotide
cttccacagatgcctcaatttcactcgctgggatccacgtgtcgtaactgccatgggaaattgagcacacagtagaatcaatgggatagaattgtggaaaagtaaagtgcagatcttttaccctttccatttacagaaagtattgggttcttggcttattttttttgtattctattcattataaagtagtaaaagcacattacgaaacatttcaaaaatag

SEQ ID NO: 61 - DNA, 218 bp, synthetic construct
  source 1..218: mol_type = other DNA; note = Description of Artificial Sequence: Synthetic polynucleotide
ggtgggaggtcagccttccctataggcaaggggactgggttacctaccaggcgcagttcaggaatggagcggctcaaagtccattcctggctattgatcacagcctctgtaacgggagaagggcagtaagaggggaggctcctggctcagaaggacccccaacactgagccacccctcctccctctgcagctttccaacccccccccaaccctgcctg

SEQ ID NO: 62 - DNA, 203 bp, synthetic construct
  source 1..203: mol_type = other DNA; note = Description of Artificial Sequence: Synthetic polynucleotide
cagagacattctgttttcccaagcttgctgtccacttaccctagttgttttgactttattcccagaggaacagctccatcctttccgatccggaagaactttacattcttctccctctagacatggctgcatatggcaccaccatttctgttccactattgaagctataagagagaaaaaaaattgactcaacatttttttct

SEQ ID NO: 63 - DNA, 173 bp, synthetic construct
  source 1..173: mol_type = other DNA; note = Description of Artificial Sequence: Synthetic polynucleotide
tttctaaggagcaaaaacatgtattatataagaagatagattttttttaaagatttatgaactgcgagtttggaaaagcacatttaattctagcatgtgaaattgttcgatttattttcccaggatctttgtcagttggttaatgctgatgctccttattggctagtgggcat

SEQ ID NO: 64 - DNA, 172 bp, synthetic construct
  source 1..172: mol_type = other DNA; note = Description of Artificial Sequence: Synthetic polynucleotide
ctacaagcaaacacaggcaggattattctcacctttacctgctctatatgtcttggcttgattcactggcatgggcgtcataggtattgggtataaaggcttgagagaattaaaaaaaaaattaacattaggcacctatgataataacaaggtattctgtcactcttagttt

SEQ ID NO: 65 - DNA, 221 bp, synthetic construct
  source 1..221: mol_type = other DNA; note = Description of Artificial Sequence: Synthetic polynucleotide
gacatcagcatccctccccccccatctcggagaacccgggcttcttacccagtgaagcctggggggctggctgtcagatgtggcaaaaaaggtcactgaagccaaggctgtgcctgcgatcaccaccacgacccagacgggaacgaggccgcctatctcatagacaccatctgcaaagggagagaaagggagctgataccttcccagcacctcaacaccca

SEQ ID NO: 66 - DNA, 229 bp, synthetic construct
  source 1..229: mol_type = other DNA; note = Description of Artificial Sequence: Synthetic polynucleotide
gacaaacgagtctcattgggatttagctctactgagttttgggtgaaatgtttagaatgatagcatcaagtatatcttataacctccattttttttctaatgccaccatagcagagacatgtgtttatcagaccttgaaaataaccacccaaaagtcgatattaagagggaaaaaaatcagaagtcactgtttaaggaccagaaatttgaagccatgttggttcagcaa

SEQ ID NO: 67 - DNA, 221 bp, synthetic construct
  source 1..221: mol_type = other DNA; note = Description of Artificial Sequence: Synthetic polynucleotide
gagctggggggggcctagggctcggggctccccagcacccgcctcctgcgctaccttagcccggagagtcacaatgcgggacaggaggccgatgaggatgtccgtgtacacgctcctctcgtgggcctcgtcgatgatcaccaccttgtaccgcagcagcaggaagtcctggggggaggtccggggtgaggcccacccttccagcctaagccccaagggcc

SEQ ID NO: 68 - DNA, 183 bp, synthetic construct
  source 1..183: mol_type = other DNA; note = Description of Artificial Sequence: Synthetic polynucleotide
aataaatcatgataggcacttgaagatgacttgtaaaattgatggctaacttatcctgcggttttctaaactctgttttttttcttcttaccacagcctcagttatgtttaatttccctgatcaagcaacagtaaaaaaagttgtctatagcttgcctcgggttggagtagggaccagctatg

SEQ ID NO: 69 - DNA, 186 bp, synthetic construct
  source 1..186: mol_type = other DNA; note = Description of Artificial Sequence: Synthetic polynucleotide
gtaatggagggtacttctccaaggtctagggagggaaggggagggggggtactggctctcccctcccctcccctattaagaaaaaaatgtacacaatgtaagaggactactcttcaagtatagaggcaagaaacaagtgcatgtcagattgtcctgaccttcatggagcaagatggctacgtcttc

SEQ ID NO: 70 - DNA, 228 bp, synthetic construct
  source 1..228: mol_type = other DNA; note = Description of Artificial Sequence: Synthetic polynucleotide
ggggcttccctcccagtgccatcatctaccttgcctaataagaggttgaacttgggcaaggatgcaggggtggctgtggcaggcggtttcactgctggactaggcacccccccaggggtgcttcgaaactggcctttcctctcccgggccctggtattctggaaccagacctgtaccactcgctttttgagccccacctcctcggagatgcagtcgagcatcttgcgt

SEQ ID NO: 71 - DNA, 207 bp, synthetic construct
  source 1..207: mol_type = other DNA; note = Description of Artificial Sequence: Synthetic polynucleotide
ctgtgcgcctgcccccccccactcctcaggccaagctttggggctggagaagggaccctggtcccctccaggaggcagtgggctccggcaggcagggggtcgccagggttggcgggaggaggcaggactgctgggcagttcaggggatccctctgagggggtcagtccgttctcatataccccagggctgtcctcatccaggggctc

SEQ ID NO: 72 - DNA, 170 bp, synthetic construct
  source 1..170: mol_type = other DNA; note = Description of Artificial Sequence: Synthetic polynucleotide
aaatcaggggtcttcttccaaccctttaatacattaacatccctgggaccagactgtaagtgaacgttttacacacatttgcattgtaaatgataattaaaaaaataagtccaggtattttttattaagcccccccctcccatttctgtacgtttgttcagtctctaggg

SEQ ID NO: 73 - DNA, 201 bp, synthetic construct
  source 1..201: mol_type = other DNA; note = Description of Artificial Sequence: Synthetic polynucleotide
gtgatcctgcaggttactactgtgggtttaaagccactgcagcgggagttaaacaaactgagtcaaccagcttccttgaaaaaaaagtgaagaagaaatttgattggacatttgaacagacagtggaagtaagtcaaccaaaaggagctgactttttttatgctatatagaacagtgaggatctttgtataatttctatac

SEQ ID NO: 74 - DNA, 220 bp, synthetic construct
  source 1..220: mol_type = other DNA; note = Description of Artificial Sequence: Synthetic polynucleotide
gaggcatgggtagcaagaaactaaaacgagtgggtttatcacaagagctgtgtgaccgtctgagtagacatcagatccttacctgtcaggtaaattttatttaacatttttattgataagttttatgcacaagttaactttataccttaatacctcttaaacttgaaatatgaaaatgtaggcttacaaaaaaaagataaatgatggtgcttttccatag

SEQ ID NO: 75 - DNA, 216 bp, synthetic construct
  source 1..216: mol_type = other DNA; note = Description of Artificial Sequence: Synthetic polynucleotide
aaactacataatttagaaatgttaactgtagaaaaatgtttagttcttatattctaaaatggacgttgtccattatcagatggaaaattgaactgacaaattagaatttctacaatgagtctcaaaatcactcctaattttatcacttctattattttttttagcctgccctcaagtatgtgttgtatacattggttggctttgtgggttttgtaa

SEQ ID NO: 76 - DNA, 209 bp, synthetic construct
  source 1..209: mol_type = other DNA; note = Description of Artificial Sequence: Synthetic polynucleotide
acgatgggtttagagcacagcttatcaaaggacattatttctattataaacaatgtcttccaagccccctgggggggatcccacacctgccagaaggacgaaaaagcaatcgagtgcaacttatgtcagtctagtatcctctgctatcagcttgcttgtgaactcctggagagactagctcctaaagaagaaagccggctggtggtaag

SEQ ID NO: 77 - DNA, 228 bp, synthetic construct
  source 1..228: mol_type = other DNA; note = Description of Artificial Sequence: Synthetic polynucleotide
actggtcttcatcagtccctgaaacaacttcaaaatatcaaatacttcaatcactagatatgtatcctagcagatttatttaaagtaagatacatttgaaaaaacacttcggatttgtaaacctcaccttcacatctgtgaacactgctattaatccatgattaatattcagaagaactgagagaaaactctgagagttcttgttctgagagtctaatttttttttcc

SEQ ID NO: 78 - DNA, 175 bp, synthetic construct
  source 1..175: mol_type = other DNA; note = Description of Artificial Sequence: Synthetic polynucleotide
ataaccatgaattattttttttagaggttagagagaaatatattgaaagttataaaatgattgtgttattcttttaggtgaaattgccaaatacatggaaacagtgaaattactagactacactgaaaaacctctttatgaaaatttacgtgacattcttttgcaaggactaaaa

SEQ ID NO: 79 - DNA, 221 bp, synthetic construct
  source 1..221: mol_type = other DNA; note = Description of Artificial Sequence: Synthetic polynucleotide
gggggtccgggctgggcctgcaagactgcaggcggtgcctgccaggaaggctggagcggcagtgtgggcacctccgcttgcggacccgatgcctgggcctgctgggggggtagctggatttgcacggctttttgggagggcggggctccctgaaagggctgctccagctggaccaaggggggagcctgcctctgggcctcctgggcaggcaggggctgcca

SEQ ID NO: 80 - DNA, 220 bp, synthetic construct
  source 1..220: mol_type = other DNA; note = Description of Artificial Sequence: Synthetic polynucleotide
cgcccatcattatatcctttaacctctatgatcaaacatctccttgtttgcagctgctctcattcgcggaaacagaaacaattgcgctcaattctccaataaccttgattggctcatcagtaaattggacagactagaatcttcctcaggtgagaattgacaggaaactataactagaaaagatgaagggttttttttttctttctgtaaaaaaagaaaa

SEQ ID NO: 81 - DNA, 225 bp, synthetic construct
  source 1..225: mol_type = other DNA; note = Description of Artificial Sequence: Synthetic polynucleotide
ttaacctgaagtcgtttacttctcaacaaaattatgttgagggtgggaggtagggattgatctttaattttcacttatttgggctgcattgttttttttttaacctctgtatttctgctatcttcaaatgacagagtacgtttcctgatgggtggacgtcatggagagtttaagttcctgcctccctctggctatgccccttgctatgaagccttacttccaaaa

SEQ ID NO: 82 - DNA, 217 bp, synthetic construct
  source 1..217: mol_type = other DNA; note = Description of Artificial Sequence: Synthetic polynucleotide
gaggcatcactgtggccacacagggtatgggctgatggggccagggctccagggttgggtggcggggggggggcaggagacacttcctctgactgcctgtttctgtctagcctcggtccccagcccagctttgccccgaagcctgtcccactgggagatgagtcgggtgagtcgccattgttaaaatgttcttccaaggttagaggaggcaaaggga

SEQ ID NO: 83 - DNA, 173 bp, synthetic construct
  source 1..173: mol_type = other DNA; note = Description of Artificial Sequence: Synthetic polynucleotide
gagactgtgcagcgtacacaattagtgcagcagcagttcttggtataatatatgacaaccaagtttactggtaagcttgaaaataattcttatgtaaaaattagagattttatgaatttctgatggttcagtaattttttttcagaaatgttatttcagtcacttaatctgcc

SEQ ID NO: 84 - DNA, 212 bp, synthetic construct
  source 1..212: mol_type = other DNA; note = Description of Artificial Sequence: Synthetic polynucleotide
ctaattgacctggttccaattttttttttcccacagagctctagtgccaaatgatgaatcttttctattaactgacccagtcttcaaaaaagaattgctagcctgagaaatgtggaatgcctggcttctctgactagtgttgacacagttgtttccagcgtgaacatacctgtacaagtgaagccatcacctgtgtatccttccttgcacag

SEQ ID NO: 85 - DNA, 171 bp, synthetic construct
  source 1..171: mol_type = other DNA; note = Description of Artificial Sequence: Synthetic polynucleotide
cctataccaaccggtaatggataaagtggaacatccacttgacctaggaaatcatctcttgtctataagagaaggaagaaaaaatataattagtaatacgctcaattcctttagaagagtttgatgagatagtgccaccattcacagaaaatgtagagttttttttttatt

SEQ ID NO: 86 - DNA, 221 bp, synthetic construct
  source 1..221: mol_type = other DNA; note = Description of Artificial Sequence: Synthetic polynucleotide
ccactatacccattattcatccattcaagcttggttttgtcagggtgtgtggccatgggtctttgcatcggtttcacaggactacttgagacaatgctggcgtcatgatatgccatactggagcttattggagtgaatgcaaacggattcctgcattcaacagggctgggggggacactactgctgcagttagaatgtggagtaccaatgggtgtatgtcg

SEQ ID NO: 87 - DNA, 224 bp, synthetic construct
  source 1..224: mol_type = other DNA; note = Description of Artificial Sequence: Synthetic polynucleotide
ttggcagagccccattccacggggaaagtggtgggtgccgggcgaagcgccgcgcattccctcacactccccgggtgcagcaggcctcgtgccctccccggggcccccaaccattggccagtccaccccccccccgacaggctgagggctcaggagtggcactatttcgttacctctctgcgcggaggcgagtgaggaggtgatgagccggacggccgccgcgc

SEQ ID NO: 88 - DNA, 226 bp, synthetic construct
  source 1..226: mol_type = other DNA; note = Description of Artificial Sequence: Synthetic polynucleotide
tgagtatacttgaaaactgcatgtctggtcagcatgacaaaagtcttaaaccagggacaacacagtttttttcacagatgaattgcttaaacaggcaggctacttaccttgctcttgtttgaagagaataccgtactaaaaaagaacagaagccacattaaacccatggtcagatttcagatgtctcacattgcctccagaaagcactcctccccgagtagacaga

SEQ ID NO: 89 - DNA, 191 bp, synthetic construct
  source 1..191: mol_type = other DNA; note = Description of Artificial Sequence: Synthetic polynucleotide
ccccgacacccgcccgctcactgccccgggccttgtgtgttgcaggaccacggaggggtgatcgtgaacatcactgccaccctggggaaccgggggcaggcgctccaggtgcatgcaggctccgccaaggccgctgtgggtatgaccacccccccccgcccaggtttgcccacgtgggtccccaatgggcc

SEQ ID NO: 90 - DNA, 198 bp, synthetic construct
  source 1..198: mol_type = other DNA; note = Description of Artificial Sequence: Synthetic polynucleotide
gacaacatcggctacgcctggattgccatcttccaggtgggcggcaagatggtgggacggggaccctggggcacggcaggggagcgggtgcaggactcgccccccccagcccagaccccaggggcacggggaggaggaggaggggtcgtgcgggcccaagtcaagccactgcccccccaggtgatcacgctggaaggc

SEQ ID NO: 91 - DNA, 225 bp, synthetic construct
  source 1..225: mol_type = other DNA; note = Description of Artificial Sequence: Synthetic polynucleotide
gcaggcgatgctctggggcaccgggtgtccatcctcccaaggtgctgtctgcaggggagggtgccagccaaaagccaccgggtagaaagaaaacaaggaccttgctaagatggggggtcatctgggcagatggggcccctgcctacctttgggggggcactggagtcatagcagtgctcagtccccagatggtccacaggtgcatcctttttgttcctcatggca

SEQ ID NO: 92 - DNA, 206 bp, synthetic construct
  source 1..206: mol_type = other DNA; note = Description of Artificial Sequence: Synthetic polynucleotide
cgcctggtgccgccccccccccaggatgaagggcggcgagggggacgcgggcgagcaggccccgctgaaccctgagggcgagagccctgcaggctcggccacgtaccgggagttcgtgcaccgcggctacctggacctcatgggggccagtcagcactcgctgcgggcgctcagctggcgccgcctctacctcagccgggccaagc

SEQ ID NO: 93 - DNA, 178 bp, synthetic construct
  source 1..178: mol_type = other DNA; note = Description of Artificial Sequence: Synthetic polynucleotide
cccatttgtgaagtgaattaaagtctttctggcctaaaataatgttctttaaaaaatgtttattaattctgtgtaattttttttttctttagtccaaagcacttagaatctccacaccactaactggtgttaggtacattaaggagaatagcccttgtgtgactccagtttctacagc

SEQ ID NO: 94 - DNA, 217 bp, synthetic construct
  source 1..217: mol_type = other DNA; note = Description of Artificial Sequence: Synthetic polynucleotide
gcccagtacgagagcaagaacaagtcagcccacaaagagatctacacccacgtcacctgcgccacggacaccaacaacatccagtttgtctttgatgctgtgacggacgtcatcatcgccaaaaacctgcggggctgtggactctactgagcccagccgccctgcccggcacccttgccctgcctggcctgccgccccccctcccctggaaccaggc

SEQ ID NO: 95 - DNA, 209 bp, synthetic construct
  source 1..209: mol_type = other DNA; note = Description of Artificial Sequence: Synthetic polynucleotide
aagtgagagcactctagtcccaggtcttggggggggggagcactcacctgtccccatgtagacagcgagggctatgaccatggcagctgtcaccactccaccccaggtcttcacttcaggctgctgcatgctgttgaagacgggcacactgctgacgtggcactgtccaggtgaagggcaccgtcatggtggggaaatgacctcaaccc

SEQ ID NO: 96 - DNA, 176 bp, synthetic construct
  source 1..176: mol_type = other DNA; note = Description of Artificial Sequence: Synthetic polynucleotide
caagtctccagggcccttctgtacctgacttcttggctgtgctctgcaccccaccagcaccaggacttccaggagagcctgtctttttactcaacagactgttgaagaagctggccaacaccccttcacttgctgcattatctaaggaaaattaaagagaaaaaaaagcatcatat

SEQ ID NO: 97 - DNA, 232 bp, synthetic construct
  source 1..232: mol_type = other DNA; note = Description of Artificial Sequence: Synthetic polynucleotide
gaggtgaatttctttggcgcgctcgagctgaccaagggcctcctgcccctgctgcgcagctcaaggggccgcatcgtgactgtggggagcccagcgggtgagtgcccccccccactggagcaaaaaggagccccctggggtgggggagggcttagggagccccttgccaaagctgagctgccccactcccaatccatccgcaggggacatgccatatccgtgcttgggggcc

SEQ ID NO: 98 - DNA, 205 bp, synthetic construct
  source 1..205: mol_type = other DNA; note = Description of Artificial Sequence: Synthetic polynucleotide
gcctccggggggctctcttggcagctcgtgcattgtgacgaatgaagtccacccccaggcctggctcctgcaggacacagcaagcgtgggtgggggcagacagcattatgttgtgtccctgaacaccccccccacaccaggagccctgcccaaccaccatctagcccccatacatcctctcaccttagcttcgggacctggtggg

SEQ ID NO: 99 - DNA, 218 bp, synthetic construct
  source 1..218: mol_type = other DNA; note = Description of Artificial Sequence: Synthetic polynucleotide
cagcctccaggcccaccctggggcaaccccaacctccagccacgcgcctccccacctcgggtgttggtagccatgtcagccactttctggaaggcatccaggaaggccacagcagccagcacggtggtcctggggagagggacaacggcatggatggtcggtggttgggcggggggggggcctctgcctgcccctcccctgctggggtgccctgggcc

SEQ ID NO: 100 - DNA, 229 bp, synthetic construct
  source 1..229: mol_type = other DNA; note = Description of Artificial Sequence: Synthetic polynucleotide
ctccacagggtcctcactgagcagcgagtcgtcccccgtgtcctctccggccaccaaccacagctcccccgccagcacacccaagcgcgtgcccatgggccctatcatcgtcccccctgggggccacagcgtgcccagcaccccccccgtggtgaccatcgctccaaccaaaaccgtgaatggtgtctggaggagtgagagccggcaggtgagtggggcggggcaggga

SEQ ID NO: 101 - DNA, 215 bp, synthetic construct
  source 1..215: mol_type = other DNA; note = Description of Artificial Sequence: Synthetic polynucleotide
gctacactgggcgcctggtggcctttcaggaacggttccacggggggggggccccagtgtggctccctcagcccacctgggcccacgtgaggaaggctgggatgtcccgtacaggaacattccttgtgagcgccttcacacgcaggttccggtcatccgtcaacagcaccacctcccgcagtagccggattggctcctctgtgggcatgagagag

SEQ ID NO: 102 - DNA, 187 bp, synthetic construct
  source 1..187: mol_type = other DNA; note = Description of Artificial Sequence: Synthetic polynucleotide
ctccggccgcgctggagcgtcccacccagtgcctacgcctggctccgcctccagcgcgaagccccgccccgagggcggtgcttcccggttggccccgccccataaggcccccccccaacaataatcgtccgggcctcggagtagctcttcccaccggaaaataagcgaacagttctgccaatcgaag

SEQ ID NO: 103 - DNA, 226 bp, synthetic construct
  source 1..226: mol_type = other DNA; note = Description of Artificial Sequence: Synthetic polynucleotide
agtaccgggatcacatccacagcctgtgggctgtcagcgtcctctggagcagccccatcatctgaagctggtgggagggcaggaagggaggagaggggccccttggagttggtagcctcatagtccatgaggagtaggggggcttctgggacaccagaggaaagctggcctggggccatgtcctccatagtcatctcagaggctggcagaggtgcaggggggggac

SEQ ID NO: 104 - DNA, 169 bp, synthetic construct
  source 1..169: mol_type = other DNA; note = Description of Artificial Sequence: Synthetic polynucleotide
atataactttaaaaaatttagtgtgccaggttatttttttttcataatgcttagatgtgaaatatgtggtgatgttttgtagaaacagttcgtgactatgagcaccagttccaccttaggctggagcaggagcgaacacagtgggcacagtatagagaatccgcagaga

SEQ ID NO: 105 - DNA, 212 bp, synthetic construct
  source 1..212: mol_type = other DNA; note = Description of Artificial Sequence: Synthetic polynucleotide
aatatattaacttttttttgatcttggagaaatttcaagggcaaatatatttgttgaacaaatggataagtaacaaaccaacctcctgcaattgaaggatttctgcttccagactcttcaatttcttttcactctctttggattgagcaaaaatctcatctctggatgcacgagcttcttctaattcacgttggtaatccttcatctgagcc

SEQ ID NO: 106 - DNA, 216 bp, synthetic construct
  source 1..216: mol_type = other DNA; note = Description of Artificial Sequence: Synthetic polynucleotide
gaggccaccgcggcctcctaaagaggacgcacacctgggactgcagggccagcacttgcttctccaggttcttcctggcctcctcctcctcctcctgctgctcctgaagactgttcttctcctcttccagctgccggatccgactgctcaggtttagtttctggcgtgtctcctcctgaagaagctcctgtgttttttttttaaagggcaacactt

SEQ ID NO: 107 - DNA, 204 bp, synthetic construct
  source 1..204: mol_type = other DNA; note = Description of Artificial Sequence: Synthetic polynucleotide
ccccagggaaaccacgtcgaaagccctcgctgacatagatcatccaagatgtgtaacggtccttcaggttgtacagcacggctggctcattcaggtgcgtcagcatggccatgtcttcgatcctgtcgaacttgggggggttcatggcgtacacatcctctggtttgaccaccagggtctaaaaaggaagaggcacagtgcttc

SEQ ID NO: 108 - DNA, 202 bp, synthetic construct
  source 1..202: mol_type = other DNA; note = Description of Artificial Sequence: Synthetic polynucleotide
ggaggtggagaaggcccctgcttggatcctacatgggctttaaaaaaaaaagtcctcaaagcaaagctgccagcaggctgagtgcgtctcctccctcccactgtctcttccagaggagagcgccatgagtagcgaccgcatggactgtggccgcaaagtccgggtggagagcggctacttctctctggagaagaccaaacag

SEQ ID NO: 109 - DNA, 189 bp, synthetic construct
  source 1..189: mol_type = other DNA; note = Description of Artificial Sequence: Synthetic polynucleotide
ggcccttctaggggatctctctggggtctgaagatctttctaaggcccctctgcatccactcccctctaggaagtcctacctcgtagcccttgagcgacggtcccaccaggatgatgggcctcatggaaggcaccacgtcataggggggcacatgctctgtctggggggggaagcagggaggggaaacc

SEQ ID NO: 110 - DNA, 225 bp, synthetic construct
  source 1..225: mol_type = other DNA; note = Description of Artificial Sequence: Synthetic polynucleotide
ctactgccctgggggtgtcagtgccagccccccacaaatcttttctgccccccccaggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcgtgagtc

SEQ ID NO: 111 - DNA, 196 bp, synthetic construct
  source 1..196: mol_type = other DNA; note = Description of Artificial Sequence: Synthetic polynucleotide
tgtcattgacttccacaaccctgcggatgtggtggaaatcaggacactgtcccggccatctgagacctgcaatgagaccaccaagcttggggactttgttcgataccattacaactgttctttgctggacggcacccagctgttcacctcgtgggtccgggggggggccgggactgggcaggtgggtgggcacagg

SEQ ID NO: 112 - DNA, 189 bp, synthetic construct
  source 1..189: mol_type = other DNA; note = Description of Artificial Sequence: Synthetic polynucleotide
aggcgaccgtaaatctccggcaatggcgagtttatagcggggacaattggcttccccagctcaacccccccccaacccatgcctccgaagtccctttggtgtaaagctccaggggtgggagggggaaggggtgcccacgcactcacccgtgctcacgtgaactttgcgcatccaggggtagacgacggg

SEQ ID NO: 113 - DNA, 203 bp, synthetic construct
  source 1..203: mol_type = other DNA; note = Description of Artificial Sequence: Synthetic polynucleotide
caaccactgtgatgcctcctaacaggctgtctgtctctgctgagctgttgcttgtcatacttccacataaagatgctttgtctacctgaccagattctgaaagatcttctcctgcagggtagtctgtttctcgtgcacctgaaaaataaatcttcttgtaacagcaaagtaaacttctataaataacattttttttactaaaa

SEQ ID NO: 114 - DNA, 188 bp, synthetic construct
  source 1..188: mol_type = other DNA; note = Description of Artificial Sequence: Synthetic polynucleotide
agccaaggcacctcgggcagcgccaacgactcccagcacgaccccgggtaagtttccagccgctgcccaccgcgccgccttgggctcgctctccttgcagcggcggggacggcggtgcgcggagccgggcatctcccgcgcccccccgcctctcccgcgcgccccccccgtggaagttacacaccttt

SEQ ID NO: 115 - DNA, 208 bp, synthetic construct
  source 1..208: mol_type = other DNA; note = Description of Artificial Sequence: Synthetic polynucleotide
agcctgggccacccttctgcctggggcgcccagcctggccttgggcgggggggggcagaaagagcagggctggggggtgcctaccatcctcatgggtgcgcaccagctgcggggtgctgcgtggcccgtccccgggggtggtgaaacacagcactgaggtgaagtactcggtgaacttcttcaggccagacacgaagcccacgtggat

SEQ ID NO: 116 - DNA, 207 bp, synthetic construct
  source 1..207: mol_type = other DNA; note = Description of Artificial Sequence: Synthetic polynucleotide
gctcaggcccccccccaccgccccaagatcggtcacctctcagcctcctcctccgtcacctccttccgcctgggcaagcccgtgctgcccccagtattgttccgggctgagtgggtggcactggagacattcttcggcagcttcccaaagccgctgctctcactgccctcacaggtggctgccttgatgatgttgctgacaggtaag

SEQ ID NO: 117 - DNA, 221 bp, synthetic construct
  source 1..221: mol_type = other DNA; note = Description of Artificial Sequence: Synthetic polynucleotide
ggccagccccgggagctgggggggtccctgagatccgcacaagggagggagcagggccctggggagcccctgaccactccaccaggtcccacctttttcccaaggacgtctttctgatctgcttctctctggtgagcccggcctccttcgagaatgttcgtgccaaggtagggcaaggctgggggcccctgtggggagagggcttgccccagtacctgccc

SEQ ID NO: 118 - DNA, 205 bp, synthetic construct
  source 1..205: mol_type = other DNA; note = Description of Artificial Sequence: Synthetic polynucleotide
cagaagtgaattcattggccagaccctcaaactgttggtgagtatcaccaccaaagatctgcgggcagtgtgtgttggggagtgtttaataaccagctcaccaggataaaaaaaaaagcccaatttgtggtatttgccaatttccaaagtgtaaatactcctgctatgctaccaatgcagtcactgaacatggagttgggaactg

SEQ ID NO: 119 - DNA, 206 bp, synthetic construct
  source 1..206: mol_type = other DNA; note = Description of Artificial Sequence: Synthetic polynucleotide
ataactagaaagtggtttcatgaaggtccaaagcacaaactgttgaaattgaataaaactaggattgaaaacaattctgaaacaaatttttctcattcttaccagataaaagttgctttataggttttgagttagaatcactaggtgctttcacataatatggataaatcttttctatggtcctgtacattaagaaaaaaaagata

SEQ ID NO: 120 - DNA, 220 bp, synthetic construct
  source 1..220: mol_type = other DNA; note = Description of Artificial Sequence: Synthetic polynucleotide
ccggaagctgaagggatgctttgaacgtgggggggctgcgtcacagttggactcccacttgcagaggacctgattatgtccagtgaccacctgaacaacagcacactgaaggaggctcagttcaaagacctgttcttaaaaaaaggtacatttcccaagaactgctatggatgatgcatgctctttcgggtttgctgtggttcttcttggagtttggtct

SEQ ID NO: 121 - DNA, 228 bp, synthetic construct
  source 1..228: mol_type = other DNA; note = Description of Artificial Sequence: Synthetic polynucleotide
aaacttacttcaaggctgcataataaaatgttccaaaccaaacaccagaagttattagatgcactggaatcagaacttttccatactgtctaaatgtcttcttgaatcgttgataaagactaatagatttgtcttgcaaaggatcaggctcttcctttttttccggagttccctgagctgtggcactggatgaaaaaaccctcctgaatgaaacatgttgcttccctt

SEQ ID NO: 122 - DNA, 173 bp, synthetic construct
  source 1..173: mol_type = other DNA; note = Description of Artificial Sequence: Synthetic polynucleotide
aaacctaaaggcttaaaaaaacaagacttgacactgagagaaggtagatctaatttagtgttacagaatctttattttgaaattgttgtacttgtgagctggcaacttgtactgacttgattttttttcttccacttagcatggaggatgaaaaggaagatagtaaagttata

SEQ ID NO: 123 - DNA, 224 bp, synthetic construct
  source 1..224: mol_type = other DNA; note = Description of Artificial Sequence: Synthetic polynucleotide
agaagaacaaagtaaacccaaattgtacactctttattgtctaatttttttttaactgaaaggaagtagcagaaccactcctggacctgaaggaaggaatagaccagttggagaacaataaaaccttgggctttatcctgtctactctcttagccattgggaactttctaaatggaactaatgtaagtcatccccatccctcatcctgtagcccccccttgtca

SEQ ID NO: 124 - DNA, 217 bp, synthetic construct
  source 1..217: mol_type = other DNA; note = Description of Artificial Sequence: Synthetic polynucleotide
atacagtcttaaattattaagttaattgaattttacatgagcaaaaaaaagtcttcctcccaccctgaatactgttgcatataaatgtttttgctgtgagaacagtaaaataataaactaagtgaaggcttactagaataggaagtagcatccatctttccaaccttaactggagaaccaatgcttttcatctcaagaccaacttcattccaaattg

SEQ ID NO: 125 - DNA, 229 bp, synthetic construct
  source 1..229: mol_type = other DNA; note = Description of Artificial Sequence: Synthetic polynucleotide
gatcatgaccttgtcaatgtctacaaggagacgtgggtcgggtcagctcgggcctcttgtacacacagggtgacctaactgcacccctcggtccttcgacctgcactgagtgcccacccagtaccaggacacaaggggacccacaccgggaacgagaagggggcgatggcgttgccggacggagaggaactgggagcccactgcctgcccccccccgggggccgctgtg

SEQ ID NO: 126 - DNA, 213 bp, synthetic construct
  source 1..213: mol_type = other DNA; note = Description of Artificial Sequence: Synthetic polynucleotide
gggcgctgctgagtcccctgcctggcggggccctcgaccgtgtcctgaaaaacctcacagcctgggctcacagcctggatgctcaggacagtctcaaggtgggaactgggggggcaggtgggcgtcctgtcacagcaaggtccaaccccattgctctgaccctatgaccttgacccccacccagatctggttcaacaacaaaggctggcactc

SEQ ID NO: 127 - DNA, 214 bp, synthetic construct
  source 1..214: mol_type = other DNA; note = Description of Artificial Sequence: Synthetic polynucleotide
ggaagccgaccggggcgtgcggagggatgtggcccggaatattggtagggggggcccgggtggcgtcatgcaggtaccccgcgctggggcctcgcctcgccgctcactttccagcgcagcggccgccgcagcggacactccagaacggcctggccctgcagcggtgcctgcacgcgacggcgacccgggctctcccgctcattcccatcgtggt

SEQ ID NO: 128 - DNA, 228 bp, synthetic construct
  source 1..228: mol_type = other DNA; note = Description of Artificial Sequence: Synthetic polynucleotide
tctttctcacgaatggcactcacctcaaatgtctctcatggcttttgttctgataatcaataccacaggactcctggttttcatgtaaactggagaaccaggaatcattccttttgaatcttactattttctgttcattggatattttttctccacaaatatcttgtgaagcaactgattcattagttttaagttgagtctcaaaacctgaaacgaaaaaaagtgaaa

SEQ ID NO: 129 - DNA, 219 bp, synthetic construct
  source 1..219: mol_type = other DNA; note = Description of Artificial Sequence: Synthetic polynucleotide
gaggaccctggggagaaagtggggtttcagtgaagctaagaggggtcatggtgttggggcgttagtgaaattgacttggggggggtcagtcatggaagggaatagggatgtggacaatcggggtcagagtgtcagaagggtcagaggtcaaagggcacagggcacataccacattgcctttggcaccaggagcaccatcaattccagtcacaccctagg

SEQ ID NO: 130 - DNA, 185 bp, synthetic construct
  source 1..185: mol_type = other DNA; note = Description of Artificial Sequence: Synthetic polynucleotide
ggcaggcgcggcggccggtgcccccagaagtggcccagcagtaccaggacatcatgcaacgcagccagtggcagagggcacagctggagcaggggggtgtgggcatccgacggggtaggggtttggagatgggcatctggtgggggaggagctccaggataggcatggggggggaggtgctcagg

SEQ ID NO: 131 - DNA, 217 bp, synthetic construct
  source 1..217: mol_type = other DNA; note = Description of Artificial Sequence: Synthetic polynucleotide
caagggagataggcaggaacaaggggggggggtcctctcccctttagggaaactgagacacagaggagtccttgagacccacattggagttcctgaggtctcgctttcccagcgggtaaactgaggcccagagcggtgggagggctccgccctcagccccacttccacctactctcccagggctccttcaccgtcatgggagtcatcagcggccccc

SEQ ID NO: 132 - DNA, 220 bp, synthetic construct
  source 1..220: mol_type = other DNA; note = Description of Artificial Sequence: Synthetic polynucleotide
tttagttactgtggatcccttgcccaacatcagagaattcatactggagagagaccctatgaatgtaaggaatgcaaaaaaaccttcaggcagcatgcacaccttgctcatcaccagagaattcacattggggagtcactgtcaccacccaacccagtcaatcaccaagtcctatagatcctgagtcctaaatgtttctagaatttatactgttttttat

SEQ ID NO: 133 - DNA, 195 bp, synthetic construct
  source 1..195: mol_type = other DNA; note = Description of Artificial Sequence: Synthetic polynucleotide
ctgttgctcctgcacgctccccctcccctgtgaccgccccccccgccccgcccttggtcccttggggccgggtagctgcctgaaggggcggggccattctctcagatataaggcttggaagccagcagctgcgactcccgagacccccccaccagaaggccatggtctccccacggatgtccgggctcctctccc

SEQ ID NO: 134 - DNA, 216 bp, synthetic construct
  source 1..216: mol_type = other DNA; note = Description of Artificial Sequence: Synthetic polynucleotide
gggttgggggggcccattactgaggccccccccagagaatcaggcactaactagcacagggagtgggtgctcacatccctggagcagggagctagggtgcagctgggatgggcagagagcatcgcccccctcgactgcgccaagctggggagaggggttgcaggcaggatcatagatagtagatttcacggagatttccatcggccgcgccttttc

SEQ ID NO: 135 - DNA, 217 bp, synthetic construct
  source 1..217: mol_type = other DNA; note = Description of Artificial Sequence: Synthetic polynucleotide
agcgtccaatagttttccaacggaatagcttttcttacctgggaatgtcccccccagatagttgacactcaggaacagcacggaacaataatggctctgcctctgtctcatcatcttcttggaaaaaatgtgagatgtcacaaagggtctcagaaacacagggtagctccctgtataccctggaaaacaacaacagaatttttactatgaatataag

SEQ ID NO: 136 - DNA, 209 bp, synthetic construct
  source 1..209: mol_type = other DNA; note = Description of Artificial Sequence: Synthetic polynucleotide
tctccctcccctgctcccagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccggtaaaggcttccaggcacccccactccttcccctcctgtcctcacggccg

SEQ ID NO: 137 - DNA, 232 bp, synthetic construct
  source 1..232: mol_type = other DNA; note = Description of Artificial Sequence: Synthetic polynucleotide
acaagacgggcaaaggtggttatggagcagctgccgggggtgccaccaggccccccccaccccgttcgaccgccacccccaaatgtcagagcctgggtgggccagcagccgcctatgccactgggaaggcctctggggctggaggggcagggggccaggcttattcccccggtcagcctcaagggcttctgggaccccaggcctacgggcaagggtttggaggggggcaggc

SEQ ID NO: 138 - DNA, 198 bp, synthetic construct
  source 1..198: mol_type = other DNA; note = Description of Artificial Sequence: Synthetic polynucleotide
ggagtccggggtgccccccccaccgccaccccgccctcaggagccctagatcccagttctccaggtcctcaggccccccgctgcatctccaggctccccctccagcatgatccctgtctgtccgcagtgggccgcctggccggcctggacgcaggcctgcaccagctgcacgtccgtctgcacgcgttggacacccgc

SEQ ID NO: 139 - DNA, 212 bp, synthetic construct
  source 1..212: mol_type = other DNA; note = Description of Artificial Sequence: Synthetic polynucleotide
gaggatcctgccctgcccccatcccctgcgtacctgttgccagagcaagcaggattaactgcagaatcctcatggcctggaggggggaggagcgggccccaggttcctctgggaacaaggagggacatggggccgcatcactttacggggaaatcgggaggggggggctggctcatgccctctcctctctccctcacctgctcccgctcccc

SEQ ID NO: 140 - DNA, 227 bp, synthetic construct
  source 1..227: mol_type = other DNA; note = Description of Artificial Sequence: Synthetic polynucleotide
cactgtccccacatggccaaaatctactgttcccctgggagggcctctatcccctgcatgcaatcaaactattccagcagggtggaaatcgcagttacacaagtggttcgacagccacatcccccggtgggcctgtacccctcctggctatgtatttttatgtgggccacaaaaaaataaactgccctttgatggaagtcctaagataacttattcaaccccccctg

SEQ ID NO: 141 - DNA, 227 bp, synthetic construct
  source 1..227: mol_type = other DNA; note = Description of Artificial Sequence: Synthetic polynucleotide
cactgtccccacatggccaaaatctactgttcccctgggagggcctctatcccctgcatgcaatcaaactattccagcagggtggaaatcgcagttacacaagtggttcgacagccacatcccccggtgggcctgtacccctcctggctatgtatttttatgtgggccacaaaaaaataaactgccctttgatggaagtcctaagataacctattcaaccccccctg

SEQ ID NO: 142 - DNA, 202 bp, synthetic construct
  source 1..202: mol_type = other DNA; note = Description of Artificial Sequence: Synthetic polynucleotide
acccacccaaatttttttttcaattactcgatgcaatatcaaatacttactaagtaatttgtagttgtactctcttcccctctgcccatgtatctgtagaagcgatgatctgctaccaccaataatttacacgtgttcttcatgggatctgggtcagctcttcttttcactcgatgaacaagctctaatatgaatttgtatg

SEQ ID NO: 143 - DNA, 225 bp, synthetic construct
  source 1..225: mol_type = other DNA; note = Description of Artificial Sequence: Synthetic polynucleotide
gccagctttcaacagtgtcttgatctcgttgcgcaggtcagcctgactagagaagtccagtttggggatgttcaatttgtggaagtatttagtgttgctatccagggtaagctgattgtttatcttgacaatcactccattactaagctccagtgtatttttttctgtgtgtaaacttgccactgtgtttgattttccctcaatagcatttccaaaaaacagcat

SEQ ID NO: 144 - DNA, 224 bp, synthetic construct
  source 1..224: mol_type = other DNA; note = Description of Artificial Sequence: Synthetic polynucleotide
tttagcaggtttccaaaaaaaagcagcccgtggcccttcatctgtgtccgtctttttctctttctgtctccaggcagcagaagcatttctagttcatctctttgaggacgcctatctcctcaccttacatgcaggccgagttactctcttcccaaaggatgtgcaactggcccggaggatccggggccttgaggagggactcggctgagctcctgcacccagtg

SEQ ID NO: 145 - DNA, 187 bp, synthetic construct
  source 1..187: mol_type = other DNA; note = Description of Artificial Sequence: Synthetic polynucleotide
tggagggagtgattgtgggagcactgaagttcttaggtgtctacttttttttttattcttcctcactgaagatggcagtgctggagattcagggcaatggagagtcctcatatgtgccacaggaagccatcagcagtgccatccagcttctagaagacccattacagagggtaaggatcttcaggat

SEQ ID NO: 146 - DNA, 207 bp, synthetic construct
  source 1..207: mol_type = other DNA; note = Description of Artificial Sequence: Synthetic polynucleotide
ctgtgctataattcccaccaaatcaatgaagattacctgtggacgattcaactggaaaacaactggtggctgaggccattcactcaggaaagaaaaaagaagcaatgatccagtgctcattggaagcttgtcggattcttgacactttgggattgcttcggcaggaagcaggtcagtatataggagccctttttttctagatttaaa

SEQ ID NO: 147 - DNA, 179 bp, synthetic construct
  source 1..179: mol_type = other DNA; note = Description of Artificial Sequence: Synthetic polynucleotide
aaagtataactaggagatacctgatctttcggaagtagaaaaaaaaaagttctacagataaagttaagaggctgtgtatcaaatcttcaccagctaacagtacataagaaaccaaagttcatcttatggtgtttgacagaagtcacacaaagcaactttacttacctggagaagtgtcc

SEQ ID NO: 148 - DNA, 188 bp, synthetic construct
  source 1..188: mol_type = other DNA; note = Description of Artificial Sequence: Synthetic polynucleotide
tcaattaatttagttgcttgcaaatataaatcaagcttgctccagttccacaaggactccaccacagtctttaggatggagaaaaatcactggttttccatgtgctcctattttgacctcttcacttagactgcggatcttcttttttttcaaatccatcacagctgcattaatattatccaccttaa

SEQ ID NO: 149 - DNA, 204 bp, synthetic construct
  source 1..204: mol_type = other DNA; note = Description of Artificial Sequence: Synthetic polynucleotide
acaaccagagaaatttattttaaagtattccttaccttttccagttctatatcttgaaatgggaattgtcctaccacctttttgtacatctcttcacttgttactggtataggactgtagttgtcagaatcaagtatgtttctataaatggaaggatagacaaagggaagggaaggatagacaaagtgggggggtaggggaggg

SEQ ID NO: 150 - DNA, 204 bp, synthetic construct
  source 1..204: mol_type = other DNA; note = Description of Artificial Sequence: Synthetic polynucleotide
ccctggtgtgtgcactgccctaaccccgacctcccactcaccttaaccaggggctcaggtttcctgggggggactctgtgtctcagtcgctttttccgtctttgaaccaagttttccaagtcagccaggttatccagattaaatctggtactgttaaatctttcaagctggggcagaagacaagaccatgccagccatcagccg

SEQ ID NO: 151 - DNA, 197 bp, synthetic construct
  source 1..197: mol_type = other DNA; note = Description of Artificial Sequence: Synthetic polynucleotide
gctgcgccgcgcccaccgctacgacctggtcaacctggcagtcaagcaggtacccaccccatgcccttcctccctctggcaactcagcacatgcttgtccagcacctcccccccccaccaaccaggtgcagccgatacccacgggtgcagccttaccaggtgccaactgcagattctgttccaatggggcagcaccc

SEQ ID NO: 152 - DNA, 189 bp, synthetic construct
  source 1..189: mol_type = other DNA; note = Description of Artificial Sequence: Synthetic polynucleotide
ttcggagacccccacctagccggcccgagctgaccctctccccaacccgtacgcccgcggaccgagctcctcaccctggtgattgaacagtctccatattctagtgaagagaattcccattctcgggcagcggaccccccccctccagacacccgggccgcctggcttcccccggctcaggctgagggg

SEQ ID NO: 153 - DNA, 212 bp, synthetic construct
  source 1..212: mol_type = other DNA; note = Description of Artificial Sequence: Synthetic polynucleotide
tggttctccagtgtaccttgttagatgtttaaaagaattatttatttcaatcactgactacaaaatttatattagtttataatctgtctggtaaataaaactgttgaattattttgaaatgtacatttttttttactacaaagaaaagcttattctacttgatttacaatgagaaaaaaaaatcatgtagcatttatttgtaaatagattta

SEQ ID NO: 154 - DNA, 177 bp, synthetic construct
  source 1..177: mol_type = other DNA; note = Description of Artificial Sequence: Synthetic polynucleotide
ctctactcattgctctctgtctgaggttgggatcattcagcatatcctctgaaaggagataggaactacaacgccttttcttgtgtatttgattggtcgtgccctaaaaaaaaaatcaattaatgtcttaagaacagaatcctaataaattgtaggtataagataaaatcttgctta

SEQ ID NO: 155 - DNA, 207 bp, synthetic construct
  source 1..207: mol_type = other DNA; note = Description of Artificial Sequence: Synthetic polynucleotide
ggaaacctatggaactaaagctgcttattgattactttttttttcctccatcaggtatgaagatgataaaaaagaatgggctgggatgttgaaggaaatacgttatgcttcaggagctagttgcctagcaacacgtttaaatctcttcaaactgtctaaactgtgaacaaggtgacaaaacataatagattgggaggtggtttgttt

SEQ ID NO: 156 - DNA, 223 bp, synthetic construct
  source 1..223: mol_type = other DNA; note = Description of Artificial Sequence: Synthetic polynucleotide
cttattaataaagtttttttccttcactctaatcaaaaccctacccttatttggtagtgttacctaatttcaattattgctactattacctctccagtcctactagcctctggtcgagataacactgatgcagaaattggtctgtatgcaactctggatccagctcggatctattaatgaaaaaaaaataaagatttcaatattaagaaagaatacaggtaag

SEQ ID NO: 157 - DNA, 219 bp, synthetic construct
  source 1..219: mol_type = other DNA; note = Description of Artificial Sequence: Synthetic polynucleotide
atttatcactattattctacaaaatacagtgaaaatgccatatctgttcctttttcttcacaggtgtgtagtaatgaagccacctgcatttgtgatttcacctgggcagggacagattgcagtatccgggatccagttaggaaccttcacccccccaaggatgaaggacccaagggtttgtgtgattttggtttcaattcatggaatactgaattcgtt

SEQ ID NO: 158 - DNA, 234 bp, synthetic construct
  source 1..234: mol_type = other DNA; note = Description of Artificial Sequence: Synthetic polynucleotide
gctcatgggcatatagtctccactgccctttcggccactgggcactggggccacccctggggacatgggcatgtagccatcatccgtgtggagggtggagctgtctgggcggtggtgccccccccgacgctccaaggggtgcatttccagaccctcctctgggtaggagcgggtgggcacgaaggcggagtgcctgtgtcccggcagtcggcctccactgccacctcctggtgg

SEQ ID NO: 159 - DNA, 199 bp, synthetic construct
  source 1..199: mol_type = other DNA; note = Description of Artificial Sequence: Synthetic polynucleotide
cccctgtcccgcccgcagcagcctcctccagccgcgagcccagctgcagggagcttctggcctgggaccccccccaggagctgcaggcagacgccgcccggctctgcgccttctatgcccgcctgtgcccggggtcccagggcgcctgtgcgcgcctggcgcaccagcagtgcctgcagcgccggcggcgatgcggtca

SEQ ID NO: 160 - DNA, 209 bp, synthetic construct
  source 1..209: mol_type = other DNA; note = Description of Artificial Sequence: Synthetic polynucleotide
ggccaaggccagtgcagccccaggttcccgagcggcaggttgggtgcggaccatggcctctcacaagctgctggtgaccccccccaaggccctgctcaagcccctctccatccccaaccagctcctgctggggcctggtccttccaacctgcctcctcgcatcatggcagccggggggctgcagatgatcgggtccatgagcaaggata

SEQ ID NO: 161 - DNA, 221 bp, synthetic construct
  source 1..221: mol_type = other DNA; note = Description of Artificial Sequence: Synthetic polynucleotide
ggtgcccggcgccactccccccccacagccaggttacctccttgtttttttccttgtccacagcagtccacacgaagccgaaggccccactgcccagcgggctcatggtactgtacttttgggagtactcgccctcacaggccgccaacccctccagttccacagccttggggggctcctcaaaccagggtctggctctgagcacctgcaatgcagaagaa

SEQ ID NO: 162 - DNA, 203 bp, synthetic construct
  source 1..203: mol_type = other DNA; note = Description of Artificial Sequence: Synthetic polynucleotide
tgggatggaatggccttggcttggccagttggttggtagtagggtttggctgaattcaccaaacgtgcttttttttttctctctctctctcgctccctccctccctctctagccttcatatgctgagtttgatattagtggaaatgtgctgggtttgatcttcaaccaaggaatgatctggtgagttatccatttcatctggt

SEQ ID NO: 163 - DNA, 184 bp, synthetic construct
  source 1..184: mol_type = other DNA; note = Description of Artificial Sequence: Synthetic polynucleotide
atcgggtatgaggcaaaatcatatggtgcttgacgtactggggcccccccagcatggtctccagtaaaaagacagtttctaagaattcaggctttgaattcatatccttctctgctaaataccacagcaattcagacccctcttgctgaaggtgagtactaagacgattgcctctgtaatctgg

SEQ ID NO: 164 - DNA, 224 bp, synthetic construct
  source 1..224: mol_type = other DNA; note = Description of Artificial Sequence: Synthetic polynucleotide
ggaccaatcaggaacttgtcttctcaggaacgtacctgtttattgcgtggatgggggggggtggggcacaggacagccgtgcacaggcgtagtagtcaccaatagactcgatgatgctggcgaccacggcactgagcatgccgatgacaccggccgcagacacggtgggcagtccccactgaactgtgggaggaaaacaaccatgaatgctcctagagaggcag

SEQ ID NO: 165 - DNA, 179 bp, synthetic construct
  source 1..179: mol_type = other DNA; note = Description of Artificial Sequence: Synthetic polynucleotide
ggagatgttggtgtaaccataactgatcctttcaagcattttgtgggaatgcttcctgaaaaagattgtcgctatgctttgtatgatgcaagctttgaaacaaaagaatccagaaaagaagagttgatgttttttttgtggtaagcatgttagaaatattgagcctctgtaaaactcat

SEQ ID NO: 166 - DNA, 223 bp, synthetic construct
  source 1..223: mol_type = other DNA; note = Description of Artificial Sequence: Synthetic polynucleotide
agtcttggccactgcttctcataagaccagtcggtattttcctctttgccagaaatgcttatcctcatgtaggactggaggcagcaggactgacaatttctgcaatagaggtagggctgttcgttctttttgcaagaggccctacaaattccactgttacccatgcatcgaaggatgtggcgtttgcctgccaaaggaaaaaaaatacaaatagttaattcac

SEQ ID NO: 167 - DNA, 215 bp, synthetic construct
  source 1..215: mol_type = other DNA; note = Description of Artificial Sequence: Synthetic polynucleotide
aatgactacagacgcattgaattcccccgaggtgttggtgatattccctacaatgacttccatgtccgaggaccccccccagtatataccaacggcaaaaaacttgatggtatttaccagtatggtcacattgagaccaacgacaacactgctcagctggggggcaaataccgatatggtgagatccttgagtccgagggaagcatcagggacct

SEQ ID NO: 168 - DNA, 227 bp, synthetic construct
  source 1..227: mol_type = other DNA; note = Description of Artificial Sequence: Synthetic polynucleotide
cctgagggtagttgggggacgtgagtcctgccccgcgtgccagctccccaaaggcccccaccgccgaccccggcgcacctgccaggacatccaccgacttgttggaggggccgcagtacaagatgcagggaccccccagccgcttctccccacggggggggcctccgggctgcacctgctcctggtttgatttatgaaaccagaatacgatgtggaggcccacgatc

SEQ ID NO: 169 - DNA, 202 bp, synthetic construct
  source 1..202: mol_type = other DNA; note = Description of Artificial Sequence: Synthetic polynucleotide
aataattatgccctggttaaattttttttcttaaattcatcaaactaaaaaaactacacaggctactaatttcataaggtacctacctccagcatctaggacaatatctactcccaggccacctgtttcttccaaacagctttcagcaacatgaactttcccattagatacatcaatcactcgggctgtaaaggacaggaaa

SEQ ID NO: 170 - DNA, 202 bp, synthetic construct
  source 1..202: mol_type = other DNA; note = Description of Artificial Sequence: Synthetic polynucleotide
tgttcattctggggcctcacattcctgggctgtaaaatggaaataaaaacacccacctcatgcagtttgggaggagcataaagtgagatacacacagtatgtgccaccaaatgattattcctcctctgcccccccccaatccccaactgtgcgcaccggcatgtccttcctgcaacctgcttctctgatgcccgttgctgag

SEQ ID NO: 171 - DNA, 229 bp, synthetic construct
  source 1..229: mol_type = other DNA; note = Description of Artificial Sequence: Synthetic polynucleotide
ccgccatcacccagcggatggtggatttcgctggcgtgaaggacaaggtgtgcatgcctgacccgttgtcagacctggaaaaagggccggctgtgggcagggagggcatgcgcactttgtcctccccaccaggtgttcacaccacgttcactgaaaacccactatcaccaggcccctcagtgcttcccagcctggggctgaggaaagacccccccagcagctcagtgag

SEQ ID NO: 172 - DNA, 183 bp, synthetic construct
  source 1..183: mol_type = other DNA; note = Description of Artificial Sequence: Synthetic polynucleotide
cacttaaaccatattctgtaaggacaggacaaattttcctcctatgagagagtggaaaaaaaaaattccagtaaccataagataataatattacctttatttctgcttagtgacagtgcaatttcagaattgttattcaaaggacggcgttttcctttccctacaagctctgtatttacaaag

SEQ ID NO: 173 - DNA, 205 bp, synthetic construct
  source 1..205: mol_type = other DNA; note = Description of Artificial Sequence: Synthetic polynucleotide
tccaacatcatcatatatatatatgattttttttttgccgtagattttactatgagcagcgattactacggaaggtcttcgaagaatggaaagaggagtggtgggttttccagcacgagtggaaactctgtgttcgagctgactgtcactacaggtcaggtttcatgttacactccttcagttttactgatgcacattatcagat

SEQ ID NO: 174 - DNA, 210 bp, synthetic construct
  source 1..210: mol_type = other DNA; note = Description of Artificial Sequence: Synthetic polynucleotide
gccttgcagaggcttgacccgcacctgaaactcatactgggtgtctggggtgagcgtctccaggcagatccattcctgcttctgcttgagagtcagcaggggggcctcctgggtcggagacaggactgtcagcgcccacctcacctcccatggcagggccaggaggctgagcatcacagaacccccccccaacccctgccagctgcaccc

SEQ ID NO: 175 - DNA, 225 bp, synthetic construct
  source 1..225: mol_type = other DNA; note = Description of Artificial Sequence: Synthetic polynucleotide
ctacccttgtctcccattcaacccaatttaacaaattaaggttctagattgaagaacacttacgtgggaagttagcatgatggaaggcaaacacgggtttaggacaaacatctccccccctcactgtaatctccttctttcggcgtagctcatcaacctcatactattgaaaaaaaatgaaagaagttagtaaactagttattctaaacattcagaatttatgtt

SEQ ID NO: 176 - DNA, 229 bp, synthetic construct
  source 1..229: mol_type = other DNA; note = Description of Artificial Sequence: Synthetic polynucleotide
gtgggataattgcttgagcccaggaggcagaggttgtagtgagccaagatcacgccactgcattccagcctggatgacagagcgaggccctgtctcaaaaaaaagagactgtctgtttttcaggtcttctttgtgatccgcctcattgctggccctgctgccaactccctgcctcccattgttgatcctgatcctctcatcccctgcgatctgatggatggtcgggatg

SEQ ID NO: 177 - DNA, 201 bp, synthetic construct
  source 1..201: mol_type = other DNA; note = Description of Artificial Sequence: Synthetic polynucleotide
gagaagattcacttaccgaataaaactcaagtgaacaccaagtgaccggtgggaccctgagcaatcaatgcaaaggaacactccataggttatgcttgcccagctgggatttttggcaccacaatcaaaacacacctgaaaaaaaatgttaaattcagttactaatttatgttagccaaataaattatcctgtaagataca

SEQ ID NO: 178 - DNA, 180 bp, synthetic construct
  source 1..180: mol_type = other DNA; note = Description of Artificial Sequence: Synthetic polynucleotide
ctgtgaacagcagcggagacgtgaggctgcttatggcaatcaggacctccacctggaaaaaaagcgcgggttgcaggaccacggccggagcctgggagcccaggacagcgggcgtggccagggctgggggggcgggcatggccgggcactcatgggcatagtcccaagctggttccctgc

SEQ ID NO: 179 - DNA, 218 bp, synthetic construct
  source 1..218: mol_type = other DNA; note = Description of Artificial Sequence: Synthetic polynucleotide
gaacagccctttcaattctgttttttttttcccaggttgattccaaagatagttctcataactccacaaactctgaatttgcagctgaagctgagggtcagaatgatacaattgaggaacccaacaaggtccagaaaaggaagagggatagacttcgagaccagggctccacaatgatctacctgaaggctatccagggcatcctggggaaatcgatg

SEQ ID NO: 180 - DNA, 223 bp, synthetic construct
  source 1..223: mol_type = other DNA; note = Description of Artificial Sequence: Synthetic polynucleotide
cagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatgtaggtttatttttttcccttcttctaccaagaaaaaaaaaattgtctct

SEQ ID NO: 181 - DNA, 220 bp, synthetic construct
  source 1..220: mol_type = other DNA; note = Description of Artificial Sequence: Synthetic polynucleotide
aacaccagtctagctcttcattgtttcacttttttttttctcattggtagaggcttttacttaataactaccatccttaaaatctgaaagtattcagtggtttaatacctcaagattttaagttagcctcttcacatttgtgacatctcatttaaaagagtattgtgattcttttcagcaccaggaatatcttagatgattttagagaagcttacttttg

SEQ ID NO: 182 - DNA, 219 bp, synthetic construct
  source 1..219: mol_type = other DNA; note = Description of Artificial Sequence: Synthetic polynucleotide
gctgggcagggaacaggaaatataaggcttaccactacagcagaaaccagaaactcctgagcaccacttatcttttttttctgttgattgaagttgtcaattatgaccccaacaaagagattcagtgtgaagaagcctccaaaaatgatgaagatgacaaagtacaaatacatgtacacgttgtcctcccacttgggttgcatgttgacctgtgaccag

SEQ ID NO: 183 - DNA, 226 bp, synthetic construct
  source 1..226: mol_type = other DNA; note = Description of Artificial Sequence: Synthetic polynucleotide
atgtagtagtacaaccagttgtagcagaaaatattcctgtaataccactgagtgacagtccccccccttcaagatggaagcctggacagaaaccttggaagccctcttatgagcgaattcaggaaatgaaagctaaaacaacccatttgctacccatccaaagcacttacagtttagcaaatattaaagagactggtagctcatcatcctaccataaaagagaaaa

SEQ ID NO: 184 - DNA, 179 bp, synthetic construct
  source 1..179: mol_type = other DNA; note = Description of Artificial Sequence: Synthetic polynucleotide
agaatttgttttcattctgtggctctacccagaaccagcctgagaatgatcaaagataacatcctttcttcctctaattttttttttctttccttatgccaaaggcttcacccaaaaaactgcctgggtgtgcgccagtttgctgagacaatgatgtgtgctgtgctgtacgacgctgc

SEQ ID NO: 185 - DNA, 223 bp, synthetic construct
  source 1..223: mol_type = other DNA; note = Description of Artificial Sequence: Synthetic polynucleotide
gtttgtggtgggggggggaccacaaaccccggccctgccctcttgcttacataggcttcccgcctagaagcgcagccagaacatgccgctacggatccggttgtagtctgggagctgctcaatggggcctgtggggatagggtggagcgtcagggtggggtacctggcctgcacagggacacctggccctgtgccgcacccagctcggagatgctgcccagag

SEQ ID NO: 186 - DNA, 233 bp, synthetic construct
  source 1..233: mol_type = other DNA; note = Description of Artificial Sequence: Synthetic polynucleotide
gtgccccttcatctctgtcccagtttgcctgaaccagtccttgccagggtctgggatgtaggcagggcccaaaaaacttacactggttgtgtgtggtgtggttcttggacttggccatgtctgcacctggaagacaaaagtggagatcagggttaagggctcgccaggctgggccacctctgccccccccattctggtcagaatgagcctgcaggcagagaatgtgcagcctc

SEQ ID NO: 187 - DNA, 192 bp, synthetic construct
  source 1..192: mol_type = other DNA; note = Description of Artificial Sequence: Synthetic polynucleotide
ggggtggagggaggggccgagagccgggggggggcgcttccgagagcccagaccgaggccccctgcccgcccgccatccgccctcaccgtggatgcccgtttggtgcgccatggctcggcgcttcgctccgtccgcgccgtcggagccctccgcttgaccctggcccggcaacgctcgctggaccaagagag

SEQ ID NO: 188 - DNA, 211 bp, synthetic construct
  source 1..211: mol_type = other DNA; note = Description of Artificial Sequence: Synthetic polynucleotide
ctcacaatacattcaggtataggcttcttagaacctgtttagatttaaacaaacaaacaaacaaacaaacaaacctttttttagaccattaaataaccttcttcataaactattttctagataatgttctactcttataataatgcagacacaagtctcttgacacagaagtcatacattttcatgtatgataaacattatgaaaaaaaac

SEQ ID NO: 189 - DNA, 208 bp, synthetic construct
  source 1..208: mol_type = other DNA; note = Description of Artificial Sequence: Synthetic polynucleotide
ttactttcttaatttgtcaaagttagtatttaaaaatttcagatgactgatcatttctttacaaatttcagatgactgacccagtcacattaaatgtaggtggacacttgtatacaacgtctctcaccacattgacgcgttacccggattccatgcttggagctatgtttgggggggacttccccacagctcgagaccctcaaggcaa

SEQ ID NO: 190 - DNA, 170 bp, synthetic construct
  source 1..170: mol_type = other DNA; note = Description of Artificial Sequence: Synthetic polynucleotide
cggggttcctgccatggctcagcttacgctgtcatccctgtaagtgttagaaccaatgattcaagtatcaactgtggcagcagcttagttaccttaggtgcaaaaaagagaaatatttttttttgttcatatatataaattgtcgattttcataatgaaatcagaagaga

SEQ ID NO: 191 - DNA, 221 bp, synthetic construct
  source 1..221: mol_type = other DNA; note = Description of Artificial Sequence: Synthetic polynucleotide
tgcatctgcacaggcttccctgggaggacttcatcaacagcacagttacacatcaaagtcctttagctctccccagctggcgcctattttcactttcactttcaatttcacagacagttttacagcactttccatttcattcttgacaatctgagctacctaaggaaaaaaaaaatgtagttaacaaactgcccagtcaaagaatgtttcccaaaccatcc

SEQ ID NO: 192 - DNA, 216 bp, synthetic construct
  source 1..216: mol_type = other DNA; note = Description of Artificial Sequence: Synthetic polynucleotide
tctttctaaccctactgtattttgcatttacaccttttccttcatactaacctggatgtcccgggcccgttcataggactgatacgcgattttttcttccatgcttgccagttcctgcttcaagttcaagatttcattctggtggagctctgttaggtcatttagctgttcttccaatcgttcacatctaaggagaaaaaaaaagtttagaatttt

SEQ ID NO: 193 - DNA, 212 bp, synthetic construct
  source 1..212: mol_type = other DNA; note = Description of Artificial Sequence: Synthetic polynucleotide
aaggccctggctgatttcatccgcaacaaactctcttccatcaaggcatatctgacaatccactcgtactcccaaatgatgatctacccttactcatatgcttacaaactcggtgagaacaatgctgagttggtaagtagcaaagtagtaggtatgacattttactattgagattttttaaattctaatcctgaaaaaaaaatcattataag

SEQ ID NO: 194 - DNA, 200 bp, synthetic construct
  source 1..200: mol_type = other DNA; note = Description of Artificial Sequence: Synthetic polynucleotide
cattaattcaagctgctgctctcgtatttccttcattctttcaatttgtggagtatattcttgattaattagattgccaacatcacagagagccgttaggaaacttttttttttctgttctaatgtatcattaagctccttaaaatactggagaacagcttccttatcgccttggatcattttctcagaatgagattttt

SEQ ID NO: 195 - DNA, 228 bp, synthetic construct
  source 1..228: mol_type = other DNA; note = Description of Artificial Sequence: Synthetic polynucleotide
tttggaaagaggtagcacataactgcagttaatttttttcccaataatattctctctagggccaagagcctggctggcagagctcgggtgggtccgtgtttccatccccagaagaggccccgtctgctgccagccctggcactgagcccactggcacctcagagccactgggaacaggcactgtctcagcagaactgtccagcccatgggctgccagggactcggagc

SEQ ID NO: 196 - DNA, 218 bp, synthetic construct
  source 1..218: mol_type = other DNA; note = Description of Artificial Sequence: Synthetic polynucleotide
tcatacctgtgatcattacagtccttatccgggctgagatgagctcttccaagacaggttttgtctcttccttcaatcgattctccaagatcagcagccccagaaatatcaggtctgattctaccgtctccctgaaaatacatatatagatgtattgaacagaaataacacatattacagctttttttttgagacggagtctcgctctgtcatccagg

SEQ ID NO: 197 - DNA, 220 bp, synthetic construct
  source 1..220: mol_type = other DNA; note = Description of Artificial Sequence: Synthetic polynucleotide
catttcggtcagtgtgctctgacacagctcctgaaaaacacgaaatccccgactctgcgatgactcacctgctgcgtgccctgagctccacgcacgggttctgtggtgtcaaggcagcgtgaggacagtgctgcgagctccctgcagcaggatggtgcccttgctgtgcgcctaccggtgggcgggggggtaagccaaggctgtggctgctcaagtcctg

SEQ ID NO: 198 - DNA, 193 bp, synthetic construct
  source 1..193: mol_type = other DNA; note = Description of Artificial Sequence: Synthetic polynucleotide
gagagcagcgacagcccgtccaccagcccgggctcagcctccagcccccctggacctcctgggacgaccccccccgggcagaagtctcccagcgggcccttctgcactccccagtcccccgtctccctcgcgcaggagggcaccgcccagatctggaagaggcagtctcaggaagtggaggccgggggcatcc

SEQ ID NO: 199 - DNA, 229 bp, synthetic construct
  source 1..229: mol_type = other DNA; note = Description of Artificial Sequence: Synthetic polynucleotide
cctagctgcccttaccaggttcttccagagatagtctctgcacatgctgtgtgggggggggccttcgtgtgcgggtgtgtgtgtgtctgtcctgctgccttccggccacccctgacagtcccacctctgcccacaggtctccgtgttggtcctcttcgccctggccttcctcacctgcgtcgtcttcctggttgtctacaaggtgtacaagtatgaccgcgcctgcccc

SEQ ID NO: 200 - DNA, 199 bp, synthetic construct
  source 1..199: mol_type = other DNA; note = Description of Artificial Sequence: Synthetic polynucleotide
aaatgctgatgattgtcaggatttgagaatccctgtccactcagacaaaccctaaacagcttgtcacatactgcaatgagcctcgtcactccagtcgtcacagtcgttgtagccattacattgcagtttcccggggatgcagattccactggcacacagaaagttctcaccccttccacagagcactaaaaaaaaaggg

SEQ ID NO: 201 - DNA, 191 bp, synthetic construct
  source 1..191: mol_type = other DNA; note = Description of Artificial Sequence: Synthetic polynucleotide
taataatgtaaaactcctatctgagatagagttccttgtggtaagtatgatcataaattaccaacaaaaaaaaactagtgatgatatataaaacccatttgtaaacattttttaatttccttgtgagaacaggatttaaaataattccaaagacctaatgataggactgaatagcatcttgcctgtaaatt

SEQ ID NO: 202 - DNA, 207 bp, synthetic construct
  source 1..207: mol_type = other DNA; note = Description of Artificial Sequence: Synthetic polynucleotide
gttagataataatcagatactgtttttttttccttcttcagtaactctctctcctttgaaaaggcatattgcgagtacaggctgaacagaattgagaatgccttgaagacaatagaaagtgccaaccagcagacagacaaactgaaggagctttatggacaagtggtaattactgcttttaaatacatgttgacagtgatactgagt

SEQ ID NO: 203 - DNA, 200 bp, synthetic construct
  source 1..200: mol_type = other DNA; note = Description of Artificial Sequence: Synthetic polynucleotide
tagaatggttctgtctgtagaagaatagaaacacctattccagatgtttcctaatttaaaaaattacctaatagcaaatataagacatttctattgttaatttttttttccgagacacttaccagcatattcttgagcgccccatcctgttacataagcagtagagccaggtggaatattctgggtagcagctgggagac

SEQ ID NO: 204 - DNA, 164 bp, synthetic construct
  source 1..164: mol_type = other DNA; note = Description of Artificial Sequence: Synthetic polynucleotide
atctaaatgatttgatagataactcaactatgaatgaattttaacataacttttttttttgtagcctataccattagaatcaagagaggtaagaatgactccacagaatttaccgtgcaattaacaaagagaagtaagtgttaagatcaggagactcagaacag

SEQ ID NO: 205 - DNA, 219 bp, synthetic construct
  source 1..219: mol_type = other DNA; note = Description of Artificial Sequence: Synthetic polynucleotide
ggaggactgtactgtgcaagttttgtttgccaatctaatgaaggttctctcatttttttttagttccttgagaatatgtatatcgggaaggaatgtaaacgtgcatgtaataagactcctataaaacgaactcaagcatagaagaatcgtaggagaatgattaggcagattttattactacgtacttggctatttctctgtctccttttaaagattaaa

SEQ ID NO: 206 - DNA, 186 bp, synthetic construct
  source 1..186: mol_type = other DNA; note = Description of Artificial Sequence: Synthetic polynucleotide
ttcccgccgcaggtggtcaatggtgatgctgttgcctgtgtctcggtcccacagacgcttattctgctccttctccagactcagctccttctcccttttgtgtagatcagcctaaaaaaagagaggaatgcttttttcctccctgctattaaataattaagataaaacatattgtctttatatcat

SEQ ID NO: 207 - DNA, 224 bp, synthetic construct
  source 1..224: mol_type = other DNA; note = Description of Artificial Sequence: Synthetic polynucleotide
tggtgtctttactgcagatgaaggatttgggggcagagcacttggcaggtcatgaaggggtccaacttctcgggttgttgaacgtctacctggaacaagaagagagattccaacctcgagaaaaagggctgagtttgattgaggctaccccggaggtaagttggcagaaaagataacttgctcaagcagctgatggggcttggaggaggtaacccccccgcccc

SEQ ID NO: 208 - DNA, 212 bp, synthetic construct
  source 1..212: mol_type = other DNA; note = Description of Artificial Sequence: Synthetic polynucleotide
gcgcagcgccttcagcaaccggagaagcatagttgcacgcgacctggtgtgtgatctccgagtgggtgggggagggtcgaggagggaaaaaaaaataagacgttgcagaagagacccggaaagggccttttttttggttgagctggtgtcccagtgctgcctccgatcctgagcctccgagcctttgcagtgcaatgtcccgcctgctgcat

SEQ ID NO: 209 - DNA, 217 bp, synthetic construct
  source 1..217: mol_type = other DNA; note = Description of Artificial Sequence: Synthetic polynucleotide
gtttatgtttcacatgaacaaaatccaaggttatagttagattctaaaataactaattttcatctaatcaacttgtctggaaacttaccagactcatgagaaattgtcttaacggtaagaagcttcacgctctctttatcagactgaggtctatttgaaaaaaaaaacatgaaattaacaccacagtaatcagcattgtatgtttgtgaaagtattg

SEQ ID NO: 210 - DNA, 218 bp, synthetic construct
  source 1..218: mol_type = other DNA; note = Description of Artificial Sequence: Synthetic polynucleotide
atataatttattggagtgtccagtccacatgttaatttttcacttgactgaagtcttatgtcttctttgtgttctttccacatttggtttggtgaacagattctcattgatcacttgtctctagaaggattatagtaaaaaaaatacaagaccgaaatatgagacatagttatgtttaaacacaacaggaaattaatgcacagtatacattttaggat

SEQ ID NO: 211 - DNA, 211 bp, synthetic construct
  source 1..211: mol_type = other DNA; note = Description of Artificial Sequence: Synthetic polynucleotide
gaaagggggggggagactggcatattgcttgttccagcgatgtgtcttttctttcggaagctcggtggaaagtgtggctgtaattcttctgtcgtgtcttgtttcacagccgcctgtccagtgttatgtagtggcaacgggcagtactccaagggccgctgcctgtgtttcagcggctggaagggcaccgagtgtgatgtgccgactaccc

SEQ ID NO: 212 - DNA, 226 bp, synthetic construct
  source 1..226: mol_type = other DNA; note = Description of Artificial Sequence: Synthetic polynucleotide
ggctggcgtggggaataaggcctcagaaaagatgacaccacacccccccagacactagctcttcagaaacgccccggtttacctctgaagctggcggaggaagtgttccaggagttgctgaataggtgtgctctcattttcggctgacaatttaaggaaaaaaaaattgaatacaagactttggtatcccttcattagagagctccaagttcacagtatctgtctg

SEQ ID NO: 213 - DNA, 199 bp, synthetic construct
  source 1..199: mol_type = other DNA; note = Description of Artificial Sequence: Synthetic polynucleotide
aagcacttcatatgcctctgaaaaaaatagattccaaatttgacaattgactccttttattgcagaatgcagcactcaactggacatagtcatagtgctggatggttccaacagtatttacccatgggacagtgttacagcttttttaaatgaccttcttgaaagaatggatattggtcctaaacagacacaggtatgt

SEQ ID NO: 214 - DNA, 169 bp, synthetic construct
  source 1..169: mol_type = other DNA; note = Description of Artificial Sequence: Synthetic polynucleotide
catcgaatgtattctcatccacttcacaatgatccaaatttggaaactgtgtatacgctttgtgaaaaaaaagcacaggtatggcagaaatttggtttttatagtagaattctatatgtttcactgaataaatatgtgtttgtcattttcttatcctgttggtttaatt

SEQ ID NO: 215 - DNA, 215 bp, synthetic construct
  source 1..215: mol_type = other DNA; note = Description of Artificial Sequence: Synthetic polynucleotide
gtcctgcaagtcggtgagtcacggggcagccgcgagccgtctgtccgtccgtcagtccctgggtggggggggggtgcccgcctttctcctgcggacagcccctagttccgcgcgcgcccgcacccctgcgggtgtcctcactggcccggacggtcctcctgagcggagcgcccatttcctccctcccttccctgccccccgtcgccccggacggg

SEQ ID NO: 216 - DNA, 168 bp, synthetic construct
  source 1..168: mol_type = other DNA; note = Description of Artificial Sequence: Synthetic polynucleotide
caagacagaaagacagacgtagatctaagagcccacataaaaaacgctctaaatcaagggagagacggaagtcaaggagtcgttcgcattcacggtgagttttagagaaattaacaataatttttttttcctcagagttctgttagtgctaagggataatattttaat

SEQ ID NO: 217 - DNA, 191 bp, synthetic construct
  source 1..191: mol_type = other DNA; note = Description of Artificial Sequence: Synthetic polynucleotide
atttgggggaatagatgggagaaaatgtgattatatttacatgatttctttttttttcaggtaacagactgggttgacccatcatttgatgattttctagagtgtagtggcgtctctactattactgccacatcattaggtgtgaataactcaagtcatagaagaaaaaatgggccttctacattagaaag

SEQ ID NO: 218 - DNA, 207 bp, synthetic construct
  source 1..207: mol_type = other DNA; note = Description of Artificial Sequence: Synthetic polynucleotide
ttctgtgtcttactgtaacctagggaccttgctgcaagaaactgcctggtaggtgaaaataatgttctgaaaatcagtgactttggaatgtctcgtcaagaggatggtggagtgtattcatcttctggcttaaagcagattcccattaaatggacagcaccggaagctcttaattatggtaagaatagaccacattttttttttaat

SEQ ID NO: 219 - DNA, 186 bp, synthetic construct
  source 1..186: mol_type = other DNA; note = Description of Artificial Sequence: Synthetic polynucleotide
gattgggctaaacactttgggcagactgaaattgtggatcttctagaatcttacaggtaaaactttgtactattttaaattaattctaccgtctcttaaaaaagagcttcaccatataaattatgggctattcaaatgtagaccatttatagtcttttatccagagatttttttttgtgattctaa

SEQ ID NO: 220 - DNA, 204 bp, synthetic construct
  source 1..204: mol_type = other DNA; note = Description of Artificial Sequence: Synthetic polynucleotide
gtttataattttaaatataatttttatgcatctactagaagtttgtaaaatttcccatttgtaactttgtcttaccgatacaagcagtgtgatgctgtgtgaaaccaggtggacatttacaggtaaacccccccagggtgttgacacagaggaactggcagttatgctgctttgtttgacattcatcaaggtctgaaattagag

SEQ ID NO: 221 - DNA, 177 bp, synthetic construct
  source 1..177: mol_type = other DNA; note = Description of Artificial Sequence: Synthetic polynucleotide
attgatggattattttttccatttctttaacaggaacatgaatctgaaggtggaagaacacctttgatgaaagctgcaagagctggtcatttgtgcactgtgcagtttcttattagcaaaggtaaagaaagggcaagtgatcatttccaagaggctacagtttatggaaaaaaaaat

SEQ ID NO: 222 - DNA, 183 bp, synthetic construct
  source 1..183: mol_type = other DNA; note = Description of Artificial Sequence: Synthetic polynucleotide
ctgccaatcgcggagagatcaaaggtatgcactacccactatggagggcctggacttgggagacaccctcaggcctgacctggtacccctctccccatagggtctgagtctgccacctacgtgcccgtggccccccccaccccagcctggcaaccagagatcaaaccggagcctgcctggcac

SEQ ID NO: 223 - DNA, 197 bp, synthetic construct
  source 1..197: mol_type = other DNA; note = Description of Artificial Sequence: Synthetic polynucleotide
atttgtcttcagagaaattattgctgatgtgaagaagtttctccaggtaagaagtcattgctacatggttagaacatcactacttgctttttttggtttagaatactgctcttcttaaagcgatatagtttgttttgtgactaccagtaccatgttaattatgatttattatttatcctctggatcctttatcattt

SEQ ID NO: 224 - DNA, 230 bp, synthetic construct
  source 1..230: mol_type = other DNA; note = Description of Artificial Sequence: Synthetic polynucleotide
ttctaactcaccagaaaaaaacctctgcacttcatcggccgagcaagaccagggcaagccccggaccttcaccacgaatccctctccaccttccgtgcccaacatcatcgtctcttacgcggtccggcgtcgaaacaaactgcaaagcggggaggaccagaactgagagcgccaattaagctgtccttcgcctccgaggcgcgcccgggcccgcaccgcccccccacgga

SEQ ID NO: 225 - DNA, 220 bp, synthetic construct
  source 1..220: mol_type = other DNA; note = Description of Artificial Sequence: Synthetic polynucleotide
aggaggctgaggcaggcaagttgcttgaacctgggaggcagaagttgcagtgagctgagatcgcgcagcctgggtgacagagcgagactctgtctcaggaagaaaaaaaaaagaaagtaagcatgtgtaattcttatttaaccttttatattgatcaacagccagaataaaagctacttggccgtaaggctaaaagaccaagatctgagagattcaaggc

SEQ ID NO: 226 - DNA, 231 bp, synthetic construct
  source 1..231: mol_type = other DNA; note = Description of Artificial Sequence: Synthetic polynucleotide
aaacccaatcaccacaagcccagttccgctgtcaaccacacaatctcagggaaaactgaaagtagcaatgcaaaaacccgcaaacaggtgctatccctcgggggggcgtccaagtccactgggcccgccgtcaatggcctcaaggtcagtggcaggttgaacccaaagtcatgcactaaggaggtgggggggcggcagctgcgggagggcctgcagctgcgggaggggctg

SEQ ID NO: 227 - DNA, 228 bp, synthetic construct
  source 1..228: mol_type = other DNA; note = Description of Artificial Sequence: Synthetic polynucleotide
aataacatattattatagttgaaagatttttttttagtgtagtgatttcaaactgtgctcatttatatgcaacccatgaacgcctcccttctcctatgtctccttttgagttgtagggccagtttgaagctttatatatcatttaccaaacttagggtggtgagtgggtgctgatggcagcctggtttgaaaatctccactggagagaagacacagcaggattcagat

SEQ ID NO: 228 - DNA, 225 bp, synthetic construct
  source 1..225: mol_type = other DNA; note = Description of Artificial Sequence: Synthetic polynucleotide
ctcagcgacagaaaaagggaagggtgtctcatcccccttcctcctctcctccctgtcctgagccttagccatggccgaggcaggggctgggctgagcgagaccgtcactgagacaacggttaccgtgacaaccgagcccgtgagaaaggcgggggggcggtgctgtttaggggtctgggagatactgggagggaggggacagggattagaagagttgttggagga

SEQ ID NO: 229 - DNA, 190 bp, synthetic construct
  source 1..190: mol_type = other DNA; note = Description of Artificial Sequence: Synthetic polynucleotide
gctgccccccccaacacccttccctgctgcataccctgtctgaagtaagaagctctcaagccggtcaagctgacccttggcctcagagccaaaatcctcagggtgagaggccagccaggttgacagtacagagatggctaccctgggagaagggaatcagccaagggtgagaggtaaagctgcagcctgg

SEQ ID NO: 230 - DNA, 229 bp, synthetic construct
  source 1..229: mol_type = other DNA; note = Description of Artificial Sequence: Synthetic polynucleotide
acttgctggaggctgaggtaagggaagcaggaggtcccccctagccggcctggcccctgggggttacggccgatccctgcccctggaatggccagactatatcctgggacctctctctggctgatagcggtggggggggggtgtgcggagggatcttggtgacatggggggcagtgggcccagaacacccgcttctcctgcctcttccatctctccccaatactccagg

SEQ ID NO: 231 - DNA, 220 bp, synthetic construct
  source 1..220: mol_type = other DNA; note = Description of Artificial Sequence: Synthetic polynucleotide
aacacaatgatctgaagccttttcttggctgtaaaatgactaaatgaactgggtgacttacaggtccctttcaagcaaaaaaaaaattctatgatttgatgatgcacttaatttgtagttgaaaacatcatgacatatactttgcttttccttcaaaggatatttgcatatctgccattaaggagaaggatatcgaagccaagctgactcaggtgattga

SEQ ID NO: 232 - DNA, 218 bp, synthetic construct
  source 1..218: mol_type = other DNA; note = Description of Artificial Sequence: Synthetic polynucleotide
gggctgccaggaaaaaaaatacaagacgtccaattaaattgaatatcagataaacaacgaataatattttagcataagtctgtcccatacaatatttgggacagacttacactatttaaagtatccagtttaactggaagtcttgtgtttttatttgccagatctggtaaccctaggtgcctccagtttgagggaggagagaggacaccccctccacc

SEQ ID NO: 233 - DNA, 224 bp, synthetic construct
  source 1..224: mol_type = other DNA; note = Description of Artificial Sequence: Synthetic polynucleotide
acggacagcacctcgttcaaatccaagccggagaaggatgttagaccaaaggggttccagtttgcattttactgcaagtaactcctcaatggttgtttgaatctattacaaaggaaaaaaatgccaggaatttatatcatgagcataaagaccacccccagttctcccactcccctccctcattttttgaagaagtctcccaccagatgggctgtggcatttgc

SEQ ID NO: 234 - DNA, 215 bp, synthetic construct
  source 1..215: mol_type = other DNA; note = Description of Artificial Sequence: Synthetic polynucleotide
ataatttcagatgttttagtatgtttgatgaatatttctttttttttcaatttaccccatctgaaattacttcatagtctttccagctagtctttccatcgttgatacataattgccaaagtagccaagttgaactccctacttttaggattcttgagtcactactttggattcttcaaaggtccttcgattctatgcaatctgggatgatacag

SEQ ID NO: 235 - DNA, 205 bp, synthetic construct
  source 1..205: mol_type = other DNA; note = Description of Artificial Sequence: Synthetic polynucleotide
ctgaccataggcaaacgcctagctcaatgtctacatccagcattaccaggtggagttcatcggaaggcgcttgaaacatatgaaattatcttcaaaataattggacctaagcgacttgccaaagatctttttttatataggtaagaataattttactatatatatacaaataatataaagaaaatgtattgctaaactattttat

SEQ ID NO: 236 - DNA, 193 bp, synthetic construct
  source 1..193: mol_type = other DNA; note = Description of Artificial Sequence: Synthetic polynucleotide
cacatacatacacaaaccctataaaaaaaaataaagtggagaggctatgagaaagagaattcaatgtcaataaagaacaagcatcacccaaaatgtcagccagttccaatattcacaccaccgtccgatgttccccacacacatagacagcactaggacgtatccgtcgctttgtgtgaccagggagctgcgg

SEQ ID NO: 237 - DNA, 208 bp, synthetic construct
  source 1..208: mol_type = other DNA; note = Description of Artificial Sequence: Synthetic polynucleotide
tgcaatgcaaaaaattaggggtatatgcttggtccagctttgattctagggaattgcgatattcacttaactttccgattgatgacaaatagtttctttgtatattatttgcattatcttctatctttgaggacataccagaagatatctgtgtattctgtgctacctgagataaatgattggaaaggtcaaatatatttttttgaat

SEQ ID NO: 238 - DNA, 183 bp, synthetic construct
  source 1..183: mol_type = other DNA; note = Description of Artificial Sequence: Synthetic polynucleotide
aatctgtgccaagcaaccaggggaaatctgcaatgaagctgacctctgtgacccacacaaagggctgtattgtgactactcagtagacaggcctaggtacgagactggagtgtgtgcatgtaagtgtcttcttctggacctgctggaaaagattgagtctagacagaatttttttttcctgca

SEQ ID NO: 239 - DNA, 223 bp, synthetic construct
  source 1..223: mol_type = other DNA; note = Description of Artificial Sequence: Synthetic polynucleotide
cagtgccccaaattcagaatagtctgattaacaatcttgtctggagatatggtcaatttaataagattttttatgatacagagtgtgcagatcatctaattttgaagcaccgtcatctagcggagtttcacttgcagtgcccaggtctccattttcatgtgtgtcagttactgccttcttattaaatcctgaaagaaaggagaaaaaaaaagcagacactttc

SEQ ID NO: 240 - DNA, 190 bp, synthetic construct
  source 1..190: mol_type = other DNA; note = Description of Artificial Sequence: Synthetic polynucleotide
caacattgattcccacttactgaaatcctgatagccaggtgaataactttgacgagatgatccagtcaaaacctgcagaaacatgtttacacaattagtatactaaaatatttaaagaaaatatttatatattatcatattttcccaaagatttatattaaaaaaaaagaactccctctttgatttttta

SEQ ID NO: 241 - DNA, 185 bp, synthetic construct
  source 1..185: mol_type = other DNA; note = Description of Artificial Sequence: Synthetic polynucleotide
gtaccttagccacatggttttagaataattttcacctacttcaatgtctctgccaggaagaaactctgctgcacatcatcataactctcatgaagaaggtaaacatcccttaggcctgaatagcctccattcactctgcaatggttttccaaggcctaaattatagaggaggaaaaaaaaaagtc

SEQ ID NO: 242 - DNA, 190 bp, synthetic construct
  source 1..190: mol_type = other DNA; note = Description of Artificial Sequence: Synthetic polynucleotide
ctgaatccagtgctttcctccgcatgttgttgtctttctaaagaagataatgtcaacctccttcatttttttttaccttgctccactgtcttggttgttttctcttccttctttccaggtacagctgcaaaacaaagataaggtttaaagaagagttccagacagaaatactgcttctgctcaaaaaaag

SEQ ID NO: 243 - DNA, 218 bp, synthetic construct
  source 1..218: mol_type = other DNA; note = Description of Artificial Sequence: Synthetic polynucleotide
atatcatgcctggtgtccatgtttatttttgtgtatatatatatatatatgaatattctgagttaaaaaaaataattcaacctattttacctgtttcaggtttgtggataccggataaagcttcactttgatgggtattctgattgctatgacttctgggtgaatgcagacgctctggatatccacccagttgggtggtgtgagaaaaccggccacaa

SEQ ID NO: 244 - DNA, 206 bp, synthetic construct
  source 1..206: mol_type = other DNA; note = Description of Artificial Sequence: Synthetic polynucleotide
cagattgatagggtgatgtcattttttttttcagattggagtaagacagctaccaatatcatcaagaaaagttctgataatctaggaaaatctttgtcttcaagctccattggttccaactcaacctatcttacatccaaatctaaatccagctctaccacctatttcaaaaggaatagccacacaggtgagtctaaagatgtcct

SEQ ID NO: 245 - DNA, 226 bp, synthetic construct
  source 1..226: mol_type = other DNA; note = Description of Artificial Sequence: Synthetic polynucleotide
ctggggtgattgtaggcaatctgcttgaactcattgttccagttgggcctcccatagaaggtcttctgctttaagcctgtaatcacgtcagtattttcgtcccagtgtaaagctatgtgaagagcctaaagtaaatttgtggcattagatgacatttagaaaaaaaaaataagacttctataaataaagaataaagtaatcatgttcatttgtagatttaattaaa

SEQ ID NO: 246 - DNA, 180 bp, synthetic construct
  source 1..180: mol_type = other DNA; note = Description of Artificial Sequence: Synthetic polynucleotide
ttactgaacccaagcaaaaaaaagtgtcttgtcctaaatagtaattataattatgttacttttcagtattgtacagtctaaaactgctacagcgttttcttggttgttatgtgattactgaactttttcgtgtaccaggtgggcgactcaaggatttgattttctacaagcaattgaacc

SEQ ID NO: 247 - DNA, 220 bp, synthetic construct
  source 1..220: mol_type = other DNA; note = Description of Artificial Sequence: Synthetic polynucleotide
gaagtggagaatgcagttactctcaaaacctccaaagaatttactagtattaaaactccagttaattctctccagtgtttagtcacagggtttttcattctgtccaaagctgactgaaggtcttgtaacacatctctgtagttatttccatagtgagcactccaggtatacagaaaatcataggcaggtttccacatcatctataaagaaaaaaaataga

SEQ ID NO: 248 - DNA, 168 bp, synthetic construct
  source 1..168: mol_type = other DNA; note = Description of Artificial Sequence: Synthetic polynucleotide
aaatacatactttagaatcccatttgctccggaataagcttctattgcattgatactggtgggcagcattgtaataattctgtcagctttttcagcaacatctgctggggaagatactacctttaaaaaaaattgcaaaggcacattatttaaattaaaatgtaagca

SEQ ID NO: 249 - DNA, 216 bp, synthetic construct
  source 1..216: mol_type = other DNA; note = Description of Artificial Sequence: Synthetic polynucleotide
aatctgtggaagtccatcccttcaaccaggttgcccagtgcttcaggctcaaacgaggtcagccctgggtcacagattttcctgggggccaaatccaagtgaggaggggccccgtggacccctgactctgagcaggcccccccaagggtcccacatccttgtcctgcacagcccccccccagccccagggactcacgcgtaggcctcaaagtcacc

SEQ ID NO: 250 - DNA, 208 bp, synthetic construct
  source 1..208: mol_type = other DNA; note = Description of Artificial Sequence: Synthetic polynucleotide
tctgtgttcttgtcccccccagcttgtggagcctcttacacccagtggagaagctcccaaccaagctctcttgaggatcttgaaggaaactgaattcaaaaagatcaaagtgctgggctccggtgcgttcggcacggtgtataaggtaaggtccctggcacaggcctctgggctgggccgcagggcctctcatggtctggtggggagc

SEQ ID NO: 251 - DNA, 214 bp, synthetic construct
  source 1..214: mol_type = other DNA; note = Description of Artificial Sequence: Synthetic polynucleotide
ggggccagcgggctgggccgctgtaggaggacctcggggctgggagaaagggggtgtccagggcgtgatcggagcctccgggagccgcaggaccgggctcgggtggggtgttgagggccccagcgcgcagtcctgccccggaccccccccccatccccacctggaagccaaggccgtcagggcccggaaccgccctcacctgtaggccaggctc

SEQ ID NO: 252 - DNA, 206 bp, synthetic construct
  source 1..206: mol_type = other DNA; note = Description of Artificial Sequence: Synthetic polynucleotide
ggagatgatgtacttagaagtcagtctctcacaaaaaagacattatatccgagttctgttaaagccccagccagcaaggcccataaaggaatgaagacataggatagattcatggtagtaaactgttccagtttagcacagagtgcgaggcagaaggctaatttaagtaacattgcaatgaggtaccaggctttttttttaatatt

SEQ ID NO: 253 - DNA, 176 bp, synthetic construct
  source 1..176: mol_type = other DNA; note = Description of Artificial Sequence: Synthetic polynucleotide
gcagcctggccaacactgaaagctccaattaggattgaaaaaaaaacctgagcaaaataacatgaggaaaagtttaagtcacattctggcattcattattatctaaaatgtgatgtatatgtaacatagtacataaaggattaagtttaggtttatcctttccttttttttttgtg

SEQ ID NO: 254 - DNA, 178 bp, synthetic construct
  source 1..178: mol_type = other DNA; note = Description of Artificial Sequence: Synthetic polynucleotide
tgctcttcccacagccactgtttccaaccagggccaccgtctgcccactctgcaccttcaggttcagacccttcaagatctaccaggacgagtgagaaaaaaacttcaaggcaattcacagacacaggatataggaactgactgttcactaggtttaaatatacatgcacttttttat

SEQ ID NO: 255 - DNA, 194 bp, synthetic construct
  source 1..194: mol_type = other DNA; note = Description of Artificial Sequence: Synthetic polynucleotide
aagtctacatacctcggttcataagctccgatgggaatagctgcaaggtcaaaaggtccaaatctttttcctatctcttcaaaagcagggcaataaccagtatctcctgcgaaaaaaaatcgattccaaggccccaagacagaccagctgccccatagcaccttgttgtcatccattagagtccttttacacca

SEQ ID NO: 256 - DNA, 227 bp, synthetic construct
  source 1..227: mol_type = other DNA; note = Description of Artificial Sequence: Synthetic polynucleotide
ggggccagacccatggcattaggctttttctgcttcccgggggctggagaataggacctctttcctatcttcacctctcccactgggagggaacaatcttccccctgagtctggggcctggagcgggctagagttctgtctttgtggggagccctggagcgggggggcgggcacagtctcccagcatcagccccggtggcttctccagagactgctggcggcttctt

SEQ ID NO: 257 - DNA, 224 bp, synthetic construct
  source 1..224: mol_type = other DNA; note = Description of Artificial Sequence: Synthetic polynucleotide
ccatctgtggcacattgttgtctccataaaaaaaaaatctacacttttccaatagcattaagtttgcttttgttcgcatctttcatagggccgatgtgtcgtgcccacaagcgattggacctgtgggagttctgttgtgggtggttcccggaactgtgggcgggcggttttacccagggctccctctgctttgtgtcttgtagaatgtcgtactctcaggaggc

SEQ ID NO: 258 - DNA, 206 bp, synthetic construct
  source 1..206: mol_type = other DNA; note = Description of Artificial Sequence: Synthetic polynucleotide
ggtaaatttccaagtgatctacaaacttgcaagtcttatgggaataatcattgacatgtctcttttttttctcccaagtgaacttgaagatgatttacttggagaagatttgctatctggcaaaaaggtaagaagtttatgtccttctgagatgaaatactagtcatttaattatacttaacatacagcagtaatcacttatacat

SEQ ID NO: 259 - DNA, 225 bp, synthetic construct
  source 1..225: mol_type = other DNA; note = Description of Artificial Sequence: Synthetic polynucleotide
ggggagttctgagtacccgcaggccttcttccggctgcttcctgaagctccgtgcaatacattccaacattttctgaaatactttctggactgtgtccaacagcattgcaacttcttcatctgaatctgttttctgaatcaaggaacttccagttatctctttcagaatgtcaagaagtaatgaaacataaaaaaatcccttcactagaaagaaaattggaatat

SEQ ID NO: 260 - DNA, 201 bp, synthetic construct
  source 1..201: mol_type = other DNA; note = Description of Artificial Sequence: Synthetic polynucleotide
tcttctttccatccatatctgcatgaattttccgagccaagaatccacttttgtacacagcagcatttggatcatgaggaatgtccaaaaatgggttagtagtacttccaatacgactgatggtctttggatgtgttccgttagctttctcctcagtactttctgagggagacttttttttctcttcatcatctctaaaag

SEQ ID NO: 261 - DNA, 186 bp, synthetic construct
  source 1..186: mol_type = other DNA; note = Description of Artificial Sequence: Synthetic polynucleotide
agaagttatattcttataaaccatgtgataagatttcttctgggagtaacatttctaaaaaaaggtacagagttccatatttctatgttctatacttgctttatgagtacttttttttctaaagagaaagaactgtcagatgttgggctatttcattggcaaaaggaagttaaatttaaaacataa

SEQ ID NO: 262 - DNA, 221 bp, synthetic construct
  source 1..221: mol_type = other DNA; note = Description of Artificial Sequence: Synthetic polynucleotide
tttgaggagctttcaaaatttcatttcaaatgtgggtgtcaaatgtcttcagaataagccacaaatgcaaaaaaaatctccgaaaccagtctgtggcaatggcagattggagggaaatgaaatctgtgattgtggtactgaggctgtaagtatgataactaggaaaattgattaataaatttattgttttaatttatctttttcataaatgtcatatatct

SEQ ID NO: 263 - DNA, 197 bp, synthetic construct
  source 1..197: mol_type = other DNA; note = Description of Artificial Sequence: Synthetic polynucleotide
atgatagtcttctcttcaaattattagaccacttaggcttatctttggtattttgttccttattgattttaggagtcctatatttgctaaacttgaaatgcatggtaaaaaaatgtatctactgtccattttttttaggttgatacccaaagatcagtattactgtggtgttctctatttcactgggagtgatattt

SEQ ID NO: 264 - DNA, 214 bp, synthetic construct
  source 1..214: mol_type = other DNA; note = Description of Artificial Sequence: Synthetic polynucleotide
caccataagtcataattcaatcagaaaatatgaagagctacttacggagtcccacagaatctggtttgccattatcattcttacttggttgcacaagtggagcaaatgaaacagcaaggatatttttactttcccaagtgttctgtccagttcgcataatctgtgttaactatttagagaaaaaaaagaaagaaagtgaacaacttaatcccaa

SEQ ID NO: 265 - DNA, 217 bp, synthetic construct
  source 1..217: mol_type = other DNA; note = Description of Artificial Sequence: Synthetic polynucleotide
ttgggcccggacgttactcggtcccgactacagcgatgaaaatgactatactcacacatcctataatgcaatgcacgtgaagcatacgtcctcgaatctggagagagaatgctcacccaagctgatgaaaaggtaaggcctgggttatcagagccccccccccagtcagaggtcaatggaattttttatttggacctattgtgaattattaattaaa

SEQ ID NO: 266 - DNA, 218 bp, synthetic construct
  source 1..218: mol_type = other DNA; note = Description of Artificial Sequence: Synthetic polynucleotide
ttagaatcctagattgcaagaacatatttggtgaaccagtaaatttgacagaaataaattcatcacagctgcagtgcctagaaggtcttttggataatttagtttcttctgattctcccctaaatataagtgaagatgaggtatagtatttactttttttttcctaatataaaaaataccttcaaatttcaataagtgaatataaattaaaaggattt

SEQ ID NO: 267 - DNA, 220 bp, synthetic construct
  source 1..220: mol_type = other DNA; note = Description of Artificial Sequence: Synthetic polynucleotide
actcagggccgcccatcaggagatgcctttatccagatgaagtctgcggacagagcatttatggctgcacagaagtgtcataaaaaaaacatgaaggacagatatgttgaagtctttcagtgttcagctgaggagatgaactttgtgttaatggggggcactttaaatcgaaatggcttatccccaccgccatgtaagttaccatgtaagtttttcttgg

SEQ ID NO: 268 - DNA, 212 bp, synthetic construct
  source 1..212: mol_type = other DNA; note = Description of Artificial Sequence: Synthetic polynucleotide
gtaaatttagaaatgtttattttcataaataacagtgtattaggtaaatattggacttacgtttgaaagcaggatacattggaaccatattggttacaaggaatgcatcatatttagcctctggtgaagagctcagatctaaacattaggaaaacaaatagtatacgttgcttaccaaaatctatatcagttaaaaaaaaaagggagagggg

SEQ ID NO: 269 - DNA, 168 bp, synthetic construct
  source 1..168: mol_type = other DNA; note = Description of Artificial Sequence: Synthetic polynucleotide
aacatactttatccaaagccagaatgaaaagtcacttacgcctttggtaggcctctttaatggcgatgatttcctagggaaggtaatttacaaaaaaatcaatattccaaggagggattaatcaataagagatacagagagaagaggcgggagaccatgtctggacac

SEQ ID NO: 270 - DNA, 205 bp, synthetic construct
  source 1..205: mol_type = other DNA; note = Description of Artificial Sequence: Synthetic polynucleotide
tgcagacatatttgtgacgtcctctaaaatgtagaatgtttcatacagaattatgatgcttgggtaaaaaaaagtaggatgtttgttgagcttcgtagattatatgtaattggaacctttggagtaaattttagtttctgagtcttaccaatatgtatttttttctattacagattgtctcctaaccctctagttttaattggac

SEQ ID NO: 271 - DNA, 198 bp, synthetic construct
  source 1..198: mol_type = other DNA; note = Description of Artificial Sequence: Synthetic polynucleotide
gccaaccccatgagcaacctggtcctgccagaggccccaaggctgcggctggctgagccactccgcggaggccccactgggccccagtccccagcttgcgtggtacccactgccaccaccaaaggtaacaggtgtggtgggtggtgggtggcagagtgggggggggcagagccctcccacctcactctaatccccctt

SEQ ID NO: 272 - DNA, 218 bp, synthetic construct
  source 1..218: mol_type = other DNA; note = Description of Artificial Sequence: Synthetic polynucleotide
gccagggtggagatcggcccccaggtgctgaatctgaaaccaggagatgggtgcagagtgggcacaggggagcaggggccagagcggggtggggggggggtggagggtgagggagggggatggaagggtgaggcgggggaagggccacctggaactgcaccgtctcctgcatggcacccaggtgcacagggatgtggggggcattggacaccagcccc

SEQ ID NO: 273 - DNA, 228 bp, synthetic construct
  source 1..228: mol_type = other DNA; note = Description of Artificial Sequence: Synthetic polynucleotide
caactgtcactcccactggaggcgctgtgtatgcagggcccggcagtaagcccagctgcctacagccaaggcccagcgacagacatcctccttcggtagcagcagcagcctgtcctcctccagccggctcaggaggcctgccacacccagcatcgcccccccccacatcccataagtgttggccgtgaagacagctacaatgaggatggtcactgcgaccaccaacag

SEQ ID NO: 274 - DNA, 221 bp, synthetic construct
  source 1..221: mol_type = other DNA; note = Description of Artificial Sequence: Synthetic polynucleotide
catgccccccccaccacccaaggtgagcagggtgccagcagcctgtttcctcttctcctgctggaccacggcgtttctgctcccgagttgggactgtggaatggtgtgggtgctgtggtctgctccatcgctggctcctccctgggtgggaccttgctggccaagcactggtgagccctccccagtgtaccctgcccacccacttgtacctgagctgcccc

SEQ ID NO: 275 - DNA, 208 bp, synthetic construct
  source 1..208: mol_type = other DNA; note = Description of Artificial Sequence: Synthetic polynucleotide
ccaccatactgtgggatgagaccacggtagtcacctgggcggaactcccttgagtatcaaagtaattccctccagtattttggctgtacatctgtgtctctgtgtaaggatacgttgttgttcggctttagaagaagaagaaaaaagaaatttactagcttatttatgtcatcttattaatttttttctaatatgaatgtaattgaaa

SEQ ID NO: 276 - DNA, 225 bp, synthetic construct
  source 1..225: mol_type = other DNA; note = Description of Artificial Sequence: Synthetic polynucleotide
agacaacagaaagttaggggtgctttcaatttattattcaacctgagctctcctgtgactcttgttctcttttgaaacatgttaagttatgaaagcttactttctggaaagcttcagaaacaagtgatgttgactggacaacatgaggaggttcctcaagctctaaacttacgtgcatttttttttcaagatcagattgtgtggagattcttaaaaaatgtggag

SEQ ID NO: 277 - DNA, 181 bp, synthetic construct
  source 1..181: mol_type = other DNA; note = Description of Artificial Sequence: Synthetic polynucleotide
tccacaagtcaatactatcagcaaatgattacaagctcttgtgtatgtttctagtcttaccttccaactatatatacttaaaaaaaaaatcaatcgctcatatgggatataaagttaggagtttaggagttattttaccttcatgctgactattgcaactcggaggttcgtcccgccaaga

SEQ ID NO: 278 - DNA, 215 bp, synthetic construct
  source 1..215: mol_type = other DNA; note = Description of Artificial Sequence: Synthetic polynucleotide
cagaagcgtagaggtcacccaggctgtttggggggggatgtccccccaccgaaaccccgtccgctgcctgctgtggagacgccgacagtgcaaactcaccaggcaccatccctgtcagcgtcggtgctgagtagctgccctgtccagcgggggggacgtgtggagggtacccggggagggtcgtgctcgccaagtcacggcctgaggaatcaaag

SEQ ID NO: 279 - DNA, 221 bp, synthetic construct
  source 1..221: mol_type = other DNA; note = Description of Artificial Sequence: Synthetic polynucleotide
acatcactcaactcattttgacagataaaaaaaaagtctgatagatattttccctttatacccaacaaattattgatttctgctatttgcatataaaaggataaatttaagttaatctttttttaactacctgaatgtatatgaaacaatttaaaagaatgttatctctacttatagatctcgatttattaatgatgcataccaggaagtactggatctac

SEQ ID NO: 280 - DNA, 223 bp, synthetic construct
  source 1..223: mol_type = other DNA; note = Description of Artificial Sequence: Synthetic polynucleotide
aatgtccaatttcagtcaagcttaccttagagaagatggttttgctgcctggtggctgttgagaagagcacttttctgttattaagccttcactcctgagctcaagatcctctgcagccattaaatagcagttctgctgtattacttccaaatctttttcattgaatttctaatgaaataaaacaaattctaaggaaattgtcatcaagaaaaaaaatattaa

SEQ ID NO: 281 - DNA, 219 bp, synthetic construct
  source 1..219: mol_type = other DNA; note = Description of Artificial Sequence: Synthetic polynucleotide
agcatttgaattagcaatgacatctttgggctctgaatcaatggcatttatacaggagcctatggtgccacaagaccagggagaataatgggaaagatgatcttcttcaaattttgtaccactcacactctctgagaactggttaaaaaaaaataaataaagccaattagtaagaagcttaaatttacaagcttgtaactaatataaagtaactattat

SEQ ID NO: 282 - DNA, 202 bp, synthetic construct
  source 1..202: mol_type = other DNA; note = Description of Artificial Sequence: Synthetic polynucleotide
catcgtggtcagcatggtggctgctaagcgtgggtgcacactccctccccgggtggtggggggggcagggtgggccgcaggctgactcagcccccccaccagcagtgctgactgtccgggtgtctgtgcccatcatcatgggtgtcaacgtaggcacatccatcaccagcaccctggtctcaatggcgcagtcaggggaccg

SEQ ID NO: 283 - DNA, 192 bp, synthetic construct
  source 1..192: mol_type = other DNA; note = Description of Artificial Sequence: Synthetic polynucleotide
gagctgatctatcacttactgatttcacataagattgatgtttatcccaacatttaaaattactctacagttttcttcagttcagagtattcaatagttaaatagcaccaatccctagtttttttcctaatcttaaaaaaaaatggatttagcatatcttactggaaatctgagtccattagtgacttcatt

SEQ ID NO: 284 - DNA, 222 bp, synthetic construct
  source 1..222: mol_type = other DNA; note = Description of Artificial Sequence: Synthetic polynucleotide
ctacctgctttgtagacatctcatcccaaagcttgaaattgtctttttttttctttcccaaaagtaccagaataggagttttcctctgtggacctgaagccttggctgaaaccctgagtaaacaaagcatctccaactctgagtctggccctcggggagtgcatttcattttcaacaaggaaaacttctaacttgtctcttccatgaggaaataaatgtggg

SEQ ID NO: 285 - DNA, 193 bp, synthetic construct
  source 1..193: mol_type = other DNA; note = Description of Artificial Sequence: Synthetic polynucleotide
tccagcccagacaccacagagcctggctacatcaacatcatggagttggcagcatctgtttgccgctatgacctagatgacatggacatcttctggcttcaggaactcaatgaagaccttgcagaaatgggtaagttcttttctcacaagtaagttgtgacttaattcatgttttttttttaagttatcagaa

SEQ ID NO: 286 - DNA, 184 bp, synthetic construct
  source 1..184: mol_type = other DNA; note = Description of Artificial Sequence: Synthetic polynucleotide
ctgttgactaacccactgcgtctgtatctgaatgctgtctccaggtcagagccggggcttcgccttcgtcgagtttagtcacttgcaggacgctacacgatggatggaagccaatcaggttgctttgccgcacttgaacccccccccaaacaaatactactttgtaatcgagcgctccccatcg

SEQ ID NO: 287 - DNA, 174 bp, synthetic construct
  source 1..174: mol_type = other DNA; note = Description of Artificial Sequence: Synthetic polynucleotide
tgctaaatgtggacagcactagtcggagtgacttatcagacatacccctcatctgcaggaagaaaggattaatggtttagttttattaacatttgaaaataggcaatataagtgaaagttttttttttcctactagcaaacttattaataattttactttgatatggcaaatta

SEQ ID NO: 288 - DNA, 217 bp, synthetic construct
  source 1..217: mol_type = other DNA; note = Description of Artificial Sequence: Synthetic polynucleotide
gcactgcctatcctcatcccccccatgagccttggtttttatgcaacctatggtcatgcagggatgcccttacaccctcccacgatgtcatgactggcaggcagctgaccagttccatcatagcagcagcctccgaagcacctgcccccaccctcaggtaagagtctgatagtgaaagaaagacctgtggagaaagtggacacaggatctgtgtgtc

SEQ ID NO: 289 - DNA, 193 bp, synthetic construct
  source 1..193: mol_type = other DNA; note = Description of Artificial Sequence: Synthetic polynucleotide
ctccacactgtccccatctggaaagctcggacgcaaggtagaggccaaaaaggtgacacccctgatgccgacaggcccccccgacagccagcctgtgggccagccagggcccccgaacgtatgctctgtgcgcactcacacctgtgcacacacaaatctgcacacaagcatacctctgtacacacctccgcac

SEQ ID NO: 290 - DNA, 194 bp, synthetic construct
  source 1..194: mol_type = other DNA; note = Description of Artificial Sequence: Synthetic polynucleotide
cgcgagttactcggtgacaaaggttctcttggcggtactttgggaggcctgtcttcatcactataggtgctcgaagtaacttctgctgaccatcttctataatctggctttactggtctaggaggtatgggaactctggggggaacctcaggtttgtcttcatcggagttaggagaagctctgtgtatacaaca

SEQ ID NO: 291 - DNA, 187 bp, synthetic construct
  source 1..187: mol_type = other DNA; note = Description of Artificial Sequence: Synthetic polynucleotide
gctgtgtattatgtcttggcagacctggtgatgctgacgctgtacttttactacaagttcaggacgcgcccctctctgtgtgagtatggggaccgctctctgtcagatgctctaccagcagcagggggcagttgtgggggggggcgcctggagtgtgggaggaagcgggtttcccatggctgggtga

SEQ ID NO: 292 - DNA, 174 bp, synthetic construct
  source 1..174: mol_type = other DNA; note = Description of Artificial Sequence: Synthetic polynucleotide
ttaccttgaaatatccccccagtaccattctgcatcctgaagagaaacagaactgtccttcattccatttggaactgctgaagtcattggcttaggtggctttggtggaagagctagaagagaaatgaatattactctgtagatgtaaattatttactgttttctaatttcctc

SEQ ID NO: 293 - DNA, 193 bp, synthetic construct
  source 1..193: mol_type = other DNA; note = Description of Artificial Sequence: Synthetic polynucleotide
ctcaccgtgtccccacagaacagataccagtcttcatcagagatgggttccacaaccactgggcgccgcctgatccatggggggttcttcctcttgtctgcaacagagcctggggggctcatcccatagcggtaatgggggggcagagtgaccttggatgccaaggccagcagggcagaaagacgcatgcctg

SEQ ID NO: 294 - DNA, 204 bp, synthetic construct
  source 1..204: mol_type = other DNA; note = Description of Artificial Sequence: Synthetic polynucleotide
ggacattaatttaatctgacaaagccatcaagtcgggggggattaaatgtttacttactcagtgcagattttgttttgtttgtaaaacttgtgtcgtgtggcaaacaaccgagaaatatacttggcattttcgatatcatccttggaaaagaagagacagagaaaaaaataaacatttgttgaattagttatacatttcattaa

SEQ ID NO: 295 - DNA, 210 bp, synthetic construct
  source 1..210: mol_type = other DNA; note = Description of Artificial Sequence: Synthetic polynucleotide
ccatggcacattttgtaaatacgtgtgtatttttcccccccccagggactactggcattcagtgatgtggtcatagaattctctccagaggagtgggcatgcctggaccctgcccagcgaaatttgtatagggatgtgatgttcgagaactacagaaacctggtctccctgggtgaggataacttcaatacacaatttatgtatttcata

SEQ ID NO: 296 - DNA, 203 bp, synthetic construct
  source 1..203: mol_type = other DNA; note = Description of Artificial Sequence: Synthetic polynucleotide
ggttacttgatgcttccacacaccaattgtgtgtgggccttggtggcaataaggtacagggggctgggcagagagaccactgggaactcaacaagattcatcccatgaattagaaccaccagtaacgccgccctacgaaggcacagtgaatagccagtgaccgtcaccaggcagtgcacttgtagaaccccccccattggtgg

SEQ ID NO: 297 - DNA, 225 bp, synthetic construct
  source 1..225: mol_type = other DNA; note = Description of Artificial Sequence: Synthetic polynucleotide
tctctgtttccttgaggacctctctcacggagacggaccacagcaagcagaggctgggggggggaaagacgaggaaagaggaggaaaacaaaagctgctacttatggaagatacaaagggtaagaccaaaattgatttgatctgaatccttaagaaaaaaaaaatagaaggaaaaataaaagcccagaccaaaaagcaagtacttgaaagctggaggcaaaaagc

SEQ ID NO: 298 - DNA, 198 bp, synthetic construct
  source 1..198: mol_type = other DNA; note = Description of Artificial Sequence: Synthetic polynucleotide
ggtttctgaaccaccatggaaggtggacttaagttgattagtgctcttctgctatagcccagattgcttgttttcttctgtaaaaccccccacattttcttgctgcttaaggaagacctagtacctttaattggtaccattttatgcttgcgccttcgatgatgggacaagttacttcgatcactgcagcggaaggaa

SEQ ID NO: 299 - DNA, 176 bp, synthetic construct
  source 1..176: mol_type = other DNA; note = Description of Artificial Sequence: Synthetic polynucleotide
cttttagatacagaacacttcatgtaacataattatttcctaactttctataagtaatttagtgcaaaaataaccttaaaaaactacttctgaacaagaaacagagataaaaatctaagtattctttttcccccccagaactaaggaaagtagaaacttacctaagatgtaagagt

SEQ ID NO: 300 - DNA, 194 bp, synthetic construct
  source 1..194: mol_type = other DNA; note = Description of Artificial Sequence: Synthetic polynucleotide
catctttctctgtgttctcttccccagcgggcgatgatgagatctcatttgaccctgatgacatcatcaccaacatcgagatgattgacgacggctggtggcgcggggtgtgcaagggccggtacgggctcttcccagccaactatgtggagctgcggcagtagggcccccagcccccccccggagctgcgccc

SEQ ID NO: 301 - DNA, 201 bp, synthetic construct
  source 1..201: mol_type = other DNA; note = Description of Artificial Sequence: Synthetic polynucleotide
ggagtcatgcgcttctttcaagctggtacctcgacagaaaggcctggaactcctgctctcttgtggcagaggcagccctcagctctgcagggtcaaaggccttggtgaggtcaatagctcggacttgtttctggaaggggagggggaggccccccccactggactcacaactgcagttgttccaagccagcagcctgaagg

SEQ ID NO: 302 - DNA, 221 bp, synthetic construct
  source 1..221: mol_type = other DNA; note = Description of Artificial Sequence: Synthetic polynucleotide
gtcctgtcagaatgactgtctccgagctggggggggagtccactccacgtgtgctcggctcacctgagcggtagctgctccgcaggcagaagaaccggaagacatcgggggaaaaggtcttcagaaagtcctggtaaagcgagagacaggcagtcacgaggctttgcttttacgcttcgcactgttcagagaatatgttactttctacaattacataagtt

SEQ ID NO: 303 - DNA, 185 bp, synthetic construct
  source 1..185: mol_type = other DNA; note = Description of Artificial Sequence: Synthetic polynucleotide
ccaggtactaaaaaagagaataacatgaaaacgcccagggttacttgaatgtttttataagataggaatatatgtcttcaccatggggggggtctcgatttcactaacgttgtatatgaaaatgtctgcaataaaaagtacttttaaactttgtaaccctccttttgtacaagtacttattttgc

SEQ ID NO: 304 - DNA, 204 bp, synthetic construct
  source 1..204: mol_type = other DNA; note = Description of Artificial Sequence: Synthetic polynucleotide
cccagagccccccccagaactgctcgcggagtaactgcaagatctcctagcggggatgggcggggccgcctgtgcagatgccaggagggctcgagctggacactcctggctggacgccaggccagtgccgcctacgtggagactgtccacacagctgcctcagaagcgccgggctttcctcacacctgagccgggtgcgaggag

SEQ ID NO: 305 - DNA, 192 bp, synthetic construct
  source 1..192: mol_type = other DNA; note = Description of Artificial Sequence: Synthetic polynucleotide
atctcggtggagcacgttggcggagtggatgtacttgaggccccgcaggatctggtagaggaagtagcagatatggtcattgctcagctgctggcttttcagcaacttgtacaggtcagtctccatcaggtcctgcacaatgtagctgaggatggttccgcagacccccccaggcagggggcagtgggaggc

SEQ ID NO: 306 - DNA, 195 bp, synthetic construct
  source 1..195: mol_type = other DNA; note = Description of Artificial Sequence: Synthetic polynucleotide
aacagttctgtgactgtcaatctttgatttctttctgtggctttttccccccccttcaatcaatgccttttctctttcctaggcaagactggtgtctttgtactttgataccaagaggtaccaggaagcattgcatttgggtaagtaagctggtaataagtcatctcagctagctaaatgaatgtattctaaacc

SEQ ID NO: 307 - DNA, 188 bp, synthetic construct
  source 1..188: mol_type = other DNA; note = Description of Artificial Sequence: Synthetic polynucleotide
gagacagggtgacgtgtcagagacaatcagagtcttctttgagcagagcaagtctttccccccagctgccaagagcctccttaccatccaggaagtggatgagttccttctgcggctgtccaagctcaccaaggaggatgagcagcaacaggccctacaggacattgcctccaggtgggggagctgcc

SEQ ID NO: 308 - DNA, 187 bp, synthetic construct
  source 1..187: mol_type = other DNA; note = Description of Artificial Sequence: Synthetic polynucleotide
ttctcggcttccagcctcaggttggtttcatcctggagcctggagtggggggacacattcctgggtccaggctcttctgaggacacttgaatacctgcctcagtctcccttgaggcagctgtcacagagaccacccccacccaggaccagtagagcagcaggaggattaagggttggggggccttag

SEQ ID NO: 309 - DNA, 209 bp, synthetic construct
  source 1..209: mol_type = other DNA; note = Description of Artificial Sequence: Synthetic polynucleotide
cgcgcagtgcatgttgaaggaactcggctcgagcccatagccggccgcgtagacgccggccgcgctctggcccgccatgccccccccgccggggtacaagccctgcatcgaggcggcgaaggaagcgcccgaacccgctccatagcccgggcgctgggggttggaagcaaacgcacaagaagtttgttctgggaaggctccggtagcga

SEQ ID NO: 310 - DNA, 190 bp, synthetic construct
  source 1..190: mol_type = other DNA; note = Description of Artificial Sequence: Synthetic polynucleotide
agcctggtgctccttcttgggggggctgacatgactaagtcgggtccacagcctctttcccacactcacccattccctggccacccctgcctagtgcagcacccatgtcacctggtggtccctgtgggcactgcgtcgtggccggtcccgaggggagaggccattgatcttgcattcgtagcaggcttca

SEQ ID NO: 311 - DNA, 231 bp, synthetic construct
  source 1..231: mol_type = other DNA; note = Description of Artificial Sequence: Synthetic polynucleotide
gctagcctgcaccaatgctcactaggtggtcactaggtgcccagagtggtcctcagagccagaacctcaaggatgaacaagtctcttaccagggggccaggagggcccatctgtccaggggctcccaggaggcctggttcaccctgagaatggaacagaacatggggtgggctgcaccctgtcagaacacagtgtggacccccccccgaccatgcaccaggggcagcccct

SEQ ID NO: 312 - DNA, 221 bp, synthetic construct
  source 1..221: mol_type = other DNA; note = Description of Artificial Sequence: Synthetic polynucleotide
cttttgattttgctgtgagaactcacccagaacttgaccaggaagaaggcatggggggggccacgatcatatagctctcggaggccaccctttttctcagggaatttgtcgtagatctgccggacgtccacactctcgagcggcggcgctccggggctggggcagtgctggctgatgtgcacgaacaggtgcctctggtactgaggagggttggccgtggg

SEQ ID NO: 313 - DNA, 201 bp, synthetic construct
  source 1..201: mol_type = other DNA; note = Description of Artificial Sequence: Synthetic polynucleotide
tttctgtggccccttcccttcctagtgacgtctctcagctctaccctctcaaaccgtcatatgacgtcactatggcgtcatggtgatggccacctacctggatgacgtaatcatgggccgtgaggttaaaccagacccccccaagaaggaaggagactgcggggagctttgggatttccgagcacaggatgatgtactggg

SEQ ID NO: 314 - DNA, 181 bp, synthetic construct
  source 1..181: mol_type = other DNA; note = Description of Artificial Sequence: Synthetic polynucleotide
agttggttggtcttgtccccccccatccaggatcctggtgaacatggacgacaacattgtgaagcattactccaatgaggacaccttccagctgcagattgaagaagccggggggtcttacaagctcaccctgacggagatctaaaggcctgcgggccacagctccccaggagttcagtgc

SEQ ID NO: 315 - DNA, 203 bp, synthetic construct
  source 1..203: mol_type = other DNA; note = Description of Artificial Sequence: Synthetic polynucleotide
gacagcctgtcttgcctcaggtatcatataaagttgctgaatagtagaagctaagtaacaagtagctccaaggttagtaaggccaacaaatctacattcagcacggacatcttcatgaggccagtaatcccatttataaggtgcatgggctgctgaagaaagagggaggaaaagatcccccccaaacaaactatgaaacacaa

SEQ ID NO: 316 - DNA, 175 bp, synthetic construct
  source 1..175: mol_type = other DNA; note = Description of Artificial Sequence: Synthetic polynucleotide
caaagaatgcaagagtcaagatgccggagcaggaatatgaattccctgagccgcgaaatctcaacaacaatatgcgtcggccttcttccccccggaagtggtactctccaatcaaggtgtgtctctttactcaaaaaatctatcatcagtcctgataaaacacttacatagtgaa

SEQ ID NO: 317 - DNA, 197 bp, synthetic construct
  source 1..197: mol_type = other DNA; note = Description of Artificial Sequence: Synthetic polynucleotide
tcttctggccactggatgccgcgtagacagggcctggcacacagctcaccactgcagggccccccccagggcacctggcggtgtcattggcacgatagaactccagggaacagagggagaggtgggcctccgtgcccacgcacgccaccccatgcagaccaaaggagtgttgctgccgttgggctagcagcctaggg

SEQ ID NO: 318 - DNA, 213 bp, synthetic construct
  source 1..213: mol_type = other DNA; note = Description of Artificial Sequence: Synthetic polynucleotide
ctttaccaaagcggattggcattatgtttaagttcttaaattacagatcaagaaaatgcatacagaagatggggggggggcacacctaattaatttttatatttagattaaagaaaataattaaatgtgtttttttgtgggattgattttcagaagctaaatgcaactagttcatctgaaggcagcacggttgatattggagctcccgccgag

SEQ ID NO: 319 - DNA, 188 bp, synthetic construct
  source 1..188: mol_type = other DNA; note = Description of Artificial Sequence: Synthetic polynucleotide
tgtcccctcgctggaggccagaggccaagatgatgtccatgctgggcggcttgcagaggtaaggggggcagttggaccccccaggtggacaaagcgcttcatctgtggctattctgagagacggacaggagtgggttttgtttgtctttcgaaggaagtgacttggatttggaggctgttcctactca

SEQ ID NO: 320 - DNA, 187 bp, synthetic construct
  source 1..187: mol_type = other DNA; note = Description of Artificial Sequence: Synthetic polynucleotide
tgggtgggtcttgccaagttccctttgagccaacaaggctaccactttaggcttttctcttctccaggggtttcccagtaagatgctgcattgtggtcaagcacctggggcgggcagagctcggcatgggtgactccaccagccagtcccccccaattaagaggtcatgcccagatgtgcaggtgag

SEQ ID NO: 321 - DNA, 172 bp, synthetic construct
  source 1..172: mol_type = other DNA; note = Description of Artificial Sequence: Synthetic polynucleotide
gatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcaca

SEQ ID NO: 322 - DNA, 228 bp, synthetic construct
  source 1..228: mol_type = other DNA; note = Description of Artificial Sequence: Synthetic polynucleotide
gaggttggggggggcctacctggcagcactggtgtaccccacacccccatccctctagctcgggttgcctgtacctggtaggggtctgtgttgagatcaaagtactctaggaagccagttgcaaattcacagaagaggaaattgtgagtctcattgatggtcctcatgcaccagtacgtgttattgttggcgctggtgcaggcacagaaaggccccactgccaaggaa

SEQ ID NO: 323 - DNA, 204 bp, synthetic construct
  source 1..204: mol_type = other DNA; note = Description of Artificial Sequence: Synthetic polynucleotide
cgtgccacgtgccaagcactgttctggggctgtgggaaccacgaagcatgggaggcggggactgcggacaagtcccagcccaagcagtgggggggggctacgcacttgaagatgtggccgtgggggtgcaccacggggttgatgtcaagtggcagcttctccttgttttcctccagaatggcctaaaaggaaacacaccggaag

SEQ ID NO: 324 - DNA, 233 bp, synthetic construct
  source 1..233: mol_type = other DNA; note = Description of Artificial Sequence: Synthetic polynucleotide
cagcccctgccacccacagacacgggctgactggggtgtctgccccccttgggggggggcagcacagggcctcaggcctgggtgccacctggcacctagaagatgcctgtgccctggttcttgctgtccttggcactgggccgaagcccagtggtcctttctctggagaggcttgtggggcctcaggacgctacccactgctctccggtgagtctggaaccctggggagacga

SEQ ID NO: 325 - DNA, 217 bp, synthetic construct
  source 1..217: mol_type = other DNA; note = Description of Artificial Sequence: Synthetic polynucleotide
caggtatcccaacaggtgctggctctcttgggggagaggtgatgcctagacctggctcaataccagcaccaacgacctctctcctctaactggacactcacctgattccccagagggtaccacctctccaggagcatctgcacagaaaggaaatcagtgatcatatacccatgatcctctggggggggagaagtcatgcttcagggaaggtggatcc

SEQ ID NO: 326 - DNA, 225 bp, synthetic construct
  source 1..225: mol_type = other DNA; note = Description of Artificial Sequence: Synthetic polynucleotide
cagaagatggaagagaacagtttaagtttggaatgcctgctgatgccaaataccacttcctttttcactagtatgaattcagcgtgatctttgatacaagccacctgtctggggaagaggaagttctcagcttcattgttactgctcagaggtaaggggggggtttaaacacttttttcaaagatgagagatttatgggaccaggaaaggatgtgtcctggttaa

SEQ ID NO: 327 - DNA, 194 bp, synthetic construct
  source 1..194: mol_type = other DNA; note = Description of Artificial Sequence: Synthetic polynucleotide
atttgaactttgaatctctgcagaataactctattccccccgtatggtaaagcagatcatatttaaaatcaacatttaaaaacatctatatcttaatattatttttaaaacataaaacatcaggctacttgccttttggcaatttctgtgcatttcccagggctagtgtaatacttccacagagaagtagacac

SEQ ID NO: 328 - DNA, 215 bp, synthetic construct
  source 1..215: mol_type = other DNA; note = Description of Artificial Sequence: Synthetic polynucleotide
atatgtttcattttctgacatacattgtttctaatgtgctttgtatcttgagttcaaatgagtgttccccccgccatattgctgtatgaatttagatgattgcctaaataaatatataaatatgtgggggggggtgttgtttattattgtttatattttctattttagatgcagcagttcttaggaagacctcttgtgaaagaatctttctttag

SEQ ID NO: 329 - DNA, 198 bp, synthetic construct
  source 1..198: mol_type = other DNA; note = Description of Artificial Sequence: Synthetic polynucleotide
atttatttattttgtaggttttcagactgtttcctttaacagccagttaggtatctagataattaacttaccagggctcatgccacatccatcatccaggaaacacaacatgaatcccccctgcagtttttcattatccactgtaagagaaagcagttattactaataaatatcgggcttaatttgttagtcagaaag

SEQ ID NO: 330 - DNA, 193 bp, synthetic construct
  source 1..193: mol_type = other DNA; note = Description of Artificial Sequence: Synthetic polynucleotide
ggccacaagaccaaagatacttacatcccgattgccagttaagagcagaagcactacatcttcctgcagggggggcacgagccccacaaaagaaagtaaggagcatttctttgacctttacagaaattccatagaattctttgaggagattagtgatgaggcttccaaattagtggataggctgacacagtca

SEQ ID NO: 331 - DNA, 182 bp, synthetic construct
  source 1..182: mol_type = other DNA; note = Description of Artificial Sequence: Synthetic polynucleotide
atttgtttcttacagtgagcgggatgcctactgggtggagaaccttatggtccaggagctggagaacttcaatccccccttcaagttgtgtcttcataagcgggacttcattcctggcaagtggatcattgacaatatcattgactccattgaaaagagccacaaaactgtctttgtgcttt

SEQ ID NO: 332 - DNA, 229 bp, synthetic construct
  source 1..229: mol_type = other DNA; note = Description of Artificial Sequence: Synthetic polynucleotide
cactgtttggccgcgtgacccccccccaggaggagagcacggcacacgcgcaacttacttcctccacagggccgggggctgctcgggttccttctccgcagagcccagggatgcgacaagtgcgaggggccaggccagcagcatcatggcagcggcaaccaggagccggaccgggaagagcgtcagtgtcatgagggccacctgcagacagaggggggcattatccaga

SEQ ID NO: 333 - DNA, 190 bp, synthetic construct
  source 1..190: mol_type = other DNA; note = Description of Artificial Sequence: Synthetic polynucleotide
ctcttctgtgcagagcctctgcattgagaacagctgcatcgcggcccacgacaagagggggaggtacgggaccctgttcacgatggaccgggtgctgacccccccaatggggactgtcatggatgtcctgaagggagacaatcgctttaggtaattagttccatccccgggtggagcttctgcccagtgg

SEQ ID NO: 334 - DNA, 203 bp, synthetic construct
  source 1..203: mol_type = other DNA; note = Description of Artificial Sequence: Synthetic polynucleotide
ccctccggtccccccccaaccggccccacgccgctgattcgctcgcagcttctcctcaccacatcctaaccatggctgtgtttctgcagctgctaccgctgctgctctcgagggcccaagggaaccctgggggtaagcgatccctgggagagttgtgatagacgcagaggggctgaagcaagataagggccgcctagtagggt

SEQ ID NO: 335 - DNA, 214 bp, synthetic construct
  source 1..214: mol_type = other DNA; note = Description of Artificial Sequence: Synthetic polynucleotide
ggagaaaaagcgggaattagaaaagcggttacaagatgtcagcggacagctcaattctactaaaaagccccccaagaaaggtgagtatatactttcatgccactacagattgactccatcctgccttcttgactgtcttttattgacaaatgaagattcagacttgaacgtctttaactttcgaatttgttctgcagcgaatgagaaaacagag

SEQ ID NO: 336 - DNA, 213 bp, synthetic construct
  source 1..213: mol_type = other DNA; note = Description of Artificial Sequence: Synthetic polynucleotide
caattgaagtttatttgtcaaacccttgttaaactcggtctttttccccccccaggagagtggaattcgtgacctgataccaggttctgtcattgatgccacaatgttcaatccttgtgggtattcgatgaatggaatgaaatcggatgtgagtagttatatattgcttcaataattatttaaatgtgaatagtctttccattcttaacactg

SEQ ID NO: 337 - DNA, 206 bp, synthetic construct
  source 1..206: mol_type = other DNA; note = Description of Artificial Sequence: Synthetic polynucleotide
aaaaataactgattacctgggcttttctggtacatcagaaggattactgcaaaatggttcctggtaaatagtctcagacaggtcatgatccaacaaagttgccataatttcttccctacttgggggggacataagtggtttcagaatgttagcagttcgaggggtgaagctgccatttttagattgtgaaggagagctagtagatc

SEQ ID NO: 338 - DNA, 179 bp, synthetic construct
  source 1..179: mol_type = other DNA; note = Description of Artificial Sequence: Synthetic polynucleotide
gtgactgcttgaacccttttggcccacattcaccttgggagcatcctaggtgtttatttatctggcctgaaaggcagcaactgtgaactcagtgtgtaatctttaatttctagctggtgtgatcccccccaattcagtgcttcattttgatgtacttctgatggatatttggaattctg

SEQ ID NO: 339 - DNA, 182 bp, synthetic construct
  source 1..182: mol_type = other DNA; note = Description of Artificial Sequence: Synthetic polynucleotide
aacacagtggtggccaatgcctccttcacctacctcaccgccctgacggactccaacatcgtagccatctgggggaggatgtagcctcacaccatcgcaaaggaccagggacacagctaactaacttattcagctttgggccgatgggggtgggggggggtctttcagtgaatatttttatt

SEQ ID NO: 340 - DNA, 204 bp, synthetic construct
  source 1..204: mol_type = other DNA; note = Description of Artificial Sequence: Synthetic polynucleotide
gaatctctcccccccccctctttttttcccttcagatccagacatttaatgtggatgcaccatgccagaccgtggaagatttaacgaatggggttgtgatggcccaggttcttcaaaagatgtaagaataaattgcttatctttatgtgtattcttatgcatggatatttgttaggttgaaaacatggattaaaaaagtactta

SEQ ID NO: 341 - DNA, 229 bp, synthetic construct
  source 1..229: mol_type = other DNA; note = Description of Artificial Sequence: Synthetic polynucleotide
gtagtcatttgctttctcccccccccagaagctcagaagaagcaattggaagagagtctagtgctgatccaagaggaatgtgtgtcagagattgcagatcactctacagaggagcctgctgaaggagggccagatgccgatggagaagagatgacagatgggatagaggaggacttcgatgaagatgggggtcatgagctggtaggaaagaaacatttggtattcacac

SEQ ID NO: 342 - DNA, 218 bp, synthetic construct
  source 1..218: mol_type = other DNA; note = Description of Artificial Sequence: Synthetic polynucleotide
aagcttccgccctcctgacctcctctcctccaacctgatccccactccaacccctgcaggctgctcctgggggcaggaccctccttcctcagcacaggggcccctggatggcctgcagggaggctcccctgacagatgaggaggctgagtaccggtgggggggggtcagggggtccctgctggggttgagctgacctggagttttgggacacggctga

SEQ ID NO: 343 - DNA, 213 bp, synthetic construct
  source 1..213: mol_type = other DNA; note = Description of Artificial Sequence: Synthetic polynucleotide
gtctggggtgggaggaagtttctcatttcctggctacttccccactatgtggaccatttcatgaaactccattgcatagttctcagtggggggggcagaaggggtgcccaagcctgcccctctctgcctcccaaaaatatggctttggaaaggagttcccaaaaatgaagaggaccctctgaatcagcaggcctacccctcacagggatgaga

SEQ ID NO: 344 - DNA, 175 bp, synthetic construct
  source 1..175: mol_type = other DNA; note = Description of Artificial Sequence: Synthetic polynucleotide
cgagtcttctgattctcaaactccaaactgtgtataaaggtggggggagaatcactcataatctcatcagccagagataaccaatgtacaaaatccaactatgggaagaaagggaaactagacccagggctcagggacccagagttactgattttcagtcacaacttgcttcatc

SEQ ID NO: 345 - DNA, 221 bp, synthetic construct
  source 1..221: mol_type = other DNA; note = Description of Artificial Sequence: Synthetic polynucleotide
cagaaaaactggtatactgtttgtgttatttaggcatatttttgatccaatatttacactatctaaatattgggggggggttgttttctaacactctagtggcatatgttgtttgcaattgtcttattgaaagtgatacatcattttaggaagctaaggttttattttgttgtttttttttaggaagctgttgataactcttcctgagctggtggaatgta

SEQ ID NO: 346 - DNA, 220 bp, synthetic construct
  source 1..220: mol_type = other DNA; note = Description of Artificial Sequence: Synthetic polynucleotide
ccagccgggaatttcgtgcgggtgctgaaggagctgcgggagccggagaagaatgaaactgcgtggagtcagcctggctgccggcttgttcttactggccctgagtctttgggggcagcctgcagaggctgcggtaagtccttcctcccctcccccgcgcccatcaccgctctttcacgctgaaattaccttttttttggggggggggtccctttatctt

SEQ ID NO: 347 - DNA, 182 bp, synthetic construct
  source 1..182: mol_type = other DNA; note = Description of Artificial Sequence: Synthetic polynucleotide
cgcaaaactgccaaagacatttgcaagatccgctgtctgtgcgaagaaaaggaaaacgtactgaatatcaactgtgagaacaaaggatttacaacagttagcctgctccagcccccccagtatcgaatctatcagctttttctcaatggaaacctcttgacaagactgtatccaaacgaatt

SEQ ID NO: 348 - DNA, 200 bp, synthetic construct
  source 1..200: mol_type = other DNA; note = Description of Artificial Sequence: Synthetic polynucleotide
agttgaaatgaaatgtttttgttttctttatgttttaaactgttacagggaccatatgatgtggtggttctaccaggaggtaatctgggcgcacagaatttatctgaggtaaaaaattctactcaattatacctcaataaagctggggggggggaaaaactaaagaatttcagcatctgcttatgttctgttaattttgt

SEQ ID NO: 349 - DNA, 199 bp, synthetic construct
  source 1..199: mol_type = other DNA; note = Description of Artificial Sequence: Synthetic polynucleotide
tcccgtctccgcctctgccccccccccggccccgcccctgccctgcgccggcgctggctccgtctgcgcctcagcctcggtcccgcctccagccctggccccgccccgccgctctccccttctccgcccctgccctggctccgcccccgcctgcccgcgcaccgcagaaggcctcctggaacttcctggacagcttctc

SEQ ID NO: 350 - DNA, 177 bp, synthetic construct
  source 1..177: mol_type = other DNA; note = Description of Artificial Sequence: Synthetic polynucleotide
atgcttcaactttggacttagagattttatcttaaaatgtttcttttccccccccccattaatcaggcaaaacacattcttggtcctaagagatttcgatgggcttgttcaagttatcattccccaggatgaggtaatagaaaatttcctgttattatctaaaagcttctttgatgc

SEQ ID NO: 351 - DNA, 205 bp, synthetic construct
  source 1..205: mol_type = other DNA; note = Description of Artificial Sequence: Synthetic polynucleotide
tgctggggctggcagcaaagctgggtaacacctcccctccctaggaccattgccccccccccacctgctctccccaccttccttatcccagacctccttcctagctcttgctcagagttgaggccttggtcgggtatgtgtgcgtgcgcggggggcggagggttacctcagctcctggggtggagggaggctctctgccaggcca

SEQ ID NO: 352 - DNA, 211 bp, synthetic construct
  source 1..211: mol_type = other DNA; note = Description of Artificial Sequence: Synthetic polynucleotide
caagacacacagaaacttgtgactgtgggagagaacgcacatggagtgtgccccagtgatgtggaactcccgtcccccccccggagaaggggtggtggcctcagagccagagggcggcccaccgggaggccacagagggcacggcgagctcaactcacagcatcagctcagcacgccactccttgtcttcctcctcagtctgactcagcac

SEQ ID NO: 353 - DNA, 202 bp, synthetic construct
  source 1..202: mol_type = other DNA; note = Description of Artificial Sequence: Synthetic polynucleotide
gggcagcggtgagccggccaccctggggaggggcgagggccgggccacacagtgtgacctccccacacggccatgtctgacctgggccagggctgggggggggttgggtgggcaggcagccaggagagcggggcccagggagagaccccgctgtctgcgcagggaggagctgccttacagccgcactggcctcctggtggag

SEQ ID NO: 354 - DNA, 195 bp, synthetic construct
  source 1..195: mol_type = other DNA; note = Description of Artificial Sequence: Synthetic polynucleotide
gtgtggccgcggcgcagcgcgcgccaacgagcccgggccggcgggagcagaggaagggcggggcgcccggcgggaacggggagggaccccccccccgcggcacaggtgcgagcgcggcggccccactcactcactcgatgaagtagctggcgccctcctccgtgaagccctcctcccagccgcggggcaggtctg

SEQ ID NO: 355 - DNA, 208 bp, synthetic construct
  source 1..208: mol_type = other DNA; note = Description of Artificial Sequence: Synthetic polynucleotide
taccagctgctacagtttagagagtgctctctcaggcgggctggtcgcgggagctggtctgtctctctgcagcttcctgcgaggtatacggggggggggtgggggacgtgtcctgcacatggtcacaaggcctgacccttttctctgggcctccaggagaggctgaggagggggctggagtccggcgctcactagggctgctaagggc

SEQ ID NO: 356 - DNA, 185 bp, synthetic construct
  source 1..185: mol_type = other DNA; note = Description of Artificial Sequence: Synthetic polynucleotide
ggggtggggggggggaaggagagatcatttagggagtgccgggaggaggggcgggctgggagcccggacgcctgggtcccggctggaggtggggagttgaatgggaggggttcagggttgaggtcagtacggggagaagaggatgggtccgtgcgcagttcggatgggtccgggggccgggtgaa

SEQ ID NO: 357 - DNA, 207 bp, synthetic construct
  source 1..207: mol_type = other DNA; note = Description of Artificial Sequence: Synthetic polynucleotide
gtcctccactgggccacctccgagttggccttggacaggacgcgctgcagctcggccttggcctctgtctcctcctcgtactgctcccgcagcaggtcgcagtcatgccgggccgactgcagtgcatgggccagggcgttcttcgcctggggaggggggggggcaccaggaggtgggagggactccctgtgccccattctctagatt

SEQ ID NO: 358 - DNA, 191 bp, synthetic construct
  source 1..191: mol_type = other DNA; note = Description of Artificial Sequence: Synthetic polynucleotide
ctgcagttgattatgttcttacttgtccctaagatgagcacttacctaggcatagctgatgccctctggtggggccggggggggggtggtgtcccaaatctgatggtggagcagcaggggatcccctgtgggtagagaccaacaggcaggctctgtgccgtgggctggctggtgtacccaggagacctcag

SEQ ID NO: 359 - DNA, 215 bp, synthetic construct
  source 1..215: mol_type = other DNA; note = Description of Artificial Sequence: Synthetic polynucleotide
ggaggggctagagctggctggggctgggtacctcagccacatgccaacggctgccttctcctgctctccacagcctcagtaacaacgggctttctgtggccggggtgcattgtgtgctgagggccgtgagtgcgtgctggaccctggcagagctgcacatcaggtgggagctccctcagaccacggtacccatccccccccccatcatgctctct

SEQ ID NO: 360 - DNA, 195 bp, synthetic construct
  source 1..195: mol_type = other DNA; note = Description of Artificial Sequence: Synthetic polynucleotide
gcggatcgcggggcgcaggagcccgatcgcgctggccatggcctccaactttaacgacatagtcaagcagggctacgtgaaaatccgcagcaggaagcttggggtgagtggctcgctcggcttgctccttccccggcgctcgttcggcccggctggctgcctggggggggggcagggagaggtgacccgtgcggg

SEQ ID NO: 361 - DNA, 185 bp, synthetic construct
  source 1..185: mol_type = other DNA; note = Description of Artificial Sequence: Synthetic polynucleotide
gaccaccgggctgagcgaggcgcgggagaaggccgtcttgctggacgaggacgatgacttgtgggtggagcttcgccacatgcatatcgcagatgtgtccaagtgcgtgcacacggggaccggatcccccccccaccgcccactgtgggcctggtagcggccttgggatccctggctgctgccaa

SEQ ID NO: 362 - DNA, 220 bp, synthetic construct
  source 1..220: mol_type = other DNA; note = Description of Artificial Sequence: Synthetic polynucleotide
atggatggcgtggtgcatgctatcgcaacccctgttctacagataaggaaactgatgcccagagagagacggaggactcttgtgaatattgggggggaccgcccctgcacctcggggacgtgaccccccccccaggctgacccgttcctcgctctgtcgtccccaggtggaggaggagctgacgcacctccagaagaaactaaaagggacagaggacgag

SEQ ID NO: 363 - DNA, 226 bp, synthetic construct
  source 1..226: mol_type = other DNA; note = Description of Artificial Sequence: Synthetic polynucleotide
aggcctcaggccgaccccagccagccagccggggcttcctgccctgggctgagagtgtgttgttggaggcggggtctcagccagtggcactgcccccttaactcacctcacgccgacagctgcccctatggcaccatgtacctctcaccacccgcggacaccagctggagaaggtcagtggctggacaccccccccccgcccccttcttgttggaaacaaaaccaa

SEQ ID NO: 364 - DNA, 230 bp, synthetic construct
  source 1..230: mol_type = other DNA; note = Description of Artificial Sequence: Synthetic polynucleotide
ccccccccccacttcctgatggcaggtgctgcaggagaaattctcagagtttgccagcgagacaggtatggcagggcgggaacggctggcagctgtgaaccagatggtggatgagctgatcgagtgtggccatacagcagcggccaccatggccgagtggaaggacggactgaacgaggcctgggctgagctgctggagctcatgggcacacgggcccagctgctggccg

SEQ ID NO: 365 - DNA, 216 bp, synthetic construct
  source 1..216: mol_type = other DNA; note = Description of Artificial Sequence: Synthetic polynucleotide
ctggaggcacaccaccatggaagagatctaggtgggggacctaagtagtcagcctgtttgatcttgaggacacgggaaccccccccccaccccaagagatctgtgaagtgtctagggcagagcctgggtggtgggtggggcatggagaccctaccggtccttgtgtagtttcagaagcctcttcacatcttctctgcccttggggaccgggccggc

SEQ ID NO: 366 - DNA, 209 bp, synthetic construct
  source 1..209: mol_type = other DNA; note = Description of Artificial Sequence: Synthetic polynucleotide
aagctgtcccacccccccccctttccagtttgctctgaaaacgagtccgaagccgaagctgaccagcagatggacaacttgtacctgaaagccttggagggtttcattgccgtggtgacccaagatggcgacatgatctttctgtcagaaaacatcagcaagttcatgggacttacacaggtgacaccctcctctatctctttcaaaag

SEQ ID NO: 367 - DNA, 232 bp, synthetic construct
  source 1..232: mol_type = other DNA; note = Description of Artificial Sequence: Synthetic polynucleotide
agcacgcccccttccccacagaggaccccccccccgcctcaccgggagacactgggcgtcctcccgcccccgcccctctgagaccagcgctcctaggaactctccgccggagggcggggtcctaagcctacctgagctgggcgaggtccgcgcggacccggcagacaccgccattgctctgcggaggaggcaggtgggtcacagctgccgcgtctgaacggaggccgccagg

SEQ ID NO: 368 - DNA, 224 bp, synthetic construct
  source 1..224: mol_type = other DNA; note = Description of Artificial Sequence: Synthetic polynucleotide
taatcagacaaaaggtagaggttgtctaagattaaaaatgtctaatacctaagttttattatctcctccccccccccttttttgttttgatcttaggacttcaaggacagcaaggaagaaaaggagaatcaggagttccaggtaaagggcaggctgcatcttcatccctcggagtgatgacggggaaggcctgccttctcagagggcaggtcttgttgacattg

SEQ ID NO: 369 - DNA, 226 bp, synthetic construct
  source 1..226: mol_type = other DNA; note = Description of Artificial Sequence: Synthetic polynucleotide
gaggagagaacaaggggggggggagaaaaacagagaagagcagagtgaagaggttaggagttccccaggagagagtgacagagtagagagggtgaggagaaaggtggaggaaagggggagccacgggatggggagggggcctgggtcagcatcctttgctgcccaggaggggcctgggtctcatgctcagggtctactccaggccgtgctctgagctgagggacgg

SEQ ID NO: 370 - DNA, 222 bp, synthetic construct
  source 1..222: mol_type = other DNA; note = Description of Artificial Sequence: Synthetic polynucleotide
ctaagttttctcttcatgattttttgttggttgtatactgagacctctgagggatttccttcctttctgcagagggcagtgatgactgttcggaaatgtgtgatggagaagagcgccaggaagcctctagctccctagcaagacgtgggtcaggtaaggacggggggggggatttctggccctgggctgcagagtgtcaggaggttgaatttggagctcgtg

SEQ ID NO: 371 - DNA, 225 bp, synthetic construct
  source 1..225: mol_type = other DNA; note = Description of Artificial Sequence: Synthetic polynucleotide
ctgctttaggttaaattattctgaagacagttcacatccaagtcagcctggtcacctttgccaccctcaagtttgggcgacatggagttctggatgttaaatgactcctcctctttcaggcaggacttgagaacctcctggattttgtggggagcactaggatcatcctggaagagggaaaaagttggggggggggagacaatgcatacagcaaccaaataataa

SEQ ID NO: 372 - DNA, 222 bp, synthetic construct
  source 1..222: mol_type = other DNA; note = Description of Artificial Sequence: Synthetic polynucleotide
cctcccactatgtggaaaagaatagctcacctctcgctctcagagtcgctggagatctcctcattcatcttgccgccacccttggatttgcccctgtcccccgcagagtcggcctgggaattatacggtcttttctttggtcaggggtccagaccaccctggcattgaccaagggcccccccccctcggttccctcagaaccccaggaatggggaaggcggt

SEQ ID NO: 373 - DNA, 200 bp, synthetic construct
  source 1..200: mol_type = other DNA; note = Description of Artificial Sequence: Synthetic polynucleotide
ggtgctgcccaacctgacagtccaggagatggactggctggtgctgggggagctgcagatggccctggagtgggcaggcaggccagggctgcgcatcagtggacacattgctgccaggaagagctgcgacggtgaggcggggggggggcctggtgcgccgggcatgcacaactgagagacacagacaagtgccagggtgg

SEQ ID NO: 374 - DNA, 206 bp, synthetic construct
  source 1..206: mol_type = other DNA; note = Description of Artificial Sequence: Synthetic polynucleotide
gcacagcaggtcagcaacaagtttattttgcagctagcaaggtaacagggtagggcatggttacatgttcaggtcaacttcctttgtcgtggttgattggtttgtctttatggggggggggtaggggaaagcgaagcagaagtaacatggagtgggtgcagcctccctgtagaacctggttacgagagcttggggcagttcacctg

SEQ ID NO: 375 - DNA, 218 bp, synthetic construct
  source 1..218: mol_type = other DNA; note = Description of Artificial Sequence: Synthetic polynucleotide
ggcgtcattcagctccatgaagatcatcttggcgtgctgctcgtacggctgccccgcagcctatgggtgacaggcggtgtaagcgcctcttataagcatgcgggggcgggcggggcaggaccccccccccgccccgcccctgaaggcctcaagcggccgcctggcagctctgctcaccttgtcggggtgcacagccagcaccgcgcggcgatagtgct

SEQ ID NO: 376 - DNA, 169 bp, synthetic construct
  source 1..169: mol_type = other DNA; note = Description of Artificial Sequence: Synthetic polynucleotide
catgatagtaatcatagcctgatgttcccagaacttcaaatggcaaataccctattatgggtggtgccctaaattacacagaaaacaatcattattataagtttttccataataagaaatatttcaactagaaataaactttggggggggggctttatttttcaaaaaa

SEQ ID NO: 377 - DNA, 178 bp, synthetic construct
  source 1..178: mol_type = other DNA; note = Description of Artificial Sequence: Synthetic polynucleotide
cagtggcatgaatcccatccagactatgaccgaagtccgagccagacaggtgaaggagatgaaccctgctcttggcatcgactgtttgcacaaggggacaaatggtgaggagctgtcacgcctctgcgtggagggggggggatgtctgatttaaactgaataatcatccactgtatgt

SEQ ID NO: 378 - DNA, 214 bp, synthetic construct
  source 1..214: mol_type = other DNA; note = Description of Artificial Sequence: Synthetic polynucleotide
ttgcatacccacccttgcacccactccacccctgccacacagcccctccacacccccccccacaacccttctcacctgagatgctaagcccacggcggaacacctggtacatggtccgggcatcatcatagtagtgggtaagtagctgagggccagacccaatcacagatcgccgtgccccgccactgtcctacaagccaagcaccggccagag

SEQ ID NO: 379 - DNA, 176 bp, synthetic construct
  source 1..176: mol_type = other DNA; note = Description of Artificial Sequence: Synthetic polynucleotide
tcagctatcattttaggcaaatgtgacccgacaagtaatcaggtgagatttatttctctgcctcctaattgttggtgtcttggcacaagtcttttaaggagatgttttttgagcctgttatgaaaactgtttggggggggggtggggcggcatatatttagttcatatatatttag

SEQ ID NO: 380 - DNA, 200 bp, synthetic construct
  source 1..200: mol_type = other DNA; note = Description of Artificial Sequence: Synthetic polynucleotide
gaagacagactcttgcggagccgctgcccgcagctcagtgggccgttcctggtcagggaaggcggctgctcccgcttcgccggcaaggctgtggaggagcaggggctgaggacggagagcccgcggccggctggccggaatctagctcgcccggcgccctctcggctcctccttcccccccccacttcctaacctcccgg

SEQ ID NO: 381 - DNA, 213 bp, synthetic construct
  source 1..213: mol_type = other DNA; note = Description of Artificial Sequence: Synthetic polynucleotide
acctccttctcctcatagaagtaaagctcccggcccccaccagaggcgaagcggaaaggaatgaactcctgggcgtggaagccatagagtggctgtggcaggagagaggggcatgggtaagaagggagccggaggagacctggctgaaggatggcgtctcaccccccccccgcctgtctccccaaatcacctcgacattctttagcttcaagg

SEQ ID NO: 382 - DNA, 220 bp, synthetic construct
  source 1..220: mol_type = other DNA; note = Description of Artificial Sequence: Synthetic polynucleotide
tctgactcctttttcttaactctagcggatcttggaaaagtcaaagcgagtaggtgaaggaagacaagcaactcacagagaagactaggggggggggccattgggtctgcacacctgcaagtaagtgggaagaagcaagcaggaagcgccagaataacctcccgcccccagctcacactcgcacctgggaatgaacttggcttcgtccccaagtcgcgcc

SEQ ID NO: 383 - DNA, 196 bp, synthetic construct
  source 1..196: mol_type = other DNA; note = Description of Artificial Sequence: Synthetic polynucleotide
ggcagccagagcggcgagctgagtaacaggggtaacacctccctgctgcccctccctctctctccctctctctccctctctctttaccctccccagggctccatctccgcctcaggggcttctccaccccaagtctggctccattcctggccttctgttggtgacagaccccccccctaaggtgctcgtttggggg

SEQ ID NO: 384 - DNA, 229 bp, synthetic construct
  source 1..229: mol_type = other DNA; note = Description of Artificial Sequence: Synthetic polynucleotide
agaagcccttctccattgtctcagtcacttcagggtgcaggaagcggctggccaaccgttcgatgtcgtccagcgtcaggcgactcagggacctctcgtaatcctggggagagaagggcaggaagattcaggggctgccagtggggggggggcatacactgcaacagaggtaggggctgggagcaactgggcctgaagaggatgcccagggttacccttctcaccctct